(19) United States
(12) Patent Application Publication
(10) Pub. No.: US 2018/0273906 A1
(43) Pub. Date: Sep. 27, 2018

(54) MICROVESICLE AND STEM CELL COMPOSITIONS FOR THERAPEUTIC APPLICATIONS

(71) Applicant: Muhammad Ashraf, Cincinnati, OH (US)

(72) Inventor: Muhammad Ashraf, Cincinnati, OH (US)

(21) Appl. No.: 15/881,693

(22) Filed: Jan. 26, 2018

Related U.S. Application Data
(63) Continuation-in-part of application No. 14/255,789, filed on Apr. 17, 2014.
Application No. 14/255,789 is a continuation-in-part of application No. 14/951,354, filed on Nov. 24, 2015.
Application No. 14/951,354 is a continuation-in-part of application No. 15/201,292, filed on Jul. 1, 2016.

(51) Int. Cl. C12N 5/074; C12N 5/077; C12N 5/071; A61K 35/12

(52) U.S. Cl. CPC C12N 5/0696; C12N 5/0657; C12N 2506/45; C12N 5/0661; A61K 35/12; C12N 5/069

(57) ABSTRACT

Provided herein are stem cell and exosome compositions having therapeutic utility to treat a variety of diseases and disorders, e.g., cardiovascular disease, Duchenne muscular dystrophy, and fibrotic disease.

### CROSS-REFERENCE TO RELATED APPLICATIONS

This application is a continuation-in-part under 35 U.S.C. § 120 of U.S. application Ser. No. 14/255,789, filed Apr. 17, 2014; U.S. application Ser. No. 14/951,354, filed Nov. 24, 2015; and U.S. Ser. No. 15/201,292, filed Jul. 1, 2016, the content of each of which is incorporated herein by reference in its entirety.

### STATEMENT OF GOVERNMENT SUPPORT

This invention was supported in part by National Institutes of Health grants, RO1 HL126516, HL134354 and RO1 AR070029. Thus, the US government has rights in this invention.

### REFERENCE TO SEQUENCE LISTING SUBMITTED VIA EFS-WEB

This application is being filed electronically via EFS-Web and includes an electronically submitted sequence listing in .txt format. The .txt file contains a sequence listing entitled “2018-06-12 ISPH_004_ST25.txt” created on Jun. 12, 2018 and is 3,537 bytes in size. The sequence listing contained in this .txt file is part of the specification and is hereby incorporated by reference herein in its entirety. The content of the sequence listing information recorded in computer readable form is identical to the written sequence listing and includes no new matter

### BACKGROUND

Throughout this disclosure, various technical and patent publications are referenced to more fully describe the state of the art to which this invention pertains, the full bibliographic citations for some of the publications are found at the end of the specification, immediately preceding the claims. All publications noted in the present specification are incorporated by reference, in their entirety, into this application.

Induced pluripotent stem (iPS) cells are important source for progenitors, such as cardiac progenitors, for drug discovery and the treatment of disease, e.g., infarcted myocardium. Due to inherent properties of iPS cells to form teratomas, it becomes very important to generate iPS cells without producing tumors for use in clinics. Given the importance of these concerns, efforts have been made to improve the reprogramming efficiency and several methods have been devised for the non-viral generation of iPS cells. Various growth factors and chemical compounds, such as DNA methyltransferase inhibitor (5′-azacytidine and RG108), histone deacetylase inhibitors (e.g., valproic acid), histone methyltransferase inhibitor (BIX-01294), Wnt3A, and ALK5 inhibitor, have been found to improve the induction efficiency. (1-6) With different experimental manipulations, iPS cells can be induced to cardiac lineage prior to their transplantation; however, these procedures are labor intense, expensive and involve multiple growth factors and/or small molecules that limit the consistency and reproducibility required for drug development and to enter the clinic. Therefore there is a need in the art for improvements to known methods and technology. This disclosure satisfies this need and provides related advantages as well.

### SUMMARY OF THE DISCLOSURE

This disclosure provides in part methods that use just one class of small molecules applied to induced pluripotent stem cells (IPSCs) to express and precondition the cells propogated from the method (e.g., cardiac progenitor cells, endothelial cells, smooth muscle cells, myocytes, myogenic progenitors, cardiomyocytes and blood vessels and the exosomes or microvesicles produced by the resultant cells). The cells and exosome or microvesicle compositions isolated from the cells activate resident stem cells, caridomyocytes, myocytes and paracrine factors. In addition, given the existing non efficient process to produce these cells and exosomes or microvesicles, this one step commerciable, scaleable and safe process may be the most economical in producing an unlimited number of cells and exosomes or microvesicles for the treatment of diseases such as CVS, Duchenne muscular dystrophy, muscle and other diseases.

Thus, in one aspect, provided herein is an isolated population or a composition comprising, or alternatively consisting essentially of, or yet further consisting of an isolated population of exosome or microvesicle, that in one aspect are isolated from a cell selected from the group of: an iPS cell, an embryonic stem cell, or a stem cell, each that had been previously contacted with an effective amount of an isoxazole compound, a derivative or an equivalent of the isoxazole compound, wherein the exosome or the microvesicle overexpresses a microRNA (miRNA or mir) selected from the group of mir-373, mir-210, mir-377, mir-367, mir-520, mir-548ah, mir-335, mir-30c, mir-214, or mir-548q, and/or one or more of a protein selected from Tsg101, CD9, Hsp70, Flotillin-1, or GAPDH. In a further aspect the exosome or the microvesicle can further overexpress one or both of mir-30c and/or mir-21. In one aspect, at least 70%, or 80%, or 85%, or 90% or 95%, or 98% of the cells express the stated mir and/or protein.

Also provided herein is a cell or an isolated population of one or more of: a cardiac progenitor cell (CPC), a cardiomyocyte, a myocyte, an endothelial cell, a smooth muscle cell, a skeletal muscle cell, each generated from a cell selected from an iPSC, an embryonic stem cell, a a stem cell, each that had previously contacted with contacted with an isoxazole compound, derivative, or an equivalent of each thereof, wherein the cells of the population overexpress one or more of mir-373, mir-210 mir-377, mir-367, mir-520, mir-548ah, mir-335, mir-30c, mir-214, or mir-548q; and/or a muscle gene selected from the group of: paZ3, pAX7, MYF5, MYOD, MYOG, or dystrophin. Also provided is a cardiomyocyte that expresses one or more of cTnT, cTnI, c MLC2V, and/or VE-cadherin. In a further aspect, provided herein is a cell or a population of cells of α-smooth muscle actin (SMA) and calponin. In one aspect, at least 70%, or 80%, or 85%, or 90% or 95%, or 98% of the cells overexpress the stated mir and/or muscle gene and/or protein. The exosomes or microvesicles can be isolated for any one of the following: a cardiac progenitor cell, a cardiomyocyte, a skeletal muscle, an endothelial cell, a myocyte, or a smooth muscle cell.

In one aspect, the population or cells further comprising, or alternatively consisting essentially of, or yet further consisting of, a pharmaceutically acceptable carrier that in one aspect, is a non-naturally occurring carrier. In another aspect, the composition also contains a preservative and/or a cryopreservative that in one aspect facilitates lyophilization and/or preservation of the composition. In a yet further aspect, the composition further comprising, or consisting essentially of, or yet further aspect, consists of, a protein that facilitates regeneration and/or improved function of a tissue or a nucleic acid that encodes the protein. Non-limiting examples of such include TGF-β, WNT protein, a cytokine, or a histone deacetylase.

Yet further provided is a composition for the repair or regeneration of damaged or diseased cardiac tissue, the composition comprising synthetic microRNA-146a. In one aspect, the composition further comprises, or alternatively consisting essentially of, or yet further consisting of, a pharmaceutically acceptable carrier that in one aspect, is a non-naturally occurring carrier. In another aspect, the composition also contains a preservative and/or a cryopreservative that in one aspect facilitates lyophilization and/or preservation of the composition.

Yet further provided is a composition comprising one or more of a synthetic microRNA-373, a micro-ma 373 mimic or micro-ma 373 exosome. In one aspect, the composition further comprises, or alternatively consisting essentially of, or yet further consisting of, a pharmaceutically acceptable carrier that in one aspect, is a non-naturally occurring carrier. In another aspect, the composition also contains a preservative and/or a cryopreservative that in one aspect facilitates lyophilization and/or preservation of the composition. These compositions are useful to treat fibrotic diseases by administering an effective amount to a subject in need thereof. A non-limiting example of the fibrotic disease is myocardial fibrosis.

The exosome or microvesicle population or the cells is isolated from a population of stem cells or progenitor cells cultured in the presence of an effective amount of an isoxazole compound or a derivative thereof. In one aspect, the isoxazole compound is selected from isoxazole-1 (isx-1), isoxazole-9 (isx-9), or Danazol.

Also provided herein are methods for one or more of providing in a subject in need thereof: regenerating damaged tissue; improving the viability of damaged tissue; facilitating the formation of new tissue, optionally a cardiac tissue, a muscle tissue, a skeletal muscle, a blood vessel, a capillary, or a myocyte; promoting cardiac regeneration; promoting cardiac regeneration in a subject suffering from an acute cardiac event; promoting cardiac regeneration in a subject suffering from a myocardial infarction; promoting cardiac regeneration in subject suffering from Duchenne muscular dystrophy or Duchenne muscular dystrophy-associated cardiomyopathy; or promoting cardiac regeneration in a subject suffering from age-related diseases selected from the group of: such as Hoyeraal-Hreidarsson syndrome, dyskeratosis congenita, pulmonary fibrosis, aplastic anemia, liver fibrosis, dyskeratosis congenita, bone marrow failure, lung disease, endocrine diseases, polycystic ovary syndrome (PCOS), Cushing's syndrome, and acromegaly, Cerebrovascular Disease (Strokes), High Blood Pressure—Hypertension, Parkinson's Disease, Vascular Dementia, dementia, macular degeneration, Alzheimer's Disease, Age-related hearing loss, Celiac disease (CD), COPD, bipolar disorder, hydroxyurea, sickle cell diseases, hypertension, atherosclerosis, arthritis, osteoporosis, osteoarthritis, vasculardementia or macular degeneration, cancer, type 2 diabetes, or diseases with telomerase dysfunction dealing with a shortened telomere length); promoting tissue regeneration in tissue damaged from one or more of stroke, arthritis, Alzheimer's, memory loss disorders, cystic fibrosis, inflammatory disorders or cancer; decreasing cardiac wall thickness in a tissue damaged from a cardiac infarction; altering gene expression of one or more of protein kinase C, iL-6, mmp, PDGF; reducing or inhibiting the expression of an inflammatory protein; that is optionally a cytokine, a chemokine, or a macrophage; directly or indirectly stimulating angiogenesis; directly or indirectly inhibiting cellular replication; promoting cardiac regeneration in a subject suffering from a disease selected from the group of: coronary artery disease, myocardialinfaction, heart failure, hypoplasic left heart syndrome, peripheral artery disease (PAD), cardiac hypertrophy, valvular heart disease (aortic stenosis), myocardial hypertrophy mi, hypertrophy fibrosis by administering to a subject in need of the method, an effective amount of a population of exosome or microvesicle and/or isolated cells as described herein, as well compositions containing the same. In a further aspect, the methods further comprise, or consist essentially of, or yet further consist of, administering an effective amount of a non-embryonic stem cell or a progenitor cell to the subject, that is optionally of the same type as the tissue in need of repair of an type different from the type of tissue of repair. The stem cells can be non-embryonic stem or progenitor cell is autologous to the subject. They also can be delivered systemically or locally to the tissue of the subject.

Also provided herein are methods for preparing a population of cells as described herein from a population of human induced pluripotent stem cells (hiPSCs), comprising contacting the hiPSCs with an effective amount of Givinostat (GIV).

Further provided are methods for one or more of: providing anti-oxidative therapy; promoting activation of local or resident cardiomyocytes; promoting the release of angiogenesis and/or paracrine factors; promoting activation of one or more of: wnt, a BMP, and/or cytoskeleton remodeling; promoting TGF-β induced emt signaling and cardiac differentiation; increasing expression of wnt5 and wnt11 or a BMP family protein, optionally BMP4; increasing expression of a cardiac transcription factor selected from the group consisting of nkx2.5, mef2c, gata4 and isl-1; promoting expression of genes for development of pip3 signaling in cardiomyocytes, muscle contraction and nf-at hypertrophy signaling pathways; reducing fibrosis and apoptosis; promoting myoangeneis and muscle differentiation; promoting the release of a cytokine selected from the group consisting of angiopoietin-2, il-6nmp, pgfbb, timp 1 or a gene identified herein or in the Figures; promoting upregulation of a gene selected from the group of wnt3a, wnt5a, wnt11; and/or promoting cytoskeletal remodeling, the method comprising, or alternatively consisting essentially of, or yet further consisting of, administering an effective amount of the exosome or microvesicle population or cells, or composition containing them, to a subject in need thereof.

Further provided are cells or populations of cells, as well as cells and populations prepared by method as disclosed herein, wherein at least 80%, or alternatively 85%, or alternatively 90%, or alternatively 95%, or alternatively 97% or alternatively 99%, or alternatively 100%, of the cells overexpress one or more protein selected from the group of: cTN1, MLC2v, cTNT, VE-cadherin, CD31, a-SMA (actin), calponin, or Cx43. Further provided is a a cell or a population of cells that overexpress skeletal myogenic genes seleted from the group of: Meox1, Meox2, Tcf15, Pax3, Pax7, MyoD1, MYF5, dystrophin, or DESMIN. Determining the expression of the genes, miRNA and/or proteins can be performed using methods known in the art and briefly described herein. The cells and/or microvesicles or exosomes isolated from the cells are useful in method for regenerating skeletal muscle cells, treating DMD.

Also provided is a population of cells that overexpress xESI myogenic genes selected from the group of: Pitx2, IS11, Nkx2.5, Hand1, GATA4, Tbx5, TnnT2, Myl7, MLC2v, Myf2e, Cdh4 or Lhx2. In one aspect, at least 80%, or alternatively 85%, or alternatively 90%, or alternatively 95%, or alternatively 97% or alternatively 99%, or alternatively 100%, of the cells overexpress the xESI myogenic genes. Determining the expression of the genes, miRNA and/or proteins can be performed using methods known in the art and briefly described herein. These populations and cells are useful in methods of regenerating cardiac muscle tissue, or yet further treating cardiac dysfunction associated with Duchenne Muscular Dystrophy (DMD), the methods comprising, or alternatively consisting essentially of, or yet further consisting of, administering to a subject in need thereof an effective amount of the population of cells as described herein and/or an effective amount of the population of exosomes or microvesicles as disclosed herein.

Further provided are populations of exosomes or microvesicles isolated from these cell populations.

Yet further provided are methods of regenerating cardiac muscle comprising, or alternatively consisting essentially of, or yet further consisting of, administering to a subject in need thereof an effective amount of the population of cells as described herein and/or an effective amount of the population of exosome or microvesicle as described herein.

Also provided herein are methods for repairing or regenerating damaged or diseased cardiac tissue in a subject in need thereof.

Further provided are compositions for the repair or regeneration of damaged or diseased cardiac tissue or for the treatment of one or more of Hoyeraal-Hreidarsson syndrome, dyskeratosis congenita, pulmonary fibrosis, aplastic anemia, liver fibrosis, dyskeratosis congenita, bone marrow failure, lung disease, endocrine diseases, polycystic ovary syndrome (PCOS), Cushing's syndrome, and acromegaly, Cerebrovascular Disease (Strokes), High Blood Pressure—Hypertension, Parkinson's Disease, Dementia, Alzheimer's Disease, Age-related hearing loss, Celiac disease (CD), COPD, bipolar disorder, hydroxyurea, sickle cell diseases, hypertension, atherosclerosis, arthritis, osteoporosis, osteoarthritis, vasculardementia or macular degeneration, cancer, type 2 diabetes, or diseases with telomerase dysfunction dealing with a shortened telomere length, the composition comprising a synthetic microRNA-195 inhibitor. Non-limiting examples of such include for example HSTUD0320 (SIGMA MISSION® Synthetic microRNA Inhibitor), Human hsa-miR-195-5p or inhibitory hsa-miR-195-5p miRNA/microRNA Lentivector (sold by ABM Good (abmgood.com)). The compositions when applied in an effective amount to bone marrow stem cells or IPS cells promote or facilitate telemore elongation and rejuvenation of aged stem cells. The methods promote cardiac regeneration of the heart in patients such as elderly patients with cardiac diseases such as heart failure, and heart attack patients.

Also provided are methods comprising, or alternatively consisting essentially of, or yet further consisting of administering one or more microRNA fragments, or derivatives thereof to the subject, wherein after administration of the one or more microRNA fragments, the one or more microRNA fragments alter gene expression in the damaged tissue, improve the viability of said damaged tissue, and facilitate the formation of new tissue in the subject. In one aspect, the microRNA fragments, or derivatives thereof, are synthetically generated. In a further aspect, wherein the microRNA fragments, or derivatives thereof are synthesized with a sequence that mimics one or more endogenous microRNA molecules. In another aspect, wherein the microRNA fragments, or derivatives thereof are modified to enhance their stability. The microRNA fragments can be administered by any appropriate method as determined by the treating physician or professional. Non-limiting examples of such comprise administration of a plurality of synthetic liposomes that comprise said one or more microRNA fragments, or derivatives thereof.

Yet further provided are methods of generating exosomes or microvesicles, the methods comprising, or alternatively consisting essentially of, or yet further consisting of, culturing a population of non-embryonic human regenerative cells in the presence of a hydrolase enzyme to induce the cells to secrete exosomes or microvesicles, thereby generating exosomes or microvesicles. In a further aspect, the methods further comprise, or alternatively consist essentially of, or yet further consist of, isolating the exosomes or microvesicles from the culture media and/or the cells.

In one aspect, the hydrolase comprises a member of the DNAse I superfamily of enzymes, a non-limiting example of such includes a sphingomyelinase. In one aspect, the sphingomyelinase is of a type selected from the group consisting of lysosomal acid sphingomyelinase, secreted zinc-dependent acid sphingomyelinase, neutral sphingomyelinase, and alkaline sphingomyelinase. In a further aspect, the neutral sphingomyelinase comprises one or more of magnesium-dependent neutral sphingomyelinase and magnesium-independent neutral sphingomyelinase. In a further aspect, the neutral sphingomyelinase comprises one or more of neutral sphingomyelinase type I, neutral sphingomyelinase type 2, and neutral sphingomyelinase type 3.

### BRIEF DESCRIPTION OF THE FIGURES

The patent or application file contains at least one drawing executed in color. Copies of this patent or patent application publication with color drawing(s) will be provided by the Office upon request and payment of necessary fee.

FIG. 1 shows characterization of IPS cells for the expression of pluripotency markers. Representative photomicrographs showing IPS clones expressing embryonic stem cells (ESC) specific markers Oct3/4, Sox2, Nanog and endogenous Oct3/4. Nuclei were stained with DAPI.

FIG. 2 shows the results of DNA methyltransferase (DNMT) activity assay. DNA methylation analysis showing significant (95%) inhibition of DNA methyltransferase activity in small molecule, isoxazole or isoxazole like compounds treated IPS cells in comparison to the nontreated IPS cells.

FIGS. 3A-3B show that small molecule, isoxazole or isoxazole like compounds treatment induces cytoprotection in vitro and that small molecule treatment prevents oxidant induced apoptosis and increases the proliferation. In FIG. 3A, apoptosis was determined by TUNEL assay. Fewer TUNEL positive cells/microscopic field were observed in small molecule, isoxazole or isoxazole like compounds pretreated IPS as compared to control non-treated IPS cells. In FIG. 3B, an increase in mitogenic response of small molecule treated IPS was significantly noted in comparison with non-treated IPS cells. All values were expressed as mean±SEM, *p<0.05 vs. control.

FIG. 4 shows cytochrome c translocation to cytoplasm (a sign of cell injury). Immunostaining of cytochrome c in non-treated and small molecule, isoxazole or isoxazole like compounds treated IPS cells after exposure to H₂O₂ (100 μmol), (merged images with DAPI), (original magnifications; 200×). Small molecule, isoxazole or isoxazole like compounds treated IPS cells show a significant decrease in cytochrome c translocation to cytoplasm as compared to the non-treated IPS cells.

FIGS. 5A-5B show small molecule, isoxazole or isoxazole like compounds mediated cardiac differentiation of IPS cells in vitro. FIG. 5A shows immunostaining of cardiac specific gene Nkx-2.5 and a Sarcomeric actin in small molecule treated IPS cells (merged images with DAPI), (original magnifications; 400×). FIG. 5B is RT-PCR analysis of cardiomyocyte specific marker, Nkx-2.5, in small molecule treated IPS cells in comparison with non-treated IPS cells. Small molecule, isoxazole or isoxazole like compounds treated IPS cells shows significant upregulation of Nkx-2.5 as isoxazole or isoxazole like compounds compared to non-treated IPS cells.

FIG. 6 shows Affymetrix array-based gene expression profiling of small molecule, isoxazole like compounds treated IPS vs non-treated IPS cells further confirmed 2-3 folds downregulation of Dnmt1, Dnmt3b and Max gene associated protein which were associated with global DNA hypomethylation and myc dependent cell transformation. In addition, there was 2-3 folds concomitant upregulation of CCL7, CXCR2, CXCR5, integral membrane protein 2A, and EphrinA3.

FIGS. 7A-7E show miR-microarray analysis of small molecule, isoxazole like compounds treated IPS vs non-treated IPS cells. Microarray analysis showing miR expression profile in non-treated IPS cells was quite different from small molecule treated IPS cells. (FIGS. 7A-7D) Critical miRs known for reprogramming and differentiation as observed in non-treated and small molecule treated IPS cells. Microarray analysis showed upregulation of cardiac specific miR-133, miR-762 and down regulation of pluripotency associated miR-290-295 cluster and let-7 family in small molecule treated IPS cells. FIG. 7E shows GPCR signaling in small molecule, isoxazole, or an isoxazole similar compound treated SIPs cells. Western blot analysis shows significant upregulation of Gα (pan) in small molecule treated IPS cells as compared to the non-treated IPS cells which was blocked by the concomitant use of GPCR blocker, pertussis toxin.

FIGS. 8A-8F show transplantation of iPS cell treated with isoxazole or isoxazole like compounds attenuated infarct size expansion and regeneration into fully developed myofibers in vivo. A total 3.6×10⁵ cells were injected per animal in the infarct and peri infarct regions after coronary artery ligation. The animals were sacrificed 7 days and 6 weeks after transplantation. A significant attenuation of infarct size and growth of new myocytes in scar tissue in hearts from transplanted animals (FIG. 8B) as compared to the control group (FIG. 8A) was observed. A significant regeneration of infarcted heart with new fibers resulted in reduced infarct size (FIGS. 8C and 8D). There were very limited number of iPS derived growing cells observed in hearts transplanted with non-treated iPS cells (not shown). There w as significant increases in LV chamber dimensions during systole (LVESD) and diastole (LVEDD) after myocardial infarction and these were significantly reduced by iPS cell treated with isoxazole or isoxazole like compounds (FIG. 8E). Similarly cardiac fraction shortening and ejection fraction were increased by the treatment (FIG. 8F).

FIGS. 9A-9I show the characterization of small juvenile stem cells (SJSCs) from heterogeneous bone marrow-derived stem cells (BMSCs) from young and old mouse described in Experiment No. 2. FIGS. 9A and 9B show that both aged and young SJSCs were positive for CD29, CD44, CD59, and CD90 but negative for CD45 and CD117 B. In FIG. 9C, cell growth curves show that the cell proliferation rate was higher in SJSCs than in BMSCs. Compared with aged BMSCs, aged SJSCs showed higher expression of pluripotency markers such as octamer-binding transcription factor 4 (Oct-4), Nanog, sex-determining region Y box 2 (Sox-2), Kruppel-like factor 4 (Klf-4), and Rex-1. FIG. 9D shows that cardiogenic differentiation markers such as Gata-4 and myocyte-specific enhancer factor 2C (Mef2c). FIG. 9E shows that antiaging markers such as sirtuin 1 (Sirt1) and telomerase reverse transcriptase (Tert) are expressed. In FIG. 9F, marker expression as examined by RT-PCR. In FIG. 9G, SJSCs from aged bone marrow showed less senescence-associated β-galactosidase (β-gal) expression compared with other cells in aged BM. FIG. 9H is confocal microscopy showing that aged SJSCs had a higher density of telomeres compared with aged BMSCs. FIG. 9I is quantitative fluorescence in situ hybridization (FISH) analysis demonstrated that SJSCs maintain longer telomeres compared with BMSCs. Telomere shortening was delayed in aged SJSCs compared with aged BMSCs.

FIGS. 10A-10D show characterization of Sca-1⁺ CSCs. FIG. 10A is a brief description of the procedure of CSCs isolation. FIG. 10B shows Sca-1⁺ expression in isolated CSCs was validated by immunocytochemistry and flowcytometry. (light grey=Sca-1⁺; dark grey=DAPI). In FIG. 10C, Discoidin domain receptor 2 (DDR2) and prolyl-4-hydroxylase beta (P4HB) antibodies were rarely positive in CSC cultures. FIG. 10D shows that expression of a hematopoietic progenitor marker (c-kit), pluripotency markers (Oct-4, Sox2, Nanog), a stem cell side population marker (Bcrp1), early cardiac lineage markers (Nkx-2.5, GATA4, MEF2C), and a vascular progenitor marker (Flk1) in isolated Sca-1⁺ cell colonies as analyzed by RT-PCR. “C” notation above the columns stands for cultured colonies and their identity numbers. They maintained about 80˜90% of Sca-1⁺ positive cells from passage 5 to 30, measured by flowcytometry. All experiments were performed within this passage. Bar=100 μm.

FIGS. 11A-11D show that connective tissue growth factor (“CTGF”) is a major downstream factor for electrically stimulated-induced focal adhesion kinase (FAK)-AKT (“FAK/AKT”) pathways. CTGF is a major downstream factor for EleS-induced FAK/AKT pathways. FIG. 11A shows the results of real-time PCR-based gene expression profiling performed for extracellular matrix (“ECM”) and cell adhesion molecules. Four of each upregulated and down-regulated genes were selected based on fold change. In FIGS. 11B and 11C, mRNA expression of connective tissue growth factor (“CTGF”) was confirmed by conventional RT-PCR and real-time PCR (n=5). FIG. 11D shows CTGF positivity by immunocytochemistry was higher in electrically stimulated cardiac stem cells (“EleS CSCs”) when compared with non-electrically stimulated cardiac stem cells (“Non-EleS CSCs”).

FIGS. 12A-12D show that “Tert” is a direct target of miR-195 in stem cell aging. FIG. 12A shows computational analysis predicted “mTert” as a potential target of mmu-miR-195.

FIGS. 12B and 12C show transfection of OMSCs with anti-miR-195 restored expression of Tert mRNA as well as telomere specific protein TRF2, as examined by reverse transcriptase polymerase chain reaction and Western blot, respectively. FIG. 12D shows cotransfection with pEZX-Luc vector containing mTert 3′ UTR and a plasmid encoding miR-195 showed decreased luciferase activity (p<0.01 vs. pEZX-miR-SC transfected cells). Transfection efficiency was normalized by Renilla luciferase activity. Acronyms: OMSCs, old mesenchymal stem cells; Tert, telomerase reverse transcriptase; UTR, untranslated region.

FIGS. 13A-13G show abrogation of miR-195 rejuvenates OMSCs by telomere relengthening and antiaging markers reactivation. FIG. 13A shows the structures of Lenti-miR-195 inhibitor and Lenti-scramble vectors which contain mCherry reporter gene (red signal). FIG. 13B shows abrogation of miR-195 significantly reduced senescence-associated b-gal expression in OSMCs as compared to scramble transfected OMSCs. FIG. 13C shows significant telomere relengthening of OMSCs upon transfection with miR-195 inhibitor. FIG. 13D shows that miR-195 inhibition markedly reduced terminal deoxynucleotidyl transferase dUTP nick end labeling-positive apoptotic cell death in OMSCs. FIG. 13E shows that expressions of TERT (not shown), SIRT1, and Bcl-2 as well as phosphorylation of p-FOXO1 and p-Akt were significantly increased by transfection of anti-miR-195 in OMSCs whereas expression of p53 and cleaved caspase 3 was reduced by miR-195 abrogation. FIGS. 13F and 13G show knockdown of miR-195 significantly restored cell proliferative abilities in OMSCs as examined by cell proliferation assay and colony formation assay. Acronyms: OMSCs, old mesenchymal stem cells; SIRT1, sirtuin (silent mating type information regulation 2 homolog) 1; Bcl-2, B cell lymphoma 2 protein, FOXO1, forkhead box protein O1 also known as forkhead in rhabdomyosarcoma is a protein, and p-FOXO1 is phospho-forkhead box protein O1, Akt, protein kinase B (PKB), also known as Akt, p-Akt is phospho-Akt.

FIGS. 14A-14H show isoxazole or isoxazole like compounds mediated generation of cardiac progenitor cells from human induced pluripotent stem cells in vitro. FIGS. 14A-14B show the characterization of human iPS cells for the expression of pluripotency markers of Oct4, Sox2, Tra-1-60, Tra-1-81, and SSEA4 by immunostaining (merged images with DAPI). FIG. 14C shows an exemplary brief description of the procedure of the generation of cardiac progenitor cells from hiPS cells and subsequent generation of cells of multiple cell lineages therefrom. FIG. 14D shows the characterization of cardiac progenitor cells with expression of the markers of Nkx2.5 and GATA4 by immunostaining (merged images with DAPI). FIG. 14E shows the differentiation of human iPS cells into beating cardiomyocytes. FIG. 14F shows in vitro differentiation of human iPS cells into vascular progenitor cells as demonstrated by the expression of VE-cadherin and CD31 by immunofluorescence analysis. FIG. 14G shows tube formation by vascular progenitor cells labeled with calcein-AM dye. FIG. 14H shows in vitro differentiation of human iPS cells into smooth muscle progenitor cells as shown by α-SMA and calpopnin immunostaining.

FIGS. 15A-15B show the effects of CXCR4 expression on muscle progenitor cell (“MPC”) migration and a schematic representation of chemotaxis experiments for cell migration. FIG. 15A shows how MPC invading the collagen gel were quantified by counting 30 optical fields per well. FIG. 15B shows MPC cell migration. Null indicates GFP/dystrophin expressing control MPC; MPCCX4 indicates GFP/dystrophin expressing MPC that overexpress CXCR4; MPCsi-CXCR4 indicates MPC treated with siRNA targeting CXCR4 (MPCsi-CXCR4), for knock-down of CXCR4 gene. The data show increased migration of MPC overexpressing CXCR4. n=4, *p<0.05 vs. control.

FIGS. 16A-16D show that cardiac fibrobrast derived human iPS cell colonies were dissociated with accutase and plated in the presence of Y27632. FIG. 16A schematically shows cell culture conditions for the generation of cardiac fibroblasts from human iPSCs. Briefly, to generate muscle progenitor cells (MPCs) from hiPSC in vitro, human Induced Pluripotent Stem (iPS) Cells (ATCC® ACS-1021™) induced from human cardiac fibroblasts were cultured with mTeSR™1 (STEMCELL Technologies Inc.) on Vitronectin XF (STEMCELL Technologies Inc.) coated 6-well plates. iPS Cells were passaged every 4 to 6 days with ReLeSR™ (STEMCELL Technologies Inc.). For differentiation of iPS Cells into MPCs, iPS Cells were dissociated into single cells with ACCUTASE™ (STEMCELL Technologies Inc.) into single cells and seeded at 1×10⁵ cells/cm² with mTeSR™1 supplemented with 5 μM RHO/ROCK pathway inhibitor (Y-27632, STEMCELL Technologies Inc.). After 24 hr, the medium was changed to fresh mTeSR™1. mTeSR™1 was refreshed daily during first 3 days. After 3 days, culture medium was changed to mTeSR™1 supplemented with 20 μM ISX-9 (MedChemExpress). The medium was refreshed every other day. After 6 days, the medium was switched to RPMI 1640 Medium (Thermo Fisher Scientific) supplemented with N-2 Supplement (Thermo Fisher Scientific) and 20 μM ISX-9 and refreshed every other day for another 3 to 6 days. Small molecules (Isx9 & GIV) were applied to initiate differentiation and analysed at day 9. FIG. 16B shows relative skeletal muscle gene expression by the treatment of Isx9 & Giv. FIGS. 16C and 16D show the muscle genes (PAX3, PAX7, MYF5, MYOG, MYOD), overexpression superiority in particular of ISX-9.

FIGS. 17A-17F show generation and characterization of hiPSC derived cardiac progenitor cells (CPCs). FIG. 17A is a schematic outline of generation of hiPSC-CPCs. FIG. 17B shows bright field of hiPSC treated with DMSO or ISX-9 in RPMI/B27 minus insulin at Day0, Day3 and Day7. FIG. 17C show time dependent expression of Nkx2.5, GATA4, ISL-1 and Mef2c.*vs. Day0, P<0.05; #vs. Day3, P<0.05, from 3 biological repeated experiments. FIG. 17D shows ISX-9 upregulated expression of Nkx2.5, GATA4, ISL-1 and Mef2c at day7 compared with DMSO group, *vs. DMSO group, P<0.05, from 3 biological repeated experiments. FIG. 17E shows fluorescence activated cell sorting analysis (FACS) showing 96.5±2.3% Nkx2.5 positive cells after ISX-9 treatment. In FIG. 17F, immunofluorescence staining showed that transcription factors (Nkx2.5, GATA4, and ISL-1) were highly upregulated in hiPSC treated with ISX-9. Scale bar represents 200 μm.***

FIGS. 18A-18G: In vitro differentiation of CPCs into three cardiac lineage cells. According to treatment outline, the CPCs expressed CM, EC, SMC specific proteins. (FIG. 18A) CM progenitors expressed α-sarcomeric actinin, cTnI, MLC2v, cTnT and Cx43, scale bar=50 μm. (FIG. 18B) Under TEM, these cells were rich in endoplasmic reticulum studded with ribosomes, developing myofilaments, mitochondria and glycogen particles. Chromatin material was uniformly distributed in the nucleoplasm. (FIG. 18C) Endothelial progenitors expressed VE-cadherin and CD31, scale bar=200 μm. LDL uptake by these cells was observed (FIG. 18E) and formed tube like structures (FIG. 18D). (FIG. 18F) Smooth muscle progenitors expressed a-SMA and calponin, scale bar=200 μm. (FIG. 18G) FACS analysis revealed 95.2+2.1% CMS, 90.3+2.5% ECs and 92.3+1.8% SMCs in basal differentiation medium.

FIGS. 19A-19C: Global mRNA and miRNA expression profile in CPCs. (FIG. 19A) Heat map of differentially expressed mRNA (Q value <10⁻¹⁵) among undifferentiated hiPSCs, DMSO or ISX-9 treated hiPSCs detected by mRNA-seq. (FIG. 19B) Heat map of differentially expressed miRNAs that exhibited significant increase or decrease individually between DMSO and ISX-9 treated hiPSCs. (FIG. 19C) Pathway enrichment analysis of upregulated and downregulated genes in CPCs induced by ISX-9 in comparison with DMSO treated hiPSCs.

FIGS. 20A-20G: Potential key signaling pathways mediated by ISX-9 treatment. (FIG. 20A) Time dependent expression of Wnt5, Wnt11 and Wnt 3a with ISX-9 treatment. *vs. Day0, P<0.05; #vs. Day3, P<0.05, n=6 from 3 biological repeated experiments. (FIG. 20B) Continuous treatment for 7 days with ISX-9 increased expression of Wnt5, Wnt11 and Wnt 3a compared with DMSO group. *vs. DMSO group, P<0.05, from 3 biological repeated experiments. Continuous treatment with ISX-9 for 7 days increased expression of Wnt5, Wnt11 (FIG. 20C) and cardiac transcription factors (Nkx2.5, Mef2c, GATA4 and ISL-1) (FIG. 20D) compared with treatment only for 3 days. *vs. 3 day treatment group, P<0.05, from 3 biological repeated experiments. (FIG. 20E) Schematic description of the protocol to study signaling pathways of cardiac differentiation induced by ISX-9. Inhibition of TGFβ signaling pathway with LY2109761 or canonical Wnt signaling pathway with XAV939 and IWP2 in the initial differentiation stage (FIG. 20F) or blockade of non-canonical Wnt signaling in late differentiation stage (FIG. 20G) with siWnt5, siWnt11 or WIF-1 along treatment with ISX-9 significantly decreased expression of cardiac transcription factors *vs. ISX-9 group, P<0.05, from 3 biological repeated experiments.

FIGS. 21A-21G: Cytoprotective effects of ISX-9 on CPCs. (FIG. 21A) Morphology of hiPSCs in RPMI/B27 medium treated with DMSO or ISX-9 under 1%02 for 12 h or 24 h. (FIG. 21B) Representative images of TUNEL staining in Mock, DMSO and ISX-9 treated groups after 24 hypoxic stresses. (FIG. 21C) Semi quantitative estimate of TUNEL positive cells. *vs. DMSO group, P<0.05. (FIG. 21D) Heat map comparing concentrations of paracrine factors released from cells with different treatments. Each row represents a cytokine and each column represents an independent condition. The heat map color scale corresponds to the absolute concentration (log 10, pg/ml) of cytokines with minimum and maximum of all values is shown on the right. (FIG. 21E) Representative images of TUNEL staining 3 days post-MI. The host cardiomyocytes were identified by α-sarcomeric actinin. (FIG. 21F) Quantitation of TUNEL positive cells in the border area of infarct with different treatments. *vs. DPBS group, P<0.05; #vs. hiPSC group, P<0.05. (FIG. 21G) Quantitation of TUNEL positive cardiomyocytes in the border area of infarct hearts from mice in different groups. *vs. DPBS group, P<0.05; #vs. hiPSC group, P<0.05.

FIGS. 22A-22H: Transplantation of CPCs reversed cardiac remodeling after MI. Temporal Changes in LVESD (FIG. 22A), LVEDD (FIG. 22B), FS (FIG. 22C) and EF (FIG. 22D) in mice post transplantation. * P<0.05 Vs. DPBS treated group at the same time point, # P<0.05 vs. hiPSC treated group at the same time point. DPBS=13, hiPSC=16 and CPC=16. (FIG. 22E) Representative echographs of M-mode and B mode echocardiography following 3M post MI. (FIG. 22F) Cardiac fibrosis was evaluated at seven levels by Masson's trichrome staining at 3M post-MI. (FIG. 22G) Sections of representative hearts are shown. (FIG. 2211) Quantification of scar tissue size. * P<0.05 Vs. DPBS treated group at the same time point, # P<0.05 vs. hiPSC treated group (n=5).

FIGS. 23A-23J: Differentiation of transplanted CPCs into cardiac lineage cells in the infarcted heart. (FIG. 23A-FIG. 23B) Engrafted CPCs were identified by PKH-26 fluorescence (red fluorescence); muscle fibers were visualized via immunostaining for human specific cTnT or α-sarcomeric actinin (Green fluorescence) at 3M post-MI. (FIG. 23C-FIG. 23D) Immunofluorescence images of PKH-26 and EC and SMC makers in the infarcted heart at 2M post-MI. Scale bar=50 μm. (FIG. 23E-FIG. 23G) Representative immunofluorescence images and quantification of transplanted CPCs (FIG. 23E) and hiPSCs (FIG. 23F) in infarct and border area 72 h post-MI using human mitochondria antigen (human-mit) tracking. * P<0.05 vs. hiPSCs treated group, n=3. (FIG. 23H-FIG. 23J) Representative immunofluorescence images and quantification of differentiated CMs in infarct and border area 3M post-MI from CPCs (FIG. 23H) and hiPSCs (FIG. 23I) treated mice using cTnI and human-mit tracking. * P<0.05 vs. hiPSCs treated group, n=3.

FIGS. 24A-24D show characterization of exosomes from iPSC and CPCISX-9. FIG. 24A shows exosomes isolated from iPSC and CPCISX-9 visualized by transmission electron microscopy (TEM). Scale bar=200 nm. FIG. 24B shows representative western blots showing that exosomes from iPSC and CPCISX-9 were enriched in Exosome (Exo)-specific markers Tsg101. Other common exosome markers included CD9, Hsp70 and Flotillin-1. Calnexin was absent in exosomes. FIG. 24C is a representative graph of size distribution of exosomes from iPSC and CPCISX-9 detecting by TRPS. FIG. 24D shows average size of exsomes as measured by TRPS. No significance difference in size between exosomes from iPSC and CPCISX-9 was observed.

FIGS. 25A-25B shows miRNA expression profiling and validation of microarray data. FIG. 25A is heatmap analysis of microarray data showing significantly upregulated miRNAs in Exo-CPCISX-9 compared with those in Exo-iPSC, Exo-EB or Exo-CPCcomm. Red or green colors indicate differentially up- or downregulated miRNAs, respectively (P<0.05). n=3. FIG. 25B is validation of microarray data using real-time PCR. Quantitative data showing significant overexpression of miR-373, miR-367, miR-520, miR-548ah, and miR-548q in Exo-CPCISX-9.RNA samples were from three biological repeat experiments. *, P<0.001.

FIGS. 26A-26E show CPC ISX-9-derived exosomes enriched with miR-373 reversed TGFβ stimulation of fibroblasts. In FIG. 26A, PKH26 labeled exosomes from CPC ISX-9 (red) were incubated together with fibroblasts and were observed within the fibroblasts (green, Calcein AM), mostly located at the perinuclear region. The white arrows show the uptaken exosomes. FIG. 26B show real-time PCR results showing anti-miR-373 (miR-373 inhibitor) at a concentration of 25 nM and 50 nM effectively downregulated miR-373 expression in CPCs ISX-9. FIG. 26C show expression of exosomal miR-373 was significantly inhibited in CPCs ISX-9 after treatment with 25 nM anti-miR-373. FIG. 26D show expression of miR-373 in fibroblasts treated with Exo-CPC ISX-9 or anti-miR-373-Exo-CPC ISX-9. Results were tabulated from three independent experiments, n=3, P<0.001. FIG. 26E show expression of fibrotic genes in fibroblasts stimulated with TGFβ after treatment with Exo-CPC ISX-9 or anti-miR-373-Exo-CPC ISX-9. * vs. Exo-CPC ISX-9 group, n=3, P<0.05.

FIGS. 27A-27D show CPCISX-9-derived exosomes promoted cardiomyocyte proliferation in mice after myocardial infarction (MI). FIG. 27A is a representative image of ki67 positive cardiomyocyte (cTnT positive) in Exo-CPCISX-9 treated mouse 30 days after MI. Bar=50 μm. FIG. 27B is semi quantitative data of proliferating cardiomyocytes in peri-infarct region 30 days after myocardial infarction as demonstrated by positivity of ki67. PBS group: n=940 cardiomyocytes from 3 hearts; Exo-iPSC group: n=950 cardiomyocytes from 3 hearts; Exo-CPCISX-9 group, n=951 cardiomyocytes from 3 hearts. * vs. PBS group, P<0.05; # vs. Exo-iPSC group, P<0.05. FIG. 27C are representative images of arterioles in peri-infarct area from mice 4 weeks after MI. Arterioles were identified by α-SMA positive staining (green) in vascular structures. Bar=100 μm. FIG. 27D are quantitative estimates of arterioles from MI mice treated with exosomes derived from different iPSC. * vs. PBS group, P<0.05; # vs. Exo-iPSC group, P<0.05, n=3.

FIGS. 28A-28G show CPC ISX-9-derived exosomes reversed cardiac remodeling in infarcted mice. FIG. 28A shows representative M mode echocardiography images from three groups at 30 days after MI. LVDs (28B) and LVDd (28C) were significantly decreased 30 days post-MI in mice with Exo-CPCISX-9 treatment. Exo-CPCISX-9 treatment significantly enhanced EF (28D) and FS (28E). * vs. PBS group, P<0.05; # vs. Exo-iPSC group, P<0.05, PBS group: n=10, Exo-iPSC group, n=9, Exo-CPCISX-9, n=11. EF, ejection fraction; FS, fractional shortening; LVDd, diastolic left ventricular dimensions; LVDs systolic left ventricular dimensions. (28F) Representative Masson's trichrome-stained sections from hearts after different treatments. (28G) Quantitative estimate of fibrosis in mice after different treatments. * vs. PBS group, P<0.05; # vs. Exo-iPSC group, P<0.05, n=4.

FIG. 29 is a chart depicting Personalized Cell-Free Therapy with exosomes generated from iPSC-derived cardiac progenitor cells.

FIGS. 30A and 30B show the effect of three small molecules (ISX9, Danzol, Givinostat) on expression of cardiac and skeletal muscle genes. Real time PCR on dystrophin on small molecule (ISX9, GIV) expression in IPS cells.

### DETAILED DESCRIPTION

### Definitions

The practice of the present invention will employ, unless otherwise indicated, conventional techniques of tissue culture, immunology, molecular biology, microbiology, cell biology and recombinant DNA, which are within the skill of the art. See, e.g., Sambrook, Fritsch and Maniatis (1989) Molecular Cloning: A Laboratory Manual, 2^{nd} edition; F. M. Ausubel, et al. eds. (1987) Current Protocols In Molecular Biology; the series Methods in Enzymology (Academic Press, Inc.): PCR 2: A Practical Approach (1995) (M. J. MacPherson, B. D. Hames and G. R. Taylor eds.); Harlow and Lane, eds. (1988) Antibodies, A Laboratory Manual; Harlow and Lane, eds. (1999) Using Antibodies, A Laboratory Manual; and R. I. Freshney, ed. (1987) Animal Cell Culture.

All numerical designations, e.g., pH, temperature, time, concentration, and molecular weight, including ranges, are approximations which are varied (+) or (−) by increments of 1.0 or 0.1, as appropriate. It is to be understood, although not always explicitly stated that all numerical designations are preceded by the term “about”. It also is to be understood, although not always explicitly stated, that the reagents described herein are merely exemplary and that equivalents of such are known in the art.

As used in the specification and claims, the singular form “a,” “an” and “the” include plural references unless the context clearly dictates otherwise.

The terms autologous transfer, autologous transplantation, autograft and the like refer to treatments wherein the cell donor is also the recipient of the cell replacement therapy. The terms allogeneic transfer, allogeneic transplantation, allograft and the like refer to treatments wherein the cell donor is of the same species as the recipient of the cell replacement therapy, but is not the same individual. A cell transfer in which the donor's cells and have been histocompatibly matched with a recipient is sometimes referred to as a syngeneic transfer. The terms xenogeneic transfer, xenogeneic transplantation, xenograft and the like refer to treatments wherein the cell donor is of a different species than the recipient of the cell replacement therapy.

A “biocompatible scaffold” refers to a scaffold or matrix for tissue-engineering purposes with the ability to perform as a substrate that will support the appropriate cellular activity to generate the desired tissue, including the facilitation of molecular and mechanical signaling systems, without eliciting any undesirable effect in those cells or inducing any undesirable local or systemic responses in the eventual host. In other embodiments, a biocompatible scaffold is a precursor to an implantable device which has the ability to perform its intended function, with the desired degree of incorporation in the host, without eliciting an undesirable local or systemic effects in the host. Biocompatible scaffolds are described in U.S. Pat. No. 6,638,369.

As used herein, a “cardiac patch” or “cardiac progenitor patch embedded in fibrin” or “Epicardial patch” is a bioengineered 2D or 3-dimensional (3D) tissue patch comprising or containing iPS cells or iPS cells derived cardiac lineage or cardiac progenitor cells.

A “cardiomyocyte” or “cardiac myocyte” is a specialized muscle cell which primarily forms the myocardium of the heart. Cardiomyocytes have five major components: 1. cell membrane (sarcolemma) and T-tubules, for impulse conduction, 2. sarcoplasmic reticulum, a calcium reservoir needed for contraction, 3. contractile elements, 4. mitochondria, and 5. a nucleus. Cardiomyocytes can be subdivided into subtypes including, but not limited to, atrial cardiomyocyte, ventricular cardiomyocyte, SA nodal cardiomyocyte, peripheral SA nodal cardiomyocyte, or central SA nodal cardiomyocyte. Stem cells can be propagated to mimic the physiological functions of cardiomyocytes or alternatively, differentiate into cardiomyocytes. This differentiation can be detected by the use of markers selected from, but not limited to, myosin heavy chain, myosin light chain, actinin, troponin, tropomyosin, GATA4, Mef2c, and Nkx-2.5.

The cardiomyocyte marker “myosin heavy chain” and “myosin light chain” are part of a large family of motor proteins found in muscle cells responsible for producing contractile force. These proteins have been sequenced and characterized, see for example GenBank Accession Nos. AAD29948, CAC70714, CAC70712, CAA29119, P12883, NP_000248, P13533, CAA37068, ABR18779, AAA59895, AAA59891, AAA59855, AAB91993, AAH31006, NP_000423, and ABC84220. The genes for these proteins has also been sequenced and characterized, see for example GenBank Accession Nos. NM_002472 and NM_000432.

The cardiomyocyte marker “actinin” is a mircrofilament protein which are the thinnest filaments of the cytoskeleton found in the cytoplasm of all eukaryotic cells. Actin polymers also play a role in actomyosin-driven contractile processes and serve as platforms for myosin's ATP hydrolysis-dependent pulling action in muscle contraction. This protein has been sequenced and characterized, see for example GenBank Accession Nos. NP_001093, NP_001095, NP_001094, NP_004915, P35609, NP_598917, NP_112267, AAI07534, and NP_001029807. The gene for this protein has also been sequenced and characterized, see for example GenBank Accession Nos. NM_001102, NM_004924, and NM_001103.

The cardiomyocyte marker “troponin” is a complex of three proteins that is integral to muscle contraction in skeletal and cardiac muscle. Troponin is attached to the protein “tropomyosin” and lies within the groove between actin filaments in muscle tissue. Tropomyosin can be used as a cardiomyocyte marker. These proteins have been sequenced and characterized, see for example GenBank Accession Nos. NP_000354, NP_003272, P19429, NP_001001430, AAB59509, AAA36771, and NP_001018007. The gene for this protein has also been sequenced and characterized, see for example GenBank Accession Nos. NM_000363, NM_152263, and NM_001018007.

“Clonal proliferation” refers to the growth of a population of cells by the continuous division of single cells into two identical daughter cells and/or population of identical cells.

CTGF, also known as CCN2 or connective tissue growth factor, is a matricellular protein of the CCN family of extracellular matrix-associated heparin-binding proteins (see also CCN intercellular signaling protein).

Telomerase reverse transcriptase (“TERT”) is a catalytic subunit of the enzyme telomerase, which, together with the telomerase RNA component (TERC), comprises the most important unit of the telomerase complex.

The miR-290-295 cluster is a pluripotent cluster codes for a family of microRNAs (miRNAs) that are expressed de novo during early embryogenesis and are specific for mouse embryonic stem cells (ESC) and embryonic carcinoma cells (ECC). Such are known in the art and described, for example, in Lichner et al. (2011) Differentiation, January 81(1):11-24.

Chemokine (C—C motif) ligand 7 (CCL7) is a small cytockine previously known as monocyte-specific chemokine 3 (MCP3). The protein sequence is available under Accession number NP_006264 and the murine sequence is available under NP_038682 (see also ncbi.nlm.nih.gov/gene/6354, last accessed on Apr. 16, 2014). An antibody and kit to detect CCL7 is available from Sino Biological Inc.

CXCR2 chemokine receptor 2 (CXCR2) is a protein encoded by this gene is a member of the G-protein-coupled receptor family. This protein is a receptor for interleukin 8 (IL8). It binds to IL8 with high affinity, and transduces the signal through a G-protein activated second messenger system. This receptor also binds to chemokine (C—X—C motif) ligand 1 (CXCL1/MGSA). Information regarding the protein and its gene is found on nchbi.nlm.nih.gov/gene/3579 (last accessed on Apr. 16, 2014).

Integral membrane protein 2A is a stem cell marker. The sequence of the human gene is reported at UniProtKB (043736) and the murine sequence is reported at Q61500 (uniprot.org/uiprot, last accessed on Apr. 16, 2014).

DNA (cytosine-5)-methyltransferase 1 is an enzyme that is encoded by the DNMT1 gene. The complete sequence of the protein and its gene is available at genecards.org/cgi-bin/carddisp.pl?gene=DNMT1, last accessed on Apr. 16, 2014. Antibodies to detect the protein are commercially available, e.g., from Cell Signaling Technologies (DNMT1 (D63A6) XP® Rabbit mAb #5032). DNA (cytosine-5)-methyltransferase 3 is an enzyme that is encoded by the DNMT3 gene.

EFNA3 or ephrin A3 is a protein receptor. The human protein sequence is reported at ncbi.nlm.nih.gov/gene/1944. Antibodies useful for the detection and analysis of the protein are available from R&D Systems and Santa Cruz Biotechnology.

“Let-7” refers to a family of microRNAs. The sequences are reported at the miRBase at mirbase.org/cgi-bin/mirna_summary.pl?fam=MIPF000002, last accessed on Apr. 16, 2014. Methods for detecting such are known in the art, e.g., U.S. Patent Application Publication No. 2014/0005251.

Max is a pluripotency marker that binds MYC. See Chappell et al. (2013) Genes & Dev. 27:725-733.

The protein encoded by the Tsg101 gene belongs to a group of apparently inactive homologs of ubiquitin-conjugating enzymes. The gene product contains a coiled-coil domain that interacts with stathmin, a cytosolic phosphoprotein implicated in tumorigenesis. The protein may play a role in cell growth and differentiation and act as a negative growth regulator. In vitro steady-state expression of this tumor susceptibility gene appears to be important for maintenance of genomic stability and cell cycle regulation. Mutations and alternative splicing in this gene occur in high frequency in breast cancer and suggest that defects occur during breast cancer tumorigenesis and/or progression.

CD9 encodes a member of the transmembrane 4 superfamily, also known as the tetraspanin family. Tetraspanins are cell surface glycoproteins with four transmembrane domains that form multimeric complexes with other cell surface proteins. The encoded protein functions in many cellular processes including differentiation, adhesion, and signal transduction, and expression of this gene plays a critical role in the suppression of cancer cell motility and metastasis.

As used herein, the term “microRNAs” or “miRNAs” refers to post-transcriptional regulators that typically bind to complementary sequences in the three prime untranslated regions (3′ UTRs) of target messenger RNA transcripts (mRNAs), usually resulting in gene silencing. Typically, miRNAs are short, non-coding ribonucleic acid (RNA) molecules, for example, 21 or 22 nucleotides long. The terms “microRNA” and “miRNA” are used interchangeably.

mir-373 is annotated as ENSG00000199143 and miRBase: has-mir-373.

mir-210 is annotated as ENSG00000199038 and miRBase: has-mir-210 and is associated with Sudden Infant Death Syndrome susceptibility.

Tcf15 encodes a basic helix-loop-helix transcription factor expressed early in development, which is believe to participate in patterning of the mesoderm and its derivative cell types. This gene is annotated as Ensembl: ENSG00000125878 and Uniprot Q12870.

mir-377 is annotated as ENSG00000199015 and miRBase: has-mir-377.

mir-367 is annotated as ENSG00000199169 and miRBase: has-mir-367.

mir-520c is annotated as ENSG00000207738 and miRBase: has-mir-520c.

mir-548ah is annotated as ENSG00000283682 and miRBase: has-mir-548ah.

Dystrophin intends a protein encoded by the gene Dmd, annotated as Ensembl: ENSG00000198947 and Uniprot: P11523. This protein is a component of the dystrophin-glycoprotein complex, which anchors the cytoskeleton to the extra-cellular matrix. Mutations are associated with Duchenne muscular dystrophy, Becker muscular dystrophy and cardiomyopathy, as well as equivalents thereof.

mir-548q is annotated as ENSG00000221331 and miRBase: has-mir-548q.

mir-548q is annotated as ENSG00000221331 and miRBase: has-mir-548q.

mir-335 encodes microRNA-335, annotated as ENSG00000199043 and miRBase: has-mir-335.

mir-21 encodes microRNA-21, annotated as ENSG00000284190 and miRBase: has-mir-21. This miRNA is expressed in stem cells and plays a role in cancer.

mir-30c1 encodes a microRNA annotated as Ensembl: ENSG00000207962 and miRBase: has-mir-30c-1, which may be involved in ECM maintenance and cancer.

mir-30c2 encodes a microRNA annotated as Ensembl: ENSG00000199094 and miRBase: has-mir-30c-2. Similar to miR-30c1, it may be involved in ECM maintenance and cancer.

Meox1 encodes the mesenchyme homeobox 1 protein, and is annotated as Ensembl: ENSG00000005102 and Uniprot: P50221. This protein plays a role in somite development. Genetic mutations are associated with Klippel-Feil Syndrome.

Meox2 encodes the mesenchyme homeobox 2 protein, and is annotated as Ensemble: ENSG00000106511 and Uniprot: P50222. Based on homology to the mouse, this protein is thought to play a role in myogenesis and limb development. Mutations are associated with craniofacial and skeletal abnormalities as well as Alzheimer's.

Pax3 is annotated as Ensembl: ENSG00000135903 and Uniprot: P23760. This gene encodes a member of the paired box family of transcription factors, which regulates proliferation and migration during neural development and myogenesis. Mutations in this gene are associated with craniofacial-deafness-hand syndrome, Rhabdomyosarcoma, and Waardenburg syndrome.

Pax7 is annotated as Ensembl: ENSG00000009709 and Uniprot: P23759. This gene encodes a member of the paired box family of transcription factors, which regulates proliferation of muscle precursor cells. It is vital for embryonic development and implicated in cancer, including Rhabdomyosarcoma.

MyoD1 is annotated as Ensembl: ENSG00000129152 and Uniprot: P15172. This gene encodes a myogenic helix-loop-helix transcription factor that regulates myocyte differentiation via inhibition of the cell cycle. This protein is known to interact with other key muscle factors, Myf5, Myf6, and MyoG.

MyoG encodes the muscle-specific basic helix-loop-helix transcription activator, myogenin.

Myh2 encodes a class II or conventional myosin heavy chain. As a motor protein, it functions in skeletal muscle contraction, and mutations are associated with inclusion-body myopathy. Myh2 is annotated as Ensembl: ENSG00000125414 and Uniprot: Q9UKX2. Numerous splice variants have been reported.

Myh6 encodes a motor protein that forms the alpha heavy chain subunit of cardiac myosin. This gene is annotated as Ensemble: ENSG00000197616 and Uniprot: P13533, and mutations are associated with atrial septal defects and hypertrophic cardiomyopathy.

Tbx1 encodes a member of the developmentally important T-box transcription factor family. It is annotated as Ensembl: ENSG00000184058 and Uniprot: Q43435. Mutations in this gene are associated with neural-crest defects, DiGeorge syndrome, and velocardiofacial syndrome.

Mesp1 encodes a basic helix-loop-helix transcription factor that is involved in development of the somatic and cardiac mesoderm, and rostrocaudal patterning of the somites. Mesp1 is annotated in Ensembl: ENSG00000166823 and Uniprot: Q0BRJ9.

Des encodes the protein Desmin, and is annotated as Ensemble: ENSG00000175084 and Uniprot: P17661. Desmin is a muscle-specific class III intermediate filament that forms a fibrous network for myofibrils. Mutations in Des are associated with cardiac and skeletal muscle myopathies.

Cnntb1 is a well-known gene that encodes the protein, β-catenin, a key component of the canonical Wnt signaling pathway. In the presence of Wnt, β-catenin translocates to the nucleus as acts as a transcriptional regulator. This protein is also involved in regulation of contact inhibition. Mutations in this gene are associated with mental retardation and colorectal cancer. The Cnntb1 gene is annotated as Ensemble: ENSG00000168036 and Uniprot: P35222.

Pax7 is annotated as Ensembl: ENSG00000009709 and Uniprot: P23759. This gene encodes a member of the paired box family of transcription factors, which regulates proliferation of muscle precursor cells. It is vital for embryonic development and implicated in cancer, including Rhabdomyosarcoma.

Myf5 gene, Ensemble: ENSG00000111049, encodes a master transcriptional regulator of muscle differentiation, that binds and promotes transcription of numerous myogenic factors (Uniprot: P13349). Mutations in Myf5 are associated with skeletal muscle cancer and Rhabdomyosarcoma.

MyoD1 is annotated as Ensembl: ENSG00000129152 and Uniprot: P15172. This gene encodes a myogenic helix-loop-helix transcription factor that regulates myocyte differentiation via inhibition of the cell cycle. This protein is known to interact with other key muscle factors, Myf5, Myf6, and MyoG.

xESI myogenic genes intend Myogenic regulatory factors (MRF) which are basic helix-loop-helix (bHLH) transcription factors that regulate myogenesis: MyoD, Myf5, myogenin, and MRF4. These proteins contain a conserved basic DNA binding domain that binds the E box DNA motif. [2] They dimerize with other HLH containing proteins through an HLH-HLH interaction.

Pitx2 is annotated as ENSG00000164093 and Uniprot: Q99697. This gene encodes Paired-like homeodomain transcription factor 2, which belongs to the bicoid family of homeodomain proteins. This protein regulates the hormone, Prolactin, and is important for development of eyes, teeth, and abdominal organs. Mutations in this gene associate with Axenfeld-Rieger Syndrome.

ISL1 is a gene (Ensembl: ENSG00000016082) that encodes the transcription factor, ISL LIM homeobox 1 (Uniprot: P61371). This protein is implicated in motor neuron and retinal ganglion cell specification, and regulating expression of the Insulin gene. Mutations are associated with maturity-onset diabetes and bladder exstrophy.

Nkx2.5 encodes a master transcription factor involved in cardiac development. Annotated as Ensembl: ENSG00000183072 and Uniprot P52952, mutations this gene can result in atrial septal defects, and a form of congenital hypothyroidism.

Hand1 is a basic helix-loop-helix transcription factor annotated as Ensembl: ENSG00000113196 and Uniprot: Q96004. During heart development, Hand1 is expressed asymmetrically with another Hand protein to direct cardiac morphogenesis and formation of the right ventricle and aortic arch arteries. Mutations in genes encoding Hand proteins are associated with congenital heart disease.

GATA4 is annotated as ENSG000001366574 in Ensembl, and encodes a member of the gata family of zinc-finger transcription factors. This protein, Uniprot: P43694, is key to embryogenesis, cardiac development, and myocardial function. Mutations are associated with septal defects and various forms of cancer.

Tbx5 is a member of the T-box gene family, which contains a conserved DNA-binding domain. Numerous transcripts of Tbx5 are curated in RefSeq and the Ensembl gene identifier is ENSG00000089225. The protein product, Uniprot: Q99593, is important for heart and limb development, and mutations in this gene are associated with Holt-Oram syndrome.

TnnT2 is a gene encoding cardiac troponin T2 (Uniprot:P45379) the tropomyosin-binding unit of the troponin complex. In response to changes in intracellular calcium levels, Tnnt2 regulates muscle contraction. Mutations in this gene, annotated as Ensembl: ENSG00000118194, are associated with familiar hypertrophic cardiomyopathy and dilated cardiomyopathy.

Myl7 is a gene encoding the calcium binding motor protein myosin light chain 7. This gene is annotated in Ensembl: ENSG00000106631 and UniProt: Q01449. Mutation at this locus are associated with Fechtner Syndrome and Familial Atrial Fibrillation.

MLC2v gene (more commonly denoted as My12, in humans), curated as Refseq: NM_00432 and Uniprot: P10916, encodes the motor protein myosin light chain 2. Calcium dependent phosphorylation of this protein results in generation of contractile forces. This protein functions in heart development and cardiac contractility, and mutations are associated with mid-left ventricular chamber hypertrophic cardiomyopathy. Antibodies are available through Invitrogen and Santa Cruz Biotechnology.

Mef2c is a gene (Ensembl ID ENSG00000081189) that produces more than 8 alternatively spliced transcripts curated in RefSeq. The protein product (Uniprot: Q06413) is a member of the MADS box transcription enhancer factor 2 family, and plays a role in vascular development, cardiac morphogenesis, myogenesis, and maintenance of the differentiated state. Genomic aberrations within this gene locus are associated with mental retardation, cerebral malformation, epilepsy, and arrhythmogenic right ventricular dysplasia 5. Cell Signaling Technologies, Novus Biologicals, and Invitrogen all provides products for detection and study of this protein.

Cdh4 gene produces three transcript variants encoding the protein, Cadherin 4. A member of the cadherin superfamily, Chd4 functions as a calcium-dependent cell adhesion molecule important for brain segmentation and neuronal outgrowth. This protein is also implicated in kidney and muscle development. Cdh4 is annotated in Refseq: NM_001252399 and Uniprot: P55283. Purified protein, antibodies, and other detection kits are widely available through sources including Invitrogen, Abcam, and R&D systems.

Lhx2 encodes the Lim homeobox 2 protein, a member of the LIM domain family, which carry a cysteine-rich zinc binding domain. Lhx2 is curated in Refseq: NM_004789 and UniProt: P50458, and believed to function as a transcriptional activator involved in cellular differentiation and development of the lymphoid and neural lineages. Antibodies and ELISA detection kits for Lhx2 are commercially available from Origene, Santa Cruz Biotechnology and Invitrogen.

Gαi is a heterotrimeric G protein subunit that inhibits the product of cAMP from ATP. An exemplary sequence is provided under GenBank Ref.: NM_002069 and UnProt P63096. Antibodies that recognize this marker are commercially available from Santa Cruz Biotechnology.

Cytoskeletal remodeling intends remodeling intends the dynamic reorganization of microfilaments (actins), microtubules (tubulin), and intermediate filaments (i.e. vimentin, keratin, desmin), which comprise the eukaryotic cytoskeleton. Though complex, this process occurs within minutes and facilitates biological functions such as cell migration, cytokinesis, and muscle contraction.

Promoting TGF-β induced emt (epithelial-mensenchymal transition) signaling intends transdifferentiation of cells with epithelial-like properties into cells with mesenchymal-like properties, as mediated by the signaling molecule TGF-β. Non-limiting biological roles for this process, referred to as EMT, include cancer, fibrosis, heart development, and cardiac differentiation. Transforming growth factor-β (TGF-β) is a potent inducer of EMT both during development and in cancer. In TGF-β induced EMT, activation of Smad proteins results in their nuclear translocation, DNA binding, and upregulation of EMT transcription factors. Non-limiting examples of EMT transcription factors include Snail, Twist, and Zeb. EMT requires cytoskeletal remodeling and cardiac differentiation intends efficient differentiation of human pluripotent stem cells (PSCs) such a IPS cells to contracting cardiomyocytes.

Promoting expression of genes for development of pip3 signaling in cardiomyocytes, muscle contraction and nf-at hypertrophy signaling pathways intends activate downstream signaling components, the most notable one being the protein kinase AKT, which activates downstream anabolic signaling pathways required for cell growth and survival. Phosphatidylinositol (3,4,5)-trisphosphate (PtdIns(3,4,5)P3), abbreviated PIP3, is the product of the class I phosphoinositide 3-kinases (PI 3-kinases) phosphorylation of phosphatidylinositol (4,5)-bisphosphate (PIP2). It is a phospholipid that resides on the plasma membrane. PIP3 signaling in cardiac myocytes.

Phosphoinositide 3-kinase (PI3K) can be activated in cardiac myocytes by the receptors with intrinsic tyrosine kinase activity, such as insulin receptor (INSR), growth factor receptors (IGF1 receptor and HGF receptor), and by the G protein-coupled receptors (GPCRs). INSR and IGF1 receptor engagement triggers receptor activation and autophosphorylation. The activated receptor can then phosphorylate several intracellular protein substrates, most notably the insulin receptor substrate (IRS1-4) proteins. Tyrosine-phosphorylated IRS1 can recruit and activate the downstream effector, PI3K, which generates phosphatidylinositol 3,4,5-trisphosphate (PIP3) using inositol-containing phospholipids resident in the plasma membrane as substrates. IRS proteins also recruit adaptors Shc and Grb-2. The protein tyrosine phosphatase PTP1B is responsible for negatively regulating INSR signaling by dephosphorylating the phosphotyrosine residues of this receptor. Hepatocyte growth factor receptor (HGF receptor) activation induces the tyrosine phosphorylation of GAB1 and its association with PI3K via the recruitment of its regulatory subunit (PI3KR class 1A) that stimulates its catalytic subunit (PI3KC class 1A). Activated adaptors Shc and Grb-2 recruit exchange factor SOS that activates H-RAS [4]. H-RAS directly stimulates PI3K catalytic subunit (PI3KC class 1A). PI3K converts phosphatidylinositol 4,5-biphosphate (PI(4,5)P2) to PIP3 [6]. PIP3 is the second messenger that activates diverse signal cascades, including PDK and AKT pathway. Phosphatase PTEN acts as a negative regulator for the PI3K/AKT signaling pathway, converting PI(3,4,5)P3 into PI(4,5)P2. AKT and PDK phosphorylate diverse proteins that mediate various insulin- and growth factor-induced cellular responses such as glycogen synthesis, protein synthesis, cell cycle initiation, and promotion of cell survival by regulation of apoptosis factors such as BAD and Bcl-x(L).

As used herein, the term “microRNAs” or “miRNAs” refers to post-transcriptional regulators that typically bind to complementary sequences in the three prime untranslated regions (3′ UTRs) of target messenger RNA transcripts (mRNAs), usually resulting in gene silencing. Typically, miRNAs are short, non-coding ribonucleic acid (RNA) molecules, for example, 21 or 22 nucleotides long. The terms “microRNA” and “miRNA” are used interchangeably.

miR-133 refers to a microRNA that has been linked to an immature or undifferentiated phenotype. Methods to detect such include, for example, microarray-RT-PCR and RNA-seq. Commercially available kits to miR-133 is available from EMD Millipore (SmartFlare™ Detection Probes) which allow for the detection of miRNA in live cells.

miR-762 is a non-coding RNA that has been linked to post-transcriptional regulation of gene expression in multicellular organisms. The miR-762 human sequence is reported under Accession No. MI0003892 (last accessed on Apr. 16, 2014). The murine sequence is reported under NR_030428.1 (see ncbi.nlm.nih.gov/gene/79103, last accessed on Apr. 16, 2014). Methods to detect such are known in the art and kits are commercially available from, for example, Origene (miR-762, see origene.com, last accessed on Apr. 16, 2014).

miR-133 refers to a microRNA that has been linked to an immature or undifferentiated phenotype. Methods to detect such include, for example, microarray-RT-PCR and RNA-seq. Commercially available kits to miR-133 is available from EMD Millipore (SmartFlare™ Detection Probes) which allow for the detection of miRNA in live cells.

miR-762 is a non-coding RNA that has been linked to post-transcriptional regulation of gene expression in multicellular organisms. The miR-762 human sequence is reported under Accession No. MI0003892 (last accessed on Apr. 16, 2014). The murine sequence is reported under NR_030428.1 (see ncbi.nlm.nih.gov/gene/79103, last accessed on Apr. 16, 2014). Methods to detect such are known in the art and kits are commercially available from, for example, Origene (miR-762, see origene.com, last accessed on Apr. 16, 2014).

miR-195 is an RNA gene, and is reported to be affiliated with the miRNA class. Diseases associated with miR-195 include tongue squamous cell carcinoma and primary peritoneal carcinoma. Among its related pathways are microRNAs in cancer and microRNAs in cardiomyocyte hypertrophy. It is also known as MIRN195, Has-MIR-195 and MiRNA 195. The sequence and homologs are reported in the genecards web page. Nucleic acid sequences are reported under GenBank Accession No. AK098506, last accessed on Nov. 18, 2015.

ILS1 refers to an insulin gene enhancer protein, which plays an important role in regulating insulin gene expression. ISL1 is also found central to the development of pancreatic cell lineages and may also be required for motor neuron generation. ISL1 is identified as a marker for cardiac progenitor cells.

Tbx-5 is a cardiac transcription factor, also known as T-box transcription factor (“TBX5”) is a protein that in humans is encoded by the TBX5 gene. As indicated on the GeneCards human gene database, this gene is a member of a phylogenetically conserved family of genes that share a common DNA-binding domain, the T-box. T-box genes encode transcription factors involved in the regulation of developmental processes. This gene is closely linked to related family member T-box 3 (ulnar mammary syndrome) on human chromosome 12. The encoded protein may play a role in heart development and specification of limb identity. Mutations in this gene have been associated with Holt-Oram syndrome, a developmental disorder affecting the heart and upper limbs. Several transcript variants encoding different isoforms have been described for this gene. The accession for the protein is Q99593 or alternatively A6ND77, or alternatively O15301, or alternatively Q96TBO. Antibodies to the protein are commercially available from R&D Systems, Browse EMD, OriGene Antibodies, and Novus Biologicals.

As used herein, the term “a protein that facilitates regeneration and/or improves function of a tissue” intends a protein that can either regenerate or regrow or improve the tissue function or bone function is a potential cytokine which improves tissue regeneration or bone function. The gel-forming property makes certain protein polymer highly suitable for biomedical applications, such as tissue regeneration in operations and wounds. Non-limiting examples of such include IGFBP5 protein which enhances periodontal tissue and PPARα which activates liver regeneration.

As used herein, “lyophilization” intends low temperature drying or freeze drying.

Cell-derived exosomes or microvesicles, also referred to as extracellular exosomes or microvesicles, are membrane surrounded structures that are released by cells in vitro and in vivo. Extracellular exosomes or microvesicles can contain proteins, lipids, and nucleic acids and can mediate intercellular communication between different cells, including different cell types, in the body. Two types of extracellular exosomes or microvesicles are exosomes or microvesicles and microvesicles. Exosomes or microvesicles are small lipid-bound, cellularly secreted exosomes or microvesicles that mediate intercellular communication via cell-to-cell transport of proteins and RNA (El Andaloussi, S. et al. (2013) Nature Reviews: Drug Discovery 12(5):347-357). Exosomes or microvesicles range in size from approximately 30 nm to about 200 nm. Exosomes or microvesicles are released from a cell by fusion of multivesicular endosomes (MVE) with the plasma membrane. Microvescicles, on the other hand, are released from a cell upon direct budding from the plasma membrane (PM) and are packaged with different factors. Microvesicles are typically larger than exosomes or microvesicles and range from approximately 200 nm to 1 μm and have different functionalities.

Cell-derived exosomes or microvesicles can be isolated from eukaryotic cells using commercially available kits as disclosed herein and available from biovision.com and novusbio.com, or using the methods described herein. Non-limiting examples of cells that cell-derived exosomes or microvesicles can be isolated from include stem cells. Non-limiting examples of such stem cells include adult stem cells, embryonic stem cells, embryonic-like stem cells, non-embryonic stem cells, or induced pluripotent stem cells.

As used herein, the terms “overexpress,” “overexpression,” and the like are intended to encompass increasing the expression of a nucleic acid or a protein to a level greater than the exosome or microvesicle naturally contains. It is intended that the term encompass overexpression of endogenous, as well as heterologous nucleic acids and proteins.

As used herein, the term “homogeneous” in reference to a population of e cell-derived exosomes or microvesicles refers to population of cell-derived exosomes or microvesicles that have a similar amount of an exogenous nucleic acid, a similar amount of an exogenous protein, are of a similar size, or combinations thereof. A homogenous population is one wherein about 65%, about 70%, about 75%, about 80%, about 85%, about 90%, about 95%, about 98%, or 100% of the cell-derived exosomes or microvesicles share at least one characteristic. Another example of a homogenous population is one wherein about 90% of the exosomes or microvesicles are less than 50 nm in diameter.

As used herein, the term “heterogeneous” in reference to a population of cell-derived exosomes or microvesicles refers to population of cell-derived exosomes or microvesicles that have differing amounts of an exogenous nucleic acid, differing amounts of an exogenous protein, are of a different size, or combinations thereof.

The term “substantially” refers to the complete or nearly complete extent or degree of a characteristic and in some aspects, defines the purity of the isolated or purified population of exosomes or microvesicles.

The term “purified population,” relative to cell populations, cell-derived exosomes or microvesicles or miRNA, as used herein refers to plurality of such that have undergone one or more processes of selection for the enrichment or isolation of the desired exosome or microvesicle or miRNA population relative to some or all of some other component with which cell-derived exosomes or microvesicles are normally found in culture media. Alternatively, “purified” can refer to the removal or reduction of residual undesired components found in the conditioned media (e.g., cell debris, soluble proteins, etc.). A “highly purified population” as used herein, refers to a population of cell-derived exosomes or microvesicles in which at least 65%, at least 70%, at least 75%, at least 80%, at least 85%, at least 90%, at least 95%, at least 98%, at least 99% or 100% of cell debris and soluble proteins (e.g., proteins derived from fetal bovine serum and the like) in the conditioned media along with the cell-derived exosomes or microvesicles or miRNA are removed. The cells, populations, exosomes or microvesicles and miRNA as described herein can be provided in isolated, purified, highly purified forms, homogeneous, substantially homogeneous and heterogenous forms.

As used herein the terms “culture media” and “culture medium” are used interchangeably and refer to a solid or a liquid substance used to support the growth of cells (e.g., stem cells). Preferably, the culture media as used herein refers to a liquid substance capable of maintaining stem cells in an undifferentiated state. The culture media can be a water-based media which includes a combination of ingredients such as salts, nutrients, minerals, vitamins, amino acids, nucleic acids, proteins such as cytokines, growth factors and hormones, all of which are needed for cell proliferation and are capable of maintaining stem cells in an undifferentiated state. For example, a culture media can be a synthetic culture media such as, for example, minimum essential media α (MEM-α) (HyClone Thermo Scientific, Waltham, Mass., USA), DMEM/F12, GlutaMAX (Life Technologies, Carlsbad, Calif., USA), Neurobasal Medium (Life Technologies, Carlsbad, Calif., USA), KO-DMEM (Life Technologies, Carlsbad, Calif., USA), DMEM/F12 (Life Technologies, Carlsbad, Calif., USA), supplemented with the necessary additives as is further described herein. In some embodiments, the cell culture media can be a mixture of culture media. Preferably, all ingredients included in the culture media of the present disclosure are substantially pure and tissue culture grade. “Conditioned medium” and “conditioned culture medium” are used interchangeably and refer to culture medium that cells have been cultured in for a period of time and wherein the cells release/secrete components (e.g., proteins, cytokines, chemicals, etc.) into the medium.

A “composition” is also intended to encompass a combination of a cell, a cell population, an exosome or microvesicle, an miRNA, or populations of such, or an active agent, and another carrier, e.g., compound or composition, inert (for example, a detectable agent or label) or active, such as an adjuvant, diluent, binder, stabilizer, buffers, salts, lipophilic solvents, preservative, adjuvant or the like. Carriers also include biocompatible scaffolds, pharmaceutical excipients and additives proteins, peptides, amino acids, lipids, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this invention, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

A preservative intends a composition that enhances the viability of an agent in a composition. Non-limiting examples include Benzoates (such as sodium benzoate, benzoic acid), Nitrites (such as sodium nitrite) and Sulphites (such as sulphur dioxide).

A cryoprotective is a compound that protects the agent during freezing and thawing procedures. Non-limiting examples of such include DMSO, Glycerol, PEG.

A “control” is an alternative subject or sample used in an experiment for comparison purpose. A control can be “positive” or “negative”. For example, where the purpose of the experiment is to determine a correlation of an altered expression level of a gene with a particular phenotype, it is generally preferable to use a positive control (a sample from a subject, carrying such alteration and exhibiting the desired phenotype), and a negative control (a subject or a sample from a subject lacking the altered expression or phenotype). Additionally, when the purpose of the experiment is to determine if an agent effects the differentiation of a stem cell or expression of an exosome or microvesicle or miRNA, it is preferable to use a positive control (a sample with an aspect that is known to affect differentiation or altered expression) and a negative control (an agent known to not have an affect or a sample with no agent added).

The term “culturing” refers to the in vitro propagation of cells or organisms on or in media of various kinds. It is understood that the descendants of a cell grown in culture may not be completely identical (i.e., morphologically, genetically, or phenotypically) to the parent cell. By “expanded” is meant any proliferation or division of cells.

As used herein, the term “detectably labeled” means that the agent (biologic or small molecule) is attached to another molecule, compound or polymer that facilitates detection of the presence of the agent in vitro or in vivo.

A “detectable label” intends a directly or indirectly detectable compound or composition that is conjugated directly or indirectly to the composition to be detected, e.g., N-terminal histadine tags (N-His), magnetically active isotopes, e.g., ¹¹⁵Sn, ¹¹⁷Sn and ¹¹⁹Sn, a non-radioactive isotopes such as ¹³C and ¹⁵N, polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g. radioisotope labels or fluorescent labels) or, in the case of an enzymatic label, may catalyze chemical alteration of a substrate compound or composition which is detectable. The labels can be suitable for small-scale detection or more suitable for high-throughput screening. As such, suitable labels include, but are not limited to magnetically active isotopes, non-radioactive isotopes, radioisotopes, fluorochromes, luminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component.

Examples of luminescent labels that produce signals include, but are not limited to bioluminescence and chemiluminescence. Detectable luminescence response generally comprises a change in, or an occurrence of, a luminescence signal. Suitable methods and luminophores for luminescently labeling assay components are known in the art and described for example in Haugland, Richard P. (1996) Handbook of Fluorescent Probes and Research Chemicals (6^{th} ed.). Examples of luminescent probes include, but are not limited to, aequorin and luciferases.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6^{th} ed.).

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in an eukaryotic cell.

“Differentially expressed” intends an up- or downward expression of a gene, exosome or microvesicle, or marker, for example, as compared to a control. In one aspect, a control is a differentiated cell as compared to a pluripotent or stem cell. “Differentially expressed” as applied to a gene, protein, cell, population, exosome or microvesicle, miRNA, or marker, refers to the differential production of the product as compared to a control such as expression level found in the native environment. Differently expressed is mRNA transcribed from the gene or the protein product encoded by the gene. A differentially expressed gene may be overexpressed or underexpressed (a.k.a. inhibited) as compared to the expression level of a normal, non-treated, native or control cell. In one aspect, it refers to overexpression that is 1.5 times, or alternatively, 2 times, or alternatively, at least 2.5 times, or alternatively, at least 3.0 times, or alternatively, at least 3.5 times, or alternatively, at least 4.0 times, or alternatively, at least 5 times, or alternatively 10 times higher (i.e., and therefore overexpressed) or lower than the expression level detected in a control sample. The term “differentially expressed” also refers to nucleotide sequences in a cell or tissue which are expressed where silent in a control cell or not expressed where expressed in a control cell.

The term “stem cell” refers to a cell that is in an undifferentiated or partially differentiated state and has the capacity for self-renewal and/or to generate differentiated progeny. Self-renewal is defined as the capability of a stem cell to proliferate and give rise to more such stem cells, while maintaining its developmental potential (i.e., totipotent, pluripotent, multipotent, etc.). The term “somatic stem cell” is used herein to refer to any stem cell derived from non-embryonic tissue, including fetal, juvenile, and adult tissue. Natural somatic stem cells have been isolated from a wide variety of adult tissues including blood, bone marrow, brain, olfactory epithelium, skin, pancreas, skeletal muscle, and cardiac muscle. Exemplary naturally occurring somatic stem cells include, but are not limited to, mesenchymal stem cells (MSCs) and neural stem cells (NSCs). In some embodiments, the stem or progenitor cells can be embryonic stem cells. As used herein, “embryonic stem cells” refers to stem cells derived from tissue formed after fertilization but before the end of gestation, including pre-embryonic tissue (such as, for example, a blastocyst), embryonic tissue, or fetal tissue taken any time during gestation, typically but not necessarily before approximately 10-12 weeks gestation. Most frequently, embryonic stem cells are pluripotent cells derived from the early embryo or blastocyst. Embryonic stem cells can be obtained directly from suitable tissue, including, but not limited to human tissue, or from established embryonic cell lines. “Embryonic-like stem cells” refer to cells that share one or more, but not all characteristics, of an embryonic stem cell.

“Differentiation” describes the process whereby an unspecialized cell acquires the features of a specialized cell such as a heart, liver, or muscle cell. “Directed differentiation” refers to the manipulation of stem cell culture conditions to induce differentiation into a particular cell type. “Dedifferentiated” defines a cell that reverts to a less committed position within the lineage of a cell. As used herein, the term “differentiates or differentiated” defines a cell that takes on a more committed (“differentiated”) position within the lineage of a cell. As used herein, “a cell that differentiates into a mesodermal (or ectodermal or endodermal) lineage” defines a cell that becomes committed to a specific mesodermal, ectodermal or endodermal lineage, respectively. Examples of cells that differentiate into a mesodermal lineage or give rise to specific mesodermal cells include, but are not limited to, cells that are adipogenic, leiomyogenic, chondrogenic, cardiogenic, dermatogenic, hematopoetic, hemangiogenic, myogenic, nephrogenic, urogenitogenic, osteogenic, pericardiogenic, or stromal.

As used herein, the term “differentiates or differentiated” defines a cell that takes on a more committed (“differentiated”) position within the lineage of a cell. “Dedifferentiated” defines a cell that reverts to a less committed position within the lineage of a cell. Induced pluripotent stem cells are examples of dedifferentiated cells.

As used herein, the “lineage” of a cell defines the heredity of the cell, i.e. its predecessors and progeny. The lineage of a cell places the cell within a hereditary scheme of development and differentiation.

A “multi-lineage stem cell” or “multipotent stem cell” refers to a stem cell that reproduces itself and at least two further differentiated progeny cells from distinct developmental lineages. The lineages can be from the same germ layer (i.e. mesoderm, ectoderm or endoderm), or from different germ layers. An example of two progeny cells with distinct developmental lineages from differentiation of a multilineage stem cell is a myogenic cell and an adipogenic cell (both are of mesodermal origin, yet give rise to different tissues). Another example is a neurogenic cell (of ectodermal origin) and adipogenic cell (of mesodermal origin).

A “precursor” or “progenitor cell” intends to mean cells that have a capacity to differentiate into a specific type of cell. A progenitor cell may be a stem cell. A progenitor cell may also be more specific than a stem cell. A progenitor cell may be unipotent or multipotent. Compared to adult stem cells, a progenitor cell may be in a later stage of cell differentiation. An example of progenitor cell includes, without limitation, a progenitor nerve cell.

A “parthenogenetic stem cell” refers to a stem cell arising from parthenogenetic activation of an egg. Methods of creating a parthenogenetic stem cell are known in the art. See, for example, Cibelli et al. (2002) Science 295(5556):819 and Vrana et al. (2003) Proc. Natl. Acad. Sci. USA 100 (Suppl. 1) 11911-6.

As used herein, a “pluripotent cell” defines a less differentiated cell that can give rise to at least two distinct (genotypically and/or phenotypically) further differentiated progeny cells. In another aspect, a “pluripotent cell” includes an Induced Pluripotent Stem Cell (iPSC) which is an artificially derived stem cell from a non-pluripotent cell, typically an adult somatic cell, that has historically been produced by inducing expression of one or more stem cell specific genes. Such stem cell specific genes include, but are not limited to, the family of octamer transcription factors, i.e. Oct-3/4; the family of Sox genes, i.e., Sox1, Sox2, Sox3, Sox 15 and Sox 18; the family of Klf genes, i.e. Klf1, Klf2, Klf4 and Klf5; the family of Myc genes, i.e. c-myc and L-myc; the family of Nanog genes, i.e., OCT4, NANOG and REX1; or LIN28. Examples of iPSCs are described in Takahashi et al. (2007) Cell advance online publication 20 Nov. 2007; Takahashi & Yamanaka (2006) Cell 126:663-76; Okita et al. (2007) Nature 448:260-262; Yu et al. (2007) Science advance online publication 20 Nov. 2007; and Nakagawa et al. (2007) Nat. Biotechnol. Advance online publication 30 Nov. 2007.

“Embryoid bodies or EBs” are three-dimensional (3D) aggregates of embryonic stem cells formed during culture that facilitate subsequent differentiation. When grown in suspension culture, EBs cells form small aggregates of cells surrounded by an outer layer of visceral endoderm. Upon growth and differentiation, EBs develop into cystic embryoid bodies with fluid-filled cavities and an inner layer of ectoderm-like cells.

An “induced pluripotent cell” intends embryonic-like cells reprogrammed to the immature phenotype from adult cells. Various methods are known in the art, e.g., “A simple newway to induce pluripotency: Acid.” Nature, 29 Jan. 2014 and available at sciencedaily.com/releases/2014/01/140129184445, last accessed on Feb. 5, 2014 and U.S. Patent Application Publication No. 2010/0041054. Human iPSCs also express stem cell markers and are capable of generating cells characteristic of all three germ layers.

As used herein, the term “a cardiac progenitor” intends a dynamic progenitor that is able to differentiate into terminally derived cardiac cell types. Cardiac progenitor cells (CPCs) represent the earliest stages of mesodermal commitment to the cardiac lineage and show a classical CPC marker pro le of KDR/PDGFR-αpos/CKITneg and are responsive to permissive conditions for proliferation as a progenitor population and/or differentiation into terminal cardiac cell.

As used herein, the term “a skeletal myogenic progenitor” intends cells which are characterized by the expression of Pax3 and Pax7 and also give rise to the satellite cells of postnatal muscle.

As used herein, a “fibroblast” intends a cell expressing the following markers Vimentin, CollA1, FSP-1.

As used herein, a “skeletal myoblast” intends a cell expressing the following markers MyoG, Desmin, m-calpain, human alpha-skeletal actin.

As used herein, a “pluripotent cell” defines a less differentiated cell that can give rise to at least two distinct (genotypically and/or phenotypically) further differentiated progeny cells.

A juvenile or young stem cells intends from 2 months or younger mice possessing antiaging genes. In vitro, the cells average from about 3-5 μm in size and express the embryonic stem cell marker, OCT4 and surface markers, CD29, CD44, and CD90.

As used herein, a “fibroblast” intends a cell expressing the following markers Vimentin, CollA1, FSP-1.

As used herein, a “skeletal myoblast” intends a cell expressing the following markers MyoG, Desmin, m-calpain, human alpha-skeletal actin.

As used herein, the term “pluripotent gene or marker” intends an expressed gene or protein that has been correlated with an immature or undifferentiated phenotype, e.g., Oct ¾, Sox2, Nanog, c-Myc and LIN-28. Methods to identify such are known in the art and systems to identify such are commercially available from, for example, EMD Millipore (MILLIPLEX® Map Kit).

A “skeletal myoblast (SM)” is an immature cell that can be isolated from between the basal lamina and sarcolemma. They account for 2-5% of sub-laminar nuclei of mature skeletal muscle. Skeletal myoblasts are activated in response to muscle damage or disease-induced muscle degeneration. Skeletal myoblasts express desmin, CD56, Pax3, Pax7, c-met, myocyte nuclear factor, M-cadherin, VCAM1, N-CAM, CD34, Leu-19, and syndecan 3 and 4. Activated skeletal myoblasts first express Myf-5 and/or MyoD, and finally myogenin and MRF4 as the cells differentiate into multinucleated myotubes.

As used herein, the term “small juvenile stem cells (SJSCs)” intends stem cells isolated from aged bone marrow-derived stem cells (BMSCs) with high proliferation and differentiation potential. See Igura et al. (2013) 305(8):H1354-62. SJSCs express mesenchymal stem cell markers, CD29(+)/CD44(+)/CD59(+)/CD90(+), but are negative for CD45(−)/CD117(−) as examined by flow cytometry analysis. SJSCs show higher proliferation, colony formation, and differentiation abilities compared with BMSCs. They also are reported to significantly express cardiac lineage markers (Gata-4 and myocyte-specific enhancer factor 2C) and pluripotency markers (octamer-binding transcription factor 4, sex-determining region Y box 2, stage-specific embryonic antigen 1, and Nanog) as well as antiaging factors such as telomerase reverse transcriptase and sirtuin 1.

A “marrow stromal cell” also referred to as “a bone marrow stromal cell” or a “mesenchymal stromal cell” is a multipotent stem cell that can differentiate into a variety of cell types. Cell types that MSCs have been shown to differentiate into in vitro or in vivo include osteoblasts, chondrocytes, myocytes, and adipocytes. Mesenchyme is embryonic connective tissue that is derived from the mesoderm and that differentiates into hematopoietic and connective tissue, whereas MSCs do not differentiate into hematopoietic cells. Stromal cells are connective tissue cells that form the supportive structure in which the functional cells of the tissue reside. While this is an accurate description for one function of MSCs, the term fails to convey the relatively recently-discovered roles of MSCs in repair of tissue. Methods to isolate such cells, propagate and differentiate such cells are known in the technical and patent literature, e.g., U.S. Patent Application Publication Nos. 2007/0224171, 2007/0054399, 2009/0010895, which are incorporated by reference in their entireties.

Adipose stem cells are also known as adipose tissue-derived stem cells (ADSC) that are routinely isolated from the stromal vascular fraction (SVF) of homogenized adipose tissue. Similar to other types of mesenchymal stem cells (MSC), ADSC remain difficult to define due to the lack of definitive cellular markers. Adipose-derived stem cells (ASCs) are a mesenchymal stem cell source with self-renewal property and multipotential differentiation.

Hematopoietic stem cells are defined as a stem cell that gives rise to all red and white blood cells and platelets. They are commonly isolated by use of the markers CD34+. In another aspect, the hematopoietic stem cell is an adult stem cell comprising the marker profile of: CD34⁺ and/or CD34⁺/Thy-1⁻ HSC). See also Andrews, R. G. et al. (1990) J. Exp. Med. 172(1):355-358, incorporated herein by reference.

Mesenchymal stem cells, or MSCs, are defined as multipotent stromal cells that can differentiate into a variety of cell types, including: osteoblasts (bone cells), chondrocytes (cartilage cells), myocytes (muscle cells) and adipocytes (fat cells).

As used herein, the term chemically induced or chemically modified pluripotent stem cell (iPSC) is intended to include an iPSC treated with a small molecule such as isoxazole or a derivative thereof, or an isoxazole similar molecule.

“Isoxazole” is a class of compounds found in some natural products, such as ibotenic acid, as well as a number of drugs, including a COX-2 inhibitor, and furoxan, a nitric oxide donor. Isoxazoles are useful isosteres of pyridine, and have been found to inhibit voltage-gated sodium channels to control pain, enable the construction of tetracycline antibiotic derivatives, and as treatments for depression. Compounds of this class available from Sigma-Aldrich and methods to synthesize such are known in the art as described for example in U.S. Pat. Nos. 5,059,614 and 8,318,951 and PCT Publication No. WO 1999/002507. Structurally, isoxazole is a five membered heterocyclic compound containing oxygen and nitrogen atoms in the 1, 2 positions. Its partially saturated analogs are called isoxazolines and completely saturated analog is isoxazolidine. Examples of isoxazole-like compounds include derivatives, non-limiting examples of such include sulfamethoxazole, sulfisoxazole, oxacillin, cycloserine and acivicin. isoxazoles, isoxazolines and isoxazolidines may be considered as useful synthons in organic synthesis. Isoxazoles may be efficiently transformed in to various classes of medicinally important molecules. For example, Anthracen-9-ylmethylene-(3,4-dimethylisoxazol-5-yl) amine may be synthesized in high yield by reaction of anthracene-9-carbaldehyde and 5-amino-3,4-dimethylisoxazole in ethanol. In an embodiment, all the derivatives of isoxazole may be considered as “isoxazole-like compound” or “similar compound”. In an embodiment, the isoxazole derivatives such as 5-Amino-3-methyl-4-isoxazolecarboxylic acid semicarbazides and thiosemicarbazides may be synthesized. The reaction of 5-amino-3-methyl-4-isoxazolecarboxylic acid hydrazide with isocyanates and isothiocyanates may be designed and conducted. The isocyanates, in the reaction of nucleophilic addition with compounds containing the primary amino group, form urea derivatives and isothiocyanates the thiourea derivatives. Only the hydrazide terminal group (—NH2) participates in this reaction. The amino group in position 5 of isoxazole ring remains not reactive under the reaction conditions. The mechanism of the reaction consists in nucleophilic attack of the nitrogen atom in the hydrazide group (—NH2) on the carbon atom of isocyanate or isothiocyanate. The intermediate forms appear which undergo amidoiminole tautomerization leading to formation of substituted 5-amino-3-methyl-4-isoxazolecarboxylic acid semicarbazides and thiosemicarbazides. In an embodiment, examples of isoxazole derivatives may comprise 5-sulfanilamido-isoxazoles of the general formula wherein R and R are lower alkyl and/or lower alkoxy alkyl groups. Sulfanilamide derivatives with the isoxazole ring attached in N-position of the sulfanilamide molecule may be generated. For example, sulfanilamide radical in 4-position of the isoxazole ring. Further, a sulfanilyl derivative of 5-amino-isoxazole namely, 5-sulfanilamido-3-methyl-isoxazole may also be considered as an isoxazole derivative. In an embodiment, both the 3- and 4-positions of the isoxazole ring of the sulfanilamide derivatives may be replaced by an alkyl and/or corresponding alkoxy alkyl radical to generate. Non-limiting examples of “isoxazole-like compound” or “similar compound” comprise 1,2-oxazole, 4-deuterio-1,2-oxazole, 1,2-oxazole; potassium, hydron; 1,2-oxazole, 1-oxido-1,2-oxazol-1-ium, 1,2-oxazole; hydrobromide, 1,2-oxazole; hydrochloride, ethane; 1,2-oxazole, potassium; 1,2-oxazole; hydroxide, 1,2-oxazole; hydrate; hydrochloride, ethane; 1,2-oxazole, 1,2-oxazole; cyanate, 2-oxido-1,2-oxazol-2-ium, carbon monoxide; chromium; 1,2-oxazole, ethane; 1,2-oxazole, ethane; 1,2-oxazole; propane, 1,2-oxazol-2-ium-2-sulfonate, carbonyl dichloride; 1,2-oxazole, isocyanic acid; 1,2-oxazole, ethoxyethane; 1,2-oxazole, 2,2-dimethylpropane; ethane; 1,2-oxazole, ethane; methoxyethane; 1,2-oxazole, ethane; 2-methylpropane; 1,2-oxazole, 1,2-oxazole; urea, ethanol; 1,2-oxazole, carbonic acid; 1,2-oxazole, 1,2-oxazol-1-ium-1-sulfonic acid, 1,2-oxazol-2-ium; iodide.

“Isoxazole 9” (ISX-9) is a small molecule inducer of adult neural stem cell differentiation both in vitro and in vivo (Schneider et al.). It has been shown to act through a calcium-activated signaling pathway dependent on myocyte-enhancer factor 2 (MEF2)-dependent gene expression (Schneider et al.; Petrik et al.). Compounds are also available from Sigma-Aldrich and StemCell Technologies. The molecular formula is C₁₁H₁₀N₂O₂S, and the chemical name is N-cyclopropyl-5-thiophen-2-yl-1,2-oxazole-3-carboxamide. The two dimensional structure is:

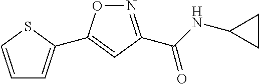 [SMILES: O=C(NC1CC1)c1cc(-c2cccs2)on1]

“Isoxazole 1” (ISX-1) is a small molecule having the structure:

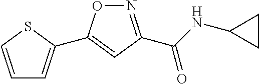 [SMILES: O=C(NC1CC1)c1cc(-c2cccs2)on1]

As used herein, the term “isoxazole-like compound” or “similar compound” intends an agent or small molecule that has the same functional property of the isoxazole as disclosed herein. Non-limiting examples include Cardionogen; CDNG1/vuc230, CDNG2/vuc198, and CDNG3/vuc247 (see Terri et al. (2011) Chem Biol., December 23 18(12):1658-1668). Non-limiting examples further include sulfisoxazole as described herein below. Yet a further example is leflunomide (Arava), also known as 5-methyl-N-[4-(trifluoromethyl)phenyl]-1,2-oxazole-4-carboxamide.

An isoxazole compound or derivative thereof can also be a compound of the formula:

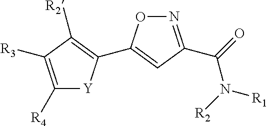

wherein R₁ and R₂ are both hydrogen or R₁ is hydrogen and R₂ is selected from the group consisting of substituted or unsubstituted C₁-C₆ alkyl, C₃-C₆ cycloalkyl, C₂-C₆ alkenyl, C₂-C₆ alkynyl, and benzyl, or where R₁ and R₂ may be joined together to form a ring selected from azetidinyl, pyrrolidinyl, piperidinyl, piperazinyl, or morpholinyl; R₂′, R₃ and R₄ are independently selected from the group consisting of hydrogen, halogen, C₁-C₆ alkyl, C₃-C₆ cycloalkyl, substituted or unsubstituted aromatic or heteroaromatic ring, cyano, nitro, and acyl; and Y is 0, NH or S.

In one aspect, an isoxazole compound has the formula:

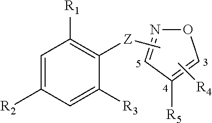

wherein R1 and R2 is each selected from C1-C4 alkyl, phenyl, benzyl, trifluoromethyl or halogen, R3 is selected from hydrogen, hydroxy, C1-C4 alkyl or alkoxy, R4, in position 3 or 5, is selected from hydrogen, trifluoromethyl, C1-C4 alkoxy, C1-C4 alkyl, or C1-C4 hydroxyalkyl, R5 is selected from hydrogen or C4-C4 alkyl or R4 and R5 together form a tetramethylene group, Z at position 3 or 5 on the heterocycle is selected from: —N(R6)-CO—, —CO—N(R6)-, —N(R6)—CO—N(R6)-, —CH(R6)-NH—CO—, or —NH—CO—CH(R6), in which R6 is selected from hydrogen or C1-C4 alkyl. Non-limiting examples include 5-(trifluoromethyl)-3-(4-methoxyphenyl)isoxazole-4-carboxylic acid, 5-(trifluoromethyl)-3-(4-fluorophenyl)isoxazole-4-carboxylic acid, 5-(thiophen-2-yl)isoxazole-3-carboxaldehyde, 5,6,7,8-tetrahydro-4h-cyclohepta[d]isoxazole-3-carboxylic acid, 4,5,6,7-tetrahydro-benzo[d]isoxazole-3-carboxylic acid, 3-amino-5-methylisoxazole, 4-amino-n-(5-methyl-3-isoxazolyl)benzenesulfonamide, 3-phenyl-isoxazole-5-boronic acid pinacol ester, 5-phenylisoxazole, 1-phenyl-1-cyclopentanecarboxylic acid, 3-phenyl-benzo[c]isoxazole-5-carboxylic acid, 5-methyl-3-phenylisoxazole-4-carboxylic acid, 3a,4,5,6,7,8,9,9a-octahydro-cycloocta[d]isoxazole-3-carboxylic acid, 5-(3-nitrophenyl)isoxazole, 3-(4-nitrophenyl)isoxazole, 3-hydroxy-5-aminomethyl-isoxazole, 5-(morpholinomethyl)isoxazole-3-carboxylic acid hydrochloride, 5-(morpholinomethyl)isoxazole-3-carbaldehyde, 3-methyl-5-(trifluoromethyl)isoxazole-4-carboxylic acid, methyl 5-(thiophen-2-yl)isoxazole-3-carboxylate, 3-(methylsulfonyl)-5-(2-thienyl)isoxazole-4-carbonitrile, 5-methyl-3-(2-pyrrolidinyl)isoxazole, 3-methyl-5-(2-pyrrolidinyl)isoxazole, 3-(1-methyl-1h-pyrazol-4-yl)-isoxazole-5-carboxylic acid, 3-(1-methyl-1h-pyrazol-4-yl)-4,5-dihydro-isoxazole-5-carboxylic acid, 5-(4-methylphenyl)isoxazole-3-carboxylic acid, 5-methyl-3-phenylisoxazole-4-carboxylic acid, 5-(4-methylphenyl)isoxazole-3-carboxaldehyde, 5-methyl-3-(4-phenoxyphenyl)isoxazole-4-carboxylic acid, 3-methyl-5-(4-methyl-1,2,3-thiadiazol-5-yl)isoxazole-4-carboxylic acid, 3-methyl-5-(5-methylisoxazol-3-yl)isoxazole-4-carboxylic acid, methyl 5-(4-methoxyphenyl)isoxazole-4-carboxylate, methyl 5-(4-methoxyphenyl)isoxazole-3-carboxylate, 5-methylisoxazole, methyl 5-(4-fluorophenyl)isoxazole-4-carboxylate, methyl 5-(4-fluorophenyl)isoxazole-3-carboxylate, methyl 5-(4-chlorophenyl)isoxazole-4-carboxylate, methyl 5-(4-bromophenyl)isoxazole-4-carboxylate, 5-(4-methoxyphenyl)isoxazole-3-carboxylic acid, 5-(3-methoxy-phenyl)-isoxazole-3-carboxylic acid, 3-(2-methoxyphenyl)isoxazole-5-carboxylic acid, 5-(4-methoxyphenyl)isoxazole-3-carboxaldehyde, 3-(4-methoxyphenyl)isoxazole-5-carbaldehyde, 3-(2-methoxyphenyl)isoxazole-5-carbaldehyde, 5-(4-methoxyphenyl)isoxazole, 3-(4-methoxyphenyl)isoxazole, 3-(2-methoxy-phenyl)-4,5-dihydro-isoxazole-5-carboxylic acid, 3-methoxy-isoxazole-5-carboxylic acid, isoxazole-5-carboxylic acid, isoxazole-4-carboxylic acid, isoxazole-5-carbothioamide, isoxazole-5-carbonyl chloride, isoxazole-3-carbonitrile, isoxazole-3-carbaldehyde, isoxazole-4-boronic acid, isoxazole, 5-cyclopropyl-4-[2-(methylsulfonyl)-4-(trifluoromethyl)benzoyl]isoxazole, 6-(5-(thiophen-2-yl)isoxazole-3-carboxamido)hexyl 5-((3as,4s,6ar)-2-oxohexahydro-1h-thieno[3,4-d]imidazol-4-yl)pentanoate, isocarboxazid 5-methyl-3-isoxazole-carboxylic acid 2-benzylhydrazide, 5-isobutyl-isoxazole-3-carboxylic acid, 4-iodo-5-methyl-isoxazole, 3,3′-iminobis(n,n-dimethylpropylamine), 3-(3-hydroxy-phenyl)-isoxazole-5-carboxylic acid methyl ester, 5-(4-hydroxy-phenyl)-isoxazole-3-carboxylic acid, 5-(3-hydroxy-phenyl)-isoxazole-3-carboxylic acid, 5-(hydroxymethyl)-3-methylisoxazole, 3-hydroxy-5-methylisoxazole, 5-(1-hydroxyethyl)-3-(4-trifluoromethylphenyl)isoxazole, 3a,4,5,6,7,7a-hexahydro-benzo[d]isoxazole-3-carboxylic acid, 5-(2-furyl)isoxazole-3-carbaldehyde, 5-furan-2-yl-isoxazole-3-carboxylic acid, 6-fluoro-3-(4-piperidinyl)benzisoxazole, 5-(4-fluorophenyl)isoxazole-3-methanol, 3-(2-fluoro-phenyl)-isoxazole-5-carboxylic acid, 5-(4-fluorophenyl)isoxazole-3-carboxaldehyde, 3-(4-fluorophenyl)isoxazole-5-carbaldehyde, 3-(3-fluorophenyl)isoxazole-5-carbaldehyde, 3-(2-fluorophenyl)isoxazole-5-carbaldehyde, 5-(4-fluorophenyl)isoxazole, 3-(4-fluorophenyl)isoxazole, 5-(3-fluoro-4-methoxy-phenyl)-isoxazole-3-carboxylic acid, ethyl 5-(trifluoromethyl)-3-(4-methoxyphenyl)isoxazole-4-carboxylate, ethyl-5-(tributylstannyl)isoxazole-3-carboxylate, ethyl 5-(thiophen-2-yl)isoxazole-3-carboxylate, 5-ethyl-isoxazole-4-carboxylic acid, 5-ethyl-isoxazole-3-carboxylic acid, ethyl 5-(4-fluorophenyl)isoxazole-4-carboxylate, ethyl 5-(4-fluorophenyl)isoxazole-3-carboxylate, ethyl 5-(2,3-dihydrobenzo[b][1,4]dioxin-7-yl)isoxazole-3-carboxylate, ethyl 3-(4-chlorophenyl)-5-(trifluoromethyl)isoxazole-4-carboxylate, ethyl 5-(4-chlorophenyl)isoxazole-3-carboxylate, ethyl 3-(4-bromophenyl)-5-(trifluoromethyl)isoxazole-4-carboxylate, ethyl 5-(4-bromophenyl)isoxazole-3-carboxylate, ethyl 5-amino-4-(4-chlorophenyl)isoxazole-3-carboxylate, ethyl 5-amino-4-(4-bromophenyl)isoxazole-3-carboxylate, ethyl 6b-acetyl-2-(acetyloxy)-4a,6a-dimethyl-2,3,4,4a,4b,5,6,6a,6b,9a,10,10a,10b,11-tetradecahydro-1h-naphtho[2′,1′:4,5]indeno[2,1-d]isoxazole-9-carboxylate, 3,5-dimethyl-4-(tributylstannyl)isoxazole, 5-(1,5-dimethyl-1h-pyrazol-4-yl)-isoxazole-3-carboxylic acid, 5-(1,3-dimethyl-1h-pyrazol-4-yl)-isoxazole-3-carboxylic acid, 5-(1,5-dimethyl-1h-pyrazol-4-yl)-isoxazole, 3,5-dimethylisoxazole-4-boronic acid pinacol ester, 3,5-dimethylisoxazole, 3-(dimethylamino)-1-(2-pyridyl)-2-propen-1-one, 5-(3,5-difluorophenyl)isoxazole, [2,6-dichloro-4-(trifluoromethyl)phenyl]hydrazine, 5-(2,5-dichlorophenyl)isoxazole-3-carboxylic acid, danazol, 3-(4-chlorophenyl)-5-(trifluoromethyl)isoxazole-4-carboxylic acid, 5-(4-chlorophenyl)isoxazole-3-propionic acid, 5-(4-chlorophenyl)isoxazole-4-carboxylic acid, 5-(4-chlorophenyl)isoxazole-3-carboxylic acid, 3-(4-chlorophenyl)isoxazole-5-carboxylic acid, 3-(3-chlorophenyl)isoxazole-5-carboxylic acid, 5-(4-chlorophenyl)isoxazole-3-carboxaldehyde, 3-(4-chlorophenyl)isoxazole-5-carbaldehyde, 3-(3-chlorophenyl)isoxazole-5-carbaldehyde, 3-(2-chlorophenyl)isoxazole-5-carbaldehyde, 5-(4-chlorophenyl)isoxazole, 3-(4-chlorophenyl)isoxazole, 5-(chloromethyl)isoxazole-4-carboxylic acid, 3-(chloromethyl)-5-(2-furyl)isoxazole, 4-chloromethyl-3,5-dimethylisoxazole, 5-(chloromethyl)-3-(4-chlorophenyl)isoxazole, 5-(3-chloro-4-methoxy-phenyl)-isoxazole-3-carboxylic acid, 3-chloro-4-fluorobenzaldehyde, 3-(5-chloro-2,4-dimethoxy-phenyl)-4,5-dihydro-isoxazole-5-carboxylic acid, 5-tert-butyl-4,5,6,7-tetrahydro-benzo[d]isoxazole-3-carboxylic acid, 3-(4-bromophenyl)-5-(trifluoromethyl)isoxazole-4-carboxylic acid, 5-(4-bromophenyl)isoxazole-3-propionic acid, 5-(4-bromophenyl)isoxazole-3-carboxylic acid hydrazide, 5-(4-bromophenyl)isoxazole-4-carboxylic acid, 5-(4-bromophenyl)isoxazole-3-carboxylic acid, 3-(4-bromophenyl)isoxazole-5-carboxylic acid, 3-(4-bromophenyl)isoxazole-5-carboxaldehyde, 5-(4-bromophenyl)isoxazole, 5-(3-bromophenyl)isoxazole, 3-(4-bromophenyl)isoxazole, 5-(bromomethyl)-3-(4-methoxyphenyl)isoxazole, 4-(bromomethyl)isoxazole, 5-(bromomethyl)-3-(4-fluorophenyl)isoxazole, 5-(bromomethyl)-3-(4-chlorophenyl)isoxazole, 5-(bromomethyl)-3-(4-bromophenyl)isoxazole, 6-bromo-3-methylbenzo[d]isoxazole, 5-bromo-3-methylbenzo[d]isoxazole, 4-bromo-5-(4-methoxyphenyl)isoxazole, 3-bromo-isoxazole, 3-bromo-5-(2-hydroxyethyl)isoxazole, 4-bromo-5-(4-fluorophenyl)isoxazole, 3-bromo-5-(4-fluorophenyl)isoxazole, 4-bromo-5-(4-chlorophenyl)isoxazole, 4-bromo-5-(4-bromophenyl)isoxazole, 6-bromo-benzo[d]isoxazole-3-carboxylic acid, benzo[d]isoxazole-3-carboxylic acid, 3-amino-5-methylisoxazole, 5-amino-3-(4-methoxyphenyl)isoxazole, 3-aminoisoxazole, 3-amino-5-(4-fluorophenyl)isoxazole, 5-amino-3-(4-chlorophenyl)isoxazole, 5-amino-4-(4-bromophenyl)isoxazole, 3-amino-5-(4-bromophenyl)isoxazole, 5-acetyl-3-(4-fluorophenyl)isoxazole, 5-acetyl-3-(3-fluorophenyl)isoxazole, 3-methyl-5-[(2s)-1-methyl-2-pyrrolidinyl]isoxazole hydrochloride, 7-methoxy-5-methyl-4,5-dihydronaphtho[2,1-d]isoxazole, 5-methyl-3-phenyl-isoxazole-4-carboxylic acid methylamide, 5-methyl-3-phenyl-isoxazole-4-carbothioic acid methylamide, 5-methyl-3-phenyl-4-(1h-pyrazol-5-yl)isoxazole, 5-benzyl-3-furan-2-yl-2-phenyl-tetrahydro-pyrrolo(3,4-d)isoxazole-4,6-dione, 5-benzyl-3-[4-(dimethylamino)phenyl]-2-phenyldihydro-2h-pyrrolo[3,4-d]isoxazole-4,6(3h,5h)-dione, 5-benzyl-3-(5-br-2-ho-phenyl)-2-ph-tetrahydro-pyrrolo(3,4-d)isoxazole-4,6-dione, 5-benzyl-3-(4-nitro-ph)-2-ph-dihydro-2h-pyrrolo(3,4-d)isoxazole-4,6(3h,5h)-dione, 5-benzyl-3-(4-methoxy-phenyl)-2-ph-tetrahydro-pyrrolo(3,4-d)isoxazole-4,6-dione, 5-benzyl-3-(4-fluorophenyl)-2-(2-methylphenyl)dihydro-2h-pyrrolo[3,4-d]isoxazole-4,6(3h,5h)-dione, 5-benzyl-3-(3-nitro-ph)-2-phenyl-tetrahydro-pyrrolo(3,4-d)isoxazole-4,6-dione, 5-benzyl-2-ph-3-(2-pyridinyl)dihydro-2h-pyrrolo(3,4-d)isoxazole-4,6(3h,5h)-dione, 5-benzyl-2-ph-3-(2-ph-vinyl)dihydro-2h-pyrrolo(3,4-d)isoxazole-4,6(3h,5h)-dione, 5-benzyl-2-(4-chlorophenyl)-3-(2-thienyl)dihydro-2h-pyrrolo[3,4-d]isoxazole-4,6(3h,5h)-dione, 5-benzyl-2,3-diphenyldihydro-2h-pyrrolo(3,4-d)isoxazole-4,6(3h,5h)-dione, 5-benzyl-2(4-cl-ph)3-(2-furyl)dihydro-2h-pyrrolo(3,4-d)isoxazole-4,6(3h,5h)dione, 5-benzyl-2(4-cl-ph)-3-(4-f-ph)dihydro-2h-pyrrolo(3,4-d)isoxazole-4,6(3h,5h)dione, 5-(p-tolyl)isoxazole, 5-(4-methylphenyl)-3-(4-nitrophenyl)-2-phenyldihydro-2h-pyrrolo[3,4-d]isoxazole-4,6(3h,5h)-dione, 5-(4-methoxyphenyl)-2-phenyl-3-(4-pyridinyl)dihydro-2h-pyrrolo[3,4-d]isoxazole-4,6(3h,5h)-dione, 5-(4-methoxyphenyl)-2-phenyl-3-(3-pyridinyl)dihydro-2h-pyrrolo[3,4-d]isoxazole-4,6(3h,5h)-dione, 5-(4-methoxy-ph)-2,3-diphenyldihydro-2h-pyrrolo(3,4-d)isoxazole-4,6(3h,5h)-dione, 5-(4-fluorophenyl)-3-(4-methoxyphenyl)-2-phenyldihydro-2h-pyrrolo[3,4-d]isoxazole-4,6(3h,5h)-dione, 5-(4-fluorophenyl)-2-(2-methylphenyl)-3-(4-pyridinyl)dihydro-2h-pyrrolo[3,4-d]isoxazole-4,6(3h,5h)-dione, 5-(4-ethoxyphenyl)-3-(4-nitrophenyl)-2-phenyldihydro-2h-pyrrolo[3,4-d]isoxazole-4,6(3h,5h)-dione, 5-(4-ethoxyphenyl)-3-(4-fluorophenyl)-2-phenyldihydro-2h-pyrrolo[3,4-d]isoxazole-4,6(3h,5h)-dione, 5-(4-ethoxyphenyl)-2-methyl-3-(4-nitrophenyl)dihydro-2h-pyrrolo[3,4-d]isoxazole-4,6(3h,5h)-dione, 5-(4-ethoxy-ph)-2-ph-3-thiophen-2-yl-4h-pyrrolo(3,4-d)isoxazole-4,6-dione, 5-(4-cl-ph)-3-(3-nitro-ph)-2-phenyl-tetrahydro-pyrrolo(3,4-d)isoxazole-4,6-dione, 5-(4-bromophenyl)-3-(4-methoxyphenyl)-2-phenyldihydro-2h-pyrrolo[3,4-d]isoxazole-4,6(3h,5h)-dione, 5-(4-bromophenyl)-3-(2-furyl)-2-(2-methylphenyl)dihydro-2h-pyrrolo[3,4-d]isoxazole-4,6(3h,5h)-dione, 5-(4-bromophenyl)-2-phenyl-3-(2-pyridinyl)dihydro-2h-pyrrolo[3,4-d]isoxazole-4,6(3h,5h)-dione, 5-(4-br-ph)2-ph-3-(2-ph-vinyl)dihydro-2h-pyrrolo(3,4-d)isoxazole-4,6(3h,5h)dione, 5-(2-cl-ph)-3-(4-dimethylamino-ph)2-o-tolyl-4h-pyrrolo(3,4-d)isoxazole-4,6-dione, 5-(2-chlorophenyl)-3-[4-(dimethylamino)phenyl]-2-phenyldihydro-2h-pyrrolo[3,4-d]isoxazole-4,6(3h,5h)-dione, 5-(2-chlorophenyl)-3-(4-methoxyphenyl)-2-phenyldihydro-2h-pyrrolo[3,4-d]isoxazole-4,6(3h,5h)-dione, 4-((3-(2-cl-ph)-5-methyl-isoxazole-4-carbonyl)-amino)-benzoic acid ethyl ester, 4,5,6,6a-tetrahydro-3ah-cyclopenta[d]isoxazole-3-carboxylic acid, 3-phenyl-3a,6a-dihydrothieno[2,3-d]isoxazole 4,4-dioxide, 3-methyl-5-(3-phenylpropyl)isoxazole, 3-methyl-4-nitro-5-[(e)-2-phenylethenyl]isoxazole, 3-methyl-4,5,8,9-tetrahydrocycloocta(d)isoxazole, 3-methyl-4,5,5a,6a,7,8-hexahydrooxireno(2′,3′:5,6)cycloocta(1,2-d)isoxazole, 3-methyl-3a,4,5,8,9,9a-hexahydrocycloocta(d)isoxazole, 3-furan-2-yl-2-phenyl-5-p-tolyl-tetrahydro-pyrrolo(3,4-d)isoxazole-4,6-dione, 3-chloro-4,5-dihydro(1)-benzothiepino(5,4-c)isoxazole, 3-[4-(dimethylamino)phenyl]-5-(4-methoxyphenyl)-2-(2-methylphenyl)dihydro-2h-pyrrolo[3,4-d]isoxazole-4,6(3h,5h)-dione, 3-(5-br-2-ho-phenyl)-2,5-diphenyl-tetrahydro-pyrrolo(3,4-d)isoxazole-4,6-dione, 3-(5-br-2-ho-ph)-5-(2-cl-ph)-2-ph-tetrahydro-pyrrolo(3,4-d)isoxazole-4,6-dione, 3-(4-meo-phenyl)-5-phenyl-2-o-tolyl-tetrahydro-pyrrolo(3,4-d)isoxazole-4,6-dione, 3-(4-fluorophenyl)-5-(4-nitrophenyl)-2-phenyldihydro-2h-pyrrolo[3,4-d]isoxazole-4,6(3h,5h)-dione, 3-(4-fluorophenyl)-5-(4-methylphenyl)-2-phenyldihydro-2h-pyrrolo[3,4-d]isoxazole-4,6(3h,5h)-dione, 3-(4-dimethylamino-ph)-5-ph-2-o-tolyl-4h-pyrrolo(3,4-d)isoxazole-4,6-dione, 3-(4-fluorophenyl)-5-(4-methoxyphenyl)-2-phenyldihydro-2h-pyrrolo[3,4-d]isoxazole-4,6(3h,5h)-dione, 3-(4-br-ph)-2-ph-5-(2-trifluoromethyl-ph)-4h-pyrrolo(3,4-d)isoxazole-4,6-dione, 3-(3-nitro-phenyl)-2,5-diphenyl-tetrahydro-pyrrolo(3,4-d)isoxazole-4,6-dione, 3-(3-br-phenyl)-2,5-diphenyldihydro-2h-pyrrolo(3,4-d)isoxazole-4,6(3h,5h)-dione, 3-(3-br-ph)-5-(2-meo-ph)-2-o-tolyl-tetrahydro-pyrrolo(3,4-d)isoxazole-4,6-dione, 3-(2-furyl)-5-[4-(4-morpholinyl)phenyl]-2-phenyldihydro-2h-pyrrolo[3,4-d]isoxazole-4,6(3h,5h)-dione, 3-(2-furyl)-2-(2-me-ph)-5-ph-dihydro-2h-pyrrolo(3,4-d)isoxazole-4,6(3h,5h)-dione, 3-(2-furyl)-2,5-diphenyldihydro-2h-pyrrolo(3,4-d)isoxazole-4,6(3h,5h)-dione, 3-(2-cl-phenyl)-5-methyl-isoxazole-4-carboxylic acid (2,5-dichloro-phenyl)-amide, 3-(2-cl-ph)-5-me-isoxazole-4-carboxylic acid (4,5-dihydro-thiazol-2-yl)-amide, 3-(2-chloro-phenyl)-5-methyl-isoxazole-4-carboxylic acid cyanomethyl-amide, 3-(2,4-dichlorophenyl)-5-(4-methoxyphenyl)-2-phenyldihydro-2h-pyrrolo[3,4-d]isoxazole-4,6(3h,5h)-dione, 3-(2,4-di-cl-ph)-2,5-diphenyldihydro-2h-pyrrolo(3,4-d)isoxazole-4,6(3h,5h)-dione, 3-(2,2-dichloro-vinyl)-5-phenyl-isoxazole, 3,5-diphenyl-isoxazole, 3,5-dimethyl-4-(1-pyrrolidinylsulfonyl)isoxazole, 3(4-dimethylamino-ph)-5-(4-eto-ph)2-o-tolyl-4h-pyrrolo(3,4-d)isoxazole-4,6-dione, 2-(4-cl-ph)5-ph-3-(2-thienyl)dihydro-2h-pyrrolo(3,4-d)isoxazole-4,6(3h,5h)-dione, 2-(4-cl-ph)-5-(3-meo-ph)-3-(3-nitro-ph)-4h-pyrrolo(3,4-d)isoxazole-4,6-dione, 2-(4-cl-ph)-3-(4-meo-ph)-5-p-tolyl-tetrahydro-pyrrolo(3,4-d)isoxazole-4,6-dione, 2-(4-chlorophenyl)-5-(4-methylphenyl)-3-(2-thienyl)dihydro-2h-pyrrolo[3,4-d]isoxazole-4,6(3h,5h)-dione, 2-(4-chlorophenyl)-3-[4-(dimethylamino)phenyl]-5-phenyldihydro-2h-pyrrolo[3,4-d]isoxazole-4,6(3h,5h)-dione, 2-(4-chlorophenyl)-3-(4-fluorophenyl)-5-(4-nitrophenyl)dihydro-2h-pyrrolo[3,4-d]isoxazole-4,6(3h,5h)-dione, 2-(4-chlorophenyl)-3-(2-thienyl)-5-[3-(trifluoromethyl)phenyl]dihydro-2h-pyrrolo[3,4-d]isoxazole-4,6(3h,5h)-dione, 2-(4-chlorophenyl)-3-(2,4-dichlorophenyl)-5-phenyldihydro-2h-pyrrolo[3,4-d]isoxazole-4,6(3h,5h)-dione, 2,3-di-ph-5-(3-(tri-f-me)ph)dihydro-2h-pyrrolo(3,4-d)isoxazole-4,6(3h,5h)-dione, danazol, and n-cyclopropyl-5-(thiophen-2-yl)isoxazole-3-carboxamide. In a further aspect, the isoxazole compound is danazol or n-cyclopropyl-5-(thiophen-2-yl)isoxazole-3-carboxamide. Additional non-limiting examples include compounds of the isoxazole compound has the general formula (I):

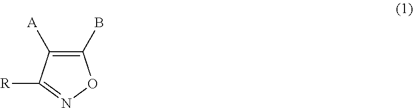

wherein A is a heterocyclic group optionally substituted with G or phenyl optionally substituted with G, wherein G is halogen, C1-C6 alkyl, or optionally substituted phenyl; B is phenyl substituted with halogen, or a heterocyclic group substituted with halogen, C1-C6 alkyl; and R is hydrogen, or C1-C6 alkyl.

Givinostate (GIV) is an orally bioavailable hydroxymate inhibitor of histone deacetylase (HDAC) with potential anti-inflammatory, anti-angiogenic, and antineoplastic activities. Givinostat inhibits class I and class II HDACs, resulting in an accumulation of highly acetylated histones, followed by the induction of chromatin remodeling and an altered pattern of gene expression. At low, nonapoptotic concentrations, this agent inhibits the production of pro-inflammatory cytokines such as tumor necrosis factor- (TNF-), interleukin-1 (IL-1), IL-6 and interferon-gamma. Givinostat has also been shown to activate the intrinsic apoptotic pathway, inducing apoptosis in hepatoma cells and leukemic cells. This agent may also exhibit anti-angiogenic activity, inhibiting the production of angiogenic factors such as IL-6 and vascular endothelial cell growth factor (VEGF) by bone marrow stromal cells.

Rho-associated kinase (ROCK) inhibitors intend Rho-associated protein kinase (ROCK) is a kinase belonging to the AGC (PKA/PKG/PKC) family of serine-threonine kinases. It is involved mainly in regulating the shape and movement of cells by acting on the cytoskeleton. Non-limiting examples of such include Thiazovivin or Y27632, which both can be purchased from Stemcell Technologies and respectively; SR3677, which can be purchased from tocris.com; and GSK429286, which can be purchased from tocris.com.

A TGF-beta type-I receptor inhibitor intends (activin A receptor type II-like kinase, 53 kDa) is an inhibitor for membrane-bound receptor protein for the TGF beta superfamily of signaling ligands. TGFBR1 is its human gene. Non-limiting examples of such include SB431542 and A8301 that can be purchased from _www.tocris.com_ and _www.esibio.com_, respectively; LY2157299, which can be purchased from www.selleckchem.com; and LY2109761, which can be purchased from www.selleckchem.com.

A DNA methyltransferase inhibitor is a small molecule or other agent the ability to inhibit hypermethylation, restore suppressor gene expression and exert antitumor effects in in vitro and in vivo laboratory models. Goffin and Eisenhauer (2002) Ann. Oncol. November 13(11):1699-16716. One non-limiting example of such an inhibitor is N-phthalyl-L-tryptopha (C19H14N2O4, sold under the tradename RG108, Sigma-Aldrich). Additional examples include 5′-azacytidine, 5-azacytidine, antisense oligonucleotides to methyltransferase 1, e.g., MG98 (see Amato (2007) Clin. Gentourin Cancer, December, 5(7):422-426 and 1-(β-D-Ribofuranosyl)-2(1H)-pyrimidinone (a nucleoside analog of cytidine, sold under the name Zebularine (Abcam®).

DNA hypomethylation intends a lower than normal level of DNA methylation.

Methods of determining the level of DNA methylation are known in the art, some of which are described herein.

“Sulfisoxazole” is a sulfonamide antibacterial with an oxazole substituent. It has antibiotic activity against a wide range of Gram-negative and Gram-positive organisms. Compounds of this class available from Sigma-Aldrich and methods to synthesize such are known in the art as described for example in U.S. Pat. No. 2,721,200. Non-limiting examples of sulfisoxazole include FDA approved drugs of AZO GANTRISIN, ERYTHROMYCIN ETHYLSUCCINATE and SULFISOXAZOLE ACETYL, ERYZOLE, GANTRISIN (with effective ingredients as SULFISOXAZOLE), GANTRISIN (with effective ingredients as SULFISOXAZOLE ACETYL), GANTRISIN (with effective ingredients as SULFISOXAZOLE ACETYL), GANTRISIN PEDIATRIC, ILOSONE SULFA, LIPO GANTRISIN, PEDIAZOLE, SOSOL, SOXAZOLE, SOXAZOLE, SULFISOXAZOLE, SULFISOXAZOLE DIOLAMINE, and SULSOXIN. (Drugs@FDA: FDA Approved Drug Products at the website of accessdata.fda.org).

Danazol (also known as 17α-Ethynyl-17β-hydroxyandrost-4-en-[2,3-d]isoxazole) is a a synthetic steroid that is used primarily in the treatment of endometriosis. The compound is commercially available and manufactured by a variety of vendors.

The term “isolated” as used herein refers to molecules or biological or cellular materials being substantially free from other materials, e.g., greater than 70%, or 80%, or 85%, or 90%, or 95%, or 98%. In one aspect, the term “isolated” refers to nucleic acid, such as DNA, RNA, miRNA, exosome or microvesicle, protein or polypeptide, or cell or cellular organelle, or tissue or organ, separated from other DNA, RNA, miRNA, exosome or microvesicle, protein or polypeptide, or cell or cellular organelle, or tissue or organ, respectively, that are present in the natural source and which allow the manipulation of the material to achieve results not achievable where present in its native or natural state, e.g., recombinant replication or manipulation by mutation. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments that are not naturally occurring as fragments and would not be found in the natural state. The term “isolated” is also used herein to refer to polypeptides which are isolated from other cellular proteins and is meant to encompass both purified and recombinant polypeptides, e.g., with a purity greater than 70%, or 80%, or 85%, or 90%, or 95%, or 98%. The term “isolated” is also used herein to refer to cells, exosomes or microvesicles, miRNA, or tissues that are isolated from other cells, exosomes or microvesicles, miRNA, or tissues and is meant to encompass both cultured and engineered cells or tissues and products produced or isolated from such.

The term “phenotype” refers to a description of an individual's trait or characteristic that is measurable and that is expressed only in a subset of individuals within a population. In one aspect of the invention, an individual's phenotype includes the phenotype of a single cell, a substantially homogeneous population of cells, a population of differentiated cells, or a tissue comprised of a population of cells.

The term pharmaceutically acceptable carrier (or medium), which may be used interchangeably with the term biologically compatible carrier or medium, refers to reagents, cells, compounds, materials, compositions, and/or dosage forms that are not only compatible with the cells and other agents to be administered therapeutically, but also are, within the scope of sound medical judgment, suitable for use in contact with the tissues of human beings and animals without excessive toxicity, irritation, allergic response, or other complication commensurate with a reasonable benefit/risk ratio. Pharmaceutically acceptable carriers suitable for use in the present invention include liquids, semi-solid (e.g., gels) and solid materials (e.g., cell scaffolds and matrices, tubes sheets and other such materials as known in the art and described in greater detail herein). These semi-solid and solid materials may be designed to resist degradation within the body (non-biodegradable) or they may be designed to degrade within the body (biodegradable, bioerodable). A biodegradable material may further be bioresorbable or bioabsorbable, i.e., it may be dissolved and absorbed into bodily fluids (water-soluble implants are one example), or degraded and ultimately eliminated from the body, either by conversion into other materials or breakdown and elimination through natural pathways.

A population of cells intends a collection of more than one cell, exosome or microvesicle, or miRNA that is identical (clonal) or non-identical in phenotype and/or genotype. The population can be purified, highly purified, substantially homogenous or heterogeneous as described herein.

The term “propagate” means to grow or alter the phenotype of a cell or population of cells. The term “growing” refers to the proliferation of cells in the presence of supporting media, nutrients, growth factors, support cells, or any chemical or biological compound necessary for obtaining the desired number of cells or cell type. In one embodiment, the growing of cells results in the regeneration of tissue. In yet another embodiment, the tissue is comprised of cardiac progenitor cells or cardiac cells.

The term “effective amount” refers to a concentration or amount of a reagent or composition, such as a composition as described herein, cell population or other agent, that is effective for producing an intended result, including cell growth and/or differentiation in vitro or in vivo, or for the treatment of a condition as described herein. It will be appreciated that the number of cells to be administered will vary depending on the specifics of the disorder to be treated, including but not limited to size or total volume/surface area to be treated, as well as proximity of the site of administration to the location of the region to be treated, among other factors familiar to the medicinal biologist.

The terms effective period (or time) and effective conditions refer to a period of time or other controllable conditions (e.g., temperature, humidity for in vitro methods), necessary or preferred for an agent or composition to achieve its intended result, e.g., the differentiation or dedifferentiation of cells to a pre-determined cell type.

A “subject,” “individual” or “patient” is used interchangeably herein, and refers to a vertebrate, preferably a mammal, more preferably a human. Mammals include, but are not limited to, murines, rats, simians, bovines, canines, felines, humans, farm animals, sport animals and pets.

“Substantially homogeneous” describes a population of cells in which more than about 50%, or alternatively more than about 60%, or alternatively more than 70%, or alternatively more than 75%, or alternatively more than 80%, or alternatively more than 85%, or alternatively more than 90%, or alternatively, more than 95%, of the cells are of the same or similar phenotype. Phenotype can be determined by a pre-selected cell surface marker or other marker.

As used herein, the terms “treating,” “treatment” and the like are used herein to mean obtaining a desired pharmacologic and/or physiologic effect. The effect can be prophylactic in terms of completely or partially preventing a disorder or sign or symptom thereof, and/or can be therapeutic in terms of a partial or complete cure for a disorder and/or adverse effect attributable to the disorder. Examples of “treatment” include but are not limited to: preventing a disorder from occurring in a subject that may be predisposed to a disorder, but has not yet been diagnosed as having it; inhibiting a disorder, i.e., arresting its development; and/or relieving or ameliorating the symptoms of disorder, e.g., cardiac arrhythmia. As is understood by those skilled in the art, “treatment” can include systemic amelioration of the symptoms associated with the pathology and/or a delay in onset of symptoms such as chest pain. Clinical and sub-clinical evidence of “treatment” will vary with the pathology, the individual and the treatment.

“Administration” or “delivery” of a cell, exosome or microvesicle, miRNA, therapeutic or other agent and compositions containing same can be effected in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician or in the case of animals, by the treating veterinarian. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, intraperitoneal, infusion, nasal administration, inhalation, injection, and topical application.

### Descriptive Embodiments

### Induced Pluripotent Stem Cells

This disclosure provides an induced pluripotent stem cell (iPSC) characterized by DNA hypomethylation. In one aspect, the cell is additionally characterized by overexpression of one or more cardiac gene or marker selected from the group of Gαi, mir-133, mir-762, CCL7, CXCR2, CXC5, integral membrane protein 2A, and ephrin A3. Nkx-2.5, ISL1, GATA4, αMHC, Sarcomeric actin, Gαi, mir-133, mir-762, CCL7, CXCR2, CXC5, integral membrane protein 2A, and ephrin A3. In another aspect, the cell under expresses one or more pluripotent genes or markers, non-limiting examples of such include one or more of miR-290-295 cluster, let-7 family, Max and under expresses one or more DNA methyltransferase genes Dnmt1, Dnmt3b. In one aspect, the one or more cardiac gene or marker is Gαi. In one aspect, the one or more cardiac gene or marker is overexpressed at least 1.5 fold, or alternatively at least 2 fold, or alternatively at least 3 fold, over that of a control cell. In another aspect, the cell is characterized by underexpression of one or more other cardiac gene or marker selected from the group of miR-290 cluster, miR-574-5P, let-7 family, Dnmt1, Dnmt3b, and Max. In one aspect, the one or more cardiac gene or marker is not expressed or underexpressed by at least 1.5 fold, or alternatively at least 2 fold, or alternatively at least 3 fold, under that of a control cell. Methods to identify and quantitate genes and markers are known in the art and described herein to supplement well known methods.

The cell can be from any animal species, such as a mammal, e.g., an equine, a murine, a bovine, a canine, a feline, or a human patient.

The iPS cell is derived from any suitable parent cell. Non-limiting examples of such include, without limitation, a cell selected from the group consisting of a bone marrow cell, a myoblast, a skin fibroblast, a cord blood cell, an adult peripheral blood, a cardiac progenitor cell, a small juvenile stem cell (SJST), a marrow stromal cell, a mesenchymal stem cell, a hematopoietic stem cell, or a mononuclear cell.

Methods to derive or prepare an iPSC from these parent cell types are known in the art. In one aspect, the iPSC was created by a method comprising contacting the parent cell with an effective amount of a DNA methyltransferase inhibitor to upregulate Oct4. Non-limiting examples of DNA methyltransferease inhibitors include 5′-azacytidine, 5-aza-2′-deoxycytidine, MG98, zebularine and RG108. In another aspect, the iPS cell was created by a method that excludes the insertion of exogenous genes into the parent cell.

In a yet further embodiment, the parent cells are progenitor cells, e.g., cardiac progenitor cells, prepared by preconditioning the cells with electrical stimulation. In one aspect, the parent cell is a stem cell that expresses Sca 1 and pluripotency and cardiac genes as described herein. The cells are subsequently contacted with isoxazole or an isoxazole similar compound as described herein.

In one aspect, the parent cell is a small juvenile stem cell or young stem cell that is not modified or programmed to an iPSC. In this aspect, the SJST is treated with the isoxazole or isoxazole similar compound and used diagnostically, in research or therapeutically. In another aspect, the disclosure provides a method to direct an immature or progenitor cell, e.g., a SJST cell, a circulating blood or heart derived stem cell) to a cardiac progenitor phenotype by contacting the cell with an effective amount of an isoxazole or isoxazole similar compound as described herein. In another aspect, the parent cells are juvenile or young stem cells that can be identified by low expression of miR-195 as compared to a cell that does not express the characteristics of a young or juvenile cell as described herein. In a further aspect, the parent cell is further characterized by low expression of one or more marker selected from miR-29b, miR-205, miR-378, and miR-542-3p as compared to a cell that does not express the characteristics of a young or juvenile cell as described herein. In a further or alternative aspect, the parent cell has been pre-conditioned with an effective amount of electrical stimulation. In another aspect, isoxazole or isoxazole similar compound is injected therapeutically in the ischemic heart where BMSC, skeletal myoblasts, peripheral blood-derived endothelial progenitor cells (EPC), resident cardiac stem/progenitor cells and fibroblasts are mobilized to the injury area by the art described (Konoplyannikov M I, Haider K H, Lai V K, Ahmed R P, Jiang S, Ashraf M Activation of diverse signaling pathways by ex-vivo delivery of multiple cytokines for myocardial repair. Stem Cells Dev. 2013 Jan. 15; 22(2):204-15) prior to treatment for maximum drug contact to induce regeneration.

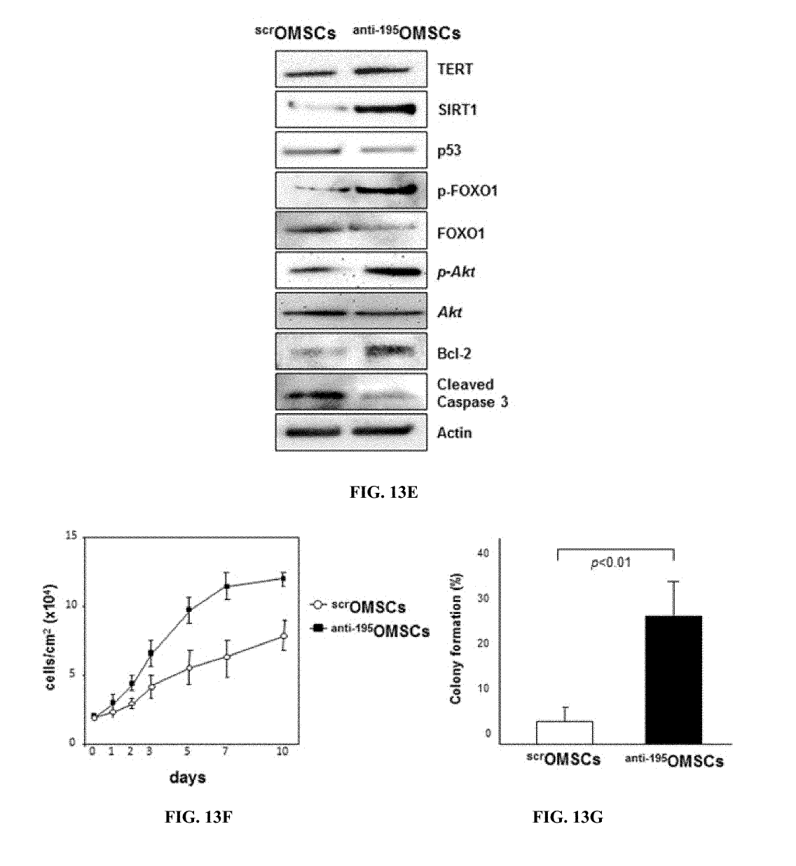

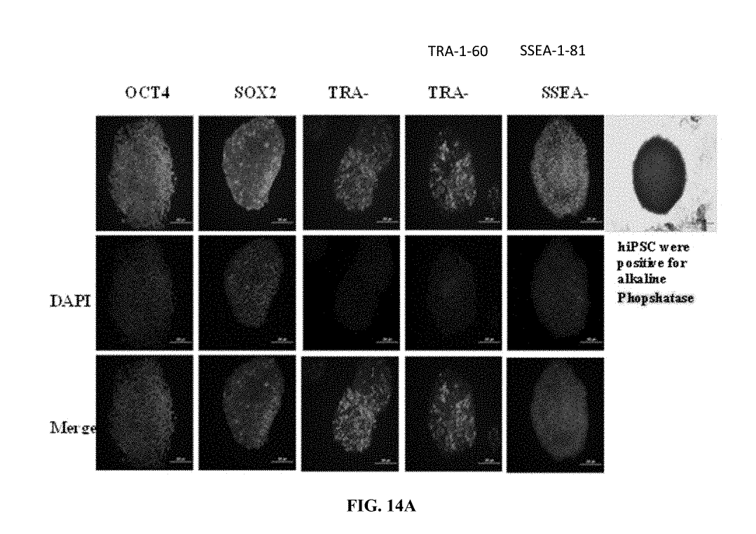

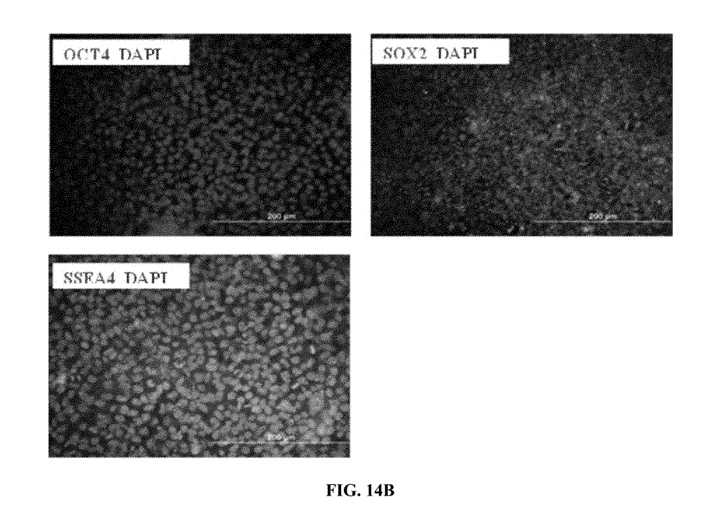

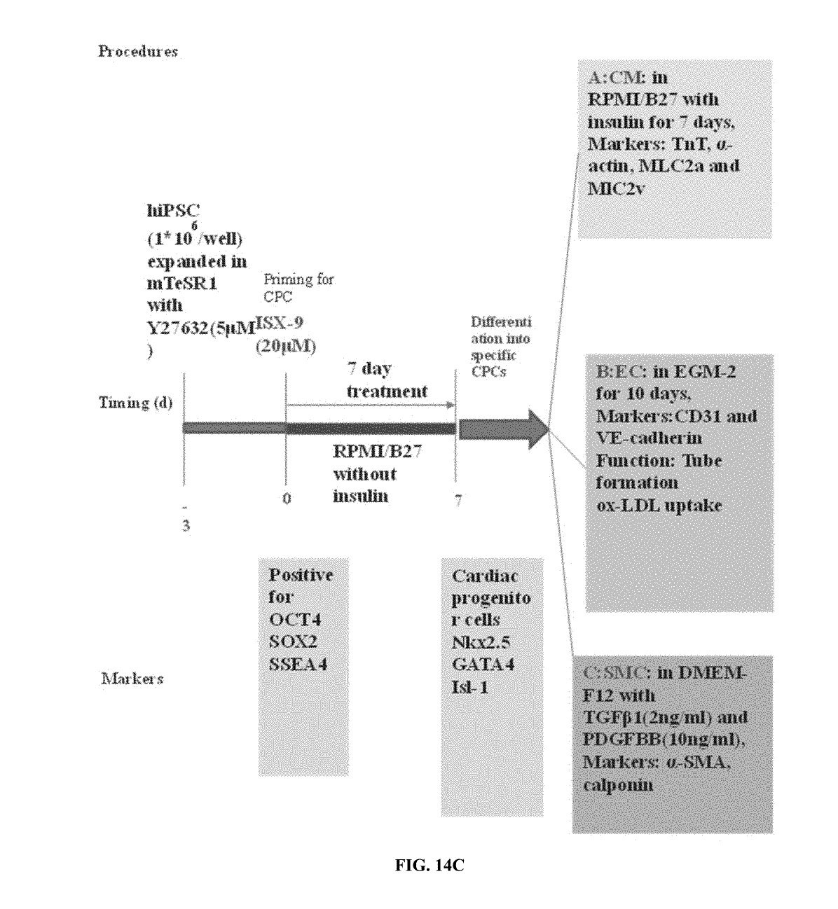

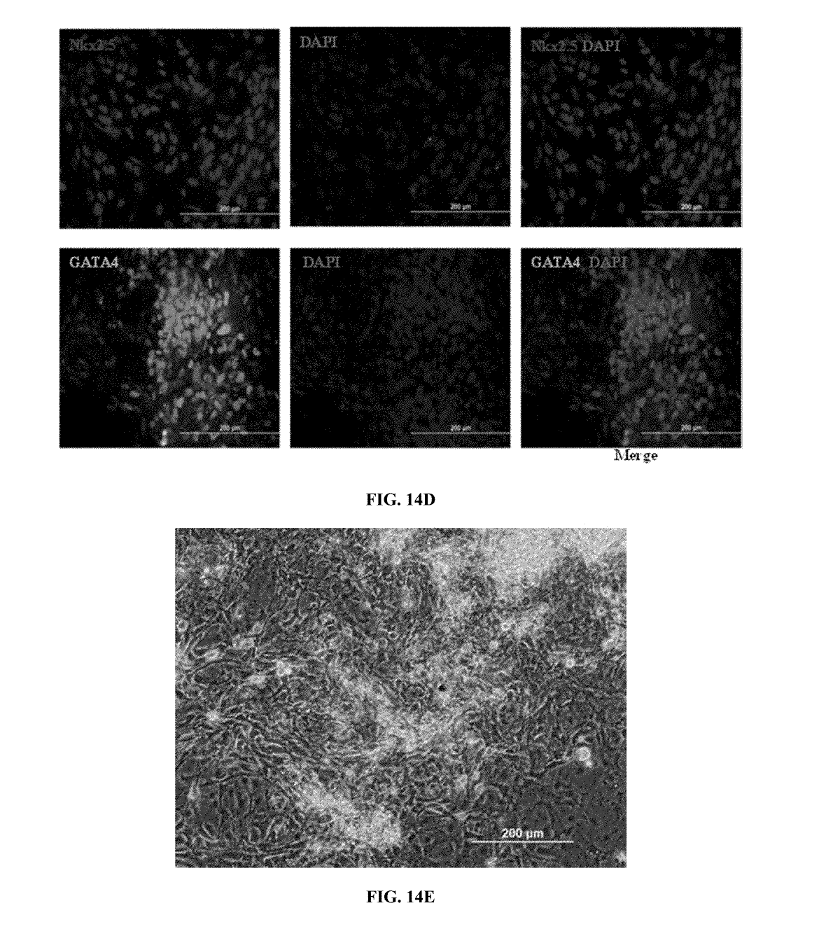

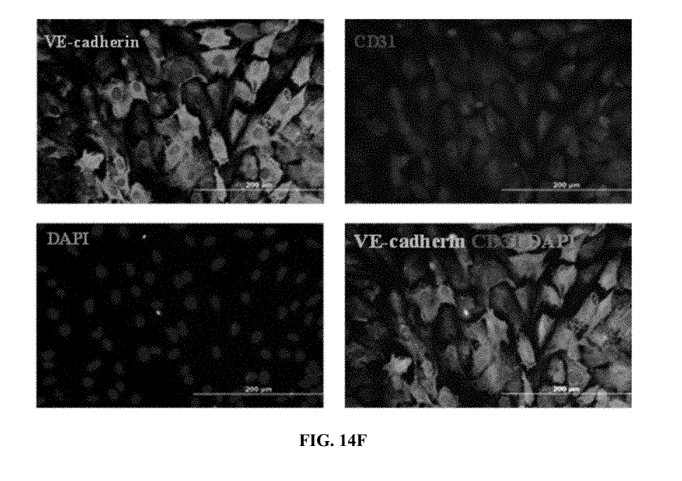

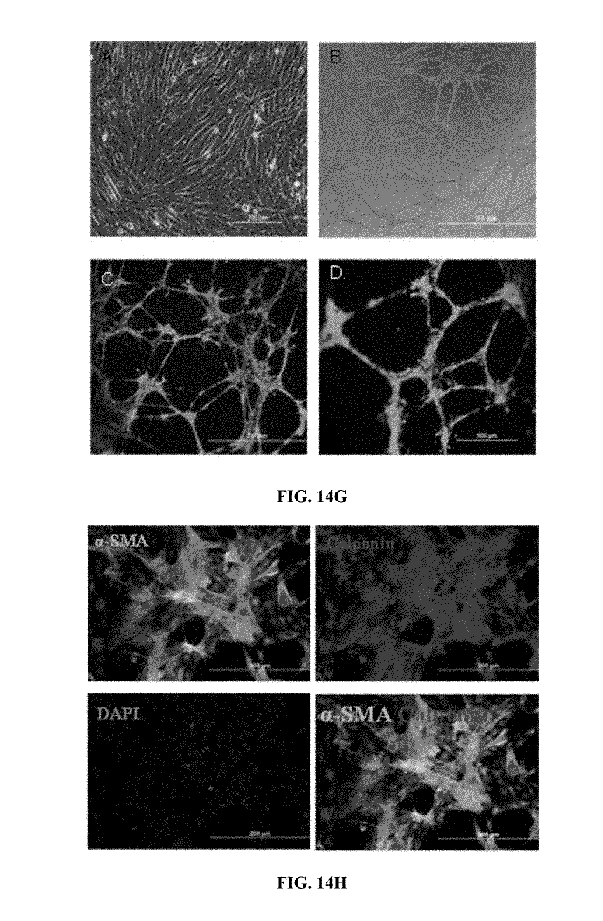

The chemically modified iPS cell as described herein is prepared by contacting the cell with an effective amount of an isoxazole or isoxazole similar compound, e.g., an amount that is selected from the group of from about 0.3 to about 30 μM; from about 0.5 to about 25 μM; from about 12 to about 25 μM and from about 0.5 μM to about 20 μM. See also FIG. 14C.

While methods to prepare an iPS cell are known in the art, Applicant has determined that an iPS cell created by a method comprising contacting a parent cell with an effective amount of a DNA methyltransferase inhibitor, e.g., 5′-azacytidine or RG108 (Sigma-Aldrich) to upregulate Oct4 is particularly useful. In one aspect, the iPS cells are additionally prepared by a method that excludes the insertion of exogenous genes into the parent cell thereby enhancing the safety of the cells for clinical use. In another aspect, the cells are modified to an iPS cell by insertion of genes and other factors known in the art.

This disclosure also provides a population of cultured cells as described herein, by culturing the chemically modified cells to expand the cells as described herein. In one aspect the population is substantially homogenous, e.g., at least 70% identical in phenotype, or alternatively at least 75% identical in phenotype, or alternatively at least 80% identical in phenotype, or alternatively at least 85% identical in phenotype, or alternatively at least 90% identical in phenotype, or alternatively at least 95% identical in phenotype, or alternatively at least 98% identical in phenotype or alternatively, a clonal population of cells. In one aspect, the population is a clonal population. In one aspect, the cells are cultured under conditions that favor differentiation into a particular cell type, e.g., a cardiac cell. These culturing conditions are known in the art.

The cells and populations of cells can be further modified for therapeutic or research use, for by example, further comprising a detectable label or exogenous polynucleotide or polypeptide. Methods and appropriate polynucleotides for therapeutic use are described in Durrani et al. (2010) Regen. Med. 5(6):919-932.

### Method for Preparing the iPS Cells

This disclosure also provides a method for preparing a cardiac lineage cell from a stem cell, comprising contacting the stem cell with an effective amount of an isoxazole or isoxazole similar compound. Stem cells useful in the method includes, without limitation, a one or more of an iPS cell, such as an iPS cell derived from a bone marrow cell, bone marrow stomal cell, a mesenchymal stem cell, a hematpoietic stem cell, a myoblast, a skin fibroblast, a cord blood cell, an adult peripheral blood, a SJSC, a cardiac progenitor cell, or mononuclear cell. Methods to derive or prepare an iPSC from these parent cell types are known in the art. The cells prepared by this method are characterized in overexpressing one or more cardiac gene or marker, e.g., Nkx-2.5, ISL1, Tbx-5, GATA4, αMHC, Sarcomeric actin, Gαi, miR-133, miR-762, CCL7, CXCR2, CXC5, integral membrane protein 2A, or ephrin A3 and/or underexpressing one or more cardiac gene or marker, e.g., miR-290-295 cluster, let-7 family, Dnmt1, Dnmt3b, and Max, each as compared to a control cell such as a cell that has not been contacted with or exposed to the isoxazole or isoxazole similar compound. In one aspect, the over- or under-expression is at least 1.5 fold over- or under- that of the control cell.

In one aspect, the cell is a small juvenile stem cell (SJSC) that is not modified or programmed to an iPSC. In this aspect, the SJST is treated with the isoxazole or isoxazole similar compound and used diagnostically, in research or therapeutically. In another aspect, the disclosure provides a method to direct an immature or progenitor cell, e.g., a SJST cell, (contained with a population of circulating blood or heart derived stem cells) to a cardiac progenitor phenotype by contacting the cell with an effective amount of an isoxazole or isoxazole similar compound as described herein. An effective amount of electrical stimulation can be applied to the cell or tissue in need of the treatment, alone or in combination with the isoxazole or isoxazole similar compound. In another aspect, the parental cell is selected for chemical modification because it is characterized by low expression of miR-195 and the cell may or may not be pre-condition by application of an effective amount electrical stimulation.

In a particular aspect, the method comprises, or alternatively consists essentially of, or yet further consists of, contacting the cells with an effective amount of the isoxazole or isoxazole similar compound for at least 3 days, or alternatively at least 4 days, or alternatively at least 5 days, or alternatively at least 6 days, or alternatively at least 7 days, in DMEM F12 supplemented with from about 10% to about 30%, or alternatively 20% Knockout Serum Replacement (KSR; Invitrogen, USA), and from about 0.05 mM to about 0.2 mM, or about 0.1 mM MEM Non-Essential Amino Acids solution (Invitrogen, CA, USA), and from about 0.1 mM to about 0.3 mM or about 0.2 mM L-glutamine (Invitrogen, USA); and from about 0.05 mM to about 0.2 mM or alternatively from about 0.1 mM β-mercaptoethanol (Invitrogen, CA, USA); and from about 750 U/ml to about 1250 U/ml or alternatively from about 1000 U/ml LIF (Millipore); and an effective amount of 0.5% penicillin and streptomycin. The cells are then kept in the media without drug for five days. The media is changed every day otherwise due to increase in cell number the pH of the media get changed and effect the cell survival and gene expression. An effective amount of the isoxazole or isoxazole similar compound comprises, or alternatively consists essentially of, or yet further consists of from about 0.3 to about 30 μM; or from about 0.5 to about 25 μM; or from about 12 to about 25 μM; or from about 0.5 μM to about 20 μM. As used herein, isoxazole or isoxazole similar intends a class of compounds as described herein.

In another particular aspect, the method comprises, or alternatively consists essentially of, or yet further consists of, contacting the cells with an effective amount of the isoxazole or isoxazole similar compound for at least 3 days, or alternatively at least 4 days, or alternatively at least 5 days, or alternatively at least 6 days, or alternatively at least 7 days, in RPMI F12 without insulin. An effective amount of the isoxazole or isoxazole similar compound comprises, or alternatively consists essentially of, or yet further consists of from about 0.3 to about 30 μM; or from about 0.5 to about 25 μM; or from about 12 to about 25 μM; or from about 0.5 μM to about 20 μM. In yet another particular aspect, an isoxazole is an ISX-9 as described herein above. As used herein, isoxazole or isoxazole similar intends a class of compounds as described above.

The cells are then kept in RPMI F12 with insulin for another about 7-10 days to generate cardiomyocyte differentiation. The cells are then kept in EGM-2 medium (Lonza, Lonza Walkersville Inc., Walkerswille Md. 21793-0127) for about 10 days to generate endothelial cell differentiation. The cells are kept in TGFβ (2 ng/ml) and PDGFBB (long/ml, R&D Systems, Inc, Minneapolis, Minn. 55413) about 10 days to generate smooth muscle cell differentiation.

Methods to identify and quantitate genes and markers are known in the art and described herein to supplement well known methods.

One can determine if the methods of this disclosure have been effective by molecular, clinical and other techniques. In one aspect, Nkx2.5, GATA4, Tbx5, ISL-1 and Mef2c upregulation is started after 3 days after contacting and expression of these markers can be determined by application histochemistry and/or PCR, as appropriate. Additional lineage identifying markers are shown in FIG. 14C. The cells can be further evaluated for 7 day treatment by qPCR. Immunofluorescence staining also showed that transcription factors (Nkx2.5, GATA4 and ISL-1) were highly expressed in hiPSC with 7 day small molecule treatment. The purity of Nkx2.5 positive cells in these small molecule treated cells by FACS was 96.5±2.5% cells. To establish that the Nkx2.5+ cells are truly committed cardiovascular precursors, Applicant found that these Nkx2.5+ cells were multipotent and directly differentiated into all three cardiovascular lineages, including CMs, ECs and SMCs in basal differentiation conditions without any specific induction signaling molecules with percentage of 95.2±2.1% CMs (TnT+), 90.3±2.5% ECs (CD31+) and 92.3±1.8% SMCs (α-SMA+). In addition, Applicant determined that the differentiated ECs exhibited phenotype and function similar to primary endothelial cells (ECs) (FIGS. 14A-14H)

The cell can be from any animal species, such as a mammal, e.g., an equine, a murine, a bovine, a canine, a feline, or a human patient. The cell can be autologous or allogeneic to the animal or patient being treated.

The contacting can be performed in vitro, e.g., in a tissue culture dish or plate as described herein or in vivo, by administering an effective amount of the isoxazole or isoxazole similar compound to a cell culture or in vivo by administering an effective amount of the compound or alternatively, compound and iPS cells to a patient or subject in need of such treatment. Methods for administering, systemically or locally, such are described below. The compounds and/or cells can be combined with a pharmaceutically acceptable carrier for ease of use. This treatment can, in one aspect, be combined with the administration of an effective amount of electrical stimulation to the tissue in need of such treatment. The electrical stimulation can be administered prior to, concurrently or subsequent to the administration of the isoxazole or isoxazole similar compound.

In one aspect, the stem cell contacted with the compound is an iPS cell. Methods to generate iPS cell from terminally differentiated cells are known in the art, e.g., by a method comprising contacting the parent cell with an effective amount of a DNA methyltransferase inhibitor, e.g., RG108 (Sigma-Aldrich) to upregulate Oct4. In another aspect, the iPS cell is created by a method that excludes the insertion of exogenous genes into the parent cell.

This disclosure also provides an isolated cell prepared by a method as described herein by further comprising isolating the cells. Yet further, the method further comprises culturing the cells to prepare a population of cells. In one aspect the population is cultured under conditions to prepare a substantially homogenous, e.g., at least 70% identical in phenotype, or alternatively at least 75% identical in phenotype, or alternatively at least 80% identical in phenotype, or alternatively at least 85% identical in phenotype, or alternatively at least 90% identical in phenotype, or alternatively at least 95% identical in phenotype, or alternatively at least 98% identical in phenotype. In one aspect, the method further comprises culturing the cells under conditions that favor clonal expansion of the cells to a clonal population. In one aspect, the cells are cultured under conditions that favor differentiation into a particular cell type, e.g., a cardiac cell. These culturing conditions are known in the art.

The methods can be further modified by inserting into the cells and populations of cells a detectable label or exogenous polynucleotide or polypeptide. Methods and appropriate polynucleotides for therapeutic use are described in Durrani et al. (2010) Regen. Med. 5(6):919-932.

### Exosome or Microvesicle Populations and Exosome or Microvesicle Compositions

This disclosure provides composition comprising, or alternatively consisting essentially of, or yet further consisting of, an isolated population of exosomes or microvesicles overexpressing a microRNA (miRNA) selected from the group of mir-373, mir-210, mir-377, mir-367, mir-520, mir-548ah, mir-335, mir-21, mir-30c, mir-214 and/or mir-548q; and/or one or more of a protein selected from the group of Tsg101, CD9, Hsp70, Flotilline-1, or GAPDH. In another aspect, the population comprises two or more, or three or more, or four or more, or five or more, or six or more, or seven or more, or eight or more, or nine or more, or ten or more, or eleven or more, or all twelve of the miRNA and/or at least one, at least two, at least three, at least four or all five of the proteins. In an alternative aspect, for the population of exosomes or microvesicles, at least 80%, or alternatively 85%, or alternatively 90%, or alternatively 95%, or alternatively 97% or alternatively 99%, or alternatively 100%, of the exosomes or microvesicles overexpress the miRNA and/or the proteins. The exosomes or microvesicles of the population are isolated from a cell selected from a cardiac progenitor cell (“CPC”), a cardiomyocyte, an endothelial cell, a myocyte, or a smooth muscle cell.

Also provided is a cell, a population of cells, or a composition comprising, or alternatively consisting essentially of, or yet further consisting of: an isolated population of one or more of: a cardiac progenitor cell a (CPSs), a cardiomyocyte, a myocyte, an endothelial cell, a smooth muscle cell, a skeletal muscle cell, each generated from a cell selected from the group of an iPSC, an embryonic stem cell, or a stem cell that was contacted with an isoxazole compound, a derivative or an equivalent thereof. The cells or population of cells are identified by overexpression of a microRNA (miRNA) selected from the group of mir-373, mir-210, mir-377, mir-367, mir-520, mir-548ah, mir-335, mir-21, mir-30c, mir-214 or mir-548q; and/or a muscle gene selected from the group of paZ3, pAX7, MYF5, MYOD, MYOG, or dystrophin. In a further aspect, provided herein is a cardiomyocyte or a population of cardiomyocytes that express on or more of cTnT, cTnI, MLC2V, and/or CX43. Further provided is a cell or a population of endothelial cells that expressCD31 and VE-cadherin. Yet further provided is a smooth muscle cell or population of these cells that express α-smooth muscle actin (SMA) and calponin.

In another aspect, the populations as described herein comprises two or more, or three or more, or four or more, or five or more, or six or more, or seven or more, or eight or more, or nine or more, or ten or more, or all eleven of the miRNA and/or at least one, at least two, at least three, at least four, or at least four, or at least five, or all six five of the muscle genes. In an alternative aspect, for the population of exosomes or microvesicles, at least 80%, or alternatively 85%, or alternatively 90%, or alternatively 95%, or alternatively 97% or alternatively 99%, or alternatively 100%, of the cells of the population overexpress the miRNA and/or the genes and/or the proteins.

Also provided is an isolated population of cells that express one or more protein selected from the group: cTnI, MLC2v, cTnT, VE-cadherin, CD31, α-SMA (actin), calponin or Cx43, wherein optionally at least two, or at least three, or at least four, or at least five, or at least six, or at least seven, or all eight of expressed proteins. In one aspect, the population of cells is selected from the group consisting of a smooth muscle cells, an endothelial cells, a blood vessel or a cardiomyocyte.

In an alternative aspect, for the population of cells, at least 80%, or alternatively 85%, or alternatively 90%, or alternatively 95%, or alternatively 97% or alternatively 99%, or alternatively 100%, of the exosomes or microvesicles express the proteins. Methods to identify protein expression are known in the art and include the use of protein-specific antibodies are hybridization techniques that monitor mRNA expression. The populations can be prepared by a method comprising, or alternatively consisting essentially of, or yet further consisting of culturing a population of iPSC in the presence of an effective amount of an isoxazole, a derivative or an equivalent thereof. In a yet further, the population is derived from a fibroblast or a skeletal myoblast. The populations can be homogenous, heterogeneous, purified, highly purified, homogenous or substantially homogeneous. In a further aspect, the exosomes or microvesicles can be detectably labeled and/or combined with a carrier, preservative or cryprotectant. The populations and compositions can be lyophilized. Methods to isolate exosome or microvesicle populations and produce isolated population of cells are known in the art, some of which are described herein. In a further aspect, the population is derived from a fibroblast or a skeletal myoblast.

In a further aspect, the population of cells is selected from the group consisting of smooth muscle cells, endothelial cells, and cardiomyocytes. As used herein, a “smooth muscle cell” intends a cell expressing the following markers SMTN (smoothelin), Cnn1 (calponin 1), telokin. As used herein, an “endothelial cell” intends a cell expressing the following markers VE-cadherin (CD144), CD31, von Willerbrand factor (vWF).

The compositions can further comprise a protein that facilitates regeneration and/or improved function of a tissue or a nucleic acid that encodes the protein and/or an agent that inhibits the expression of an inflammatory protein, that is optionally a cytokine. In one aspect, the protein is selected from the group of a transforming growth factor beta (TGF-beta), a WNT protein, a cytokine, or a histon deacetylase.

In one aspect, the cells are cultured in serum-free media. In another aspect, the effective amount of the isoxazole compound, derivative or equivalent thereof, is an amount that results in overexpressed of the mir. In one aspect, the effective amount is from about 5 μM to about 25 μM. In another aspect, the effective amount is 8 to 20 or from about 10 to about 15 or from about 5 to about 20 μM and from about 10 μM to about 25 μM.

These compositions are useful in a method for one or more of: regenerating damaged tissue such as muscle and/or cardiac tissue; improving the viability of damaged tissue, such as muscle and/or cardiac tissue; facilitating the formation of new tissue, such as cardiac tissue, muscle tissue, skeletal muscle tissue, a blood vessel, a capillary, or a myocyte; promoting cardiac regeneration; promoting cardiac regeneration in a subject suffering from an acute cardiac event; promoting cardiac regeneration in a subject suffering from a myocardial infarction; promoting cardiac regeneration in subject suffering from Duchenne Muscular Dystrophy (DMD) or Duchenne Muscular Dystrophy (“DMD”)-associated cardiomyopathy, promoting cardiac regeneration in a subject suffering from age-related diseases, such as for example, chronic obstructive pulmonary disease (“COPD”), arthritis, osteoporosis, osteoarthritis, diabetes, vascular dementia, or macular degeneration; promoting tissue regeneration in tissue damaged from one or more of stroke, arthritis, Alzheimer's, memory loss disorders, cystic fibrosis, inflammatory disorders and cancer; decreasing cardiac wall thickness in a tissue damaged from a cardiac infarction; altering gene expression of one or more of protein kinase C, iL-6, mmp, PDGF; reducing or inhibiting the expression of an inflammatory protein that is optionally a chemokine, macrophage or a cytokine; directly or indirectly stimulating angiogenesis; promoting cardiac regeneration in a subject suffering from a disease selected from the group of coronary artery disease, myocardial infarction, heart failure, hypoplasic left heart syndrome, peripheral artery disease (PAD), cardiac mypertrophy, valvular heart disease (aortic stenosis), myocardial hypertrophy, hypertrophy fibrosis; and/or directly or indirectly inhibit cellular replication. The methods are accomplished by administering an effective amount of the exosomes or microvesicles, cells, populations or compositions containing the same to a subject in need thereof. The methods can further comprise administering an effective amount of non-embryonic stem cell or a progenitor cell to the subject that is optionally the same or different type of tissue that is in need of repair. In one aspect, the non-embryonic stem or progenitor cell is autologous to the subject. In another aspect, the non-embryonic stem or progenitor cell is allogeneic to the subject. The exosomes or microvesicles, cells, populations can be locally or systemically delivered to the subject, e.g., via an intramyocardialor an intracoronary route. Subjects suitably treated by these methods include animals, mammals and human patients. Methods to determine the effectiveness of the therapy are known in the art, some of which are described herein.

The compositions also are useful in methods for one or more of the following therapies: anti-oxidative therapy; provding anti-oxidative therapy; promoting activation of local or resident cardiomyocytes; promoting the release of angiogenesis and/or paracrine factors; promoting activation of wnt, a BMP, and/or cytoskeleton remodeling; promoting TGF-β induced emt signaling and cardiac differentiation; increasing expression of wnt5 and wnt11 or a BMP family protein, optionally BMP4; increasing expression of a cardiac transcription factor selected from the group consisting of nkx2.5, mef2c, gata4 and isl-1; promoting expression of genes for development of pip3 signaling in cardiomyocytes, muscle contraction and nf-at hypertrophy signaling pathways; reducing fibrosis and apoptosis; promoting myoangeneis and muscle differentiation; promoting the release of a cytokine selected from the group consisting of angiopoietin-2, il-6nmp, pgfbb, timp 1 or a gene identified in the Figures as disclosed herein; promoting upregulation of a gene selected from the group consisting of wnt3a, wnt5a, wnt11; and/or promoting cytoskeletal remodeling, by administering an effective amount of the exosome or microvesicle, cells, populations or compositions as described above to a subject in need thereof.

Further provided is an isolated population of cells that express one or more protein selected from the group of cTnI, MLC2v, cTnT, VE-cadherein, CD31, α-SMA (actin), calponin or Cx43.

In another aspect, the above noted compositions further comprise a protein that facilitates regeneration and/or improved function of a tissue or a nucleic acid that encodes the protein. Non-limiting examples of such proteins include for example, Bmp7 in kidney, Fgf-21 in liver, Op-1 in bone regeneration, etc. In a yet further aspect, the compositions further comprise a agent that inhibits the expression of an inflammatory protein. Non-limiting examples of these agents include for example Phospholipase A2 inhibitors, p38 Mitogen-Activated Protein (MAP) kinase inhibitors, etc. In a further aspect, the protein is a cytokine, non-limiting examples of such cytokines include interleukin and interferon.

In one aspect, the populations as described herein further comprise a carrier, wherein in one aspect, a non-naturally occurring carrier. In another aspect, the populations further comprise a preservative or cryoprotectant, such as polyethylene glycol.

In a further aspect, the populations as described herein are lyophilized.

In a further aspect, the compositions described herein are isolated from a population of stem cells or progenitor cells cultured in the presence of an effective amount of an isoxazole compound or a derivative thereof. In one aspect, the isoxazole compound is selected from isoxazole-1 (isx-1) or isoxazole-9 (isx-9). In another aspect, the isoxazole derivative is a compound having the formula:

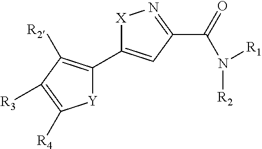

wherein R₁ and R₂ are both hydrogen or R₁ is hydrogen and R₂ is selected from the group consisting of substituted or unsubstituted C₁-C₆ alkyl, C₃-C₆ cycloalkyl, C₂-C₆ alkenyl, C₂-C₆ alkynyl, and benzyl, or where R₁ and R₂ may be joined together to form a ring selected from azetidinyl, pyrrolidinyl, piperidinyl, piperazinyl, or morpholinyl; R₂′, R₃ and R₄ are independently selected from the group consisting of hydrogen, halogen, C₁-C₆ alkyl, C₃-C₆ cycloalkyl, substituted or unsubstituted aromatic or heteroaromatic ring, cyano, nitro, and acyl; X is 0, NH or S; and Y is 0, NH or S.

In a further aspect, the isoxazole compound has the formula:

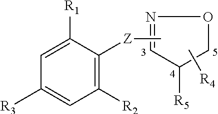

wherein R1 and R2 is each selected from C1-C4 alkyl, phenyl, benzyl, trifluoromethyl or halogen, R3 is selected from hydrogen, hydroxy, C1-C4 alkyl or alkoxy, R4, in position 3 or 5, is selected from hydrogen, trifluoromethyl, C1-C4 alkoxy, C1-C4 alkyl, or C1-C4 hydroxyalkyl, R5 is selected from hydrogen or C4-C4 alkyl or R4 and R5 together form a tetramethylene group, Z at position 3 or 5 on the heterocycle is selected from: —N(R6)-CO—, —CO—N(R6)-, —N(R6)-CO—N(R6)-, —CH(R6)-NH—CO—, or —NH—CO—CH(R6), in which R6 is selected from hydrogen or C1-C4 alkyl.

In a yet further aspect, the isoxazole compound or a derivative thereof is selected from the group: 5-(trifluoromethyl)-3-(4-methoxyphenyl)isoxazole-4-carboxylic acid, 5-(trifluoromethyl)-3-(4-fluorophenyl)isoxazole-4-carboxylic acid, 5-(thiophen-2-yl)isoxazole-3-carboxaldehyde, 5,6,7,8-tetrahydro-4h-cyclohepta[d]isoxazole-3-carboxylic acid, 4,5,6,7-tetrahydro-benzo[d]isoxazole-3-carboxylic acid, 3-amino-5-methylisoxazole, 4-amino-n-(5-methyl-3-isoxazolyl)benzenesulfonamide, 3-phenyl-isoxazole-5-boronic acid pinacol ester, 5-phenylisoxazole, 1-phenyl-1-cyclopentanecarboxylic acid, 3-phenyl-benzo[c]isoxazole-5-carboxylic acid, 5-methyl-3-phenylisoxazole-4-carboxylic acid, 3a,4,5,6,7,8,9,9a-octahydro-cycloocta[d]isoxazole-3-carboxylic acid, 5-(3-nitrophenyl)isoxazole, 3-(4-nitrophenyl)isoxazole, 3-hydroxy-5-aminomethyl-isoxazole, 5-(morpholinomethyl)isoxazole-3-carboxylic acid hydrochloride, 5-(morpholinomethyl)isoxazole-3-carbaldehyde, 3-methyl-5-(trifluoromethyl)isoxazole-4-carboxylic acid, methyl 5-(thiophen-2-yl)isoxazole-3-carboxylate, 3-(methylsulfonyl)-5-(2-thienyl)isoxazole-4-carbonitrile, 5-methyl-3-(2-pyrrolidinyl)isoxazole, 3-methyl-5-(2-pyrrolidinyl)isoxazole, 3-(1-methyl-1h-pyrazol-4-yl)-isoxazole-5-carboxylic acid, 3-(1-methyl-1h-pyrazol-4-yl)-4,5-dihydro-isoxazole-5-carboxylic acid, 5-(4-methylphenyl)isoxazole-3-carboxylic acid, 5-methyl-3-phenylisoxazole-4-carboxylic acid, 5-(4-methylphenyl)isoxazole-3-carboxaldehyde, 5-methyl-3-(4-phenoxyphenyl)isoxazole-4-carboxylic acid, 3-methyl-5-(4-methyl-1,2,3-thiadiazol-5-yl)isoxazole-4-carboxylic acid, 3-methyl-5-(5-methylisoxazol-3-yl)isoxazole-4-carboxylic acid, methyl 5-(4-methoxyphenyl)isoxazole-4-carboxylate, methyl 5-(4-methoxyphenyl)isoxazole-3-carboxylate, 5-methylisoxazole, methyl 5-(4-fluorophenyl)isoxazole-4-carboxylate, methyl 5-(4-fluorophenyl)isoxazole-3-carboxylate, methyl 5-(4-chlorophenyl)isoxazole-4-carboxylate, methyl 5-(4-bromophenyl)isoxazole-4-carboxylate, 5-(4-methoxyphenyl)isoxazole-3-carboxylic acid, 5-(3-methoxy-phenyl)-isoxazole-3-carboxylic acid, 3-(2-methoxyphenyl)isoxazole-5-carboxylic acid, 5-(4-methoxyphenyl)isoxazole-3-carboxaldehyde, 3-(4-methoxyphenyl)isoxazole-5-carbaldehyde, 3-(2-methoxyphenyl)isoxazole-5-carbaldehyde, 5-(4-methoxyphenyl)isoxazole, 3-(4-methoxyphenyl)isoxazole, 3-(2-methoxy-phenyl)-4,5-dihydro-isoxazole-5-carboxylic acid, 3-methoxy-isoxazole-5-carboxylic acid, isoxazole-5-carboxylic acid, isoxazole-4-carboxylic acid, isoxazole-5-carbothioamide, isoxazole-5-carbonyl chloride, isoxazole-3-carbonitrile, isoxazole-3-carbaldehyde, isoxazole-4-boronic acid, isoxazole, 5-cyclopropyl-4-[2-(methylsulfonyl)-4-(trifluoromethyl)benzoyl]isoxazole, 6-(5-(thiophen-2-yl)isoxazole-3-carboxamido)hexyl 5-((3as,4s,6ar)-2-oxohexahydro-1h-thieno[3,4-d]imidazol-4-yl)pentanoate, isocarboxazid 5-methyl-3-isoxazole-carboxylic acid 2-benzylhydrazide, 5-isobutyl-isoxazole-3-carboxylic acid, 4-iodo-5-methyl-isoxazole, 3,3′-iminobis(n, n-dimethylpropylamine), 3-(3-hydroxy-phenyl)-isoxazole-5-carboxylic acid methyl ester, 5-(4-hydroxy-phenyl)-isoxazole-3-carboxylic acid, 5-(3-hydroxy-phenyl)-isoxazole-3-carboxylic acid, 5-(hydroxymethyl)-3-methylisoxazole, 3-hydroxy-5-methylisoxazole, 5-(1-hydroxyethyl)-3-(4-trifluoromethylphenyl)isoxazole, 3a,4,5,6,7,7a-hexahydro-benzo[d]isoxazole-3-carboxylic acid, 5-(2-furyl)isoxazole-3-carbaldehyde, 5-furan-2-yl-isoxazole-3-carboxylic acid, 6-fluoro-3-(4-piperidinyl)benzisoxazole, 5-(4-fluorophenyl)isoxazole-3-methanol, 3-(2-fluoro-phenyl)-isoxazole-5-carboxylic acid, 5-(4-fluorophenyl)isoxazole-3-carboxaldehyde, 3-(4-fluorophenyl)isoxazole-5-carbaldehyde, 3-(3-fluorophenyl)isoxazole-5-carbaldehyde, 3-(2-fluorophenyl)isoxazole-5-carbaldehyde, 5-(4-fluorophenyl)isoxazole, 3-(4-fluorophenyl)isoxazole, 5-(3-fluoro-4-methoxy-phenyl)-isoxazole-3-carboxylic acid, ethyl 5-(trifluoromethyl)-3-(4-methoxyphenyl)isoxazole-4-carboxylate, ethyl-5-(tributylstannyl)isoxazole-3-carboxylate, ethyl 5-(thiophen-2-yl)isoxazole-3-carboxylate, 5-ethyl-isoxazole-4-carboxylic acid, 5-ethyl-isoxazole-3-carboxylic acid, ethyl 5-(4-fluorophenyl)isoxazole-4-carboxylate, ethyl 5-(4-fluorophenyl)isoxazole-3-carboxylate, ethyl 5-(2,3-dihydrobenzo[b][1,4]dioxin-7-yl)isoxazole-3-carboxylate, ethyl 3-(4-chlorophenyl)-5-(trifluoromethyl)isoxazole-4-carboxylate, ethyl 5-(4-chlorophenyl)isoxazole-3-carboxylate, ethyl 3-(4-bromophenyl)-5-(trifluoromethyl)isoxazole-4-carboxylate, ethyl 5-(4-bromophenyl)isoxazole-3-carboxylate, ethyl 5-amino-4-(4-chlorophenyl)isoxazole-3-carboxylate, ethyl 5-amino-4-(4-bromophenyl)isoxazole-3-carboxylate, ethyl 6b-acetyl-2-(acetyloxy)-4a,6a-dimethyl-2,3,4,4a,4b,5,6,6a,6b,9a,10,10a,10b,11-tetradecahydro-1 h-naphtho[2′,1′:4,5]indeno[2,1-d]isoxazole-9-carboxylate, 3,5-dimethyl-4-(tributylstannyl)isoxazole, 5-(1,5-dimethyl-1h-pyrazol-4-yl)-isoxazole-3-carboxylic acid, 5-(1,3-dimethyl-1h-pyrazol-4-yl)-isoxazole-3-carboxylic acid, 5-(1,5-dimethyl-1h-pyrazol-4-yl)-isoxazole, 3,5-dimethylisoxazole-4-boronic acid pinacol ester, 3,5-dimethylisoxazole, 3-(dimethylamino)-1-(2-pyridyl)-2-propen-1-one, 5-(3,5-difluorophenyl)isoxazole, [2,6-dichloro-4-(trifluoromethyl)phenyl]hydrazine, 5-(2,5-dichlorophenyl)isoxazole-3-carboxylic acid, danazol, 3-(4-chlorophenyl)-5-(trifluoromethyl)isoxazole-4-carboxylic acid, 5-(4-chlorophenyl)isoxazole-3-propionic acid, 5-(4-chlorophenyl)isoxazole-4-carboxylic acid, 5-(4-chlorophenyl)isoxazole-3-carboxylic acid, 3-(4-chlorophenyl)isoxazole-5-carboxylic acid, 3-(3-chlorophenyl)isoxazole-5-carboxylic acid, 5-(4-chlorophenyl)isoxazole-3-carboxaldehyde, 3-(4-chlorophenyl)isoxazole-5-carbaldehyde, 3-(3-chlorophenyl)isoxazole-5-carbaldehyde, 3-(2-chlorophenyl)isoxazole-5-carbaldehyde, 5-(4-chlorophenyl)isoxazole, 3-(4-chlorophenyl)isoxazole, 5-(chloromethyl)isoxazole-4-carboxylic acid, 3-(chloromethyl)-5-(2-furyl)isoxazole, 4-chloromethyl-3,5-dimethylisoxazole, 5-(chloromethyl)-3-(4-chlorophenyl)isoxazole, 5-(3-chloro-4-methoxy-phenyl)-isoxazole-3-carboxylic acid, 3-chloro-4-fluorobenzaldehyde, 3-(5-chloro-2,4-dimethoxy-phenyl)-4,5-dihydro-isoxazole-5-carboxylic acid, 5-tert-butyl-4,5,6,7-tetrahydro-benzo[d]isoxazole-3-carboxylic acid, 3-(4-bromophenyl)-5-(trifluoromethyl)isoxazole-4-carboxylic acid, 5-(4-bromophenyl)isoxazole-3-propionic acid, 5-(4-bromophenyl)isoxazole-3-carboxylic acid hydrazide, 5-(4-bromophenyl)isoxazole-4-carboxylic acid, 5-(4-bromophenyl)isoxazole-3-carboxylic acid, 3-(4-bromophenyl)isoxazole-5-carboxylic acid, 3-(4-bromophenyl)isoxazole-5-carboxaldehyde, 5-(4-bromophenyl)isoxazole, 5-(3-bromophenyl)isoxazole, 3-(4-bromophenyl)isoxazole, 5-(bromomethyl)-3-(4-methoxyphenyl)isoxazole, 4-(bromomethyl)isoxazole, 5-(bromomethyl)-3-(4-fluorophenyl)isoxazole, 5-(bromomethyl)-3-(4-chlorophenyl)isoxazole, 5-(bromomethyl)-3-(4-bromophenyl)isoxazole, 6-bromo-3-methylbenzo[d]isoxazole, 5-bromo-3-methylbenzo[d]isoxazole, 4-bromo-5-(4-methoxyphenyl)isoxazole, 3-bromo-isoxazole, 3-bromo-5-(2-hydroxyethyl)isoxazole, 4-bromo-5-(4-fluorophenyl)isoxazole, 3-bromo-5-(4-fluorophenyl)isoxazole, 4-bromo-5-(4-chlorophenyl)isoxazole, 4-bromo-5-(4-bromophenyl)isoxazole, 6-bromo-benzo[d]isoxazole-3-carboxylic acid, benzo[d]isoxazole-3-carboxylic acid, 3-amino-5-methylisoxazole, 5-amino-3-(4-methoxyphenyl)isoxazole, 3-aminoisoxazole, 3-amino-5-(4-fluorophenyl)isoxazole, 5-amino-3-(4-chlorophenyl)isoxazole, 5-amino-4-(4-bromophenyl)isoxazole, 3-amino-5-(4-bromophenyl)isoxazole, 5-acetyl-3-(4-fluorophenyl)isoxazole, 5-acetyl-3-(3-fluorophenyl)isoxazole, 3-methyl-5-[(2s)-1-methyl-2-pyrrolidinyl]isoxazole hydrochloride, 7-methoxy-5-methyl-4,5-dihydronaphtho[2,1-d]isoxazole, 5-methyl-3-phenyl-isoxazole-4-carboxylic acid methylamide, 5-methyl-3-phenyl-isoxazole-4-carbothioic acid methylamide, 5-methyl-3-phenyl-4-(1h-pyrazol-5-yl)isoxazole, 5-benzyl-3-furan-2-yl-2-phenyl-tetrahydro-pyrrolo(3,4-d)isoxazole-4,6-dione, 5-benzyl-3-[4-(dimethylamino)phenyl]-2-phenyldihydro-2h-pyrrolo[3,4-d]isoxazole-4,6(3h,5h)-dione, 5-benzyl-3-(5-br-2-ho-phenyl)-2-ph-tetrahydro-pyrrolo(3,4-d)isoxazole-4,6-dione, 5-benzyl-3-(4-nitro-ph)-2-ph-dihydro-2h-pyrrolo(3,4-d)isoxazole-4,6(3h,5h)-dione, 5-benzyl-3-(4-methoxy-phenyl)-2-ph-tetrahydro-pyrrolo(3,4-d)isoxazole-4,6-dione, 5-benzyl-3-(4-fluorophenyl)-2-(2-methylphenyl)dihydro-2h-pyrrolo[3,4-d]isoxazole-4,6(3h,5h)-dione, 5-benzyl-3-(3-nitro-ph)-2-phenyl-tetrahydro-pyrrolo(3,4-d)isoxazole-4,6-dione, 5-benzyl-2-ph-3-(2-pyridinyl)dihydro-2h-pyrrolo(3,4-d)isoxazole-4,6(3h,5h)-dione, 5-benzyl-2-ph-3-(2-ph-vinyl)dihydro-2h-pyrrolo(3,4-d)isoxazole-4,6(3h,5h)-dione, 5-benzyl-2-(4-chlorophenyl)-3-(2-thienyl)dihydro-2h-pyrrolo[3,4-d]isoxazole-4,6(3h,5h)-dione, 5-benzyl-2,3-diphenyldihydro-2h-pyrrolo(3,4-d)isoxazole-4,6(3h,5h)-dione, 5-benzyl-2(4-cl-ph)3-(2-furyl)dihydro-2h-pyrrolo(3,4-d)isoxazole-4,6(3h,5h)dione, 5-benzyl-2(4-cl-ph)-3-(4-f-ph)dihydro-2h-pyrrolo(3,4-d)isoxazole-4,6(3h,5h)dione, 5-(p-tolyl)isoxazole, 5-(4-methylphenyl)-3-(4-nitrophenyl)-2-phenyldihydro-2h-pyrrolo[3,4-d]isoxazole-4,6(3h,5h)-dione, 5-(4-methoxyphenyl)-2-phenyl-3-(4-pyridinyl)dihydro-2h-pyrrolo[3,4-d]isoxazole-4,6(3h,5h)-dione, 5-(4-methoxyphenyl)-2-phenyl-3-(3-pyridinyl)dihydro-2h-pyrrolo[3,4-d]isoxazole-4,6(3h,5h)-dione, 5-(4-methoxy-ph)-2,3-diphenyldihydro-2h-pyrrolo(3,4-d)isoxazole-4,6(3h,5h)-dione, 5-(4-fluorophenyl)-3-(4-methoxyphenyl)-2-phenyldihydro-2h-pyrrolo[3,4-d]isoxazole-4,6(3h,5h)-dione, 5-(4-fluorophenyl)-2-(2-methylphenyl)-3-(4-pyridinyl)dihydro-2h-pyrrolo[3,4-d]isoxazole-4,6(3h,5h)-dione, 5-(4-ethoxyphenyl)-3-(4-nitrophenyl)-2-phenyldihydro-2h-pyrrolo[3,4-d]isoxazole-4,6(3h,5h)-dione, 5-(4-ethoxyphenyl)-3-(4-fluorophenyl)-2-phenyldihydro-2h-pyrrolo[3,4-d]isoxazole-4,6(3h,5h)-dione, 5-(4-ethoxyphenyl)-2-methyl-3-(4-nitrophenyl)dihydro-2h-pyrrolo[3,4-d]isoxazole-4,6(3h,5h)-dione, 5-(4-ethoxy-ph)-2-ph-3-thiophen-2-yl-4h-pyrrolo(3,4-d)isoxazole-4,6-dione, 5-(4-cl-ph)-3-(3-nitro-ph)-2-phenyl-tetrahydro-pyrrolo(3,4-d)isoxazole-4,6-dione, 5-(4-bromophenyl)-3-(4-methoxyphenyl)-2-phenyldihydro-2h-pyrrolo[3,4-d]isoxazole-4,6(3h,5h)-dione, 5-(4-bromophenyl)-3-(2-furyl)-2-(2-methylphenyl)dihydro-2h-pyrrolo[3,4-d]isoxazole-4,6(3h,5h)-dione, 5-(4-bromophenyl)-2-phenyl-3-(2-pyridinyl)dihydro-2h-pyrrolo[3,4-d]isoxazole-4,6(3h,5h)-dione, 5-(4-br-ph)2-ph-3-(2-ph-vinyl)dihydro-2h-pyrrolo(3,4-d)isoxazole-4,6(3h,5h)dione, 5-(2-cl-ph)-3-(4-dimethylamino-ph)2-o-tolyl-4h-pyrrolo(3,4-d)isoxazole-4,6-dione, 5-(2-chlorophenyl)-3-[4-(dimethylamino)phenyl]-2-phenyldihydro-2h-pyrrolo[3,4-d]isoxazole-4,6(3h,5h)-dione, 5-(2-chlorophenyl)-3-(4-methoxyphenyl)-2-phenyldihydro-2h-pyrrolo[3,4-d]isoxazole-4,6(3h,5h)-dione, 4-((3-(2-cl-ph)-5-methyl-isoxazole-4-carbonyl)-amino)-benzoic acid ethyl ester, 4,5,6,6a-tetrahydro-3ah-cyclopenta[d]isoxazole-3-carboxylic acid, 3-phenyl-3a,6a-dihydrothieno[2,3-d]isoxazole 4,4-dioxide, 3-methyl-5-(3-phenylpropyl)isoxazole, 3-methyl-4-nitro-5-[(e)-2-phenylethenyl]isoxazole, 3-methyl-4,5,8,9-tetrahydrocycloocta(d)isoxazole, 3-methyl-4,5,5a,6a,7,8-hexahydrooxireno(2′,3′:5,6)cycloocta(1,2-d)isoxazole, 3-methyl-3a,4,5,8,9,9a-hexahydrocycloocta(d)isoxazole, 3-furan-2-yl-2-phenyl-5-p-tolyl-tetrahydro-pyrrolo(3,4-d)isoxazole-4,6-dione, 3-chloro-4,5-dihydro(1)-benzothiepino(5,4-c)isoxazole, 3-[4-(dimethylamino)phenyl]-5-(4-methoxyphenyl)-2-(2-methylphenyl)dihydro-2h-pyrrolo[3,4-d]isoxazole-4,6(3h,5h)-dione, 3-(5-br-2-ho-phenyl)-2,5-diphenyl-tetrahydro-pyrrolo(3,4-d)isoxazole-4,6-dione, 3-(5-br-2-ho-ph)-5-(2-cl-ph)-2-ph-tetrahydro-pyrrolo(3,4-d)isoxazole-4,6-dione, 3-(4-meo-phenyl)-5-phenyl-2-o-tolyl-tetrahydro-pyrrolo(3,4-d)isoxazole-4,6-dione, 3-(4-fluorophenyl)-5-(4-nitrophenyl)-2-phenyldihydro-2h-pyrrolo[3,4-d]isoxazole-4,6(3h,5h)-dione, 3-(4-fluorophenyl)-5-(4-methylphenyl)-2-phenyldihydro-2h-pyrrolo[3,4-d]isoxazole-4,6(3h,5h)-dione, 3-(4-dimethylamino-ph)-5-ph-2-o-tolyl-4h-pyrrolo(3,4-d)isoxazole-4,6-dione, 3-(4-fluorophenyl)-5-(4-methoxyphenyl)-2-phenyldihydro-2h-pyrrolo[3,4-d]isoxazole-4,6(3h,5h)-dione, 3-(4-br-ph)-2-ph-5-(2-trifluoromethyl-ph)-4h-pyrrolo(3,4-d)isoxazole-4,6-dione, 3-(3-nitro-phenyl)-2,5-diphenyl-tetrahydro-pyrrolo(3,4-d)isoxazole-4,6-dione, 3-(3-br-phenyl)-2,5-diphenyldihydro-2h-pyrrolo(3,4-d)isoxazole-4,6(3h,5h)-dione, 3-(3-br-ph)-5-(2-meo-ph)-2-o-tolyl-tetrahydro-pyrrolo(3,4-d)isoxazole-4,6-dione, 3-(2-furyl)-5-[4-(4-morpholinyl)phenyl]-2-phenyldihydro-2h-pyrrolo[3,4-d]isoxazole-4,6(3h,5h)-dione, 3-(2-furyl)-2-(2-me-ph)-5-ph-dihydro-2h-pyrrolo(3,4-d)isoxazole-4,6(3h,5h)-dione, 3-(2-furyl)-2,5-diphenyldihydro-2h-pyrrolo(3,4-d)isoxazole-4,6(3h,5h)-dione, 3-(2-cl-phenyl)-5-methyl-isoxazole-4-carboxylic acid (2,5-dichloro-phenyl)-amide, 3-(2-cl-ph)-5-me-isoxazole-4-carboxylic acid (4,5-dihydro-thiazol-2-yl)-amide, 3-(2-chloro-phenyl)-5-methyl-isoxazole-4-carboxylic acid cyanomethyl-amide, 3-(2,4-dichlorophenyl)-5-(4-methoxyphenyl)-2-phenyldihydro-2h-pyrrolo[3,4-d]isoxazole-4,6(3h,5h)-dione, 3-(2,4-di-cl-ph)-2,5-diphenyldihydro-2h-pyrrolo(3,4-d)isoxazole-4,6(3h,5h)-dione, 3-(2,2-dichloro-vinyl)-5-phenyl-isoxazole, 3,5-diphenyl-isoxazole, 3,5-dimethyl-4-(1-pyrrolidinylsulfonyl)isoxazole, 3(4-dimethylamino-ph)-5-(4-eto-ph)2-o-tolyl-4h-pyrrolo(3,4-d)isoxazole-4,6-dione, 2-(4-cl-ph)5-ph-3-(2-thienyl)dihydro-2h-pyrrolo(3,4-d)isoxazole-4,6(3h,5h)-dione, 2-(4-cl-ph)-5-(3-meo-ph)-3-(3-nitro-ph)-4h-pyrrolo(3,4-d)isoxazole-4,6-dione, 2-(4-cl-ph)-3-(4-meo-ph)-5-p-tolyl-tetrahydro-pyrrolo(3,4-d)isoxazole-4,6-dione, 2-(4-chlorophenyl)-5-(4-methylphenyl)-3-(2-thienyl)dihydro-2h-pyrrolo[3,4-d]isoxazole-4,6(3h,5h)-dione, 2-(4-chlorophenyl)-3-[4-(dimethylamino)phenyl]-5-phenyldihydro-2h-pyrrolo[3,4-d]isoxazole-4,6(3h,5h)-dione, 2-(4-chlorophenyl)-3-(4-fluorophenyl)-5-(4-nitrophenyl)dihydro-2h-pyrrolo[3,4-d]isoxazole-4,6(3h,5h)-dione, 2-(4-chlorophenyl)-3-(2-thienyl)-5-[3-(trifluoromethyl)phenyl]dihydro-2h-pyrrolo[3,4-d]isoxazole-4,6(3h,5h)-dione, 2-(4-chlorophenyl)-3-(2,4-dichlorophenyl)-5-phenyldihydro-2h-pyrrolo[3,4-d]isoxazole-4,6(3h,5h)-dione, 2,3-di-ph-5-(3-(tri-f-me)ph)dihydro-2h-pyrrolo(3,4-d)isoxazole-4,6(3h,5h)-dione, danazol, and n-cyclopropyl-5-(thiophen-2-yl)isoxazole-3-carboxamide.

In one aspect, the effective amount comprises an amount that results in overexpression of the miRNA, such as for example, about 5 μM to about 25 μM.

In a further aspect, the cells are cultured in serum-free media.

In one aspect, the stem cell or progenitor cell is selected from the group of an iPSC-derived cardiac progenitor cell or an iPSC-derived embryoid body. Methods to derive iPSC-cardiac progenitor cells from stem cells are known in the art and described herein. Methods to derive stem cells from an iPSC-derived embryoid body are known in the art as described for example in Michael W. Nestor, et al., Differentiation of serum-free embryoid bodies from human induced pluripotent stem cells into networks, In Stem Cell Research, Volume 10, Issue 3, 2013, Pages 454-463, ISSN 1873-5061, https://doi.org/10.1016/j.scr.2013.02.001.; Steven D. Sheridan, et al., “Analysis of Embryoid Bodies Derived from Human Induced Pluripotent Stem Cells as a Means to Assess Pluripotency,” Stem Cells International, vol. 2012, Article ID 738910, 9: 2012; Heming Wei, et al., “One-step derivation of cardiomyocytes and mesenchymal stem cells from human pluripotent stem cells” Stem Cell Research, Volume 9, Issue 2, 2012, Pages 87-100, ; and Di Pasquale E., et al., J. Vis Exp. 2013 Jun. 28; (76). “Generation of human cardiomyocytes: a differentiation protocol from feeder-free human induced pluripotent stem cells”

In one aspect, the isoxazole compound is selected from isoxazole-1 (isx-1) or isoxazole-9 (isx-9). In another aspect, the isoxazole derivative is compound of the formula:

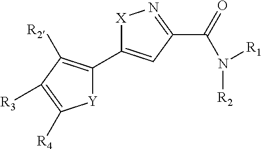

wherein R₁ and R₂ are both hydrogen or R₁ is hydrogen and R₂ is selected from the group consisting of substituted or unsubstituted C₁-C₆ alkyl, C₃-C₆ cycloalkyl, C₂-C₆ alkenyl, C₂-C₆ alkynyl, and benzyl, or where R₁ and R₂ may be joined together to form a ring selected from azetidinyl, pyrrolidinyl, piperidinyl, piperazinyl, or morpholinyl; R₂′, R₃ and R₄ are independently selected from the group consisting of hydrogen, halogen, C₁-C₆ alkyl, C₃-C₆ cycloalkyl, substituted or unsubstituted aromatic or heteroaromatic ring, cyano, nitro, and acyl; X is 0, NH or S; and Y is 0, NH or S.

In a yet further aspect, the isoxazole compound has the formula:

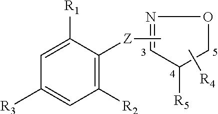

wherein R1 and R2 is each selected from C1-C4 alkyl, phenyl, benzyl, trifluoromethyl or halogen, R3 is selected from hydrogen, hydroxy, C1-C4 alkyl or alkoxy, R4, in position 3 or 5, is selected from hydrogen, trifluoromethyl, C1-C4 alkoxy, C1-C4 alkyl, or C1-C4 hydroxyalkyl, R5 is selected from hydrogen or C4-C4 alkyl or R4 and R5 together form a tetramethylene group, Z at position 3 or 5 on the heterocycle is selected from: —N(R6)-CO—, —CO—N(R6)-, —N(R6)—CO—N(R6)-, —CH(R6)-NH—CO—, or —NH—CO—CH(R6), in which R6 is selected from hydrogen or C1-C4 alkyl.

In a yet further aspect, the isoxazole compound or a derivative thereof is selected from the group: 5-(trifluoromethyl)-3-(4-methoxyphenyl)isoxazole-4-carboxylic acid, 5-(trifluoromethyl)-3-(4-fluorophenyl)isoxazole-4-carboxylic acid, 5-(thiophen-2-yl)isoxazole-3-carboxaldehyde, 5,6,7,8-tetrahydro-4h-cyclohepta[d]isoxazole-3-carboxylic acid, 4,5,6,7-tetrahydro-benzo[d]isoxazole-3-carboxylic acid, 3-amino-5-methylisoxazole, 4-amino-n-(5-methyl-3-isoxazolyl)benzenesulfonamide, 3-phenyl-isoxazole-5-boronic acid pinacol ester, 5-phenylisoxazole, 1-phenyl-1-cyclopentanecarboxylic acid, 3-phenyl-benzo[c]isoxazole-5-carboxylic acid, 5-methyl-3-phenylisoxazole-4-carboxylic acid, 3a,4,5,6,7,8,9,9a-octahydro-cycloocta[d]isoxazole-3-carboxylic acid, 5-(3-nitrophenyl)isoxazole, 3-(4-nitrophenyl)isoxazole, 3-hydroxy-5-aminomethyl-isoxazole, 5-(morpholinomethyl)isoxazole-3-carboxylic acid hydrochloride, 5-(morpholinomethyl)isoxazole-3-carbaldehyde, 3-methyl-5-(trifluoromethyl)isoxazole-4-carboxylic acid, methyl 5-(thiophen-2-yl)isoxazole-3-carboxylate, 3-(methylsulfonyl)-5-(2-thienyl)isoxazole-4-carbonitrile, 5-methyl-3-(2-pyrrolidinyl)isoxazole, 3-methyl-5-(2-pyrrolidinyl)isoxazole, 3-(1-methyl-1h-pyrazol-4-yl)-isoxazole-5-carboxylic acid, 3-(1-methyl-1h-pyrazol-4-yl)-4,5-dihydro-isoxazole-5-carboxylic acid, 5-(4-methylphenyl)isoxazole-3-carboxylic acid, 5-methyl-3-phenylisoxazole-4-carboxylic acid, 5-(4-methylphenyl)isoxazole-3-carboxaldehyde, 5-methyl-3-(4-phenoxyphenyl)isoxazole-4-carboxylic acid, 3-methyl-5-(4-methyl-1,2,3-thiadiazol-5-yl)isoxazole-4-carboxylic acid, 3-methyl-5-(5-methylisoxazol-3-yl)isoxazole-4-carboxylic acid, methyl 5-(4-methoxyphenyl)isoxazole-4-carboxylate, methyl 5-(4-methoxyphenyl)isoxazole-3-carboxylate, 5-methylisoxazole, methyl 5-(4-fluorophenyl)isoxazole-4-carboxylate, methyl 5-(4-fluorophenyl)isoxazole-3-carboxylate, methyl 5-(4-chlorophenyl)isoxazole-4-carboxylate, methyl 5-(4-bromophenyl)isoxazole-4-carboxylate, 5-(4-methoxyphenyl)isoxazole-3-carboxylic acid, 5-(3-methoxy-phenyl)-isoxazole-3-carboxylic acid, 3-(2-methoxyphenyl)isoxazole-5-carboxylic acid, 5-(4-methoxyphenyl)isoxazole-3-carboxaldehyde, 3-(4-methoxyphenyl)isoxazole-5-carbaldehyde, 3-(2-methoxyphenyl)isoxazole-5-carbaldehyde, 5-(4-methoxyphenyl)isoxazole, 3-(4-methoxyphenyl)isoxazole, 3-(2-methoxy-phenyl)-4,5-dihydro-isoxazole-5-carboxylic acid, 3-methoxy-isoxazole-5-carboxylic acid, isoxazole-5-carboxylic acid, isoxazole-4-carboxylic acid, isoxazole-5-carbothioamide, isoxazole-5-carbonyl chloride, isoxazole-3-carbonitrile, isoxazole-3-carbaldehyde, isoxazole-4-boronic acid, isoxazole, 5-cyclopropyl-4-[2-(methylsulfonyl)-4-(trifluoromethyl)benzoyl]isoxazole, 6-(5-(thiophen-2-yl)isoxazole-3-carboxamido)hexyl 5-((3as,4s,6ar)-2-oxohexahydro-1h-thieno[3,4-d]imidazol-4-yl)pentanoate, isocarboxazid 5-methyl-3-isoxazole-carboxylic acid 2-benzylhydrazide, 5-isobutyl-isoxazole-3-carboxylic acid, 4-iodo-5-methyl-isoxazole, 3,3′-iminobis(n,n-dimethylpropylamine), 3-(3-hydroxy-phenyl)-isoxazole-5-carboxylic acid methyl ester, 5-(4-hydroxy-phenyl)-isoxazole-3-carboxylic acid, 5-(3-hydroxy-phenyl)-isoxazole-3-carboxylic acid, 5-(hydroxymethyl)-3-methylisoxazole, 3-hydroxy-5-methylisoxazole, 5-(1-hydroxyethyl)-3-(4-trifluoromethylphenyl)isoxazole, 3a,4,5,6,7,7a-hexahydro-benzo[d]isoxazole-3-carboxylic acid, 5-(2-furyl)isoxazole-3-carbaldehyde, 5-furan-2-yl-isoxazole-3-carboxylic acid, 6-fluoro-3-(4-piperidinyl)benzisoxazole, 5-(4-fluorophenyl)isoxazole-3-methanol, 3-(2-fluoro-phenyl)-isoxazole-5-carboxylic acid, 5-(4-fluorophenyl)isoxazole-3-carboxaldehyde, 3-(4-fluorophenyl)isoxazole-5-carbaldehyde, 3-(3-fluorophenyl)isoxazole-5-carbaldehyde, 3-(2-fluorophenyl)isoxazole-5-carbaldehyde, 5-(4-fluorophenyl)isoxazole, 3-(4-fluorophenyl)isoxazole, 5-(3-fluoro-4-methoxy-phenyl)-isoxazole-3-carboxylic acid, ethyl 5-(trifluoromethyl)-3-(4-methoxyphenyl)isoxazole-4-carboxylate, ethyl-5-(tributylstannyl)isoxazole-3-carboxylate, ethyl 5-(thiophen-2-yl)isoxazole-3-carboxylate, 5-ethyl-isoxazole-4-carboxylic acid, 5-ethyl-isoxazole-3-carboxylic acid, ethyl 5-(4-fluorophenyl)isoxazole-4-carboxylate, ethyl 5-(4-fluorophenyl)isoxazole-3-carboxylate, ethyl 5-(2,3-dihydrobenzo[b][1,4]dioxin-7-yl)isoxazole-3-carboxylate, ethyl 3-(4-chlorophenyl)-5-(trifluoromethyl)isoxazole-4-carboxylate, ethyl 5-(4-chlorophenyl)isoxazole-3-carboxylate, ethyl 3-(4-bromophenyl)-5-(trifluoromethyl)isoxazole-4-carboxylate, ethyl 5-(4-bromophenyl)isoxazole-3-carboxylate, ethyl 5-amino-4-(4-chlorophenyl)isoxazole-3-carboxylate, ethyl 5-amino-4-(4-bromophenyl)isoxazole-3-carboxylate, ethyl 6b-acetyl-2-(acetyloxy)-4a,6a-dimethyl-2,3,4,4a,4b,5,6,6a,6b,9a,10,10a,10b,11-tetradecahydro-1 h-naphtho[2′,1′:4,5]indeno[2,1-d]isoxazole-9-carboxylate, 3,5-dimethyl-4-(tributylstannyl)isoxazole, 5-(1,5-dimethyl-1h-pyrazol-4-yl)-isoxazole-3-carboxylic acid, 5-(1,3-dimethyl-1h-pyrazol-4-yl)-isoxazole-3-carboxylic acid, 5-(1,5-dimethyl-1h-pyrazol-4-yl)-isoxazole, 3,5-dimethylisoxazole-4-boronic acid pinacol ester, 3,5-dimethylisoxazole, 3-(dimethylamino)-1-(2-pyridyl)-2-propen-1-one, 5-(3,5-difluorophenyl)isoxazole, [2,6-dichloro-4-(trifluoromethyl)phenyl]hydrazine, 5-(2,5-dichlorophenyl)isoxazole-3-carboxylic acid, danazol, 3-(4-chlorophenyl)-5-(trifluoromethyl)isoxazole-4-carboxylic acid, 5-(4-chlorophenyl)isoxazole-3-propionic acid, 5-(4-chlorophenyl)isoxazole-4-carboxylic acid, 5-(4-chlorophenyl)isoxazole-3-carboxylic acid, 3-(4-chlorophenyl)isoxazole-5-carboxylic acid, 3-(3-chlorophenyl)isoxazole-5-carboxylic acid, 5-(4-chlorophenyl)isoxazole-3-carboxaldehyde, 3-(4-chlorophenyl)isoxazole-5-carbaldehyde, 3-(3-chlorophenyl)isoxazole-5-carbaldehyde, 3-(2-chlorophenyl)isoxazole-5-carbaldehyde, 5-(4-chlorophenyl)isoxazole, 3-(4-chlorophenyl)isoxazole, 5-(chloromethyl)isoxazole-4-carboxylic acid, 3-(chloromethyl)-5-(2-furyl)isoxazole, 4-chloromethyl-3,5-dimethylisoxazole, 5-(chloromethyl)-3-(4-chlorophenyl)isoxazole, 5-(3-chloro-4-methoxy-phenyl)-isoxazole-3-carboxylic acid, 3-chloro-4-fluorobenzaldehyde, 3-(5-chloro-2,4-dimethoxy-phenyl)-4,5-dihydro-isoxazole-5-carboxylic acid, 5-tert-butyl-4,5,6,7-tetrahydro-benzo[d]isoxazole-3-carboxylic acid, 3-(4-bromophenyl)-5-(trifluoromethyl)isoxazole-4-carboxylic acid, 5-(4-bromophenyl)isoxazole-3-propionic acid, 5-(4-bromophenyl)isoxazole-3-carboxylic acid hydrazide, 5-(4-bromophenyl)isoxazole-4-carboxylic acid, 5-(4-bromophenyl)isoxazole-3-carboxylic acid, 3-(4-bromophenyl)isoxazole-5-carboxylic acid, 3-(4-bromophenyl)isoxazole-5-carboxaldehyde, 5-(4-bromophenyl)isoxazole, 5-(3-bromophenyl)isoxazole, 3-(4-bromophenyl)isoxazole, 5-(bromomethyl)-3-(4-methoxyphenyl)isoxazole, 4-(bromomethyl)isoxazole, 5-(bromomethyl)-3-(4-fluorophenyl)isoxazole, 5-(bromomethyl)-3-(4-chlorophenyl)isoxazole, 5-(bromomethyl)-3-(4-bromophenyl)isoxazole, 6-bromo-3-methylbenzo[d]isoxazole, 5-bromo-3-methylbenzo[d]isoxazole, 4-bromo-5-(4-methoxyphenyl)isoxazole, 3-bromo-isoxazole, 3-bromo-5-(2-hydroxyethyl)isoxazole, 4-bromo-5-(4-fluorophenyl)isoxazole, 3-bromo-5-(4-fluorophenyl)isoxazole, 4-bromo-5-(4-chlorophenyl)isoxazole, 4-bromo-5-(4-bromophenyl)isoxazole, 6-bromo-benzo[d]isoxazole-3-carboxylic acid, benzo[d]isoxazole-3-carboxylic acid, 3-amino-5-methylisoxazole, 5-amino-3-(4-methoxyphenyl)isoxazole, 3-aminoisoxazole, 3-amino-5-(4-fluorophenyl)isoxazole, 5-amino-3-(4-chlorophenyl)isoxazole, 5-amino-4-(4-bromophenyl)isoxazole, 3-amino-5-(4-bromophenyl)isoxazole, 5-acetyl-3-(4-fluorophenyl)isoxazole, 5-acetyl-3-(3-fluorophenyl)isoxazole, 3-methyl-5-[(2s)-1-methyl-2-pyrrolidinyl]isoxazole hydrochloride, 7-methoxy-5-methyl-4,5-dihydronaphtho[2,1-d]isoxazole, 5-methyl-3-phenyl-isoxazole-4-carboxylic acid methylamide, 5-methyl-3-phenyl-isoxazole-4-carbothioic acid methylamide, 5-methyl-3-phenyl-4-(1h-pyrazol-5-yl)isoxazole, 5-benzyl-3-furan-2-yl-2-phenyl-tetrahydro-pyrrolo(3,4-d)isoxazole-4,6-dione, 5-benzyl-3-[4-(dimethylamino)phenyl]-2-phenyldihydro-2h-pyrrolo[3,4-d]isoxazole-4,6(3h,5h)-dione, 5-benzyl-3-(5-br-2-ho-phenyl)-2-ph-tetrahydro-pyrrolo(3,4-d)isoxazole-4,6-dione, 5-benzyl-3-(4-nitro-ph)-2-ph-dihydro-2h-pyrrolo(3,4-d)isoxazole-4,6(3h,5h)-dione, 5-benzyl-3-(4-methoxy-phenyl)-2-ph-tetrahydro-pyrrolo(3,4-d)isoxazole-4,6-dione, 5-benzyl-3-(4-fluorophenyl)-2-(2-methylphenyl)dihydro-2h-pyrrolo[3,4-d]isoxazole-4,6(3h,5h)-dione, 5-benzyl-3-(3-nitro-ph)-2-phenyl-tetrahydro-pyrrolo(3,4-d)isoxazole-4,6-dione, 5-benzyl-2-ph-3-(2-pyridinyl)dihydro-2h-pyrrolo(3,4-d)isoxazole-4,6(3h,5h)-dione, 5-benzyl-2-ph-3-(2-ph-vinyl)dihydro-2h-pyrrolo(3,4-d)isoxazole-4,6(3h,5h)-dione, 5-benzyl-2-(4-chlorophenyl)-3-(2-thienyl)dihydro-2h-pyrrolo[3,4-d]isoxazole-4,6(3h,5h)-dione, 5-benzyl-2,3-diphenyldihydro-2h-pyrrolo(3,4-d)isoxazole-4,6(3h,5h)-dione, 5-benzyl-2(4-cl-ph)3-(2-furyl)dihydro-2h-pyrrolo(3,4-d)isoxazole-4,6(3h,5h)dione, 5-benzyl-2(4-cl-ph)-3-(4-f-ph)dihydro-2h-pyrrolo(3,4-d)isoxazole-4,6(3h,5h)dione, 5-(p-tolyl)isoxazole, 5-(4-methylphenyl)-3-(4-nitrophenyl)-2-phenyldihydro-2h-pyrrolo[3,4-d]isoxazole-4,6(3h,5h)-dione, 5-(4-methoxyphenyl)-2-phenyl-3-(4-pyridinyl)dihydro-2h-pyrrolo[3,4-d]isoxazole-4,6(3h,5h)-dione, 5-(4-methoxyphenyl)-2-phenyl-3-(3-pyridinyl)dihydro-2h-pyrrolo[3,4-d]isoxazole-4,6(3h,5h)-dione, 5-(4-methoxy-ph)-2,3-diphenyldihydro-2h-pyrrolo(3,4-d)isoxazole-4,6(3h,5h)-dione, 5-(4-fluorophenyl)-3-(4-methoxyphenyl)-2-phenyldihydro-2h-pyrrolo[3,4-d]isoxazole-4,6(3h,5h)-dione, 5-(4-fluorophenyl)-2-(2-methylphenyl)-3-(4-pyridinyl)dihydro-2h-pyrrolo[3,4-d]isoxazole-4,6(3h,5h)-dione, 5-(4-ethoxyphenyl)-3-(4-nitrophenyl)-2-phenyldihydro-2h-pyrrolo[3,4-d]isoxazole-4,6(3h,5h)-dione, 5-(4-ethoxyphenyl)-3-(4-fluorophenyl)-2-phenyldihydro-2h-pyrrolo[3,4-d]isoxazole-4,6(3h,5h)-dione, 5-(4-ethoxyphenyl)-2-methyl-3-(4-nitrophenyl)dihydro-2h-pyrrolo[3,4-d]isoxazole-4,6(3h,5h)-dione, 5-(4-ethoxy-ph)-2-ph-3-thiophen-2-yl-4h-pyrrolo(3,4-d)isoxazole-4,6-dione, 5-(4-cl-ph)-3-(3-nitro-ph)-2-phenyl-tetrahydro-pyrrolo(3,4-d)isoxazole-4,6-dione, 5-(4-bromophenyl)-3-(4-methoxyphenyl)-2-phenyldihydro-2h-pyrrolo[3,4-d]isoxazole-4,6(3h,5h)-dione, 5-(4-bromophenyl)-3-(2-furyl)-2-(2-methylphenyl)dihydro-2h-pyrrolo[3,4-d]isoxazole-4,6(3h,5h)-dione, 5-(4-bromophenyl)-2-phenyl-3-(2-pyridinyl)dihydro-2h-pyrrolo[3,4-d]isoxazole-4,6(3h,5h)-dione, 5-(4-br-ph)2-ph-3-(2-ph-vinyl)dihydro-2h-pyrrolo(3,4-d)isoxazole-4,6(3h,5h)dione, 5-(2-cl-ph)-3-(4-dimethylamino-ph)2-o-tolyl-4h-pyrrolo(3,4-d)isoxazole-4,6-dione, 5-(2-chlorophenyl)-3-[4-(dimethylamino)phenyl]-2-phenyldihydro-2h-pyrrolo[3,4-d]isoxazole-4,6(3h,5h)-dione, 5-(2-chlorophenyl)-3-(4-methoxyphenyl)-2-phenyldihydro-2h-pyrrolo[3,4-d]isoxazole-4,6(3h,5h)-dione, 4-((3-(2-cl-ph)-5-methyl-isoxazole-4-carbonyl)-amino)-benzoic acid ethyl ester, 4,5,6,6a-tetrahydro-3ah-cyclopenta[d]isoxazole-3-carboxylic acid, 3-phenyl-3a,6a-dihydrothieno[2,3-d]isoxazole 4,4-dioxide, 3-methyl-5-(3-phenylpropyl)isoxazole, 3-methyl-4-nitro-5-[(e)-2-phenylethenyl]isoxazole, 3-methyl-4,5,8,9-tetrahydrocycloocta(d)isoxazole, 3-methyl-4,5,5a,6a,7,8-hexahydrooxireno(2′,3′:5,6)cycloocta(1,2-d)isoxazole, 3-methyl-3a,4,5,8,9,9a-hexahydrocycloocta(d)isoxazole, 3-furan-2-yl-2-phenyl-5-p-tolyl-tetrahydro-pyrrolo(3,4-d)isoxazole-4,6-dione, 3-chloro-4,5-dihydro(1)-benzothiepino(5,4-c)isoxazole, 3-[4-(dimethylamino)phenyl]-5-(4-methoxyphenyl)-2-(2-methylphenyl)dihydro-2h-pyrrolo[3,4-d]isoxazole-4,6(3h,5h)-dione, 3-(5-br-2-ho-phenyl)-2,5-diphenyl-tetrahydro-pyrrolo(3,4-d)isoxazole-4,6-dione, 3-(5-br-2-ho-ph)-5-(2-cl-ph)-2-ph-tetrahydro-pyrrolo(3,4-d)isoxazole-4,6-dione, 3-(4-meo-phenyl)-5-phenyl-2-o-tolyl-tetrahydro-pyrrolo(3,4-d)isoxazole-4,6-dione, 3-(4-fluorophenyl)-5-(4-nitrophenyl)-2-phenyldihydro-2h-pyrrolo[3,4-d]isoxazole-4,6(3h,5h)-dione, 3-(4-fluorophenyl)-5-(4-methylphenyl)-2-phenyldihydro-2h-pyrrolo[3,4-d]isoxazole-4,6(3h,5h)-dione, 3-(4-dimethylamino-ph)-5-ph-2-o-tolyl-4h-pyrrolo(3,4-d)isoxazole-4,6-dione, 3-(4-fluorophenyl)-5-(4-methoxyphenyl)-2-phenyldihydro-2h-pyrrolo[3,4-d]isoxazole-4,6(3h,5h)-dione, 3-(4-br-ph)-2-ph-5-(2-trifluoromethyl-ph)-4h-pyrrolo(3,4-d)isoxazole-4,6-dione, 3-(3-nitro-phenyl)-2,5-diphenyl-tetrahydro-pyrrolo(3,4-d)isoxazole-4,6-dione, 3-(3-br-phenyl)-2,5-diphenyldihydro-2h-pyrrolo(3,4-d)isoxazole-4,6(3h,5h)-dione, 3-(3-br-ph)-5-(2-meo-ph)-2-o-tolyl-tetrahydro-pyrrolo(3,4-d)isoxazole-4,6-dione, 3-(2-furyl)-5-[4-(4-morpholinyl)phenyl]-2-phenyldihydro-2h-pyrrolo[3,4-d]isoxazole-4,6(3h,5h)-dione, 3-(2-furyl)-2-(2-me-ph)-5-ph-dihydro-2h-pyrrolo(3,4-d)isoxazole-4,6(3h,5h)-dione, 3-(2-furyl)-2,5-diphenyldihydro-2h-pyrrolo(3,4-d)isoxazole-4,6(3h,5h)-dione, 3-(2-cl-phenyl)-5-methyl-isoxazole-4-carboxylic acid (2,5-dichloro-phenyl)-amide, 3-(2-cl-ph)-5-me-isoxazole-4-carboxylic acid (4,5-dihydro-thiazol-2-yl)-amide, 3-(2-chloro-phenyl)-5-methyl-isoxazole-4-carboxylic acid cyanomethyl-amide, 3-(2,4-dichlorophenyl)-5-(4-methoxyphenyl)-2-phenyldihydro-2h-pyrrolo[3,4-d]isoxazole-4,6(3h,5h)-dione, 3-(2,4-di-cl-ph)-2,5-diphenyldihydro-2h-pyrrolo(3,4-d)isoxazole-4,6(3h,5h)-dione, 3-(2,2-dichloro-vinyl)-5-phenyl-isoxazole, 3,5-diphenyl-isoxazole, 3,5-dimethyl-4-(1-pyrrolidinylsulfonyl)isoxazole, 3(4-dimethylamino-ph)-5-(4-eto-ph)2-o-tolyl-4h-pyrrolo(3,4-d)isoxazole-4,6-dione, 2-(4-cl-ph)5-ph-3-(2-thienyl)dihydro-2h-pyrrolo(3,4-d)isoxazole-4,6(3h,5h)-dione, 2-(4-cl-ph)-5-(3-meo-ph)-3-(3-nitro-ph)-4h-pyrrolo(3,4-d)isoxazole-4,6-dione, 2-(4-cl-ph)-3-(4-meo-ph)-5-p-tolyl-tetrahydro-pyrrolo(3,4-d)isoxazole-4,6-dione, 2-(4-chlorophenyl)-5-(4-methylphenyl)-3-(2-thienyl)dihydro-2h-pyrrolo[3,4-d]isoxazole-4,6(3h,5h)-dione, 2-(4-chlorophenyl)-3-[4-(dimethylamino)phenyl]-5-phenyldihydro-2h-pyrrolo[3,4-d]isoxazole-4,6(3h,5h)-dione, 2-(4-chlorophenyl)-3-(4-fluorophenyl)-5-(4-nitrophenyl)dihydro-2h-pyrrolo[3,4-d]isoxazole-4,6(3h,5h)-dione, 2-(4-chlorophenyl)-3-(2-thienyl)-5-[3-(trifluoromethyl)phenyl]dihydro-2h-pyrrolo[3,4-d]isoxazole-4,6(3h,5h)-dione, 2-(4-chlorophenyl)-3-(2,4-dichlorophenyl)-5-phenyldihydro-2h-pyrrolo[3,4-d]isoxazole-4,6(3h,5h)-dione, 2,3-di-ph-5-(3-(tri-f-me)ph)dihydro-2h-pyrrolo(3,4-d)isoxazole-4,6(3h,5h)-dione, danazol, and n-cyclopropyl-5-(thiophen-2-yl)isoxazole-3-carboxamide.

Also provided are cell populations prepared by the above methods as well as exosomes or microvesicles isolated and/or purified from these cell populations. The populations can be homogenous, heterogeneous, purified, highly purified, homogenous or substantially homogeneous. In one aspect, at least 80%, or alternatively 85%, or alternatively 90%, or alternatively 95%, or alternatively 97% or alternatively 99%, or alternatively 100%, of the cells express the protein or proteins.

Further provided are cell populations and exosomes or microvesicles isolated from these populations, wherein the populations are cardiac or skeletal myogenic progenitors prepared from a population of human induced pluripotent stem cells (hiPSCs) by culturing the hiPSCs with an effective amount of Givinostat (GIV) for an effective amount of time. In one aspect the effective amount of GIV comprises from about 100 nM to about 30 nM per 1×10⁶ cells, or from about 100 nM to about 50 nM per 1×10⁶ cells, or from about 90 nM to about 30 nM per 1×10⁶ cells, or from about 30 nM to about 75 nM per 1×10⁶ cells, or from about 70 nM to about 50 nM per 1×10⁶ cells. In a further aspect, the cells and exosome or microvesicle populations are prepared by methods that further comprise culturing the cells in the presence of serum-free medium and Rho-associated kinase (ROCK) inhibitors. Non-limiting examples of the Rho-associated kinase (ROCK) inhibitors are Thiazovivin or Y27632, which are commercially available from R & D Systems, Sigma Aldrich and Stemgent. Methods to isolate the compositions are known in the art, some of which are described herein.

The cells and exosomes or microvesicles are useful in regenerating skeletal muscle or treating DMD by administering to cells and/or exosomes or microvesicles to a subject in need thereof. Methods to determine the effectiveness of the therapy are known in the art, some of which are described herein.

In another aspect, the populations are prepared by culturing the cells in the presence of serum-free medium and a TGF-beta type-I receptor inhibitor, non-limiting examples of such TGF-beta type-I receptor inhibitor include SB431542 and A8301, which are commercially available from Cayman Chemical and Sigma Aldrich; LY2157299, which can be purchased from AdooQ Bioscience (Catalog No. A11017); and LY2109761, which can be purchased from AdooQ Bioscience and Selleckchem (Catalog No. S2704). The cell populations can be heterogeneous, homogeneous, substantially homogeneous or highly purified. In one aspect, the populations comprise at least 80%, or alternatively 85%, or alternatively 90%, or alternatively 95%, or alternatively 97% or alternatively 99%, or alternatively 100%, of the cells express the exosomes or microvesicles. Methods to isolate the populations are known in the art, some of which are described herein.

These cells and exosomes or microvesicles are useful for regenerating cardiac muscle by administering to a subject in need thereof an effective amount of the population or exosomes or microvesicles. They also are useful for treating cardiac dysfunction associated with Duchenne Muscular Dystrophy (DMD), by administering to a subject in need thereof an effective amount of the population of cells or the exosomes or microvesicles. Methods to determine the effectiveness of the therapy are known in the art, some of which are described herein.

In a further aspect, the population of cells overexpress at least skeletal myogenic genes selected from the group of: Meox1, Meox2, Tcf15, Pax3, Pax7, MyoD1, dystrophin, Myf5, or DESMIN. In one aspect, the populations overexpress, at least one, or at least two, or at least three, or at least four, or at least five, or at least at least six, or at least seven, or at least eight, or at all nine eighteen skeletal myogenic genes. The cells are prepared by culturing or contacting an iPSC in the presence of an effective amount of Givinostat (GIV), and optionally in serum free media. The effective amount of GIV is from about 100 nM to about 30 nM per 1×10⁶ cells. In a further aspect, the cells are also cultured in the presence of or contacted in the presence of an effective amount of ROCK inhibitors, non-limited examples of such include Thiazovivin, Y27632, SR3677, or GSK429286. In a yet further aspect, the cells are cultured (or contacted) in the presence of a TGF-beta type I inhibitor. Non-limiting examples of such include SB431542, A8301, LY2157299, or LY2109761. The cell populations can be heterogeneous, homogeneous, substantially homogeneous or highly purified. In one aspect, the populations comprise at least 80%, or alternatively 85%, or alternatively 90%, or alternatively 95%, or alternatively 97% or alternatively 99%, or alternatively 100%, of the cells express the genes.

The cells and/or exosomes or microvesicles isolated from the cells are useful in methods for regenerating skeletal muscle or for treating Duchenne Muscular Dystrophy (DMD) by administering to a subject in need thereof an effective amount of the population of cells and/or exosomes or microvesicles isolated from the cells. Methods to determine the effectiveness of the therapy are known in the art, some of which are described herein.

In a further aspect, also provided is a population of cells that overexpress xESI myogenic genes selected from the group of: Pitx2, ISL1, Nkx2.5, Hand1, GATA4, Tbx5, TnnT2, Myl7, MLC2v, Myf2c, Cdh4, or Lhx2 as well as exosomes or microvesicles isolated from the cells. In one aspect, the populations overexpress, at least one, or at least two, or at least three, or at least four, or at least five, or at least at least six, or at least seven, or at least eight, or at least nine, or at least ten, or at least eleven, or all twelve xESI myogenic genes. The cell or exosome or microvesicle populations can be heterogeneous, homogeneous, substantially homogeneous or highly purified. In one aspect, the populations comprise at least 80%, or alternatively 85%, or alternatively 90%, or alternatively 95%, or alternatively 97% or alternatively 99%, or alternatively 100%, of the cells overexpress the genes. Methods to isolate cells overexpressing the on e or more genes, and exosomes or microvesicles isolated from these populations are known n the art some of which are described herein.

The populations and exosomes or microvesicles are useful for regenerating cardiac muscle and/or for cardiac dysfunction associated with Duchenne Muscular Dystrophy (DMD) by administering to a subject in need thereof an effective amount of the population of cells and/or exosomes or microvesicles isolated from the cells. Methods to determine the effectiveness of the therapies are known in the art, some of which are described herein.

Also provided is a composition for the repair or regeneration of damaged or diseased cardiac tissue or for the treatment of one or more of Hoyeraal-Hreidarsson syndrome, dyskeratosis congenita, pulmonary fibrosis, aplastic anemia, liver fibrosis, dyskeratosis congenita, bone marrow failure, lung disease, endocrine diseases, polycystic ovary syndrome (PCOS), Cushing's syndrome, and acromegaly, Cerebrovascular Disease (Strokes), High Blood Pressure—Hypertension, Parkinson's Disease, Dementia, Alzheimer's Disease, Age-related hearing loss, Celiac disease (CD), COPD, bipolar disorder, hydroxyurea, sickle cell diseases, hypertension, atherosclerosis, arthritis, osteoporosis, osteoarthritis, vasculardementia or macular degeneration, cancer, type 2 diabetes, or diseases with telomerase dysfunction dealing with a shortened telomere length, the composition comprising, or alternatively consisting essentially of, or yet further consisting of, a synthetic microRNA-146a and a pharmaceutically acceptable carrier, and optionally administration of an effective amount of Danazol. These compositions are useful for regenerating tissue in a subject in need thereof, by administering one or more microRNA fragments, or derivatives thereof to the individual, wherein after administration of the one or more microRNA fragments, the one or more microRNA fragments alter gene expression in the damaged tissue, improve the viability of said damaged tissue, and facilitate the formation of new tissue in the subject. Methods to determine the effectiveness of the therapy are known in the art, some of which are described herein.

For the purposes of this disclosure, the exosomes or microvesicles have an average diameter of from about 20 nm to about 90 nm.

### Compositions

This invention also provides compositions containing the cells, exosomes or microvesicles, miRNA, and populations containing the same, as described above, in combination with a carrier, such as a biocompatible scaffold or a pharmaceutically acceptable carrier. In a further aspect the compositions also contain a preservative and/or a cryoprotectant. The compositions can be freeze-dried or lyophilized and provided in one or more dosage formulations. In one embodiment, the composition is intended for therapeutic use and therefore, an effective amount of the modified cell, exosomes or microvesicles, miRNA, and populations are provided, alone or in combination with the isoxazole or isoxazole similar compound, in the composition.

Methods for Preparing Certain Cell Populations, Exosomes or Microvesicles and miRNA Populations, and Uses Thereof

Also provided herein are methods for preparing an isolated population of exosomes or microvesicles that overexpress a microRNA (miRNA) selected from the group of mir-373, mir-210 mir-377, mir-367, mir-520, mir-548ah, mir-335, mir-30c, mir-214, or mir-548q and/or one or more protein selected from Tsg101, CD9, Hsp 70, Flotillin-1, or GAPDH. In one aspect, at least 70%, or 80%, or 85%, or 90% or 95%, or 98% of the cells express the stated mir and/or protein.

In one aspect, the following method is provided. Briefly, more than 50 mice with MI, and 3 time points of cardiac function assessment during one month observation period were assessed for the Applicant's exosome data. miRNAs secreted by exosomes derived from specific cardiac progenitor cells exerted a strong therapeutic effect on myocardial infarction. Paracrine effects of cardiac exosomes derived from progenitors on cell survival, migration and differentiation play an important role in cardiac regeneration. miRNA signatures are unique in exosomes amongst different parent cells. Therefore, Applicant isolated and characterized exosomes from iPSC and isoxazole induced cardiac progenitor cells (Exo-CPC^{iso}) and determined their effects on cardiac cells in vitro and on mice hearts following myocardial infarction (MI). Applicant characterized the exosomes isolated from iPSC and CPCs. Transmission electron microscopy (TEM) of exosomes isolated from iPSC and CPC^{ISX-9} showed typical morphology. Western blot further confirmed that exosomes from iPSC and CPC^{ISX-9} were enriched in Exosome (Exo)-specific markers Tsg101. Other common exosome proteins including CD9, Hsp70 and Flotillin-1. Calnexin was absent in exosomes. No significant difference in average size of exosomes from iPSC and CPC^{ISX-9} was observed.

Applicant also investigated the miRNA cargo contents in the exosomes from CPCs. We found miR-373, miR-367, miR-520 and miR-548 were significantly overexpressed in Exo-CPC^{iso} compared with exosomes from iPSC, commercial CPCs and embryoid body (EB) and these miRNA microarray data were confirmed with real-time PCR. Go enrichment analysis based on miRNA-targeted genes showed that biological process included organelle, molecular function, response to stress and mitotic cell cycle. Applicant further studied the effects of Exo-CPC^{iso} on cultured fibroblasts. PKH26 labeled exosomes from CPC^{ISX-9} were observed inside the fibroblasts Calcein AM), mostly located at the perinuclear region. Interestingly, expression of fibrotic genes (CTCF, FN1, TIMP1, TIMP2, MMP2 and MMP9) in cultured fibroblasts stimulated with TGF-β was significantly downregulated after treatment with Exo-CPCiso. Loss-of-function analysis using miR-373 inhibitor confirmed that enrichment of miR-373 was the major contributor to reversion of TGF-β stimulation in fibroblasts. Applicant also tested the therapeutic effects in murine MI model. Applicant found intramyocardial injection of Exo-CPC′ exerted favorable cardioprotective effects on cardiomyocyte proliferation, angiogenesis and preservation of cardiac function in mice one month post-MI compared with PBS and Exo-iPSC treated mice. ki67 positive CM (cTnT positive) in Exo-CPC^{SX-9} treated mouse 30 days after MI and quantitative analysis of proliferating CMs revealed that Exo-CPC^{SX-9} significantly increased proliferation of CMs in peri-infarct region 30 days after MI as determined by Ki67 (data not shown). Quantitate analysis of arterioles density showed Exo-CPC^{SX-9} significantly increased arterioles density after MI. Furthermore, functional measurement demonstrated Exo-CPC^{ISX-9} treatment significantly improved EF and FS (data not shown). Quantitative analysis showed smaller fibrosis area from Exo-CPC^{ISX-9} treated mice after MI (data not shown). Hence, a Micro-RNA 373 mimic would overexpress Micro-RNA 373 which would reduce scar tissue in fibrotic diseases.

The exosome or microvesicle populations can be heterogeneous, homogeneous, substantially homogeneous or highly purified. In one aspect, the populations comprise at least 80%, or alternatively 85%, or alternatively 90%, or alternatively 95%, or alternatively 97% or alternatively 99%, or alternatively 100%, of the exosomes or microvesicles contain express the miRNA.

Also provided are methods to prepare and isolate population of one or more of: a cardiac progenitor cell, a cardiomyocyte, a myocyte, an endothelial cell, a smooth muscle cell, a skeletal muscle cell, each generated from a cell selected from the group of: an iPSC, an embryonic stem cell or a stem cell that was contacted with an isoxazole compound, derivative, or an equivalent of each thereof, wherein the cells or the population containing same overexpress one or more of mir-373, mir-210 mir-377, mir-367, mir-520, mir-548ah, mir-335, mir-30c, mir-214, or mir-548q; and/or a muscle gene selected from the group of: paZ3, pAX7, MYF5, MYOD, MYOG, or dystrophin. In one aspect, at least 70%, or 80%, or 85%, or 90% or 95%, or 98% of the cells express the stated mir and/or muscle gene. The exosomes or microvesicles can be isolated for any one of the following: a cardiac progenitor cell, a cardiomyocyte, a skeletal muscle, an endothelial cell, a myocyte, or a smooth muscle cell.

Also provided herein are methods for preparing cells that overexpress one or more of mir-373, mir-210 mir-377, mir-367, mir-520, mir-548ah, mir-335, mir-30c, mir-214, or mir-548q; and/or a muscle gene selected from the group of: paX3, pAX7, MYF5, MYOD, MYOG, or dystrophin. In one aspect, at least 70%, or 80%, or 85%, or 90% or 95%, or 98% of the cells overexpress the stated mir and/or muscle gene. Expression levels are determined using methods described herein and known in the art, e.g., high-throughput assays, ELISA and hybridization techniques. One or more of the mir and/or genes, as described above can be overexpressed.

Also provided are methods for preparing compositions containing the exosomes or microvesicles and/or cells. The compositions can further comprise a protein that facilitates regeneration and/or improved function of a tissue or a nucleic acid that encodes the protein and/or an agent that inhibits the expression of an inflammatory protein, that are optionally a cytokine. Examples of such are described herein.

The populations are prepared by culturing a population of stem cells or progenitor cells cultured in the presence of an effective amount of an isoxazole compound, a derivative thereof or an equivalent thereof, and for an effective amount of time. Exemplary isoxazole and derivatives there of are provided herein. In one aspect, the effective amount comprises an amount that results in overexpression of the miRNA, such as for example from about 5 μM to about 25 μM or ranges in between as described above. Non-limiting exemplary stem cell or progenitor cells are selected from the group of an iPSC-derived CPC, a cardiomyocyte, a skeletal muscle cell, an endothelial cell, a myocyte, a smooth muscle cell, or an iPSC-derived embryoid body.

After the cells have been cultured under the appropriate conditions, the exosomes or microvesicles are isolated from the cells or cell culture supernatant using methods described herein and methods known in the art. Depending on the purification methods, the compositions can be heterogeneous, purified, highly purified, homogenous, or substantially homogeneous with the desired level of purity as described herein. The exosomes or microvesicles and cells can be detectably labeled by adding to the cells and/or exosomes or microvesicles a detectable label by conjugating to them the label using methods described herein or known in the art.

Depending on the purification methods, the cell compositions can be purified, highly purified, homogenous, or substantially homogeneous and having the desired percentage purity. The cells can be detectably labeled by adding to the cells a detectable label by conjugating to them the label using methods described herein or known in the art.

The cells can be admixed or combined with a carrier wherein the carrier is optionally a non-naturally occurring carrier.

A preservative or cryoprotectant can be combined or admixed with the cells or compositions containing them. These compositions can be lyophilized using methods known in the art and/or formulated into appropriate dosage forms for ease of use.

The cells and/or exosomes or microvesicles can be admixed or combined with a carrier wherein the carrier is optionally a non-naturally occurring carrier. In one aspect, the population of cells is cultured in serum-free media.

A preservative or cryoprotectant can be combined or admixed with the cells, exosomes or microvesicles or compositions containing them. These compositions can be lyophilized using methods known in the art and/or formulated into appropriate dosage forms for ease of use.

The cells and compositions prepared by these methods and comprising a microRNA (miRNA) selected from the group of mir-373, mir-210, mir-377, mir-367, mir-520, mir-548ah, mir-335, mir-21, mir-30c, mir-214 or mir-548q; and/or one or more of a protein selected from: Tsg101, CD9, Hsp70, Flotillin-1 or GAPDH, or alternatively cells or a population of cells that overexpress one or more of mir-373, mir-210 mir-377, mir-367, mir-520, mir-548ah, mir-335, mir-30c, mir-214, or mir-548q; and/or a muscle gene selected from the group of: paX3, pAX7, MYF5, MYOD, MYOG, or dystrophin are useful for one or more of providing in a subject in need thereof regenerating damaged tissue such as muscle and/or cardiac tissue; improving the viability of damaged tissue, such as muscle and/or cardiac tissue; facilitating the formation of new tissue, such as cardiac tissue, muscle tissue, skeletal muscle tissue, a blood vessel, a capillary, or a myocyte; promoting cardiac regeneration; promoting cardiac regeneration in a subject suffering from an acute cardiac event; promoting cardiac regeneration in a subject suffering from a myocardial infarction; promoting cardiac regeneration in subject suffering from Duchenne Muscular Dystrophy (DMD) or Duchenne Muscular Dystrophy (“DMD”)-associated cardiomyopathy, promoting cardiac regeneration in a subject suffering from age-related diseases, such as for example, chronic obstructive pulmonary disease (“COPD”), arthritis, osteoporosis, osteoarthritis, diabetes, vascular dementia, or macular degeneration; promoting tissue regeneration in tissue damaged from one or more of stroke, arthritis, Alzheimer's, memory loss discorders, cystic fibrosis, inflammatory disorders and cancer; decreasing cardiac wall thickness in a tissue damaged from a cardiac infarction; altering gene expression of one or more of protein kinase C, iL-6, mmp, PDGF; reducing or inhibiting the expression of an inflammatory protein that is optionally a chemokine, macrophage or a cytokine; directly or indirectly stimulating angiogenesis; promoting cardiac regeneration in a subject suffering from a disease selected from the group of coronary artery disease, myocardial infarction, heart failure, hypoplasic left heart syndrome, peripheral artery disease (PAD), cardiac mypertrophy, valvular heart disease (aortic stenosis), myocardial hypertrophy, hypertrophy fibrosis; and/or directly or indirectly inhibit cellular replication. The methods are accomplished by administering an effective amount of the exosomes or microvesicles, cells, populations or compositions containing the same to a subject in need thereof. The methods can further comprise administering an effective amount of non-embryonic stem cell or a progenitor cell to the subject that is optionally the same or different type of tissue that is in need of repair. In one aspect, the non-embryonic stem or progenitor cell is autologous to the subject. In another aspect, the non-embryonic stem or progenitor cell is allogeneic to the subject. The exosomes or microvesicles, cells, populations can be locally or systemically delivered to the subject. Subjects suitably treated by these methods include animals, mammals and human patients. Methods to determine the effectiveness of the therapy are known in the art, some of which are described herein, by administering an effective amount of the composition of cells, exosomes or microvesicles or compositions containing the same to the subject in need thereof. As noted above, methods for determining the effectiveness of the therapy are known in the art, some of which are described herein.

The methods can further comprise administering an effective amount of a non-embryonic stem cell or progenitor cell to the subject, that is optionally of the same type as the tissue in need of repair of a type different from the type of tissue of repair. The cells can be non-embryonic stem or progenitor cell is autologous or allogeneic to the subject.

Any appropriate method of administration can be used, e.g., topical, by infusion or intravenously, as determined by the treating veterinarian or physician. The appropriate route of administration and dosage also depends on the age, health and gender of the subject being treated as well as the formulation. Effective amounts can be determined empirically by the treating veterinarian or physician. In one aspect, the cells, exosomes or microvesicles or compositions are delivered locally or systemically to the tissue of the subject or by an intramyocardial or an intracoronary route.

Also provided are methods of preparing a population of cardiac or skeletal myogenic progenitors from a population of human induced pluripotent stem cells (hiPSCs) by contacting the hiPSCs with an effective amount of Givinostat (GIV) or culturing the cells in the presence of an effective amount of Givinostat (GIV) for an effective amount of time. In one aspect, the effective amount of GIV comprises from about 100 nM to about 30 nM per 1×10⁶ cells and ranges in between as described herein. In a further aspect, the cells are cultured in the presence of serum-free medium and an effective amount of a Rho-associated kinase (ROCK) inhibitors for an effective amount of time. Non-limiting examples of such are described above and incorporated herein by reference. In a further aspect, an effective amount of a TGF-beta type-1 receptor inhibitor is added to the culture medium, non-limiting examples of the TGF-beta type-1 receptor inhibitor are described above, and incorporated herein by reference.

After the cells have been cultured under the appropriate conditions, the cells are isolated from the cell culture supernatant using methods described herein and methods known in the art.

Use of the markers can be used to isolated and purify the compositions, Depending on the purification methods, the cell compositions can be purified, highly purified, homogenous, or substantially homogeneous and/or having the desired percentage purity as described above. The cells can be detectably labeled by adding to the cells a detectable label by conjugating to them the label using methods described herein or known in the art. In a further aspect, exosome or microvesicle are isolated from the cells using methods described herein and known in the art.

The cells and/or exosomes or microvesicles can be admixed or combined with a carrier wherein the carrier is optionally a non-naturally occurring carrier.

A preservative or cryoprotectant can be combined or admixed with the cells, exosomes or microvesicles or compositions containing them. These compositions can be lyophilized using methods known in the art and/or formulated into appropriate dosage forms for ease of use.

The cells, exosomes or microvesicles and composition containing them are useful in methods for regenerating skeletal muscle, or regenerating cardiac muscle, and/or treating DMD by administering to a subject in need thereof an effective amount of the population of cells. Any appropriate method of administration can be used, e.g., topical, by infusion or intravenously, as determined by the treating veterinarian or physician. It also depends on the age, health and gender of the subject being treated as well as the formulation. Effective amounts can be determined empirically by the treating veterinarian or physician. In one aspect, the cells, exosomes or microvesicles or compositions are delivered locally or systemically to the tissue of the subject or by an intramyocardial or an intracoronary route. Methods to determine the effectiveness of the therapy are known in the art, some of which are described herein.

Also provided are compositions for the repair or regeneration of damaged or diseased cardiac tissue, the composition comprising, or alternatively consisting essentially of, or yet consisting of synthetic microRNA-146a (miRNA-146a) or a fragment thereof, and optionally a pharmaceutically acceptable carrier, and optionally wherein the composition is purified, highly purified, or substantially homogeneous. In a further aspect, the composition can be heterogeneous. Synthetic mRNA-146a is commercially available from vendors such as Sigma Aldrich or can be manufactured using conventional techniques. In one aspect, the microRNA fragments, or derivatives thereof, are synthetically generated using techniques such as Artificial microRNA (amiRNA) technology which exploits microRNA (miRNA) biogenesis pathway to produce artificially designed small RNAs using miRNA gene backbone vis the following steps: Step 1: predict the target miRNAs from gene coding sequence. Step 2: predict artificial miR* sequence from miR. Step 3: predict primers from the secondary structure of pre-microRNA. In a further aspect, the microRNA fragments, or derivatives thereof are synthesized with a sequence that mimics one or more endogenous microRNA molecules using techniques such as miRNA mimics which are small, chemically modified double-stranded RNAs that mimic endogenous miRNAs and enable miRNA functional analysis by up-regulation of miRNA activity. miRNA inhibitors are small, chemically modified single-stranded RNA molecules designed to specifically bind to and inhibit endogenous miRNA molecules and enable miRNA functional analysis by down-regulation of miRNA activity. In a yet further aspect, the wherein the microRNA fragments, or derivatives thereof are modified to enhance their stability using techniques such as anti-miRNA oligonucleotides which inhibit miRNA activity to fine-tune specific signaling pathways or to block miRNA function induced by disease. These anti-miRNA oligonucleotides may be modified to enhance stability, target affinity, and promote cellular uptake. AntagomiRs are anti-miRNAs that are modified in one or more of the following ways: (1) 2′-O-methylation of the ribose sugar to enhance nuclease resistance and improve the binding affinity to target RNAs; (2) phosphorothioate linkage between nucleotides to provide stability, with the balance between phosphodiester and phosphorothioate enhancing oligonucleotide stability against nucleases; and (3) cholesterol conjugation at the 3′UTR to facilitate cellular uptake. Locked nucleic acid (LNA-)-modified oligonucleotides are anti-miRNAs with a 2′ sugar modification in which the ribose is locked in a C3′-endo conformation by a 2′-O, 4′-C methylene bridge that strongly increases the affinity for complementary RNA and increases the duplex melting temperature miRNA inhibition is achieved by anti-miRNAs, which are single-stranded oligonucleotides complementary to target miRNAs. AntagomiRs and LNA oligonucleotides are anti-miRNAs modified to improve uptake and stability. Examples of Micro-ma 195 inhibitors are known and are sold by Sigma-Aldrich (HSTUD0320 SIGMA MISSION® Synthetic microRNA Inhibitor, Human hsa-miR-195-5p) and abmgood.com (e.g., inhibitory hsa-miR-195-5p miRNA/microRNA Lentivector). Hence, a Micro-RNA 373 mimic would overexpress micro-RNA 373 which would reduce scar tissue in fibrotic diseases.

Examples of micro-ma 373 mimic's are known and sold by Sigma-Aldrich under MISSION® microRNA Mimic hsa-miR-373* (sigmaaldrich.com/catalog/product/sigma/hmi0531?lang=en&region=US). Additional Micro-ma 373 mimic's, mir-373 inhibitors, mir-373 oglio's, mir-373 expression vector's, mir-373 precursers are known and sold by: Switchgear genomics (switchgeargenomics.com/products/lightswitch-mirna-mimics-inhibitors/inhibitors/mir-300-399 (mir373 inhibitors; switchgeargenomics.com/products/lightswitch-mirna-mimics-inhibitors/mir-300-399. (miR373 mimics (switchgeargenomics.com/products/synthetic-3utr-goclone-reporters/mir-300-399 (miR 3′UTR reporter)); (addgene.org/78127/(miR-373 expression vector); Thermo-Fischer (thermofisher.com/us/en/home/life-science/epigenetics-noncoding-rna-research/mirna-analysis/mirna-mimics-inhibitors/mirvana-mimics-inhibitors.html and thermofisher.com/us/en/home/life-science/epigenetics-noncoding-rna-research/mirna-analysis/mirna-mimics-inhibitors/ambion-pre-mir-precursors.html.

IDT oligos can be used and are commercially available (see idtdna.com/pages/decoded/decoded-articles/product-spotlight/decoded/2012/04/11/inhibiting-mirnas-using-antisense-oligonucleotides). Others can be purchase from Qiagen (qiagen.com/us/shop/genes-and-pathways/mirna-details.aspx?mirnaid=7344).

The miRNA compositions can be detectably labeled by adding to the miRNAs a detectable label by conjugating to them the label using methods described herein or known in the art. The miRNA s can be admixed or combined with a carrier wherein the carrier is optionally a non-naturally occurring carrier. A preservative or cryoprotectant can be combined or admixed with the miRNA or compositions containing them. These compositions can be lyophilized using methods known in the art and/or formulated into appropriate dosage forms for ease of use.

In a further aspect, the methods to generate these compositions are encapsulated or conjugated to synthetic liposomes using techniques known in the art. “Liposomes” are microscopic vesicles consisting of concentric lipid bilayers. Structurally, liposomes range in size and shape from long tubes to spheres, with dimensions from a few hundred Angstroms to fractions of a millimeter. Vesicle-forming lipids are selected to achieve a specified degree of fluidity or rigidity of the final complex providing the lipid composition of the outer layer. These are neutral (cholesterol) or bipolar and include phospholipids, such as phosphatidylcholine (PC), phosphatidylethanolamine (PE), phosphatidylinositol (PI), and sphingomyelin (SM) and other types of bipolar lipids including but not limited to dioleoylphosphatidylethanolamine (DOPE), with a hydrocarbon chain length in the range of 14-22, and saturated or with one or more double C═C bonds. Examples of lipids capable of producing a stable liposome, alone, or in combination with other lipid components are phospholipids, such as hydrogenated soy phosphatidylcholine (HSPC), lecithin, phosphatidylethanolamine, lysolecithin, lysophosphatidylethanol-amine, phosphatidylserine, phosphatidylinositol, sphingomyelin, cephalin, cardiolipin, phosphatidic acid, cerebrosides, distearoylphosphatidylethan-olamine (DSPE), dioleoylphosphatidylcholine (DOPC), dipalmitoylphosphatidylcholine (DPPC), palmitoyloteoylphosphatidylcholine (POPC), palmitoyloleoylphosphatidylethanolamine (POPE) and dioleoylphosphatidylethanolamine 4-(N-maleimido-triethyl)cyclohexane-1-carboxylate (DOPE-mal). Additional non-phosphorous containing lipids that can become incorporated into liposomes include stearylamine, dodecylamine, hexadecylamine, isopropyl myristate, triethanolamine-lauryl sulfate, alkyl-aryl sulfate, acetyl palmitate, glycerol ricinoleate, hexadecyl stereate, amphoteric acrylic polymers, polyethyloxylated fatty acid amides, and the cationic lipids mentioned above (DDAB, DODAC, DMRIE, DMTAP, DOGS, DOTAP (DOTMA), DOSPA, DPTAP, DSTAP, DC-Chol). Negatively charged lipids include phosphatidic acid (PA), dipalmitoylphosphatidylglycerol (DPPG), dioteoylphosphatidylglycerol and (DOPG), dicetylphosphate that are able to form vesicles. Typically, liposomes can be divided into three categories based on their overall size and the nature of the lamellar structure. The three classifications, as developed by the New York Academy Sciences Meeting, “Liposomes and Their Use in Biology and Medicine,” December 1977, are multi-lamellar vesicles (MLVs), small uni-lamellar vesicles (SUVs) and large uni-lamellar vesicles (LUVs). The biological active agents can be encapsulated in such for administration in accordance with the methods described herein and described for example in U.S. Pat. Nos. 5,512,295; 7,401,707, and 9,554,999. These compositions are further provided herein.

The synthetic miRNA compositions are useful to regenerate tissue in a subject in need thereof, by administering one or more microRNA fragments, or derivatives thereof to the subject wherein after administration of the one or more microRNA fragments, the one or more microRNA fragments alter gene expression in the damaged tissue, improve the viability of said damaged tissue, and facilitate the formation of new tissue in the subject.

Any appropriate method of administration can be used, e.g., topical, by infusion or intravenously, as determined by the treating veterinarian or physician. It also depends on the age, health and gender of the subject being treated as well as the formulation. Effective amounts can be determined empirically by the treating veterinarian or physician. Methods to determine if the methods are effective are known in the art some of which are described herein.

Also provided are methods to isolate a population of exosomes or microvesicles, comprising culturing a population of non-embryonic human regenerative cells in the presence of a hydrolase enzyme to induce the cells to secrete exosomes or microvesicles, thereby generating exosomes or microvesicles. In a further aspect, the secreted exosomes or microvesicles are isolated or purified from the culture media. In one aspect, the hydrolase comprises a member of the DNAse I superfamily of enzymes, non-limiting examples of such include a sphingomyelinase, selected from the group of lysosomal acid sphingomyelinase, secreted zinc-dependent acid sphingomyelinase, neutral sphingomyelinase, and alkaline sphingomyelinase. In another aspect, the neutral sphingomyelinase comprises one or more of magnesium-dependent neutral sphingomyelinase and magnesium-independent neutral sphingomyelinase. Non-limiting examples of such include neutral sphingomyelinase type I, neutral sphingomyelinase type 2, and neutral sphingomyelinase type 3.

### Methods and Uses of Cell Populations

Yet another embodiment of the invention is a method for restoring cardiac function in a tissue or host in need thereof. This and other therapeutic, diagnostic, and research uses are described herein.

In one embodiment, the invention provides methods for one or more of: regenerating cardiac muscle tissue that in one aspect, is scar tissue in the damaged or diseased heart; improving cardiac function or for treating a cardiac disease or condition in a patient in need thereof. The methods comprise contacting the tissue to be regenerated with an effective amount of isoxazole or isoxazole similar compound or by administering to a subject in need thereof, and/or an effective amount of the chemically modified cell or population of chemically modified cells described above. In a further aspect, the isolated cell, e.g., an iPS cell or other progenitor or stem cell is locally administered within an effective amount of isoxazole or isoxazole similar compound. In one aspect, the treated iPS cells were differentiated into myocytes forming myofibers in the scar tissue of the heart. The cells can be autologous or allogeneic to the host or patient. The subject and cells can be any species as described herein.

Yet another embodiment of the invention is a method for regenerating cardiac muscle tissue in a suitable host by administering to the host an effective amount of the chemically modified cell or population of chemically modified cells as described above. The cells can be autologous or allogeneic to the host or patient. The subject and cells can be any species as described above.

In another aspect, the method comprises, or alternatively consists essentially of, or yet further consists of administering to a patient in need thereof an effective amount of an iPS cell and an effective amount an isoxazole or isoxazole similar compound. Administration can be local to the site of damage, and can include direct injection of the cells and isoxazole or isoxazole similar compound into the heart of the patient. The method also includes combination therapy as described herein. For example, the method can be combined with an effective amount of electrical stimulation, before, after or concurrent to this therapy. The method can also be combined with an effective amount of other cardiac-inducing small molecules including:

1) Wnt/beta-catenin inhibitors, e.g., IWR-1, IWP-1. Include an FDA approved drug called Pyrvinium (common brand name is Vanquin). Another novel small molecule includes ICG-001 (Chemical Name: (6S,9aS)-Hexahydro-6-[(4-hydroxyphenyl)methyl]-8-(1-naphthalenylmethyl)-4,7-dioxo-N-(phenylmethyl)-2H-pyrazino[1,2-a]pyrimidine-1(6H)-carboxamide) that targets the Wnt/β-catenin pathway.

2) TGF-β inhibitors, such as the small molecule ITD-1 defined as Chemical Name: 4-[1,1′-Biphenyl]-4-yl-1,4,5,6,7,8-hexahydro-2,7,7-trimethyl-5-oxo-3-quinolinecarboxylic acid ethyl ester),

3) Prostaglandins and COX-2. Activation of cyclooxygenase 2 (COX-2) and subsequent production of prostaglandin E2 (PGE2) induced by MI (PGE2 is an FDA-approved treatment for induction of labor under the brand name Dinoprostone. Another example includes small molecule ONO-1301—a small molecule agonist of PGI2 with a synymom of 7,8-Dihydro-5-[](E)-[[α-(3 pyridyl)benzylidene]aminooxy]ethyl]-1-naphthyloxy]acetic acid and supplied by Sigma-Aldrich,

4) DPP-IV inhibitors in combination with granulocyte colony stimulating factor or G-CSF. (This approach combines two molecules: a small molecule inhibitor of dipeptidylpeptidase IV (DPP-IV), an enzyme that degrades SDF-1α, and granulocyte colony-stimulating factor (G-CSF), a biological molecule that enhances the release of stem cells from the bone marrow through matrix metalloproteinase 2,

5) Angiotensin (1-7) and Mas receptor (formula: C₄₁H₆₂N₁₂O₁₁ and supplied by Tochris).

The combination therapy can be sequential or concurrent, as determined by the condition being treated, the health of the patient and the selection of the particular combination therapy.

In one aspect, the stem cells or iPS cells are administered in the form of a cardiac patch derived from stem cells (e.g., adult or bone marrow derived progenitors cells, iPS cells, iPS cells derived cardiac lineage cells, small juvenile stem cells and/or engineered tissue cardiac patch transplantation for the heart repair in heart diseases). Thus, in one aspect, the stem cells comprise very small juvenile stem cells that are present in the bone marrow (that are comprised within bone marrow stem cells) and/or peripheral blood cells (that are comprised within circulating blood and heart-derived stem cells). These cells are with significantly high proliferative and differentiation potentials and can also be easily reprogrammed and/or converted to cardiac progenitors with isoxazole or isoxazole similar compounds or Wnt inhibitors. Thus, this method would not require the derivation of iPS cells for safe cell transplantation. This disclosure also provides a method for promoting stem cell survival and differentiation by applying cardiac patches with derivatives of iPS cells treated with isoxazole or isoxazole similar compounds and Wnt inhibitors administered over scar area allowing better penetration, migration and integration of patch cells (myocytes, endothelial cells, smooth muscle cells. (FIGS. 14A-14H) with host heart.

This disclosure also provides a method for promoting stem cell survival and differentiation in vitro, comprising applying an effective amount of electrical stimulation to the stem cell. In one aspect, the effective amount comprises wherein the electrical stimulation comprises from about 1.0V/1.5 cm to about 2.0V/2.0 cm for about 1 to about 5 hours.

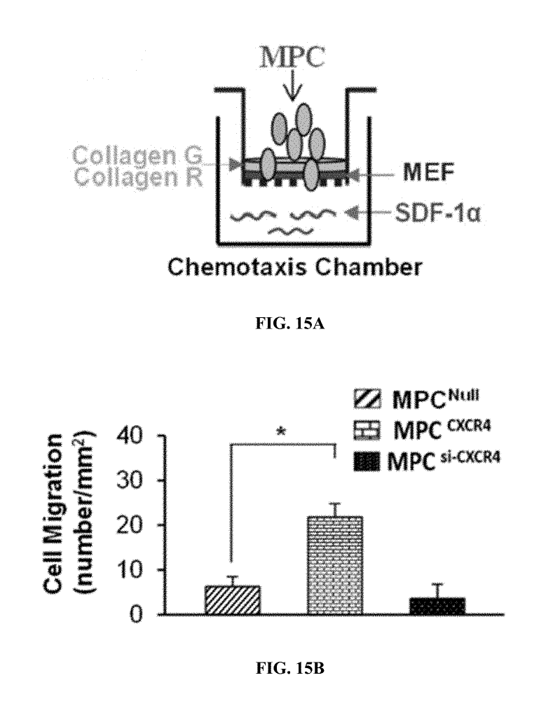

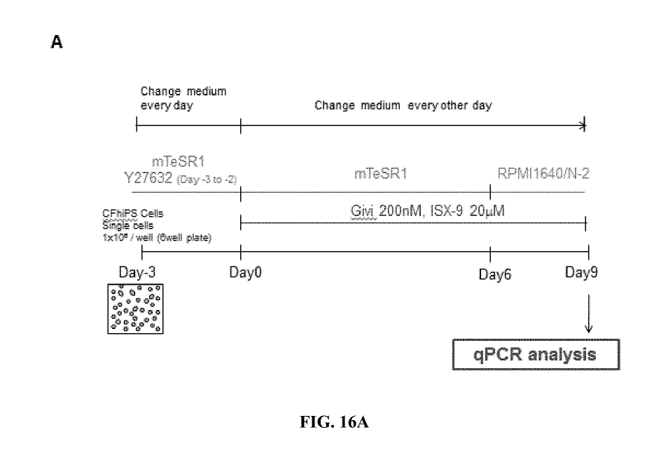

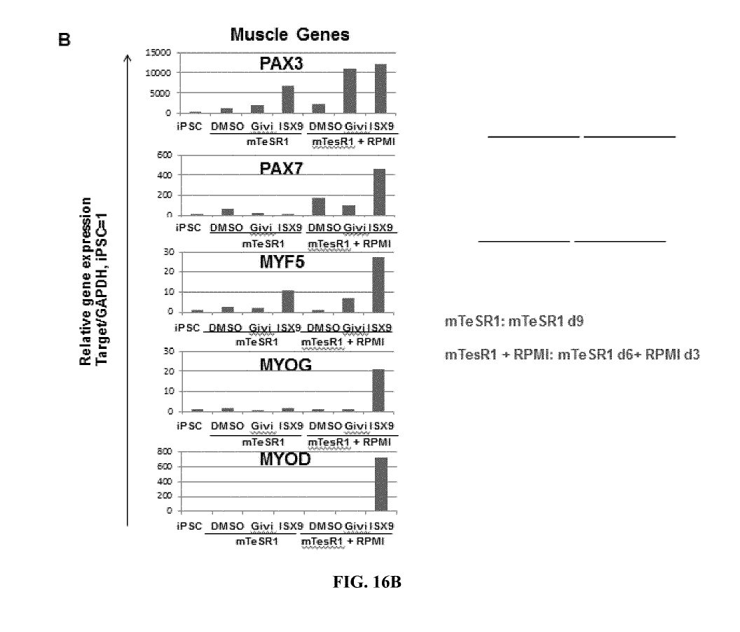

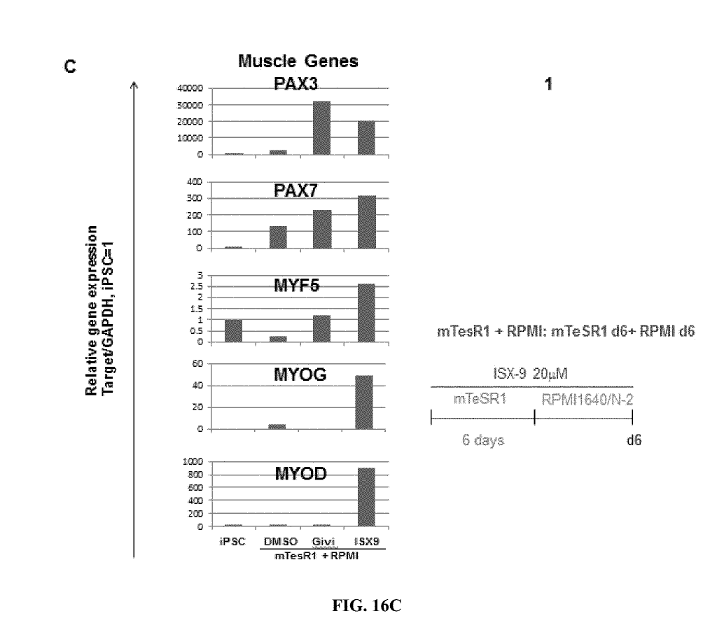

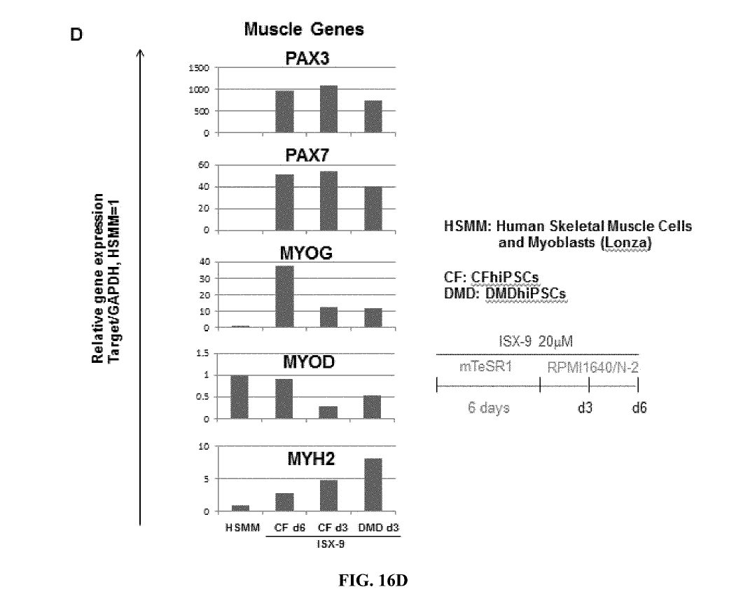

This disclosure also provides a method for generating iPSC derived muscle progenitor cells (MPC) by isoxazole and isoxazole like compounds and their preconditioning or treatment of skeletal muscle that will induce functional CXCR4 expression in MPC to facilitate mobilization and engraftment of progenitor cells in dystrophic skeletal muscles. See FIGS. 15A-15B. For the MPC to be a viable therapeutic option for DMD, dystrophin gene expression must be sufficient to restore muscle contractility. Hence MPC from the disclosed methods provide an abundant source of cells for transplantation and provide an effective and safe therapy for regeneration of dystrophic muscle. Transplantation of iPSC-derived progenitors coupled with methods to optimize the host muscle microenvironment, such as treatment with an effective amount of a steroid will more effectively ameliorate dystrophic pathology and improve the quality of life for patients with DMD.

Patients suitably treated by this method include those suffering from a disease or disorder associated with cardiac malfunction including, but not limited to, congestive heart failure, isolated diastolic heart failure, myocardial infarction, and cardiac arrhythmia. There are several forms of cardiac arrhythmia that can be treated including, but not limited to, sick sinus syndrome, bradyarrhythmia, abnormal sinus node function, atrioventricular block, and atrial and ventricular tachyarrhythmia.

Local or systemic administration, by use of a catheter or cardiac patch with isoxazole or similar compound treated iPS cells, of the cells or compositions can be effected in one dose, continuously or intermittently throughout the course of treatment. Methods of determining the most effective means and dosage of administration are known to those of skill in the art and will vary with the composition and/or cells used for therapy, the purpose of the therapy and the subject being treated. Single or multiple administrations can be carried out with the dose level and pattern being selected by the treating physician. Suitable dosage formulations and methods of administering the cell or agents are known in the art. In a further aspect, the cells and composition of the invention can be administered in combination with other treatments.

The cells and populations of cell are administered to the host using methods known in the art and described, for example, in U.S. Pat. No. 6,638,369. This administration of the cells or compositions of the invention can be done to treat disease as noted herein, and to produce an animal model of the desired disease, disorder, or condition for experimental and screening assays.

### Screening Assays

The present invention provides methods for screening various agents that modulate the therapeutic functions as described herein as well as gene expression, cell differentiation, exosome or microvesicle expression. For the purposes of this invention, an “agent” is intended to include, but not be limited to a biological or chemical compound such as a simple or complex organic or inorganic molecule, a peptide, a protein (e.g., antibody), a polynucleotide (e.g., anti-sense) or a ribozyme. A vast array of compounds can be synthesized, for example polymers, such as polypeptides and polynucleotides, and synthetic organic compounds based on various core structures, and these are also included in the term “agent.” In addition, various natural sources can provide compounds for screening, such as plant or animal extracts, and the like. It should be understood, although not always explicitly stated that the agent is used alone or in combination with another agent, having the same or different biological activity as the agents identified by the inventive screen.

To practice the screening method in vitro, suitable cell cultures or tissue cultures containing the modified cell(s) are first provided. When the agent is a composition other than a DNA or RNA, such as a small molecule as described above, the agent can be directly added to the cell culture or added to culture medium for addition. As is apparent to those skilled in the art, an “effective” a mount must be added which can be empirically determined. When agent is a polynucleotide, it can be directly added by use of a gene gun or electroporation. Alternatively, it can be inserted into the cell using a gene delivery vehicle or other method as described above. Positive and negative controls can be assayed to confirm the purported activity of the drug or other agent.

Also provided herein is a method for selecting a cell or cell population for reprogramming, comprising determining the expression level of miR-195 in a sample wherein low expression of miR-195 selects the cell or population and lack of low expression of miR-195 does not select the cell or population. In a further aspect, the method further comprises determining the expression level of one or more of marker selected from miR-29b, miR-205, miR-378, and miR-542-3p, wherein low expression of the one or more marker selects the cell or population and lack of low expression of miR-195 does not select the cell or population.

The following methods are useful in executing the inventions as described herein.

### Experiment No. 1

In Vitro Generation of Muscle Progenitor Cells (MPCs) from hiPSC

Human Induced Pluripotent Stem (iPS) Cells (ATCC® ACS-1021™) induced from human cardiac fibroblasts were cultured with mTeSR™1 (STEMCELL Technologies Inc.) on Vitronectin XF (STEMCELL Technologies Inc.) coated 6-well plates. iPS Cells were passaged every 4 to 6 days with ReLeSR™ (STEMCELL Technologies Inc.).

For differentiation of iPS Cells into MPCs iPS Cells were dissociated into single cells with ACCUTASE™ (STEMCELL Technologies Inc.) into single cells and seeded at 1×10⁵ cells/cm² with mTeSR™1 supplemented with 5 μM RHO/ROCK pathway inhibitor (Y-27632, STEMCELL Technologies Inc.). After 24 hr, the medium was changed to fresh mTeSR™1. mTeSR™1 was refreshed daily during first 3 days. After 3 days, culture medium was changed to mTeSR™1 supplemented with 20 μM ISX-9 (MedChemExpress). The medium was refreshed every other day. After 6 days, the medium was switched to RPMI 1640 Medium (Thermo Fisher Scientific) supplemented with N-2 Supplement (Thermo Fisher Scientific) and 20 μM ISX-9 and refreshed every other day for another 3 to 6 days.

Methods for Exosome or Microvesicle and Cpc Generation Include Cell Culture, hiPSC Maintenance.

Human iPSC cells (ACS-1021, ATCC, USA) were maintained in mTeSR1 media (Stem Cell Technology) on vitronectin coated six-well plates with daily medium changes. Cells were passaged with ReLeSR™ reagent every 4-7 days according to the manufacturer's protocol (Stem Cell Technology).

### CPC Generation.

Briefly, hiPSCs maintained on vitronectin coated six-well plate in mTeSR1 media (Stem Cell Technology) were dissociated into single cells using Accutase solution (Invitrogen) at 37° C. for 10 min and then were seeded on to a vitronectin-coated six-well plate at 1×106 cell/well in mTeSR1 supplemented with 5 μM ROCK inhibitor (Y-27632, Stem Cell Technology) for 24 h. The second day, cells were cultured in mTesR1 with daily change for 3 days. Afterwards the medium was switched to RPMI/B27 minus insulin supplemented with ISX-9 (20 uM, dissolved in DMSO, Stem Cell Technology) for 7 days.

### EB Generation

Applicant generated EBs using hanging drop method in RPMI/B27 minus insulin medium.

Generation and Isolation of Exosomes or Microvesicles from Cardiac Progenitor Cells, EB or hiPSCs

Exosomes or microvesicles were generated from Human iPSC cell line ACS-1021 (ATCC, USA), Embryoid bodies (EB) and CPCs induced by ISX-9. CPCs were generated as described in Experiment No. 10. Conditioned media was collected from hiPSC and CPCs. The conditioned media was centrifuged at 3000 rpm for 30 min to remove cells and debris, followed by filtration through a 0.22 μm filter to remove the remaining debris. Then the medium was further concentrated to 500 μl using Amicon Ultra-15 100 kDa centrifugal filter units (Millipore). Isolation of exosomes or microvesicles in the concentrated medium was used qEV size exclusion columns (Izon science). Exosome or microvesicle fractions were collected and concentrated by Amicon Ultra-4 10 KDa centrifugal filter units to a final volume of <100 μl. The purified exosomes or microvesicles were stored at −80° C. and subsequently characterized by nanotracking analysis, protein and ultrastructure analysis.

Particle Size and Concentration Distribution Measurement with Tunable Resistive Pulse Sensing

Particle size and concentration distribution of exosomes or microvesicles isolated analysis were performed using tunable resistive pulse sensing method with a qNano instrument (Izon Science). Briefly, the number of particle was counted at least 600 to 1000 events using 20 mbar pressure and NP200 nanopore membranes stretched between 46.5-47.5 mm. Calibration was performed using known concentration of beads CPC200 (diameter: 210 nm). Data were processed using Izon Control Suite software.

### Transmission Electron Microscopy

Exosomes or microvesicles pellet were fixed with 4% paraformaldehyde (PFA). Following a total of 8 washes using distilled water, grids were contrasted with a uranyl-oxalate solution for 5 minutes, and transferred to methyl-cellulose-uranyl acetate for 10 minutes on ice according to the description in previous study. Samples were examined on a JEOL JEM-1220 transmission electron microscope (TEM) (JEOL USA, Inc.)

### Selected Exemplary Embodiments

Applicant has discovered that iPSc can be chemically induced to DNA hypomethylation causing upregulation of cardiac genes and allowing successful propagation in the diseased heart with no or limited chances for tumorgenecity.

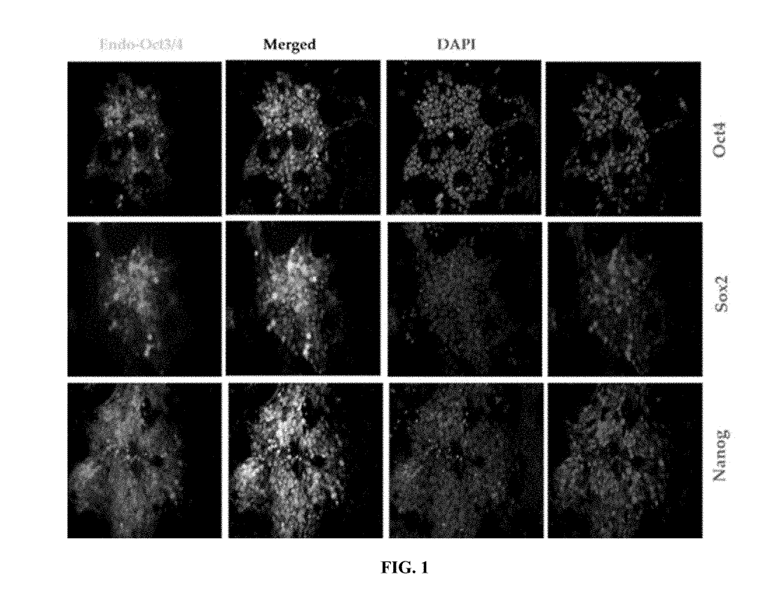

Skeletal myoblasts (SMs) purified from young male Oct3/4-GFP⁺ transgenic mouse were treated with DNA methyltransferase inhibitor 500 μM RG-108 in 0.5% DMSO in knock out DMEM for 5-days. Two weeks later, GFP⁺ colonies of SM derived iPS cells (SiPS) expressing GFP and morphological features of mouse embryonic stem cells were isolated and propagated in vitro. SiPS were positive for alkaline phosphatase activity, expressed SSEA1 and displayed a panel of pluripotency markers (FIG. 1) similar to ES cells and developed teratomas in nude mice. Although the research described herein was conducted in a murine model, the use of the same markers and agents will provide similar if not identical results in human cells and tissue.

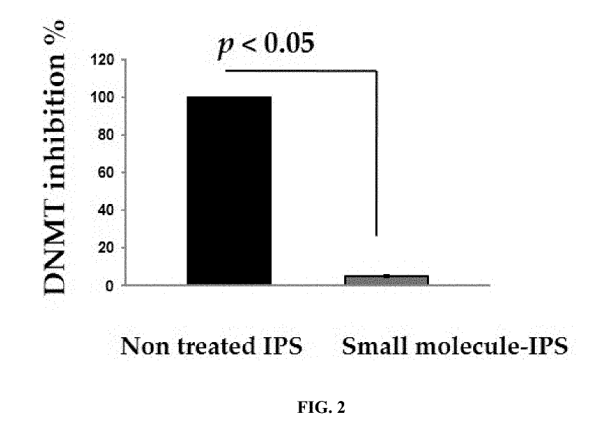

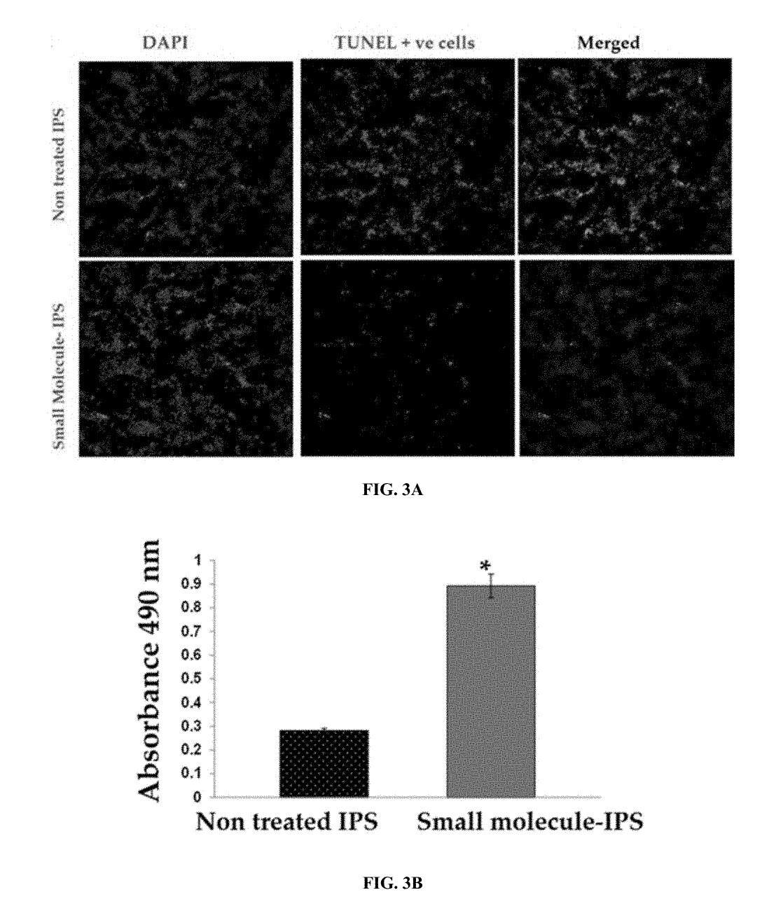

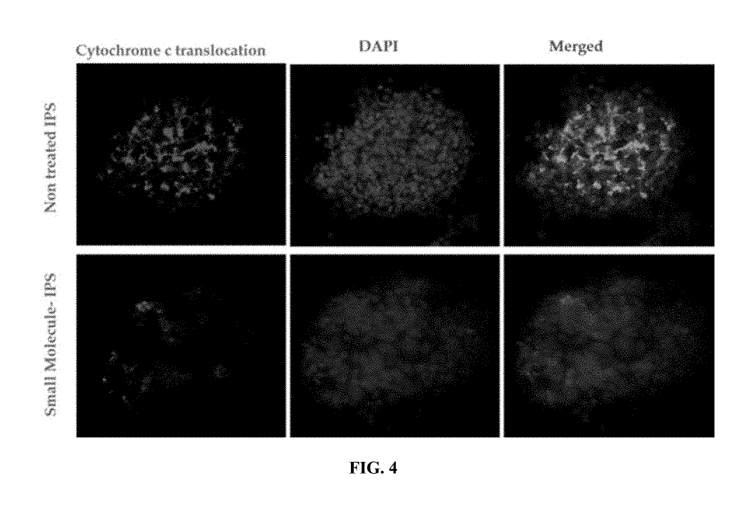

In order to direct SiPS towards cardiac lineage cells, they were treated with a small molecule (Isoxazole, 20 uM, Sigma-Aldrich or Isox 9, StemCell Technologies, Inc Vancouver, BC) for five days and analyzed for DNA methyltransferase (Dnmt) activity, cell proliferation, and cardiac gene expression. DNMT activity was completely abolished with 95% reduction in global DNA methylation in small molecule treated SiPS (FIG. 2). These SiPS showed increased proliferative activity (p<0.01 vs. non treated Sips) evaluated by cell proliferation assay and also become tolerant to apoptosis (FIGS. 3A-3B) an important consideration for preventing cell death in the ischemic environment. Small molecule treated IPS cells show a significant decrease in cytochrome c translocation to cytoplasm as compared to the untreated IPS cells (FIG. 4).

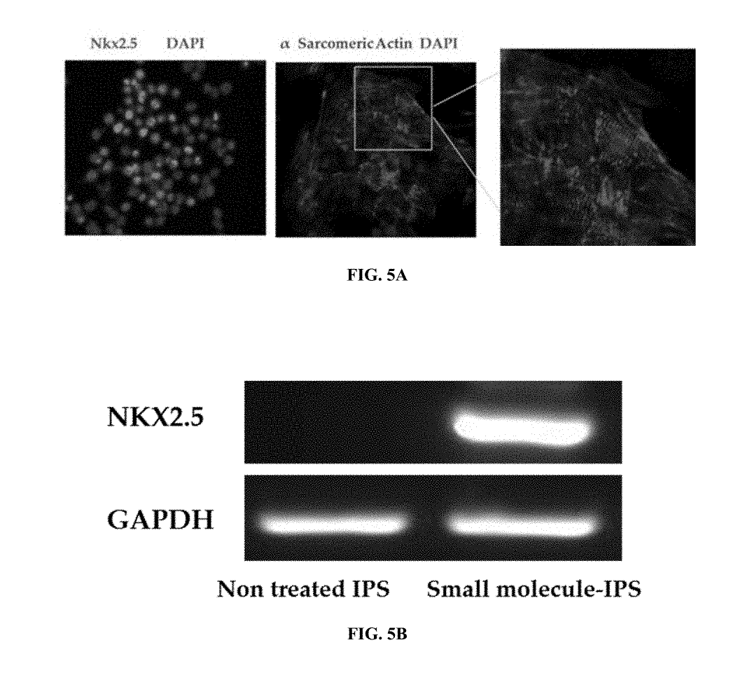

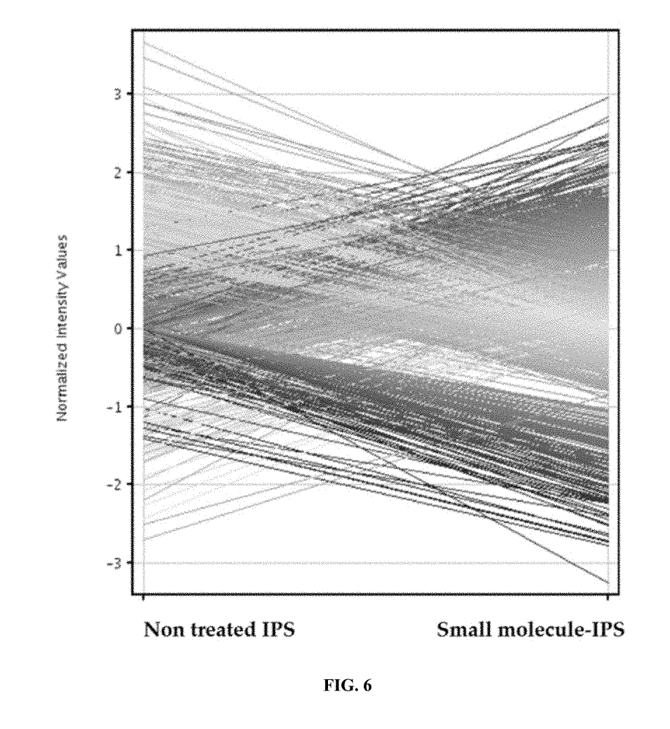

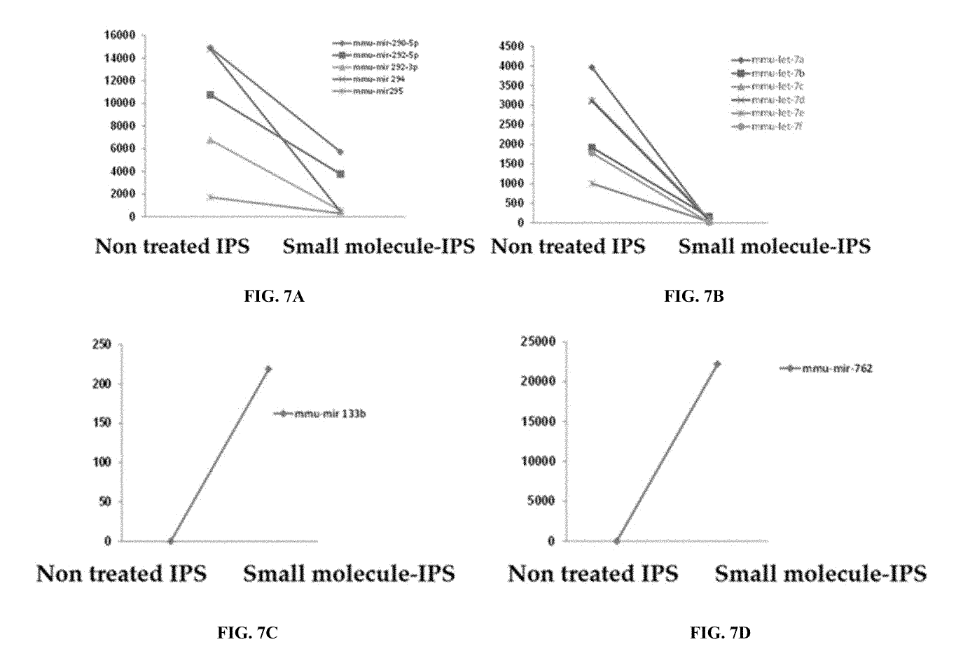

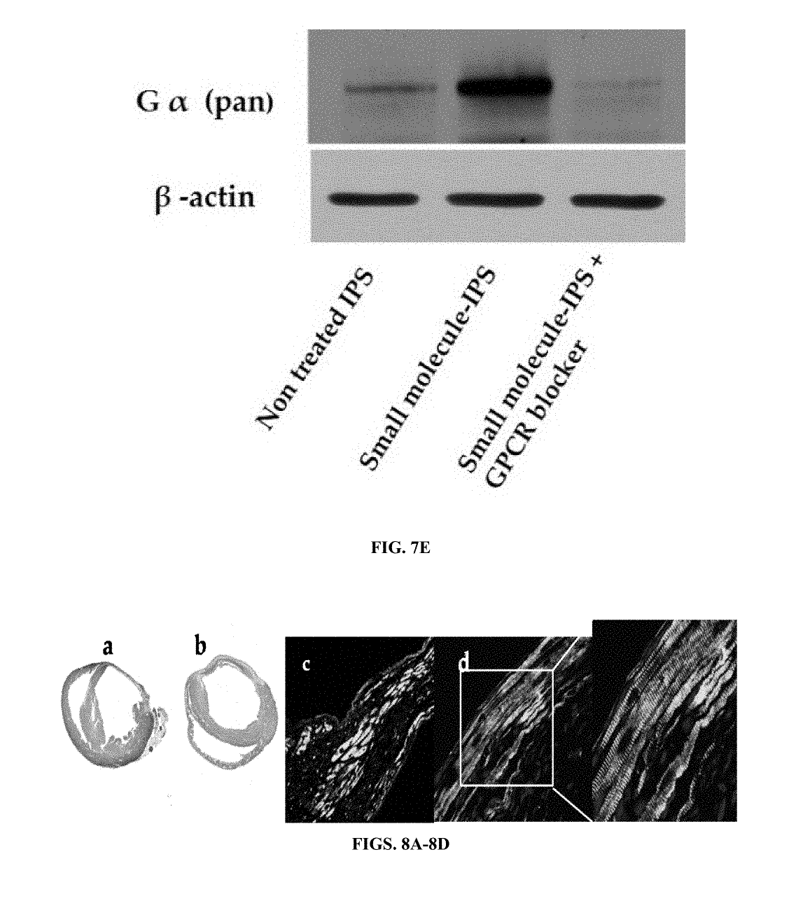

RT PCR analysis showed cardiomyocyte-like gene expression profile with significant upregulation of Nkx-2.5 (p<0.01 vs non treated IPS; see FIGS. 5A-5B). Approximately 60% treated iPS cells were positive for Nkx-2.5. Given that posttranscriptional regulation is crucial for gene expression and cell survival molecular phenotypic analysis was performed, Affymetrix array-based gene expression profiling further confirmed 2-3 folds downregulation of Dnmt1, Dnmt3b and Max gene associated protein which were associated with global DNA hypomethylation and myc dependent cell transformation (see Table 1). Additionally, there was a 2-3 fold concomitant upregulation in the CCL7, CXCR2, CXCR5, integral membrane protein 2A, and ephrin A3. (FIG. 6). These were associated with DNA synthesis, cell proliferation, cell matrix interaction and chemoattraction. miR microarray analysis showed upregulation of cardiac specific miR-133, 762 and down regulation of pluripotency associated miR-290 cluster, miR-574-5p and let-7 family (see FIGS. 7A-7D). Western blot analysis showed significant upregulation of Gαi protein levels as compared to untreated IPS cells (see FIG. 7E).

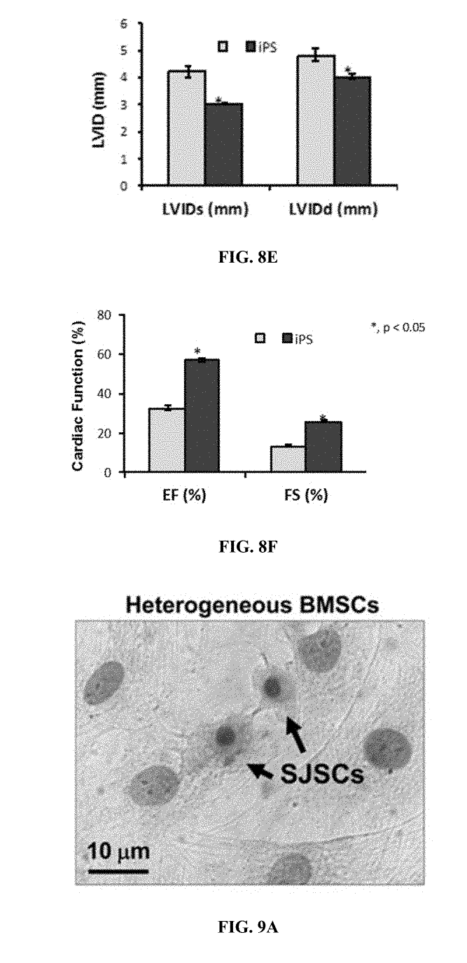

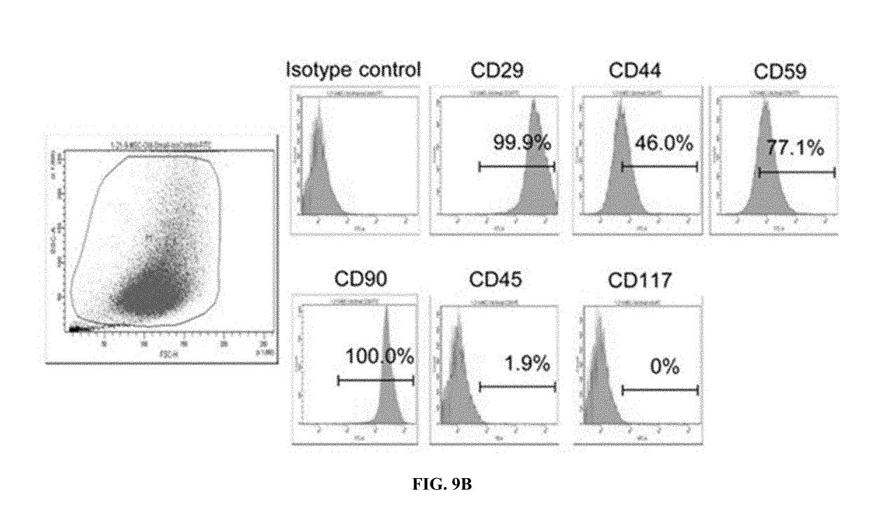

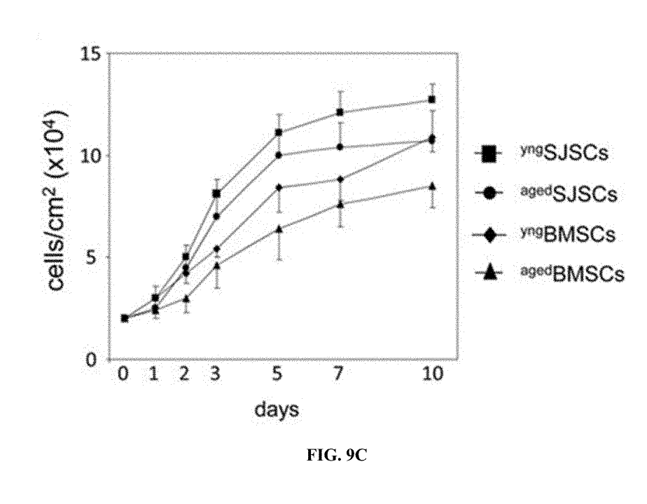

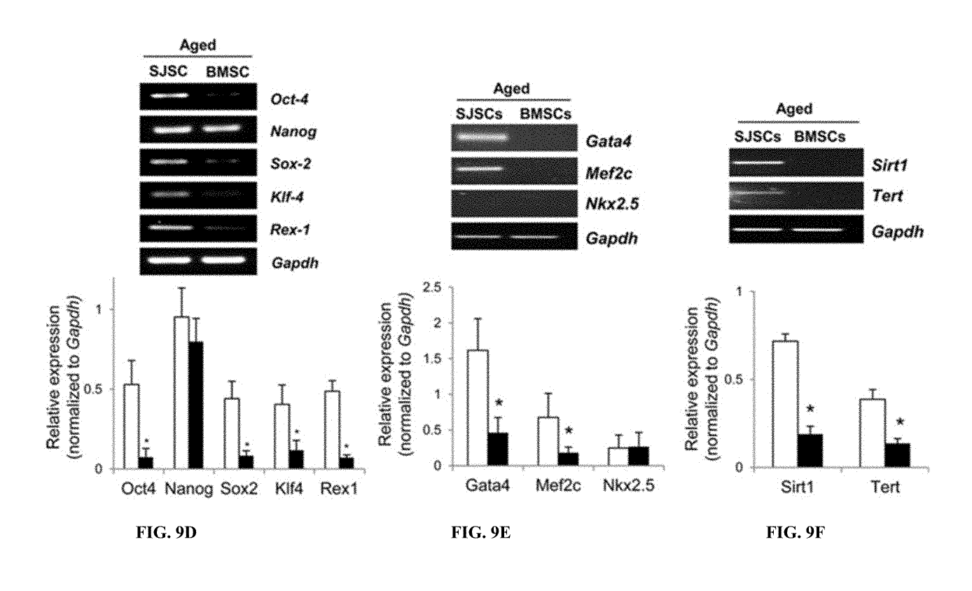

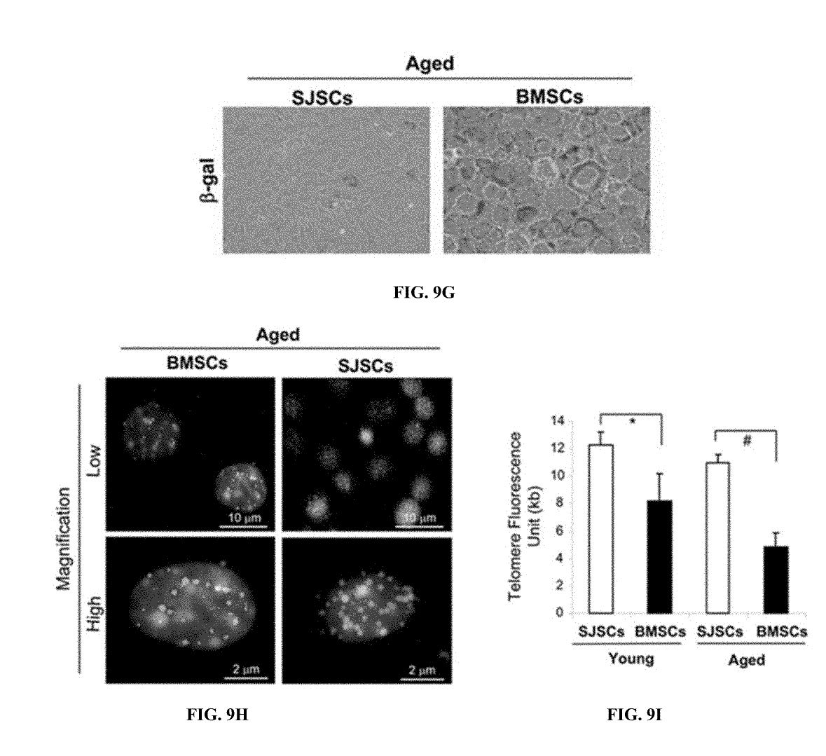

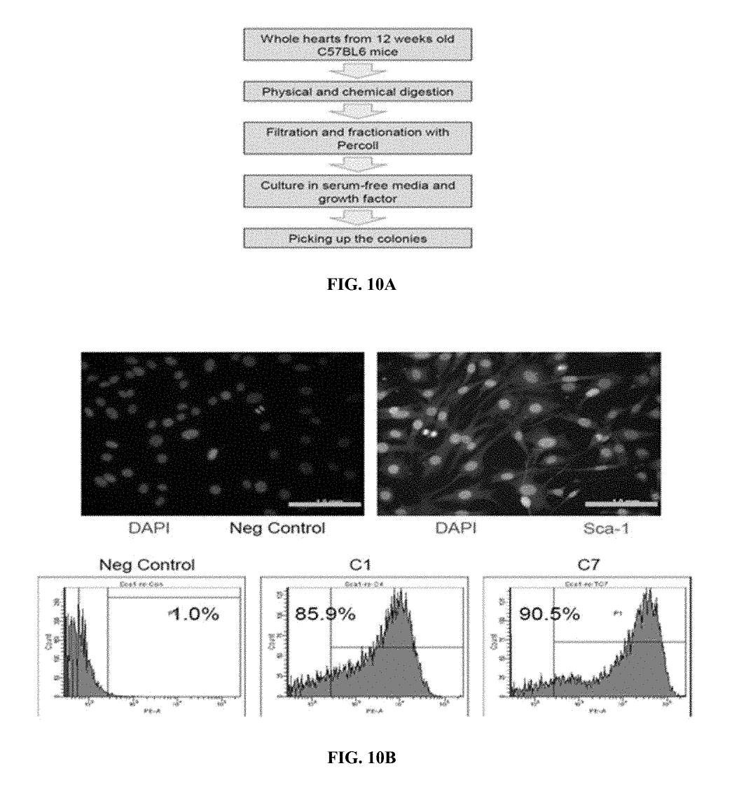

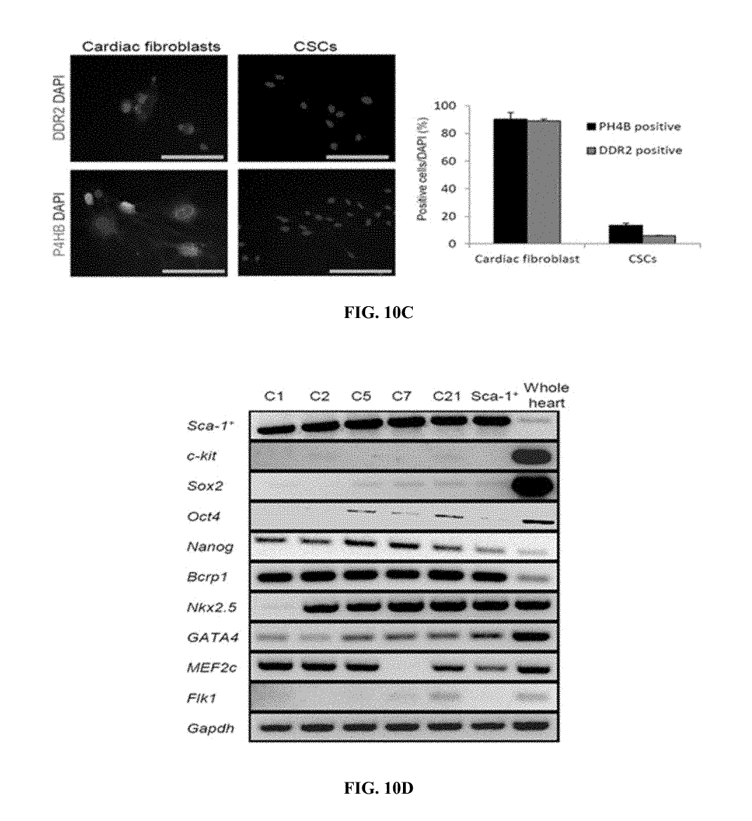

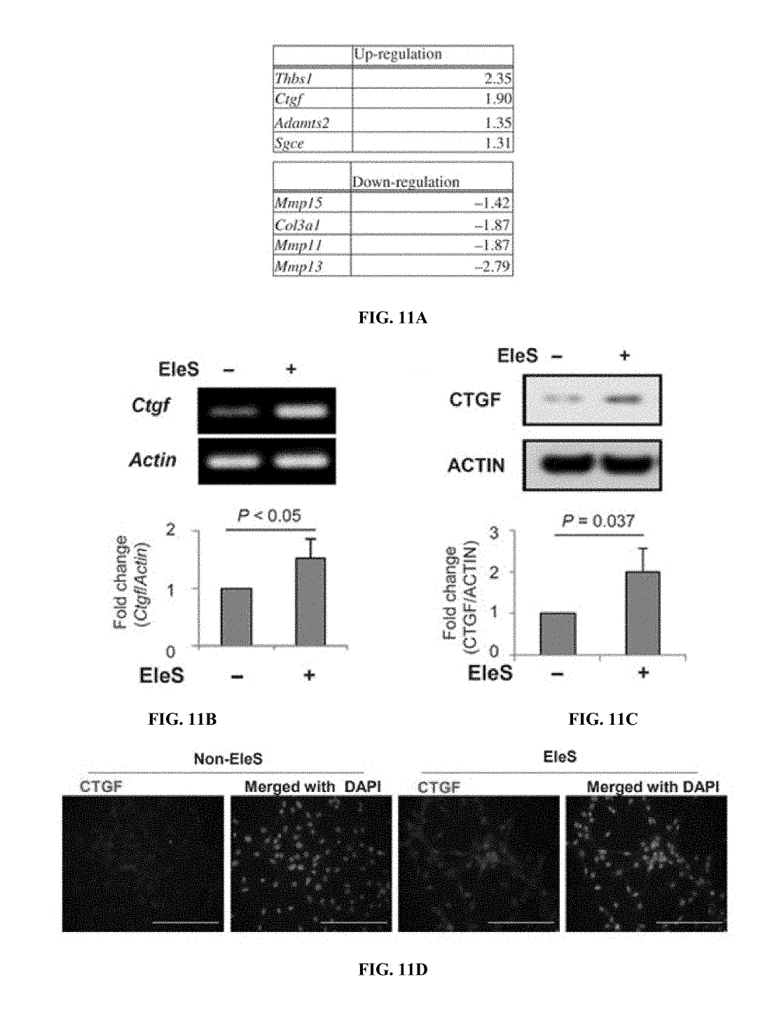

The SiPs treated with the small molecule were stained with PKH 26 for locating them in the heart and transplanted in the myocardium after 30 minutes of coronary artery ligation for 6 weeks. Before harvesting the hearts for visualizing the fate of transplanted SiPs, cardiac function was monitored with echocardiography. The treated SiPs were differentiated into myocytes forming myofibers in the scar tissue. The scar area in the left ventricle was muscularized with the treatment with isox treated SiPS (FIGS. 8B, 8C, 8D) compared to untreated/control hearts (FIG. 8A) These are significant findings and have been reported previously in an initial patent application as only new pictures are graphically shown in figures (see FIGS. 8A-8F).

Cardiac function: The temporal changes in global heart function including left ventricle ejection fraction (LVEF) and left ventricle fractional shortening (LVFS) were measured in control infarcted and treated infarcted hearts. The pathological remodeling of left ventricle chamber dimensions during systole (LVDs) and diastole (LVDd) was also significantly reduced in CPs treated hearts (2.97 mm and 3.99 mm) as compared with DMEM treated hearts (4.77 mm and 4.78). See FIG. 8E. Transplantation of CPs significantly improved LVEF and LVFS (56.8+−1.32%; 25.5+−0.4%) in comparison with the DMEM injected infarcted hearts (n=4; 32.42+−1.03% and 13.0+−0.4% respectively). FIG. 8F.

Applicant also discovered that isoxazoles induced DNA hypomethylation and myc dependent cell transformation in the iPS cells and were associated with DNA synthesis, cell proliferation, cell matrix interaction and chemotexis. The isoxazoles compound upregulated the CXC chemokine receptors and integral membrane proteins, Epherin family and related receptors that specifically involved in the development of erythropoiesis.

Applicant further discovered that isoxazoles upregulated cardiac specific miR-133, miR-762 and down regulation of pluripotency associated miR-290 cluster and let-7 family in iPS cells as compared to untreated iPS cells.

Thus, the above reports that small molecule-mediated modification of iPS cells can grow in the infarcted heart and replace the scar tissue with working myocytes coupled together with electrical connections (gap junctions). The sulfonyl-hydrazone family of small molecules can induce cardiac genes in iPS cells derived from myoblasts. These small molecules are known to induce muscle differentiation in Notch activated epicardium derived progenitors (Russell, J. L. et al. (2012) ACS Chem Biol. 7(6):1067-1076). Small molecule induced iPS cells were engrafted in post ischemic model and improved global cardiac function compared to non-treated iPS cells. Recovery of cardiac function was dependent on the survival of the iPS cell-derived progenitors cells. Small molecules are innovative in inducing the tissue-specific gene expression in iPS cell towards tissue differentiation, as well as determining the temporal and spatial patterns of development.

Applicant has discovered that the induced pluripotent stem cells, if treated with the appropriate small molecule, can pharmacologically inhibit the DNA methylation, hence a critical player in the regulation of cardiac developmental genes. Without being bound by theory, the effect of small molecule on epherin family may manipulate the process of hematopoietic progenitors generation and could be beneficial for clinical hematopoietic malignancies.

Applicant also discovered that isoxazoles upregulated cardiac specific miR-133, miR-762 and down regulation of pluripotency associated miR-290 cluster and let-7 family in iPS cells as compared to nontreated iPS cells.

Eric Oslon in U.S. Pat. No. 8,318,951 (951 patent) previously reported the use of these compounds in neurogenesis, epicardial progenitors cells. Regardless of these studies, the findings reported herein are unique and innovative in that the findings address the small molecule induced epigenetic changes that can be manipulated for rendering the iPS cells for propagation in the infarcted heart. This work emphasizes the iPS cells which can be made from a patient cell (in this case skeletal muscle) and reintroduced into the same patient after pretreatment with small molecule. These findings are distinct from the work of Oslon since iPS cells of the present disclosure were therapeutically predesigned (or pretreated) for propagation in the scar tissue of the mice heart after coronary artery ligation or heart attack. There was significant regeneration in the scar tissue by iPS cells. On the other hand, Olson injected the drug into live mice to act on heart progenitors present in the heart. It is Applicant's belief that the drug worked on different cells of the heart. Here, the composition and methods predesigned the iPS cells into cardiac progenitors in the dish and then reintroduced into damaged hearts.

Applicant also found that IPS pretreatment with small molecule reduced the apoptosis which is also novel finding. For example, less apoptosis was observed in small molecule induced iPS cells as compared to non-induced iPS cells. The results reported in the '951 patent were generated only using the Trypan blue exclusion assay for cell viability not cell apoptosis which is critical in cell survival under ischemic condition.

Applicant also found that DNA methyltransferase (DNMT) activity was completely abolished in the chemically modified IPS cells. There was 2-3 folds downregulation of Dnmt1, Dnmt3b and Max gene associated protein in small molecule modified iPS cells which are associated with DNA hypomethylation and cell transformation. There was 2-3 folds upregulation of CCL7, CXCR2, CXCR5, integral membrane protein 2A, and EphrinA3 which are associated with cell mobilization, chemotaxis, cancer and erythropoiesis was also observed. Applicant further observed upregulation of cardiogenic specific miR-133, miR-762 and down regulation of pluripotency markers and pluripotency associated miR-290-295 cluster and let-7 family which confirms the induction of the cardiac regulatory genes by Micro RNAs by down regulating the pluripotency genes.

In addition, the '951 patent didn't observe Gα protein level, as compared to the work reported here, which did observe the Gα protein level upregulation which was blocked when the chemically modified iPS cells were treated with GPCR blocker and abolished the all in vitro effect in small molecule induced iPS cells. Oslon previously has reported the use of these compounds in neurogenesis, epicardial progenitors cells. Regardless of the previous studies, it is Applicant's belief that the current findings are unique and innovative that address the small molecule induced epigenetic changes that can be manipulated for rendering the iPS cells suitable for propagation in the infarcted heart. Another novelty is shown here that initially iPS cells primed with isoxazoles and similar small molecules can be converted into vascular (endothelial) progenitors and smooth muscle cells as well besides myocytes.

To the best of Applicant's knowledge and the results reported in Example 2, below, that isoxazoles and other similar small molecules work on other stem cells from the body. For example, stem cells derived from the bone marrow and the heart can be directly reprogrammed into myoctyes or endothelial cells and smooth muscles with small molecule drugs like isoxazoles. Thus, isoxazoles and similar small molecules can be directly administered, e.g., by injection into the hearts of patients, e.g., heart attack patients, for converting inflammatory cells and stem cells mobilized to the ischemic sites into cardiac cells in order to replace the scar tissue. The net effect is to assist with the regrowth of the damaged heart after the heart attack. The disclosed methods have the advantage of being non-viral based, using a previously approved compound, that simplifies clinical adoptions. Moreover, it is Applicant's belief that the disclosed methods will dramatically increase cardiac progenitors in one step treatment of iPSC in mono layer without coverting them into embryoid bodies within a few short weeks after treating with isoxazoles or other similar small molecules like cardionogin; CDNG1/vuc230, CDNG2/vuc198, and CDNG3/vuc247.

Current approaches in using iPS cells include limitations such as genetic mutations and or tumor (i.e., cancer) growth versus Applicant's methods, approach and technique. The methods are beneficial in that genetic mutations are not necessary, improving safety for clinical use.

It is Applicant's belief and to the best of his knowledge, the current reported study provides the first evidence that iPS cells can be therapeutically rendered safe for use by altering their chromatin configuration for upregulation of cardiac genes. These so called cardiac progenitors can propagate and regenerate the dying myocardium with limited cell death. Chemical reprogramming iPS cells to desired cardiac lineage would be smart strategy in cardiac therapeutics.

### Experimental Methods

### Maintenance of Mouse SiPS

SiPS were maintained on mitomycin C-treated mouse embryonic fibroblasts (MEFs) dishes in Knock out Dulbecco's Modified Eagle's Medium (knock out-DMEM, Invitrogen, CA, USA) supplemented with 20% Knockout Serum Replacement (KSR; Invitrogen, USA), 0.1 mM MEM Non-Essential Amino Acids solution (Invitrogen, CA, USA), 0.2 mM L-glutamine (Invitrogen, USA), 0.1 mM β-mercaptoethanol (Invitrogen, CA, USA) and 1000 U/ml LIF (Millipore) 0.5% penicillin and streptomycin. The colonies thus generated were detached regularly at an interval of 3-4 days with 0.2% collagenase-1V (Invitrogen, CA, USA) dissociated into single cell suspension with 0.025% trypsin (Sigma Aldrich, MO, USA) and re-plated onto MEFs for propagation.

### Cell Proliferation Assay

The cell proliferation assay was performed with the use of the MTS [3-(4,5-dimethylthiazol-2-yl)-5-(3-carboxymethoxyphenyl)-2-(4-sulfophenyl)-2H-tetrazolium] assay according to the manufacturer's recommendations (Promega). The plates were read at 490 nm using an automated ELISA plate-reader for the quantity of formazan product which was directly proportional to the number of living cells in culture.

### DNA Methyltransferase (DNMT) Activity Assay

Nuclear extracts were isolated using the NE-PER Nuclear and Cytoplasmic Extraction Kit (Thermo Scientific, IL USA). Total DNMT activity was determined using an EpiQuik DNA methyltransferase activity assay kit (Epigentek, Brooklyn, N.Y.) per manufacturer's protocol. Enzyme activity for samples and controls was measured on a microplate reader (Hidex Chameleon, Finland) at 450 nm and DNMT activity (OD/h/ml) was calculated according to the formula: (Sample OD−blank OD)/(sample volume)×1000.

### RT-PCR and Quantitative RT-PCR

Total RNA from small molecule treated and non-treated SiPS cells was isolated using RNeasy mini kit (Qiagen, Maryland, USA) and Omniscript Reverse Transcription kit (Qiagen, Maryland, USA) was used for the respective cDNA synthesis per manufacturer's instructions. For PCR amplification, 1 μg of the cDNA from the reverse transcription reaction was added to PCR mix containing the suggested quantity of the PCR buffer, Q solution, dNTP mix, reverse and forward primers, Taq DNA polymerase and distilled water. PCR conditions included initial denaturation at 95° C. for 4 minutes, 32 cycles of denaturation at 95° C. for 1 minute, annealing at 55° C. for 1 minute, extension at 72° C. for 1 minute and final extension at 72° C. for 7 minutes. The PCR products were separated on 1.5% agarose gel, stained with ethidium bromide and visualized and photographed on a UV transluminator (Bio-Rad, USA).

### Myocardial Infarction Model

The animals were anesthetized with (ketamine/xylazine 0.05 ml intra-peritonealy). A midline cervical skin incision was performed for intubation. The animals were mechanically ventilated with room air supplemented with oxygen (1.5 L/min) using a rodent ventilator (Model 683, Harvard Apparatus, MA, USA). Body temperature was carefully monitored with a probe (Cole-Parmer Instrument, IL, USA) and was maintained at 37° C. throughout the surgical procedure. The heart was exposed by left-sided limited thoracotomy and the left anterior descending (LAD) coronary artery was ligated with a prolene #9-0 suture. Myocardial ischemia was confirmed by color change of the left ventricular wall. The animals were grouped (n=12 per group) for intramyocardial injection of 10 μL of basal DMEM without cells (group-1) or containing 3.6×10̂5 SiPS (group-2) or 3.6×10̂5 SiPS-CPs (group-3). The cells were injected 10 minutes after coronary artery ligation at multiple sites (3-4 sites per heart) around the periphery of ischemic area of the free wall of the left ventricle under direct vision. For post-engraftment tracking of the transplanted cells and determination of their fate, the cells were labeled with PKH26 (Sigma, Product# PKH26-GL) according to manufacturer's instructions. The chest was closed and the animals were allowed to recover. To alleviate pain, Buprinex (0.05 ml) was injected subcutaneously in first 24 hours of surgery. The animals were euthanized on 7 days 4 weeks and 6-8 weeks after transthoracic echocardiography for the heart function evaluation. The hearts were frozen or fixed with 10% formalin solution and processed for embedding in paraffin for immunohistological studies.

### Transthoracic Echocardiography

The animals (n=8 per group) were anesthetized and lightly secured in the supine position on a warm pad. After the chest was shaven, Acoustic gel was applied and transthoracic echocardiography was performed using HDI-5000 SONOS-CT (HP) ultrasound machine with a 7-MHz transducer. The heart was imaged in the two-dimensional mode in the parasternal long-axis and/or parasternal short-axis views which were subsequently used to position the M-mode cursor perpendicular to the ventricular septum and left ventricle posterior wall, after which M-mode images were obtained. For each animal, measurements were obtained from 4-5 consecutive heart cycles. Measurements of ventricular septal thickness (VST), left ventricle internal dimension (LVID), and left ventricle posterior wall thickness (LVPW) were made from two-dimensionally directed M-mode images of the left ventricle in both systole and diastole. The average value from all measurements in an animal were used to determine the indices of left ventricle contractile function, i.e., left ventricle fractional shortening (LVFS) and left ventricle ejection fraction (LVEF) using the following relations LVFS=(LVEDd2LVESd)/LVEDd6100 and LVEF=[(LVEDd32LVESd3)/LVEDd3] 6100 and expressed as percentages.

### Immunocytochemistry

For immunocytochemistry, differentiated colonies of SiPS were immunostained with respective specific primary antibodies (anti-Oct3/4, anti-Sox2, anti Nanog antibodies, all at 1:100 concentration; Cell Signaling, Danvers, USA). Small molecule treated SIPs were seeded on 0.1% gelatin coated chambered slides for immunostaining. The cells were fixed with PBS containing 4% paraformaldehyde for 10 minutes at room temperature. After washing with PBS, the cells were blocked for 45 minutes at room temperature by CAS block (Invitrogen, CA, USA) and were immunostained with antigen specific primary antibodies Nkx-2.5, Gata 4, α Sarcomeric actin. (Santa Cruz, Calif., USA). The primary antibody-antigen reaction was detected with fluorescently conjugated specific secondary antibodies. Nuclei were stained with 5 μg/ml 4′6-diamidino-2-phenyl indole (DAPI; Invitrogen, CA, USA) staining. Fluorescence signals were observed and photographed using fluorescence microscopy (Olympus; Tokyo, Japan).

### Gene Expression Profiling

Affymetrix array-based gene expression profiling further confirmed 2-3 folds downregulation of Dnmt1, Dnmt3b and Max gene associated protein which were associated with global DNA hypomethylation and myc dependent cell transformation. In addition, there was 2-3 folds concomitant upregulation of CCL7, CXCR2, CXCR5, integral membrane protein 2A, and EphrinA3.

TABLE 1

| Fold change | | | | |
|---|---|---|---|---|
| | IPS + small | | | |
| IPS | molecule | Regulation | Genes description | Genes symble |
| 1.7296195 | 3.3164034 | up | Chemokine (C-C motif) ligand 7 | Cd7 |
| 1.4497509 | 2.7316089 | up | Chemokine (C—X—C motif) ligand 2 | Cxd2 |
| 1.0954261 | 2.136762 | up | Chemokine (C—X—C motif) ligand 5 | Cxd5 |
| 1.1732435 | 2.2551816 | up | integral membrane protein 2A | Itm2a |
| 1.1245799 | 2.1803806 | up | integral membrane 2B | Itm2b |
| 1.0622039 | 2.088119 | up | ephrin A3 | Efna3 |
| 1.5880175 | 3.0063593 | up | COX16 cytochrome c oxidase | Cox16 |
| | | | assembly homolog | |
| 1.3481197 | 2.5458012 | up | cytochrome c oxidase, subunit | Cox6b1 |
| | | | V1b polypeptide 1/Electron | |
| | | | Transport Chain | |
| 1.2005587 | 2.2982864 | up | ubiquinol-cytochrome c | Uqcr10 |
| | | | reductase, complex III subunit X | |
| 3.056267 | −1.6117706 | down | DNA methyltransferase | Dnmt1 |
| 2.431835 | −1.2820454 | down | DNA methyltransferase 3B | Dnmt3b |
| 2.1499553 | −1.1043067 | down | MAX gene associated protein | Mga |

### Experiment No. 2

Applicant has identified a bone marrow stem cell population named small juvenile stem cells (SJSCs) from aged mouse which express pluripotency and cardiac markers. Applicant expects when these cells are treated with isoxazole compound, would be more appropriate for cardiac differentiation and can be used as an alternate to IPS cells (Igura, K. et al. (2013) Am J Physiol Heart Circ Physiol. 305(9):H1354-H1362).

### Experiment No. 3

This experiment discloses an alternate method of generating cardiac progenitors by preconditioning with electrical stimulation supplemented with cardiogenic small molecules. Applicant identified another cell population (Kim, S. W. et al. (2013) Cardiovasc Res. 100(2):241-251) that expresses hematopoietic progenitor marker (c-kit), pluripotency markers (Oct-4, Sox2, Nanog), a stem cell side population marker (Bcrp1), early cardiac lineage markers (Nkx-2.5, GATA4, MEF2C), and a vascular progenitor marker (Flk1). Pre-conditioning (PC) of stem cells either through a brief period of ischaemia/anoxia or treatment with alternative mimetic improves their post-engraftment survival and differentiation characteristics. However there is no known report regarding the role of PC with electric stimulation (EleS) in stem cell survival, adhesion and cardiac differentiation. The heart generates a constant electrical field, the effect of which has not been explored in stem cells prior to transplantation. This study demonstrated that EleS provided PC effects on the survival of cardiac stem cells (Sca-1⁺ CSCs) through an increase in cell adhesion via focal adhesion kinase (FAK) activation, and releasing connective tissue growth factor (CTGF) by miR-378 down-regulation. It was found that connective tissue growth factor (Ctgf) was responsible for EleS-induced CSC (^{EleS}CSCs) survival and adhesion. Importantly, knockdown of Ctgf abolished EleS-induced cytoprotective effects and recovery of cardiac function. Furthermore, miR-378 was identified as a potential Ctgf regulator in ^{EleS}CSCs. This is another stem cell type which expresses both pluripotency and cardiac genes, and these cells can be further exploited with isoxazoles for cardiac progenitors.

### Isolation of Sca-1⁺ CSCs

C57BL6 mice (Harlan) were used for isolation of CSCs. 12 weeks old C57BL6 mice were anesthetized by intraperitoneal injection of ketamine/xylazine (87-100 mg and 13-15 mg/kg, respectively). The depth of anesthesia was monitored by positive toe pinch and muscle relaxation. Hearts were extracted and washed with ice-cold PBS to remove blood cells. After removal of aorta, pulmonary artery, and pericardium, the whole hearts were minced and digested for 20 min at 37° C. with 0.1% type-II collagenase (Invitrogen) and 0.01% DNase I (Worthington Biochemical Corporation). The cells obtained were passed through 40 μm filter to remove the debris, fractionated with 70% Percoll (Fluka) and cultured in maintenance medium containing serum-free DMEM/F12 (Invitrogen) supplemented with B27 (Invitrogen), 20 ng/ml EGF (Sigma), and 40 ng/ml bFGF (basic fibroblast growth factor, Peprotech). One week later, the cells were transferred to new dishes with serum-free maintenance medium with a density of 100 cells/cm² to initiate colony formation and each colony was mechanically picked up for individual sub-culture in 24 well dishes in DMEM/F12 (Invitrogen) supplemented with 2% FBS, B27 supplement, 20 ng/ml EGF, 40 ng/ml bFGF, and 10 ng/ml LIF (Leukemia inhibitory factor, Millipore). Colony-derived cells were re-seeded on new dishes at 90% confluence and were maintained with DMEM/F12 with 2% FB S.

### EleS of CSCs

Twenty four hours after seeding at a cell density of 3×10⁵ cells/35 mm dish, the cells were serum-starved for 15 hr followed by EleS (^{Eles}CSCs) using a culture cell pacer system (IonOptix). Cells were subjected to EleS for 0, 1, and 3 hr at 1.5 V/1.8 cm with biphasic square pulse (5 ms) at 5 Hz frequency. Cells without EleS (^{Non-EleS}CSC) were used as baseline controls. The cells were later harvested and used for various molecular and cellular studies.

### Experiment No. 4

Isolation of Old Mesenchymal Stem Cells (OMSCs) and Young Mesenchymal Stem Cells (YMSCs) from Bone Marrow

This study followed the Guide for the Care and Use of Laboratory Animals published by the US National Institutes of Health (NIH Publication #85-23, revised 1985) and protocol approved by the Institutional Animal Care and Use Committee, University of Cincinnati. BMSCs were isolated from bone marrow of C57BL/6 mice (young; 2 months, old; 24 months, respectively) as described previously in Igura K, et al. (2013) Am J Physiol. Heart Circ. Physiol 305:H1354-H1362. Briefly, BMSCs were cultured in 100 mm dishes with low glucose Dulbecco's modified Eagle's medium (DMEM) (HyClone Laboratories, Logan, Utah, http://www.hyclone.com) supplemented with 10% fetal bovine serum (FBS) for 6-10 days. The nonadherent hematopoietic bone marrow cells were discarded during the routine fresh medium replacement. YMSCs were isolated from old bone marrow by sieving through wells with 3-μm pores (cell culture insert; BD Bioscience, San Diego, Calif., http://www.bdbiosciences.com). The homogenicity of YMSC population was confirmed with surface marker expression by fluorescence activated cell sorting analysis as described previously in Igura, K. et al. (2013) Am J Physiol Heart Circ Physiol 305:H1354-H1362.

### Quantitative Fluorescence In Situ Hybridization for Telomere Length Measurement

Telomere was detected by fluorescence in situ hybridization (FISH) with PNA Telomere Cy3-labeled probe (F1002; TelC-Cy3, PNA Bio) according to manufacturer's instructions. Briefly, cells were fixed with 4% paraformaldehyde for 1 hour. After washing with phosphate buffer solution (PBS), cells were incubated with RNase 100 ng/1 L for 20 minutes and 0.005% pepsin for 5 minutes at 37° C. After dehydration with 70%, 85%, and 100% of cold ethanol, cells were incubated with 200 nM PNA Telomere probe for 10 minutes at 85° C. and incubated for 2 hours at room temperature. Then, cells were incubated with 2 3 saline-sodium citrate (SSC) buffer for 10 minutes at 60° C. 4′,6-diamidino-2-phenylindole (DAPI) and Cy3 signals were acquired simultaneously into separate channels using a confocal microscope (Fluoview FV1000, Olympus, Tokyo, http://www.olympus-global.com), and maximum projection from image stacks (10 sections at steps of 1 mm) were generated for image quantification. Telomere length was analyzed by using TFL-TeloV2-2 free software (Vancouver, Canada). With the program, the integrated fluorescence intensity value for each telomere, which is proportional to the number of hybridized probes, is calculated and presented. TFL-Telo is an application program used to estimate the length of telomeres from captured images of metaphases that have been stained for quantitative FISH (Q-FISH) analysis as described in Poon et al. (1999) Cytometry 36:267-278.

### Reverse Transcription-Polymerase Chain Reaction Analysis

Total RNA was isolated from various treatment groups of the cells with RNeasy Mini Kit (Qiagen, MD, http://www1.qiagen.com), and cDNA was prepared using Omniscript-RT Kit (Qiagen), according to the manufacturer's instructions. For polymerase chain reaction (PCR) amplification, 1 μg of the cDNA from the reverse transcription reaction was then added to a PCR mix containing the suggested quantity of Qiagen PCR buffer, Q-Solution, dNTP mix, reverse and forward primers, Taq DNA polymerase, and distilled water. Each PCR reaction was performed with specific primers.

Isolation and Detection of miRNA

Extraction of miRNAs was performed by using mirVana miRNA Isolation Kit, and miR-195 expression was detected by using mirVana qRT-PCR miRNA Detection Kit (Ambion, Life Technologies, Austin, Tex., http://www.ambion.com) and QuantiTect SYBR green PCR kit (Qiagen) as previously described in Kim et al. (2012) J. Mol. Med. 90:997-1010. Specific miRNA primers were purchased from Ambion.

miRNA Microarray

Total RNA samples obtained from OMSCs and YMSCs were sent to Exiqon (Denmark, http://www.exiqon.com/) for miRNA microarray profiling. Data were analyzed by Exiqon with in-house developed computer programs. Intensity values were transformed into log 2 scale, and fold changes were given in log 2 scale. A t test was performed between OMSCs and YMSCs profiling, and statistically significant difference was considered at p<0.01.

FISH to Detect miR-195

In situ detection of miR-195 was performed in OMSCs and YMSCs plated on chamber slide. Samples were fixed in 4% para-formaldehyde for cell culture at room temperature for 20 minutes followed by two washes in PBS. Fixed cells were then prehybridized in hybridization solution (BioChain, CA) for 3 hours at room temperature before hybridization. Probe (3 pmol; LNA-modified and fluorescein isothiocyanate (FITC)-labeled oligonucleotide; purchased from Exiqon) complementary to miR-195 was hybridized to the cells for 13-16 hours at 22° C. lower than predicted Tm of the probe. Subsequent to post hybridization washes with SSC buffer, in situ hybridization signals were detected with confocal microscope (Fluoview FV1000, Olympus).

### Luciferase Activity Assay

Precursor miR-195 expression vector was constructed in a feline immunodeficiency virus-based lentiviral vector system (Geneco-poeia). Luciferase reporter constructs containing 3′-untranslational region (UTR) of mouse Tert was designed to encompass mmu-miR-195 binding sites. Cells were plated into 24-well plates in triplicate and cotransfected with miR-195 vector (or scramble vector) and reporter construct with Lipofectamine 2000 (Invitrogen, Carlsbad, Calif., http://www.invitrogen.com). Firefly luciferase activities were measured by using Dual Luciferase Reporter Assay System kit (Promega, Madison, Wis., http://www.promega.com) per manufacturer's instructions. Transfection efficiency was normalized by Renilla luciferase activity.

Lentivirus-Mediated miR-195 Inhibition in OMSCs

Lentivirus containing miR-195 inhibitor-expressing vector was generated by using Lenti-Pac HIV Expression Packaging System (GeneCopoeia) per manufacturer's protocol. Briefly, 2.5 μl of lentiviral miR-195 inhibitor expression plasmid or scramble, 5.0 mL of EndoFectin Lenti and EndoFectin Lenti reagent were added in Opti-MEM I, and formed the DNA-EndoFectine complex. After incubating the complex at room temperature for 10-25 minutes, the DNA-EndoFectine complex was added to 293Ta cells in DMEM with 10% FBS, and then, incubated in 5% CO2 at 37° C. overnight. The culture medium was replaced with fresh DMEM with 5% FBS and 1/500 volume of the TiterBoost reagent to the culture medium. The virus pseudovirus-containing culture medium was collected at 48 hours post-transfection and concentrated after filtration. For the transduction of OMSCs with lentivirus, 10×10⁶ of OMSCs was plated, and 20 μl of virus suspension was added. To enhance lentiviral transduction efficiency, cells were placed at 4° C. for 2 hours and then incubated in a 5% CO2 at 37° C. for 48 hours/Senescence-Associated β-Galactosidase (gal) Staining Senescence-associated β-gal was detected by Senescence Detection Kit (BioVision, CA, http://www.biovision.com/) per manufacturer's instruction.

Terminal Deoxynucleotidyl Transferase dUTP Nick End Labeling Assay

Apoptotic cell death was detected by terminal deoxynucleotidyl transferase dUTP nick end labeling (TUNEL) according to instructions of the manufacturer (TMR Red; Roche Applied Science, http://www.roche-applied-science.com). For quantification, the numbers of TUNEL positive cells were counted in at least five randomly selected high-power fields (magnification 3 200) with three independent samples.

### Western Blot Analysis

Western blot was carried out as previously described in Kim et al. (2009) Cardiovasc Res. 100:241-251. Briefly, cells were lysed in lysis buffer, pH 7.4 [(in mM) 50 HEPES, 5 EDTA, 50 NaCl], 1% Triton X-100, protease inhibitors [(10 mg/mL aprotinin, 1 mM phenylmethylsulfonyl fluoride, 10 mg/mL leupeptin) and phosphatase inhibitors [(in mM) 50 sodium fluoride, 1 sodium orthovanadate, 10 sodium pyrophosphate]. The protein samples (40 mg) were electrophoresed using SDS-polyacrylamide gel and electroimmunoblotted. The specific antibodies used for the detection of Tert (sc-68720) was purchased from Santa Cruz (Santa Cruz, Calif., http://www.scbt.com), p53 (#2524), Sirt-1 (#2028), Phospho-FoxO1 (#2599), Bcl-2 (#3498), cleaved caspase-3 (#9661), Akt (#2920), Phospho Akt (#4060), and Actin (#4968) were purchased from Cell Signaling (Beverly, Mass., http://www.cellsignal.com). All antibodies were used as diluted with 1:1,000.

### Determination of Cell Proliferation Rate

Cell proliferation rate was determined by cell growth assay and colony formation assay as previously reported in Igura, K. et al. (2013) Heart Circ. Physiol. 305:H1354-1362.

### Experimental Model of Acute Myocardial Infarction and Cell Transplantation

In vivo cell transplantation using mouse acute myocardial infarction (AMI) model was performed in accordance with Applicant's previous report in Kim et al. (2013) Cardiovasc. Res. 100:241-251. Briefly, C57BL/6 mice (male 24 months old, 30-35 g b. wt.; n 5 10 animals per group) were prepared for left anterior descending (LAD) coronary artery ligation. Intraperitoneal anesthesia was administered with 0.1% of ketamine and 0.02% of xylene per body weight (g) for anesthesia. After endotracheal intubation and mechanical ventilation using Rodent Ventilator (Harvard Apparatus Model 683), the heart was exposed by left-sided minimal thoracotomy and LAD coronary artery was ligated with 6-0 silk. Immediately after ligation, 20 μl DMEM without cells or containing 2×10⁵ cells (scrOMSCs or anti-195OMSCs) were injected into infarction and border zones. Following injection, the opened chests of mice were sutured and all mice were allowed to recover.

miR-195 Expression is Markedly Upregulated in OMSCs

To profile miRNA expression induced with aging, Applicant isolated total RNA from OMSCs and YMSCs, and performed microarray analysis in OMSCs and YMSCs). Per these results, the expression of miR-140, miR-146a/b, and miR-195 was significantly upregulated in OMSCs whereas expression of miR-29b, miR-205, miR-378, and miR-542-3p was down regulated in OMSCs. Reverse transcriptase PCR (RT-PCR) and real time PCR confirmed microarray result that miR-195 expression was significantly upregulated in OMSCs as compared to YMSCs. FISH analysis for miR-195 visualization further confirmed that miR-195 was highly expressed in OMSCs. This result led Applicant to hypothesize that miR-195 was induced with aging, and its abrogation in OMSCs may restore the regenerative capacity of OMSCs. Applicant transfected OMSCs with anti-miR-195 and confirmed that transfected OMSCs showed reduced expression of miR-195 while scramble did not affect it, as shown by real-time RT-PCR, indicating that miR-195 inhibitor Applicant used was specific for reduction of miR-195 expression in stem cells.

Tert is a Direct Target of miR-195 in Aging Stem Cells

To investigate the biological relevance of miR-195 induction during stem cell aging and its participation in OMSCs senescence, Applicant first carried out target prediction analysis by in silico search to find potential target genes of miR-195 responsible for aging and senescence.

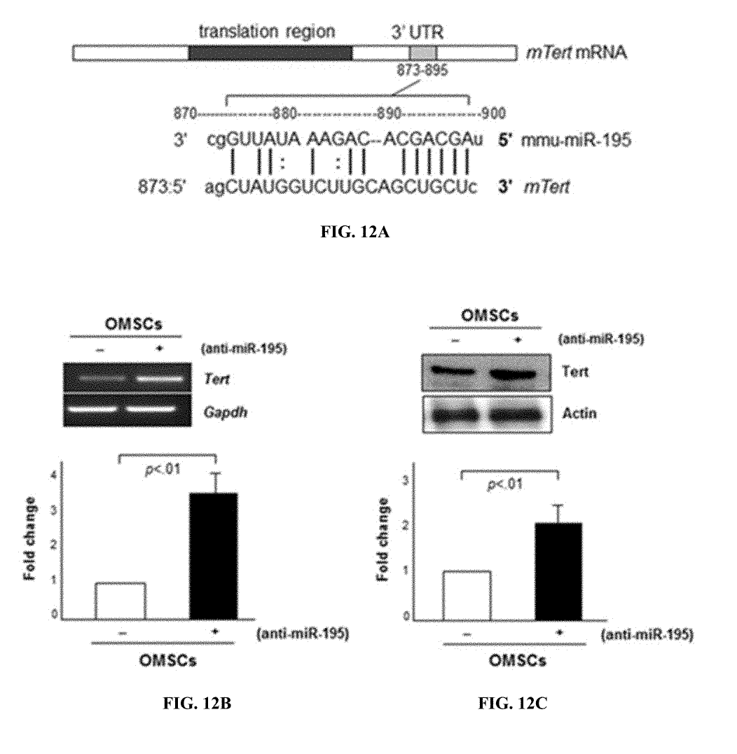

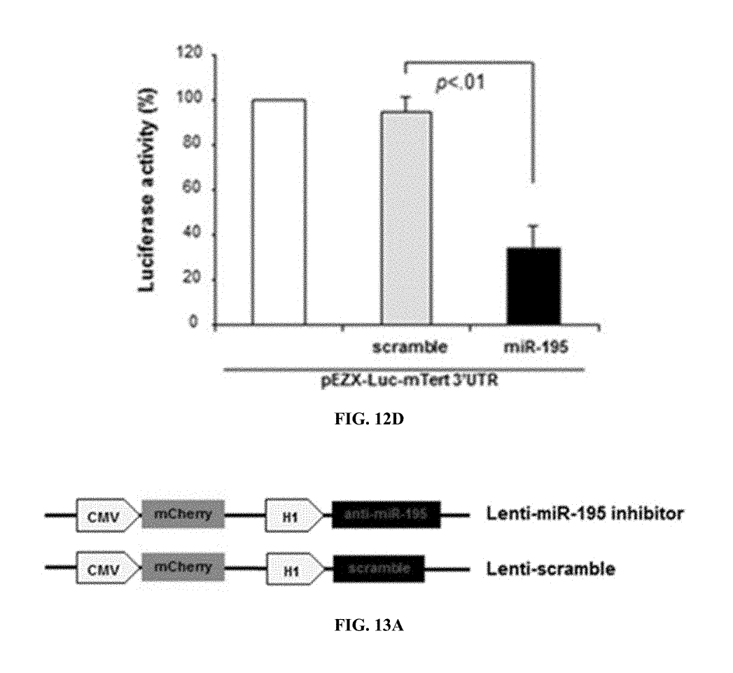

Interestingly, computational analysis predicted that mmu-miR-195 directly binds to 3′-UTR of mouse Tert gene. To experimentally validate this result, Applicant performed RT-PCR and Western blot analysis to examine whether Tert expression is altered by miR-195 knockdown in OMSCs. Interestingly, expression of both mRNA and protein of Tert was significantly increased by miR-195 abrogation in OMSCs (FIGS. 12B and 12C), indicating that Tert is a putative target of miR-195. Tert as a target gene of miR-195 was confirmed by luciferase activity assay which showed that cotransfection of a miR-195 expression vector (pEZX-miR-195) with the vector containing 3′-UTR of Tert gene significantly reduced luciferase activity in comparison to cotransfection with miR-scramble vector (pEZX-miR-Sc) (FIG. 12D). These results demonstrated that Tert is a direct target of miR-195 in stem cell aging.

Abrogation of miR-195 Rejuvenates OMSCs Through Telomere Relengthening and Antiaging Markers Reactivation

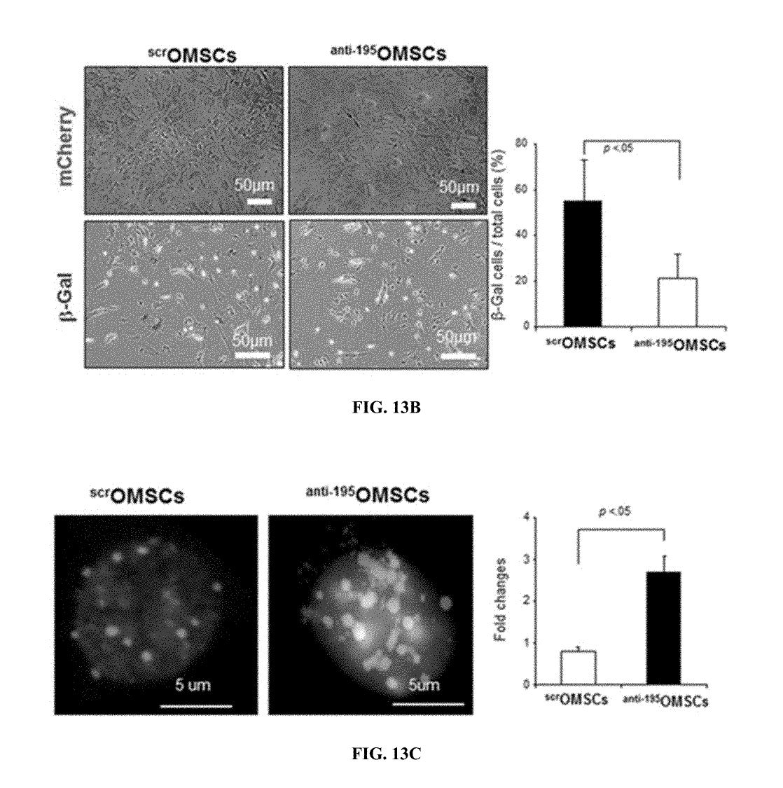

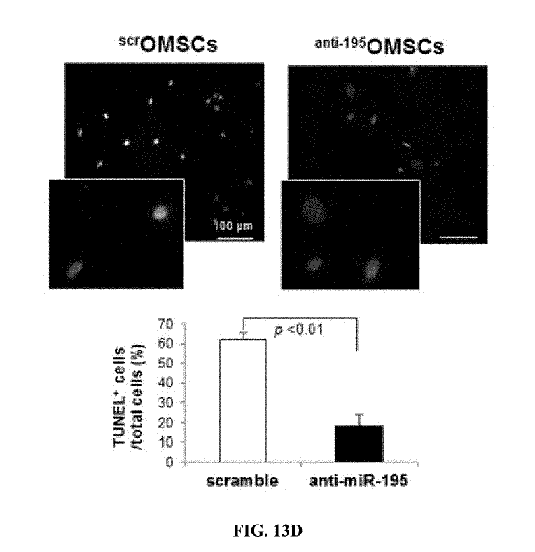

To examine the mechanistic participation of miR-195 in stem cell aging, Applicant knocked down miR-195 expression by using a lentiviral miR-195 inhibitor vector (Lenti-anti-miR-195) which could successfully transfect OMSCs to achieve abrogation of miR-195 (FIG. 4). mCherry signal (red fluorescence) in this vector system allowed Applicant to recognize the transfected OSMCs with miR-195 inhibitor or scramble vector (FIG. 13B, upper panel). Interestingly, abrogation of miR-195 significantly reduced senescence-associated β-gal expression in OSMCs as compared to scramble transfected OMSCs (FIG. 13B, 20.6% 6 4.9% vs. 54.5% 6 8.1%, p<0.01). More importantly, OMSCs transfected with miR-195 inhibitor induced significant telomere relengthening (2.9-fold higher as compared to scramble transfection, FIG. 13C). Furthermore, inhibition of miR-195 reduced TUNEL-positive apoptotic cell death in OMSCs (FIG. 13D, 19.7% 6 4.5% vs. 60.8% 6 2.7%, p<0.01), suggesting that rejuvenated OMSCs by miR-195 abrogation survive better under apoptotic condition such as ischemia. Furthermore, expression of antiaging makers (Tert and Sirt-1) and prosurvival markers (p-Akt and Bcl-2) were significantly increased by transfection of anti-miR-195 in OMSCs whereas expression of senescence-associated markers (p53) and proapoptotic marker (cleaved caspase-3) was markedly reduced by miR-195 abrogation (FIG. 13E), supporting Applicant's hypothesis that inhibition of age-induced miR-195 can rejuvenate OMSCs by reactivation of antiaging factors and suppression of senescence-associated markers. It is important to note that knockdown of miR-195 significantly restored the impaired cell proliferative abilities in OMSCs as evaluated by cell proliferation assay and colony formation assay (FIGS. 13F and 13G). These results demonstrated that miR-195 induced during stem cell aging plays critical roles in their fate and behaviors.

### Experiment No. 5

Generation of Cardiac Progenitor Cells from Human iPS Cells (hiPSCs) and Generation of Cells of Multiple Cell Lineages Therefrom

Cells of Human iPSC line (ACS-1021™) from American Type Culture Collection, Manassas, Va., 20110 USA (ATCC) were maintained on a vitronectin coated six-well plate in mTeSR1 medium and dissociated into single cells using accutase (Invitrogen) at 37° C. for 10 min. Afterwards, the cells were seeded on to a vitronectin-coated six-well plate at 1×106 cell/well in mTeSR1 supplemented with 5 μM ROCK inhibitor (Y-27632, StemCell Technologies, Vancouver, British Columbia, Canada) (day-3) for 24 hours. Further, the pluripotency of those iPS cells were confirmed by immunostaining, for example, to confirm the marker expression for OCT4, SOX2, TRA-1-60, TRA-1-81 and SSEA4 (FIGS. 14A-14B).

The cells then were then cultured in mTesR1 medium, which was changed daily according to an exemplary schematic outline as shown in FIG. 14C. At day 0, the medium was changed to RPMI/B27 medium minus insulin supplemented with 20 μM ISX-9 (StemCell Technologies) for 7 days. At the end of day 7 treatment, the expression of CPC markers including Nkx2.5 and GATA4 were observed by RT-PCR and Immunostaining (FIG. 14D).

For cardio myocyte differentiation, after 7 days ISX-9 treatment, the medium was switched to RPMI/B27 medium with insulin for another 7-10 days. At the end of 20 days, all the cells began spontaneous beating as shown in FIG. 14E.

For endothelial cells differentiation, 2×105/cm2 ISX-9 induced CPC were cultured in EGM-2 medium (Lonza, Lonza Walkersville Inc., Walkerswille Md. 21793-0127) for 10 days. At the end of treatment, the endothelia cell makers, CD31 and VE-cadherin were expressed (FIG. 14F). To analyze tube formation on matrigel in vitro, cells were seeded on top of a thin layer of matrigel at a density of 1.2×105 cells/well of a 24-well plate. After 16 hours, cells were labeled with calcein AM (Corning, Tewksbury Mass. 01876, USA) and were examined under the fluorescent microscope to visualize formation of tube-like structures (FIG. 14G).

For smooth muscle cells differentiation, induced CPC were cultured in DMEM-F12 medium supplemented with TGFβ (2 ng/ml) and PDGFBB (long/ml, R&D Systems, Inc, Minneapolis, Minn. 55413) for 10 days. At the end of this period, α-SMA and calponin expression were observed by immunostaining (FIG. 14H).

### Experiment No. 6

One Step Generation of Cardiac Progenitors from Monolayer Human Induced Pluripotent Stem Cells Using Isoxazole or Isoxazole Like Compounds

Currently in order to generate cardiac progenitors (CPC) from induced pluripotent stem cells (iPSC) in large numbers to repair heart after sudden heart attack or chronically weakened heart due to congestive heart failure, iPSC have to be converted into embryoid bodies or treated with multiple small molecules. These steps are costly and time consuming. Despite of these developments the progress is slow and at the same time, the purity of CPC preparations is not guaranteed. The one step use of isoxazole or isoxazole compounds such as ISX-9 has been extremely efficient in generating CPCs without the use of other growth factors (activin, BMP4), glycogen synthase kinase 3 inhibitor, Wnt inhibitor as well other small molecules. This small molecule has been previously reported for neuronal cell differentiation (Schneider et al. (2008) Nat Chem Biol. 4(7):408-10. doi: 10.1038/nchembio.95) and for reprogramming of fibroblasts into neurons in combination with other small molecules (Li et al. (2015) Cell Stem Cell 17(2): 195-203).

To test this rationale, Applicant also purchased human iPSC line (ACS-1021™) from American Type Culture Collection, Manassas, Va., 20110 USA (ATCC). iPSC maintained on vitronectin coated six-well plate in mTeSR1 medium were dissociated into single cells using accutase (Invitrogen) at 37° C. for 10 min and then were seeded on to a vitronectin-coated six-well plate at 1×10⁶ cell/well in mTeSR1 supplemented with 5 μM ROCK inhibitor (Y-27632, StemCell Technologies, Vancouver, British Columbia, Canada) (day-3) for 24 h (FIG. 14A). iPSC were confirmed for their pluripotency by Immunostaining (FIG. 14A, B). Cells then were cultured in mTesR1 medium, which was changed daily according to our schematic outline (FIG. 14C). At day 0, the medium was changed to RPMI/B27 medium minus insulin supplemented with 20 μM ISX-9 (StemCell Technologies) for 7 days. At the end of day 7 treatment, the expression of CPC markers including Nkx2.5 and GATA4 were observed by RT-PCR and Immunostaining (FIG. 14D). For cardiomyocyte differentiation, after 7 days ISX-9 treatment, the medium was switched to RPMI/B27 medium with insulin for another 7-10 days. At the end of 20 days, all cells began spontaneous beating (FIG. 14E). For endothelial cells differentiation, 2×10⁵/cm² ISX-9 induced CPC were cultured in EGM-2 medium (Lonza, Lonza Walkersville Inc., Walkerswille Md. 21793-0127) for 10 days. At the end of treatment, the endothelia cell makers, CD31 and VE-cadherin were expressed (FIG. 14F). To analyze tube formation on matrigel in vitro, cells were seeded on top of a thin layer of matrigel at a density of 1.2×10⁵ cells/well of a 24-well plate. After 16 hr, cells were labeled with calcein AM (Corning, Tewksbury Mass. 01876, USA) and were examined under the fluorescent microscope to visualize formation of tube-like structures (FIG. 14G). For smooth muscle cells differentiation, induced CPC were cultured in DMEM-F12 medium supplemented with TGFβ (2 ng/ml) and PDGFBB (long/ml, R&D Systems, Inc., Minneapolis, Minn. 55413) for 10 days. At the end of this period, α-SMA and calponin expression were observed by immunostaining (FIG. 14H).

In conclusion, Isx-9 was very effective small molecule for converting iPSC consistently into cardiac progenitors including endothelial/vascular progenitors and smooth muscle cells.

### Experiment No. 7

Cardiac progenitor cells (CPCs) being multipotent offer a promising source for cardiac repair due to their ability to proliferate and multiply into cardiac lineage cells. In this experiment, Applicant explored a novel strategy for human CPCs generation from human induced pluripotent stem cells (hiPSCs) using a cardiogenic small molecule, isoxazole (ISX-9) and their ability to grow in the scar tissue for functional improvement in the infarcted myocardium.

### Method and Results

Briefly, CPCs were induced from hiPSCs with ISX-9. CPCs were characterized by immunocytochemistry and RT-PCR. The CPC survival and differentiation in the infarcted hearts were determined by in vivo transplantation in immunodeficient mice (NOD/SCID mice) following LAD ligation and their effects were determined on fibrosis and functional improvement. Applicant found ISX-9 simultaneously induced expression of cardiac transcription factors, Nkx2.5, ISL1, GATA4, Mef2c hiPSCs within 3 days of treatment and successfully differentiated into three cardiac lineages in vitro. mRNA and miRNA-sequencing results showed ISX-9 targeted multiple cardiac differentiation, proliferation signaling pathways and upregulated myogenesis and cardiac hypertrophy related-miRNAs. CPCs transplantation promoted myoangiogenesis, attenuated fibrosis and led to functional improvement in treated mice.

This study demonstrates a novel technique of producing pure CPCs from hiPSCs using a single cardiogenic small molecule, ISX-9 with robust muscle differentiation potential through diverse signaling pathways. These CPCs were multipotent and differentiated into three cardiac lineages to form new CMs and vessels in the infarcted heart, thus reducing fibrosis and improving functional indices.

Stem cell-based therapies hold great promise and potential for cardiac regeneration. Human Induced pluripotent stem cells (hiPSC) could potentially generate unlimited functional human cell types in vitro for autologous and personalized medicine. Previous studies have reported that iPSC-derived cardiomyocytes (CMs) transplantation provided functional benefits in the rodent models of myocardial infarction (MI). See Wang Y. et al., Medical physics. 2016); Wang Y. et al., IEEE International Conference on: IEEE; 2011). However, poor engraftment of cardiomyocytes after transplantation to the infarcted heart is a common problem which impacts the final outcome. The beneficial effects of CMs transplantation is believed to be due to paracrine mechanism rather than graft cell integration with the host myocardium. These limitations are possibly attributed to poor cell survival and retention under ischemic conditions, limited proliferative capacity of differentiated CMs and lack of vascularization in the graft. In addition, the arrhythmogenic risks after cardiac transplantation due to immaturity and heterogeneity of iPSC-CMs or embryonic stem cells (ESCs)-CMs remain to be resolved prior to their full scale application. See Li H. et al., Bioinformatics. 2009; Robinson M D et al., Bioinformatics. 2010); Benjamini Y et al., Journal of the Royal Statistical Society Series B-Methodological. 1995.)

Cardiac progenitor cells (CPCs) offer a promising avenue for cardiac repair due to their multipotency and ability to proliferate. Previous studies demonstrated that CPCs derived from hiPSCs or ESCs are capable of differentiation into multiple cardiac lineages without teratoma formation. Moreover, transplantation of CPCs derived from hiPSCs and murine iPSCs more effectively improved cardiac function compared with iPSCs-CMs (Kozomara A. et al. Nucleic Acids Res. 2014); (Xuan W et al., Cardiovasc Res. 2011). Recently two studies reported the CPCs derived from mouse fibroblasts spontaneously differentiated into CMs, ECs and SMCs in infarcted mouse hearts and improved cardiac function following MI. (Zhang et al., Cell stem cell, 2016; Vol. 18; pgs. 368-381); (Lalit P A et al., Cell stem cell, 2016; Vol. 18; pgs. 354-367). Furthermore, several clinical trials on cell therapies for cardiac diseases are ongoing using adult stem cells. (Sheridan C. Cardiac, Nature biotechnology, 2013; Vol. 31; pgs. 5-6); (Malliaras et al., Stem cells translational medicine, 2014; Vol. 3; pgs. 2-6). For example, the European Consortium CARE-MI is using human CPCs isolated from the right atria appendage of donors for acute myocardial infarction treatment. (Gomes-Alves et al., Translational research: the journal of laboratory and clinical medicine, 2016; Vol. 171; pgs. 96-110 & 111-113). It has been reported recently that SSEA-1⁺ CPCs derived from human ESCs were delivered into the infarcted area of a single patient suffering with severe heart failure, and they led to cardiac functional improvement. See Menasche et al., European heat journal, 2015; Vol. 36; pgs. 2011-(2017). This clinical study demonstrated a potential feasibility of generating a clinical-grade population of human ESC-derived CPCs and provided strong encouragement for future clinical application of hiPSCs or ESCs-derived CPCs. Current strategies of human CPC generation include isolation from atria appendage of donors and expansion in vitro. (Koninckx et al., Cardiovascular research, 2013; Vol. 97; pgs. 413-423), derivation from hiPSCs or ESCs, which have to be converted into embryoid bodies or treated with multiple small molecules and growth factors (activin, BMP4). (Yang et al., Nature, 2008; Vol. 453; pgs. 524-528); (Lei et al., Journal of visualized experiments: JoVE 2015: 52047; Moretti et al., FASEB journa: official publication of the Federation of American Societies for Experimental Biology, 2010; Vol. 24; pgs. 700-711). These strategies are labor-intensive and time-consuming, with high production costs, which limit clinical application. Additionally, heart tissue-derived CPCs are limited by availability of limited human tissue source. Therefore, novel platform for human CPCs generation in large scale is needed for safety, high reproducibility, high purity, cost-effectiveness and ease of production for further clinical application.

ISX-9 which was referred as ISX1 in some reports. (Russell et al., ACS chemical biology, 2012; Vol. 7; pgs. 1067-1076; Burchfield et al., Journal of investigative medicine: the official publication of the American Ferderation for Clinical Research, 2016; Vol. 64; psg. 50-62.) ISX-9 is a small molecule belonging to Isoxazole family. ISX-9 was reported as a cardiogenic small molecule. A previous study showed that direct injection of ISX-9 failed to regenerate infarcted myocardium and mitigate scar tissue formation or improve ventricular function after MI. (Russell et al., ACS chemical biology, 2012; Vol. 7; pgs. 1067-1076). More recently this small molecule is reported to precondition adipose derived stem cells, which enhanced their effects in the ischemic heart. (Burchfield et al., Journal of investigative medicine: the official publication of the American Ferderation for Clinical Research, 2016; Vol. 64; psg. 50-62). In addition, ISX-9 induced neuronal differentiation in neural progenitor cell line. (Zhang et al., Differentiation; research in biological diversity, 2011; Vol. 81; pgs. 98-104). However, it remains unknown whether ISX-9 could direct cardiac differentiation in hiPSCs. Here, Applicant successfully generated multipotent CPCs from hiPSCs using one single small molecule-ISX-9. Unexpectedly, this small molecule initiated simultaneous upregulation of all cardiac transcription factors in hiPSC and allowed differentiation of all three cardiac lineage cells resulting in cardiac regeneration, reduced fibrosis and improved cardiac function following myocardial infarction.

### Materials and Methods

In Vitro Cardiac Progenitor Cells Differentiation from hiPSC and Characterization of hiPSC-Derived Cardiac Cells

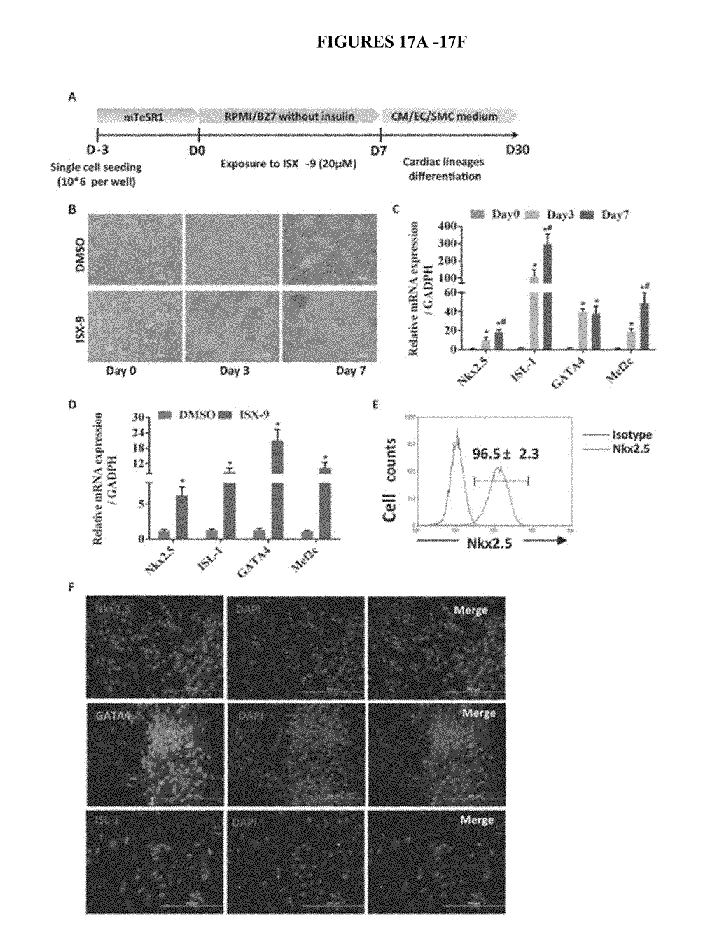

The Human iPSC cell line (ACS-1021™) induced from human fibroblasts was purchased from ATCC Company. Briefly, iPSCs maintained on vitronectin coated six-well plate in mTeSR1 were dissociated into single cells using accutase (Invitrogen) at 37° C. for 10 min and then were seeded on to a vitronectin-coated six-well plate at 1×10⁶ cell/well in mTeSR1 supplemented with 5 μM ROCK inhibitor (Y-27632, Stem Cell Technology) for 24 h. Afterwards cells were cultured in mTesR1, which was changed daily. At day 0, the medium was changed to RPMI/B27 minus insulin supplemented with ISX-9 (20 uM, dissolved in DMSO) for 7 days. Schematic of protocol for generation of cardiac progenitors is shown in FIG. 17A. At day 3 and 7, cardiac genes were analyzed by RT-PCR and immunostaining. The purity of Nkx2.5 positive cells in IXS-9 treated cells was analyzed by FACS. For cardiomyocyte (CM) differentiation, after 7 day of ISX-9 treatment, culture medium was switched to RPMI/B27 with insulin for another 10 and 30 days. For endothelial cell (EC) differentiation, culture medium was switched to EGM-2V medium (Lonza) for another 10 days. For smooth muscle cell (SMC) differentiation, culture medium was replaced by DMEM-F12 medium supplemented with TGFβ (2 ng/ml, R&D) and PDGFBB (long/ml, R&D) for 10 days. CMs, ECs and SMCs were characterized and detailed in the supplemental appendix. To determine the potential mechanism of cardiac differentiation induced by ISX-9 in hiPSCs, cells were treated with TGFβ signaling pathway inhibitor, 5 μM LY2109761 (Selleck) or Wnt signaling pathway inhibitors, 3 μM XAV939 (Selleck) or 5 μM IWP2 (Selleck) at Day −1 and Day 0, recombinant Human WIF-1 Protein (R&D, 200 ng/ml), siWnt5 and siWnt11 at Day 3 and Day 4 respectively.

### Murine Model of Myocardial Infarction and Cell Transplantation

Myocardial Infarction model was induced in 8-9-week-old NOD/SCID mice (The Jackson Laboratory) by left anterior descending artery (LAD) ligation under 2% inhaled isoflurane anesthesia (See also paragraph [0335]). Briefly, the heart was exposed by left-sided limited thoracotomy and the LAD was ligated with a prolene #8-0 suture. Myocardial ischemia was confirmed by color change of the left ventricular wall. Mice were randomized into 3 groups: (1) Ischemic control group injected with DPBS (2) Ischemic group injected with 1×10⁶ hiPSCs, (3) Ischemic group injected with 1×10⁶ CPCs. The cells were injected 10 minutes after LAD ligation at 2 sites of the infarcted areas under direct vision. For post-engraftment tracking of the transplanted cells and determination of their fate, the cells were labeled with PKH26 (Sigma, Product#PKH26-GL) according to manufacturer's instructions. The chest was closed and the mice were allowed to recover. The experimental protocols were approved by the University of Illinois at Chicago Animal Care and Use Committee and the methods were performed in accordance with the guide for the Care and Use of Laboratory Animals by the Institute of Animal Resources.

Cardiac function measurement by echocardiography was performed at different time points till 3 months after MI. Mice were sacrificed 3 months after MI and hearts were removed for scar tissue measurement and histological analysis.

### Effect of Different Stages of Developing CMs on Engraftment in Infarcted Mouse Heart.

CMs derived from embryonic stem cells (ESC) or iPSC are good reliable sources for cardiac repair in vivo but also might serve as a platform for drug safety screening, personalized and precision cardiovascular medicine in vitro. The developmental stage at which the differentiated cells should be used for cardiac regenerative purposes remains elusive. Here Applicant further differentiated CPCs induced by isoxazole into CMs by culturing in RPMI/B27 with insulin medium. Applicant investigated the engraftment capability of different stages of CMs in the infarcted mouse heart. Engrafted CMs were identified by staining with human specific cTnT. The cardiac cells were identified by immunostaining for α-sarcomeric actinin or cTnI. Immunostaining results showed that both day 15 and day 35 CMs after transplantation were observed in the infarcted hearts as identified by double staining of cTnI and human mitochondria antigen or human specific cTnT and α-actinin (data not shown). Quantitative analysis showed that high percentage CMs of human origin was present in the infarcted zone while mouse origin CMs were observed in the peri-infarct area of hearts transplanted with day 35 CMs than mice with day 15 CMs (data not shown). These results suggest that day 35 CMs might be optimal for transplantation.

Next, Applicant analyzed fibrosis area and cardiac function in the mice post-MI with day 35 CMs transplantation. Serial echocardiographic examination of untreated mice showed a time-dependent increases in LVEDD and LVESD after MI, as well as a progressive decline in LVFS over the follow-up period of 3 months. In contrast, transplantation of differentiated CMs significantly slowed the progression of left ventricle enlargement (LVESD, 2.39±0.52 mm vs. 3.42±0.62 mm; LVEDD, 3.63±0.47 mm vs. 4.25±0.70 mm) and improved cardiac function (LVFS, 34.68±7.12% vs. 20.17±4.00%) in mice with MI compared with control mice (data not shown). Moreover, smaller scar size was observed in mice transplanted with CMs than in untreated mice (data not shown).

### Statistical Analysis

Data are expressed as mean±SEM. Statistical analysis of differences was compared by ANOVA with Bonferroni's correction for multiple comparisons. A probability value of P<0.05 was considered statistically significant.

### Results

Cardiac Differentiation and Characterization of iPSC-Derived Cardiac-Lineage CMs, ECS and SMCs

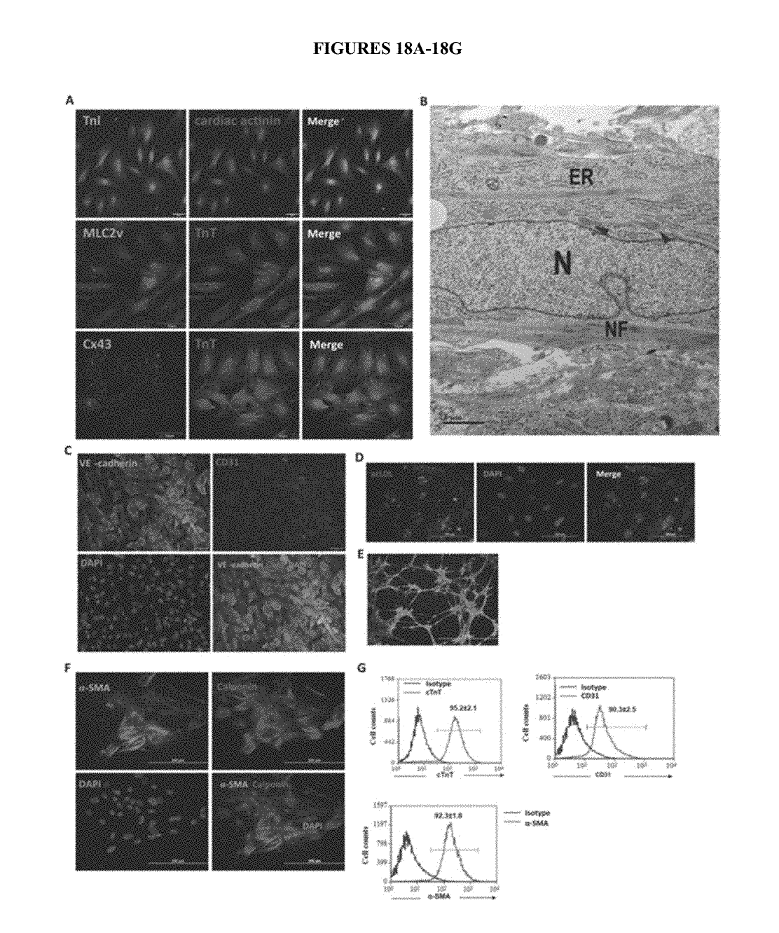

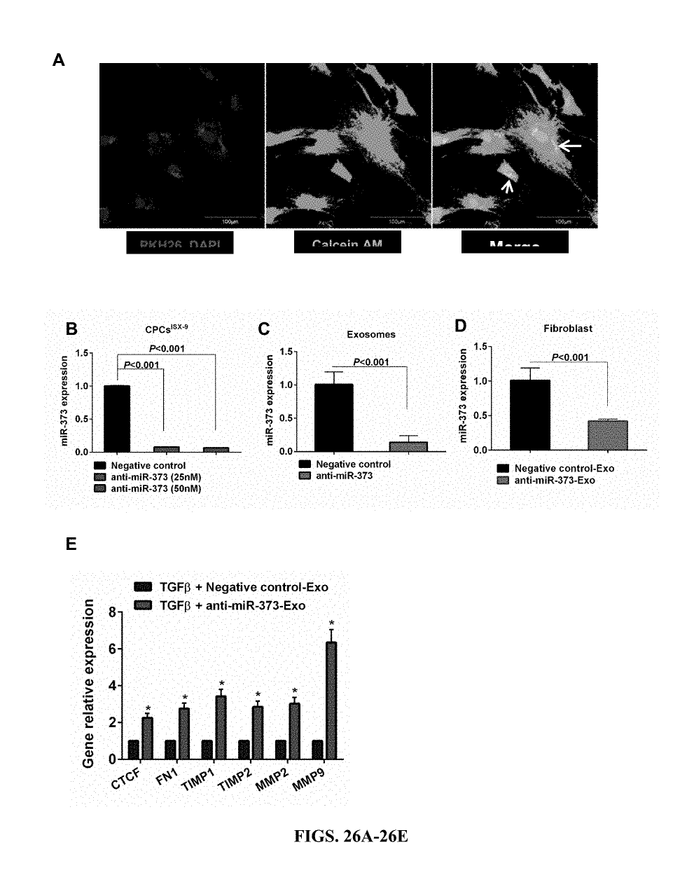

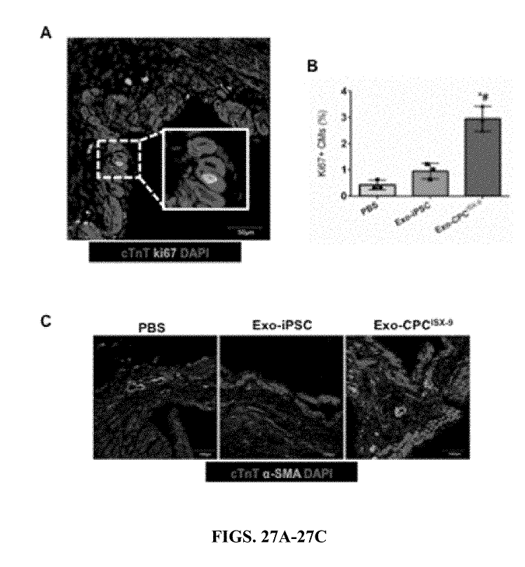

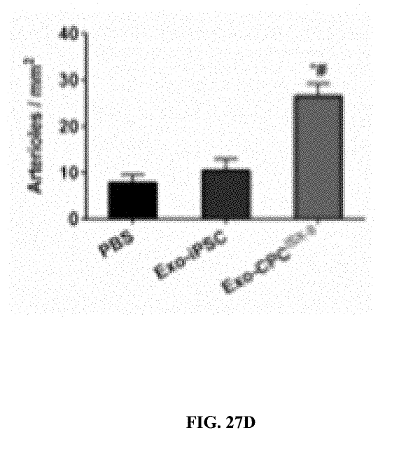

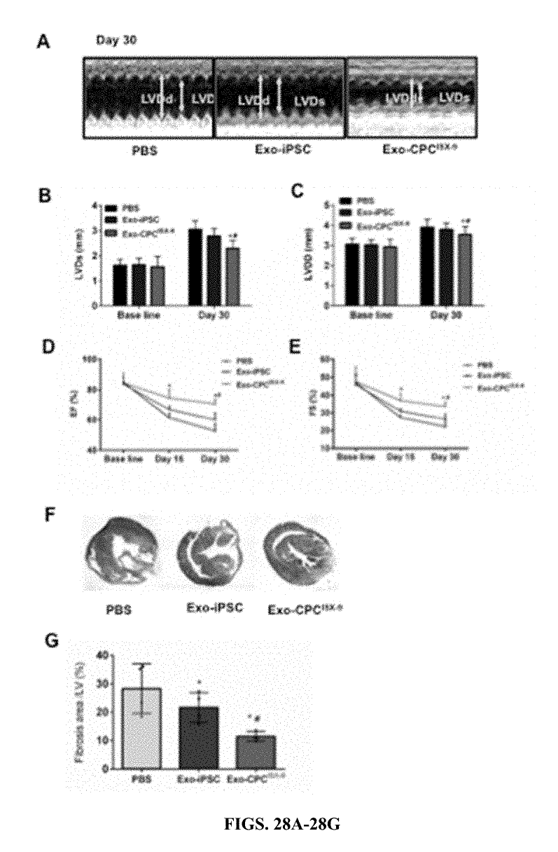

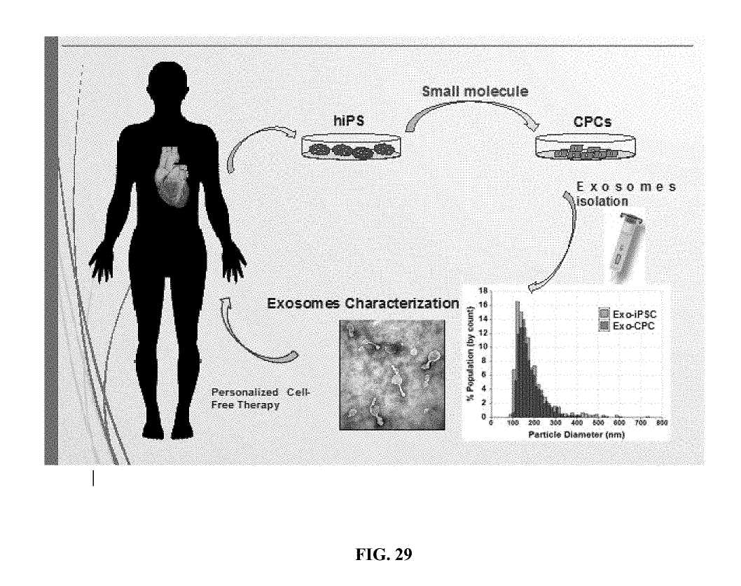

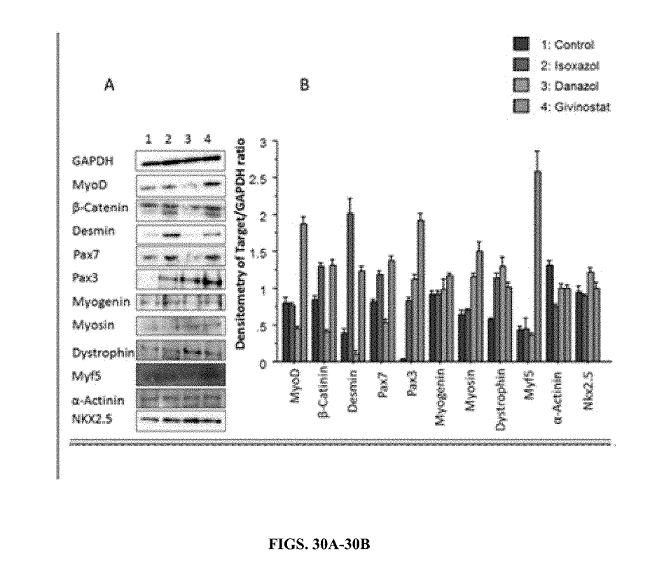

Compared with DMSO treated hiPSCs, ISX-9 treatment promoted formation of clusters of proliferating cells in RPMI/B27 differentiation medium (FIG. 17B). Real-time PCR results showed that ISX-9 dramatically induced the expression of multiple CPCs related genes in hiPSC. As shown in FIG. 17C, Nkx2.5, GATA4, ISL-1 and Mef2c upregulation was observed after 3-day ISX-9 treatment and further enhanced after 7-day treatment (except GATA4). As compared with DMSO treated cells, ISX-9 increased expression of the above cardiac transcription factors (FIG. 17D). Immunofluorescence staining also showed that transcription factors (Nkx2.5, GATA4 and ISL-1) were highly expressed in hiPSC with 7-day ISX-9 treatment (FIG. 17F and FIG. 28) and by FACS 96.5±2.5% cells were Nkx2.5 were positive, suggesting the high purity of CPC generation (FIG. 17F). These Nkx2.5+ cells were multipotent and directly differentiated into all three cardiovascular lineages, including CMs, ECs and SMCs in basal differentiation conditions without any specific induction signaling molecules (FIG. 18A). These cardiac cells expressed CM-, EC- and SMC-specific proteins. By transmission electron microscopy (TEM), the developing CMs were rich in endoplasmic reticulum with ribosomes, developing myofilaments, mitochondria and glycogen particles. Chromatin material was uniformly distributed in the nucleoplasm (FIG. 18B). In addition, the differentiated ECs exhibited a similar phenotype and function to primary ECs. Formation of tube-like structures and LDL-uptake were observed in differentiated ECs (FIG. 18D and FIG. 18E). To characterize cells more precisely after CPCs differentiation, we analyzed the percentage of CMs (TnT⁺), ECs (CD31⁺) and SMCs (α-SMA⁺) in CM, EC and SMC differentiation conditions for 10 days by FACS. FACS analysis revealed high efficiency in cardiac lineages differentiation from these induced CPC. We can obtain about 95.2±2.1% CMs, 90.3±2.5% ECs and 92.3±1.8% SMCs in basal differentiation medium respectively (FIG. 18G). Moreover, ISX-9 acts as a very strong differentiation inducer in hiPSCs even when hiPSCs were cultured in mTeSR1 undifferentiation medium (FIG. 26). In mTeSR1 undifferentiation medium, ISX-9 could still induce Nkx2.5, GATA4 and ISL-1 expression in part of the cultured hiPSCs (FIG. 27). Taken together, these results suggest that successful generation of CPCs by a novel small molecule ISX-9 is remarkable.

Potential Key Signaling Pathways and miRNAs Expression Mediated by ISX-9 Treatment

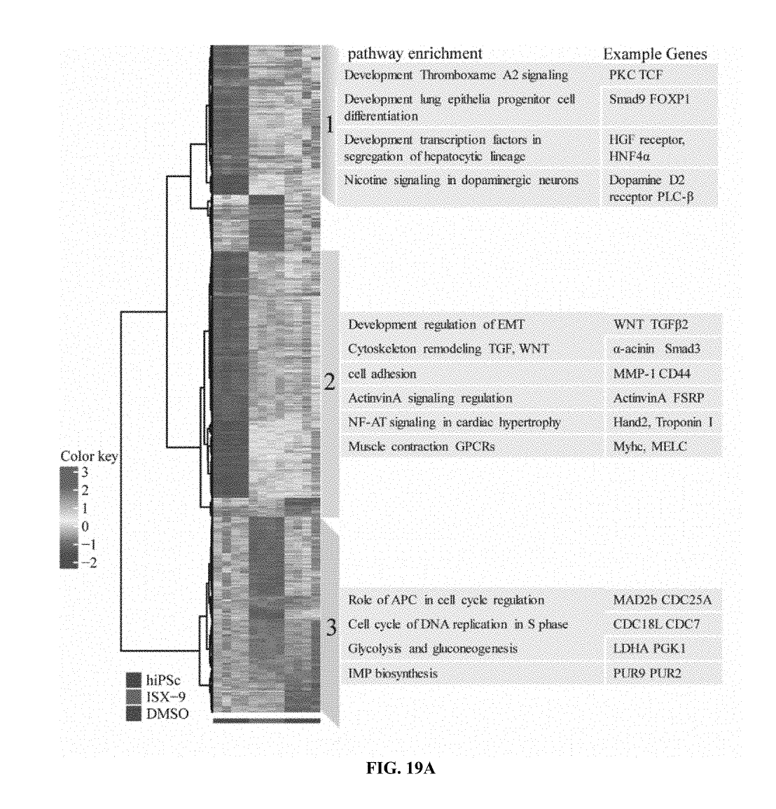

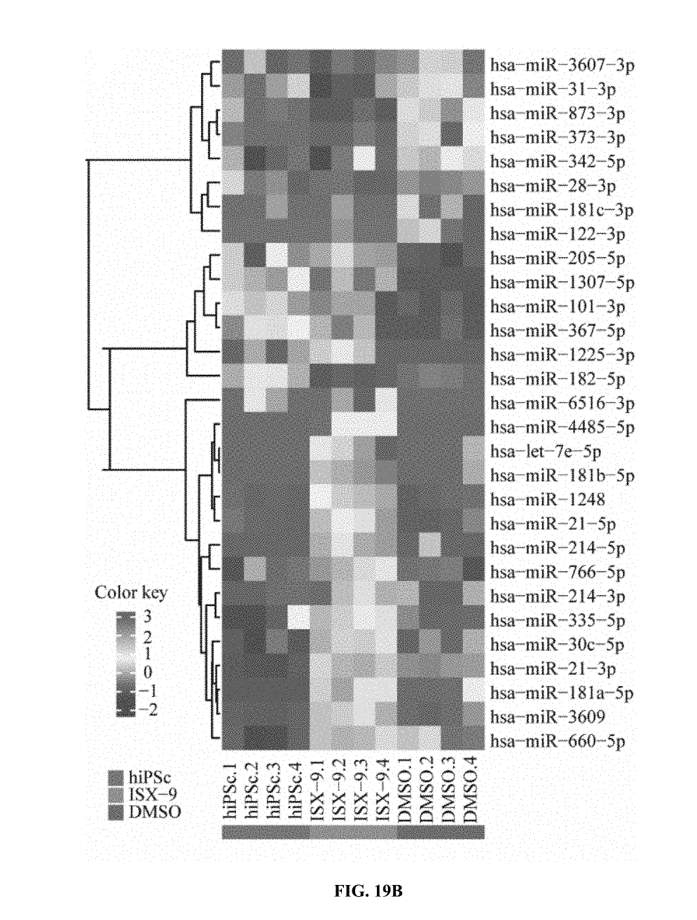

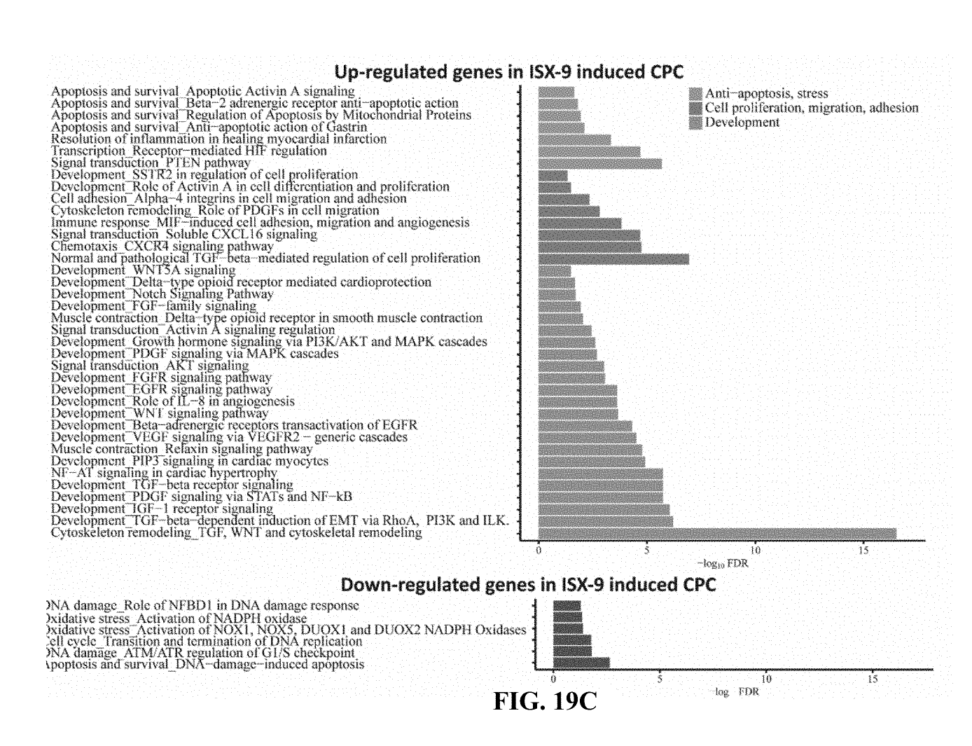

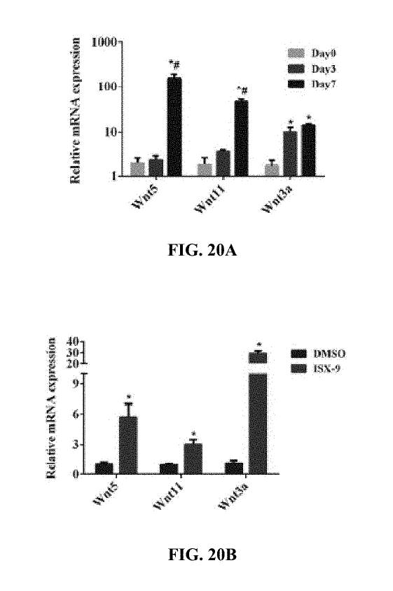

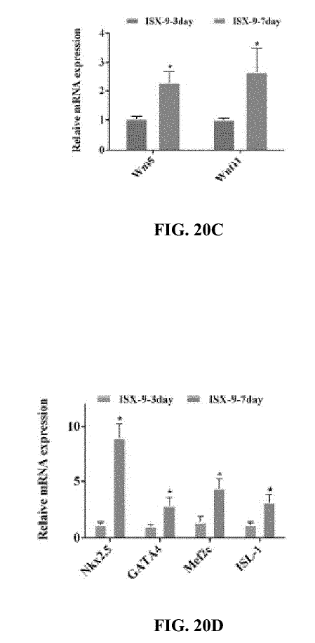

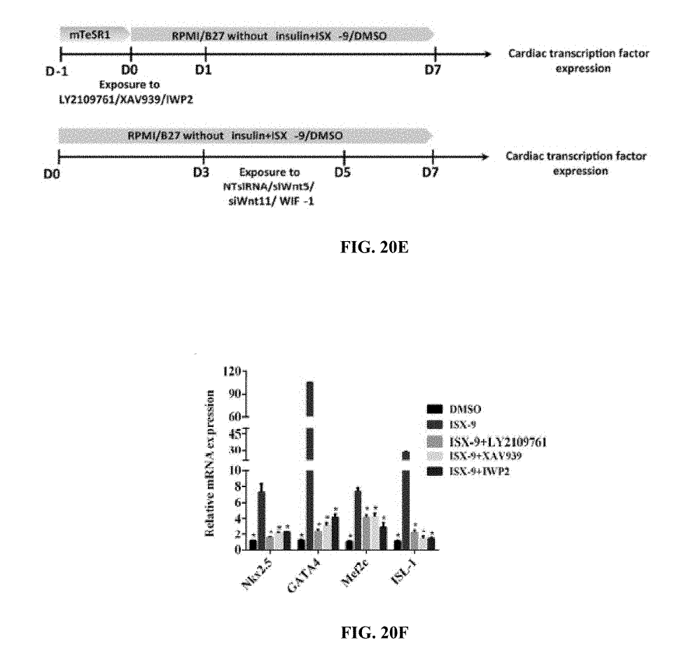

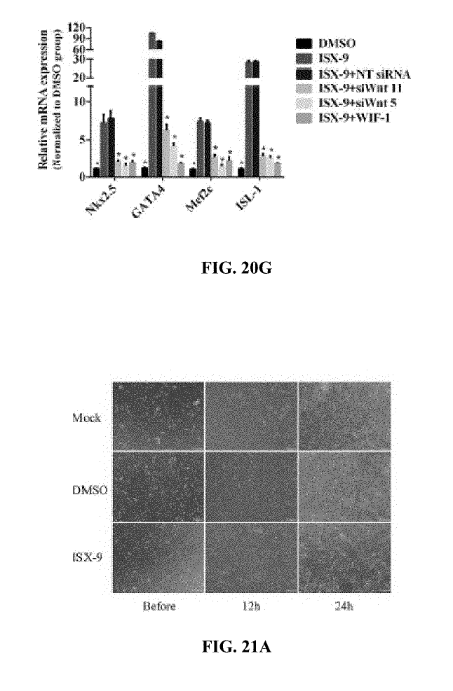

Applicant performed transcriptome analysis to reveal differences in gene expression in hiPSC by ISX-9 treatment. mRNA-sequencing showed different gene expression profile amongst groups (FIG. 19A). Signaling pathway enrichment analyses of upregulated and downregulated genes in different groups were shown in FIG. 19C. As compared with DMSO treatment and undifferentiated hiPSCs (FIG. 19C and FIG. 20), ISX-9 promoted WNT and cytoskeleton remodeling and TGF-β induced EMT signaling, which were involved in cardiac differentiation. The expression of Wnt3a, the key activator of canonical Wnt signaling pathway, was dramatically upregulated at day3 and day7 compared with undifferentiated hiPSCs. Wnt3a expression was not significantly different at day 3 and day 7. However, the expression of Wnt5a and Wnt11, the trigger molecules of non-canonical Wnt signaling pathway were upregulated at day7 but not at day 3 (FIG. 20A). In comparison to DMSO-treated group, ISX-9 induced upregulation of Wnt3a, Wnt5a and Wnt11 (FIG. 20B). Continuous treatment with ISX-9 for 7 days increased expression of Wnt5, Wnt11 and cardiac transcription factors (Nkx2.5, Mef2c, GATA4 and ISL-1) compared with treatment only for 3 days (FIG. 20C and FIG. 20D). Applicant further verified the mRNA-sequencing results using TGFβ signaling pathway inhibitor, LY2109761, Wnt signaling pathway inhibitors, XAV939 and IWP2. Schematic description of the protocol is shown in FIG. 20E. The real-time PCR results showed that inhibition of TGFβ signaling pathway and canonical Wnt signaling pathway in the initial differentiation stage significantly decreased expression of cardiac transcription factors (Nkx2.5, Mef2c, GATA4 and ISL-1) (FIG. 20F), suggesting the critical role of TGF-β induced EMT signaling and canonical Wnt signaling in ISX-9 induced cardiac differentiation. Knock down of Wnt5a or Wnt11 using siRNA at late differentiation stages significantly inhibited expression of cardiac transcription factors in ISX-9 treated cells (FIG. 20G). Efficiency of knock down for Wnt5a and Wnt11 is shown in FIG. 20. In addition, treatment with WIF-1 during the late stage, a non-canonical Wnt signaling pathway inhibitor, showed similar results (FIG. 20G), suggesting non-canonical Wnt signaling pathway was involved in ISX-9 induced cardiac differentiation. Moreover, signaling pathway enrichment analyses also revealed other beneficial effects by ISX-9 on cell proliferation, migration, and anti-apoptosis. A series of genes related to migration, proliferation and anti-apoptosis signaling were upregulated by ISX-9, meanwhile genes related to DNA damage and oxidative stress were downregulated. In addition, miRNA-sequencing showed several myogenesis related miRNAs and also upregulation of cardiac hypertrophy related-miRNAs, including miR-335, miR-21, miR-30c, and miR-214 (FIG. 19B).

Cytoprotection of hiPSC-CPCs Against Ischemia

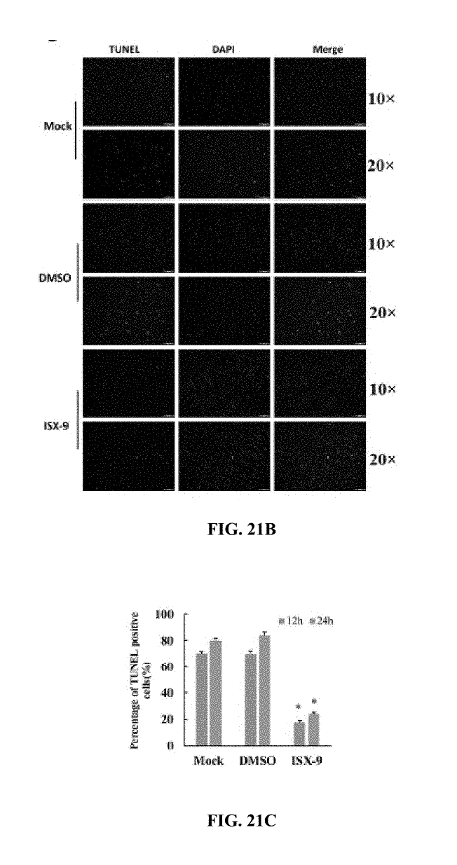

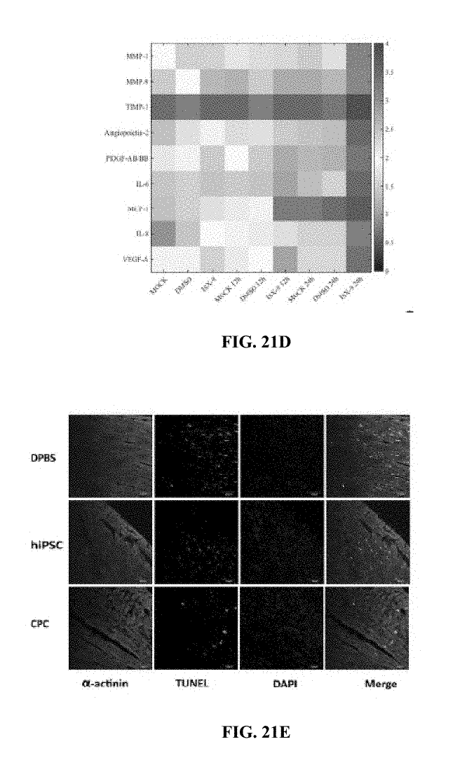

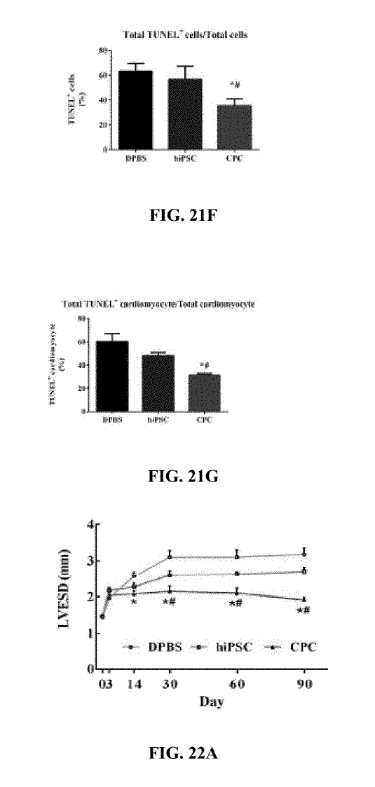

To determine whether the induced CPCs exhibit cytoprotective effects, Applicant cultured hiPSC with different treatments under hypoxic condition. Culturing of these cells under 1% O₂ for 12 h and 24 h resulted in cell death with or without DMSO treatment. TUNEL assay revealed dramatic increase in apoptotic cells in hiPSC from mock and DMSO control groups due to hypoxia of 1% O₂, while fewer apoptotic cells were noted in ISX-9 treated cells (FIG. 21A and FIG. 21B). Approximately 70%±2.5% and 80%±6.5% cells underwent apoptosis in DMSO group after 12 h and 24 h 1% O₂ exposure, respectively. However, ISX-9 treatment reduced the apoptotic cells to 15%±3.5% and 24%±4.3% after 12 h and 24 h 1% O₂ exposure, respectively (FIG. 21C). Furthermore, CPCs transplantation reduced apoptosis in the border area of infarcted hearts after 3 days post-MI (FIGS. 21E-21G). Applicant then performed multiple cytokines assays to identify paracrine factors which might be responsible for these cytoprotective effects. Several cytokines were significantly increased with ISX-9 treatment under normoxia or hypoxic stress (1% O₂) for 12 h or 24 h respectively, compared with DMSO treatment (P<0.05). These protective cytokines included anti-apoptosis factors (angiopoietin-2, IL-6, MMP-1, PDGF-BB, and TIMP-1), cell migration inducing factors (angiopoietin-2, IL-8, MCP-1, MMP-9 and VEGF-A) and angiogenesis factors (angiopoietin-2, PDGF-BB and VEGF-A). Compared with normoxia condition, 24 h hypoxic stress further increased concentrations of all these protective cytokines (P<0.05) (FIG. 20 and FIG. 21).

Transplantation of hiPSC-Derived CPCs Attenuated Cardiac Remodeling after MI

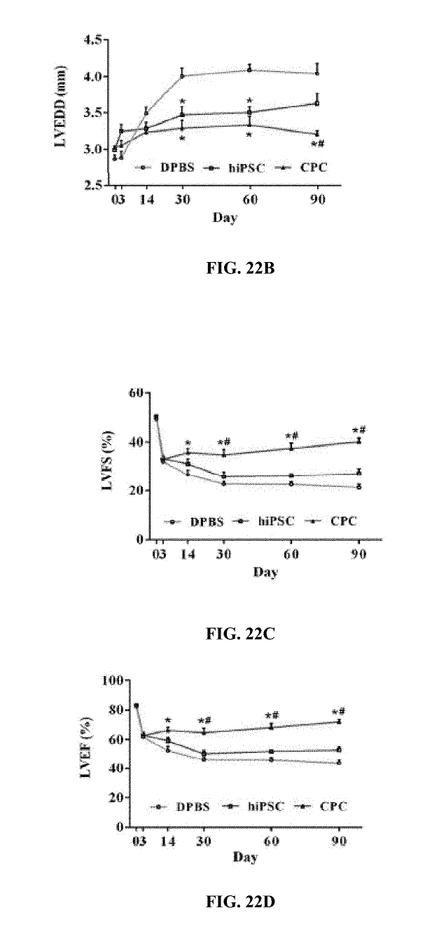

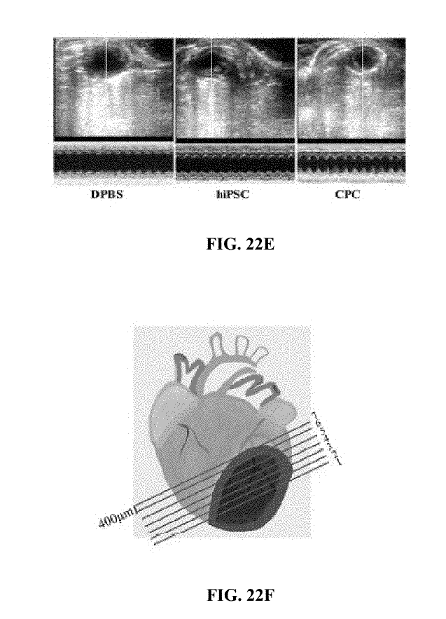

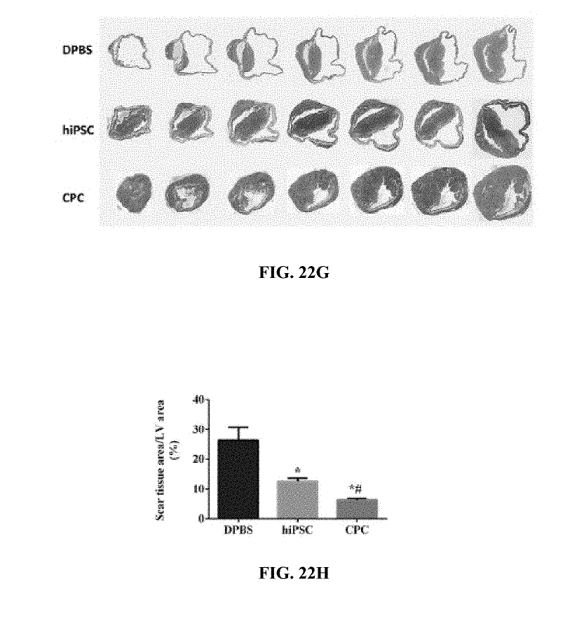

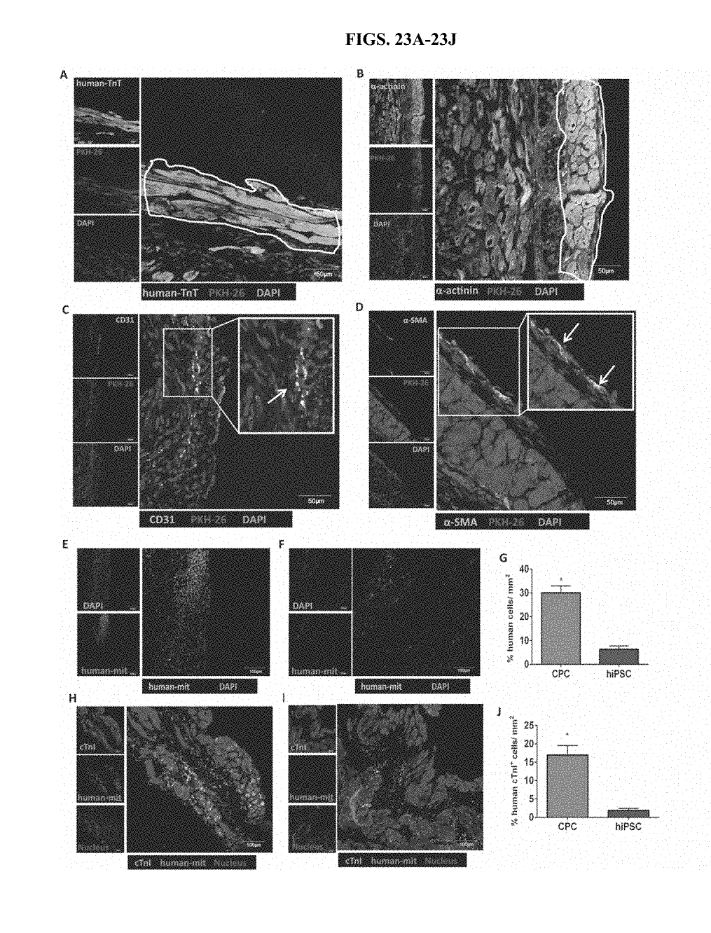

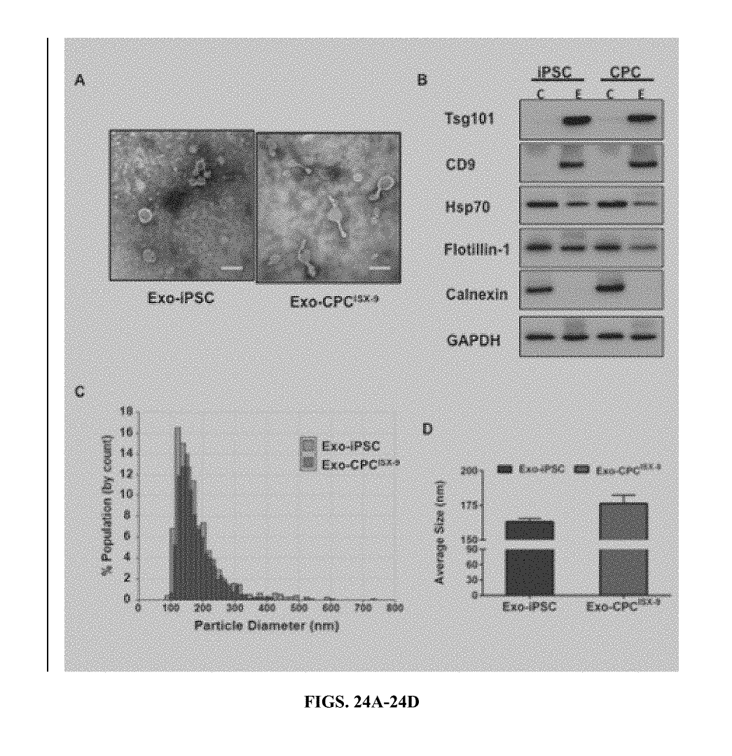

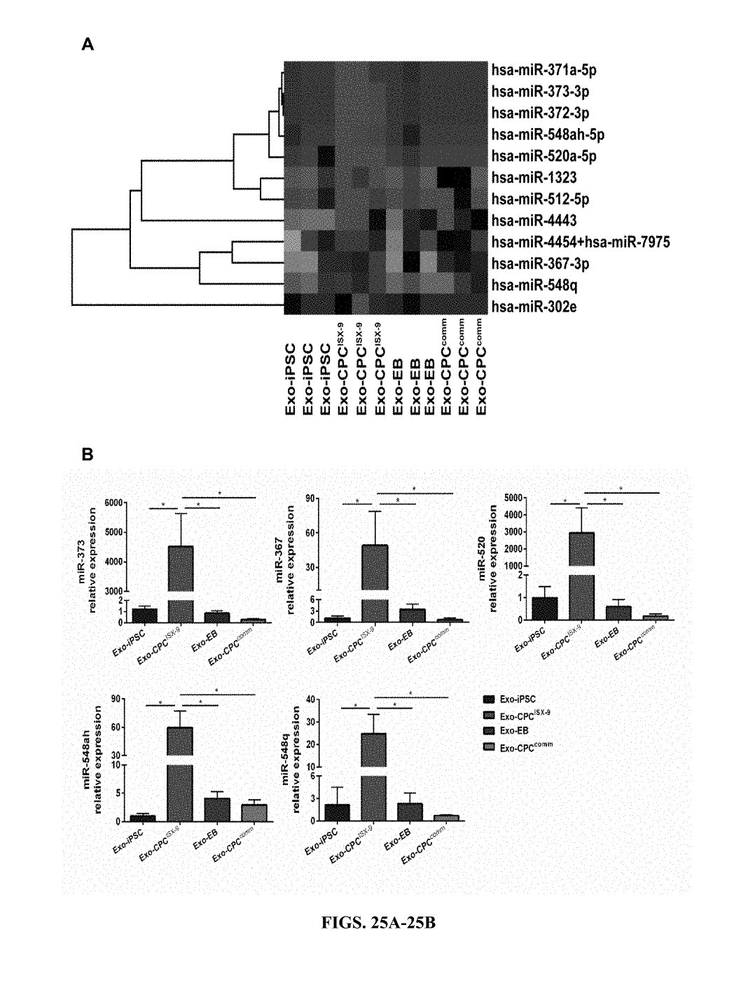

Serial echocardiographic examination of DPBS and hiPSC treated mice showed a time-dependent increase in LVEDD and LVESD after MI, as well as a progressive decline in LVFS and EF over the follow-up period of 3 months. In contrast, transplantation of CPCs significantly slowed the progression of LV enlargement (LVESD and LVEDD) and LVFS depression in mice with MI, and these differences became more obvious over time (FIGS. 22A-22D). Transplantation of CPCs significantly increased LVEF to 71.95±1.53% from 43.39±2.31% and LVFS to 40.14±1.53% from 21.24±1.30% in comparison with the DPBS treated mice. The pathological remodeling of left ventricle chamber dimensions during systole (LVESD) and diastole (LVEDD) was also significantly reduced in CPCs treated hearts as compared with DPBS and hiPSCs treated mice (FIG. 22E). In parallel to functional indices, significant smaller scar size in mice transplanted with CPCs 3 months post-MI was observed in comparison with DPBS and hiPSCs treated mice (FIGS. 22F-22H). These data suggest that transplantation of CPCs attenuates cardiac remodeling after MI.

In Vivo Differentiation and Growth of hiPSC-CPCs after Transplantation into Infarcted Myocardium

Applicant directly injected hiPSC and hiPSC-CPCs along the infarcted area of left ventricle. Applicant observed extensive survival, proliferation, and differentiation of PKH26 labeled cardiac progenitors 2 months and 3 months after transplantation in the infarcted region. Immunofluorescence analyses showed co-localization of PKH-26 red fluorescence and α-sarcomeric actinin or human specific cTnT staining (green fluorescence) at 2 months (FIG. 23) and 3 months (FIG. 23A and FIG. 23B) after MI, suggesting that the transplanted CPCs grew into new CMs which replaced scar tissue. The total number of transplanted human cells retained in the peri-infarcted area was significantly higher in CPCs treated mice when compared with hiPSCs treated mice 72 h post-MI using human mitochondria antigen tracking (30.03±1.69% vs. 6.27±0.85%) (FIGS. 23E-23G). Moreover, Applicant analyzed the number of differentiated CMs from transplanted CPCs or hiPSCs in the peri-infarcted area by staining myocardial sections for human mitochondrial antigen and cardiac troponin I expression. At 3 months post-MI, the number of differentiated CMs from transplanted CPCs were detected in the peri-infarcted area by 8-fold when compared with hiPSCs transplanted hearts (FIGS. 231I-23J). In addition, engrafted CPCs differentiated into ECs and SMCs which formed vascular structures in the border area of infarcted hearts (FIG. 23C and FIG. 23D). No tumor formation was observed in any of the transplanted animals used in this study (n=16). These observations support the idea that CPCs generated by ISX-9 successfully engrafted and differentiated into three cardiac lineages, which formed new CMs and blood vessels in the infarcted heart.

Transplantation of hiPSC-CPCs Enhanced Vasculogenesis in Infarcted Area

Angiogenesis could contribute to cardiac function improvement after ischemia. CPCs significantly increased vascular density and arteriole density in the infarct and border areas compared with DPBS and hiPSC treated groups (FIG. 23).

### Discussion

In the present study, Applicant successfully generated human CPCs from human iPS cells using a simple novel method without using cumbersome intermediate cocktails of small molecules and growth factors. These CPCs protected themselves by releasing cytokines against ischemic environment. After transplantation, they differentiated into multiple cardiac lineage cells and covered the scar area with new myofibers and vessels in the infarcted heart. These CPCs also showed long term beneficial effects on cardiac function improvement reflecting higher FS and EF.

ISX-9 is a cardiogenic small molecule and neural differentiation inducer. A previous study showed that direct injection of ISX-9 failed to mitigate scar formation or ventricular functional decline after MI. (Russell J L et al., ACS chemical biology, 2012; Vol. 7; pgs. 1067-1076). Recently it has also been shown that preconditioning of adipose-derived stem cells with ISX-9 promoted potential differentiation toward a cardiomyocyte phenotype, but this study also found the negative effect of Nkx2.5 on myocyte differentiation due to persistent stimulation of Nkx2.5. (Burchfield J S et al., Journal of investigative medicine: the official publication of the American Federation for Clinical Research, 2016; Vol. 64; pgs. 50-62). It was reported that the expanded ISL1 and Nkx2.5 cardiac fate maps were remarkably similar and multipotent Isl1+/Nkx2.5+ progenitor might represent a common cardiac progenitor of CM, SMC, and EC lineages in all four cardiac chambers. (Ma Q et al., Developmental biology, 2008; Vol. 27; pgs. 1050-1056). According to Applicant's study, as early as 3 days treatment with ISX-9 promoted simultaneous upregulation of multiple cardiac transcription factors in hiPSCs with ISX-9. Therefore, induction of several transcription factors with ISX-9 (ISL1, Nkx2.5, GATA4, and Mef2c) initiated multilineage differentiation program in hiPSC. At the end of 7 days treatment with ISX-9, these CPCs can be further propagated into myocytes, EC and smooth muscle progenitors.

The common problems associated with iPSC-based myocardial repair include limited proliferation and differentiation of transplanted hiPSC-CMs, lack of vascularization, arrhymogenic risk and tumorigenesis. Lam J T et al., Pediatric cardiology, 2009; Vol. 30; pgs. 690-698); (Knoepfler P S. Stem cells, 2009; Vol. 27; pgs. 1050-1056). Compared with other cells, CPCs are ideal candidates for myocardial repair due to their multipotency and ability to proliferate. Previous studies attempted to develop different techniques to produce pure population of CPC using EB generation, various growth factors, and WNT signaling pathway inhibitors. Koninckx R et al., Cardiovascular research, 2013; Vol. 97; Vol. 413-423); (Moretti A et al., FASEB journal: official publication of the Federation of American Societies for Experimental Biology, 2010; Vol. 24; pgs. 700-711); (Schmeckpeper J et al., Journal of molecular and cellular cardiology, 2015; Vol. 85; pgs. 215-225). To the best of Applicant's knowledge, the present study is the first to use a single molecule to produce CPCs in large numbers with high purity from iPSCs, which can further facilitate generation of three cardiac lineages including CMs and vascular cells. Here, Applicant used this specific molecule to generate cardiac specific progenitor cells up to 96%.

The primary aim of cell regenerative medicine is the replacement of the dead cells with the new cells in order to restore the cardiac structure and function. The regenerative field at the present is controversial due to conflicting results ranging from no new cell formation to sparse newly formed cells in the infarcted tissue. (Ong S G et al., Circulation, 2015; Vol. 132; pgs. 762-771); (Chong J J et al., Nature, 2014; Vol. 510; pgs. 273-277; Zhang Y et al., Cell stem cell, 2016; Vol. 18; pgs. 368-381). However, the majority of studies provide alternative explanation of cell therapy due to beneficial effects caused by paracrine factors which reduce cell death and stimulate cell migration and proliferation. Ye L et al., Cell stem cell, 2014; Vol. 15; pgs. 750-761); (Gouadon E et al., Stem cells, 2016; Vol. 34; pgs. 34-43). This study supports both these concepts which are complimentary to each other. These results demonstrate that CPCs were tolerant to ischemic condition and bestowed additional protection to resident CMs in ischemic heart by preventing cell death. Furthermore, these CPCs also released angiogenesis factors in a paracrine manner. Applicant found CPCs transplantation increased vessel density in the infarcted heart, which is partly due to these angiogenesis factors released from CPCs. Consistent with cytokine and signaling pathway enrichment analyses, ISX-9 provided additional beneficial effects on cell proliferation, migration, and apoptosis, thus assuring cell survival and successful engraftment of transplanted CPCs. Poor engraftment of transplanted CMs in previous studies might be due to lack of oxygen and nutrition in the infarcted myocardium. Here, Applicant's CPCs showed anti-oxidant effects, which might partly overcome the problem of poor cell survival in ischemic heart. Previously it was reported that approximately 10% of cells were retained in the border area. (Sharma S et al., Circulation research, 2017; Vol. 120; pgs. 816-834). However, in Applicant's study the higher rate of CPC retention and survival suggest that Applicant's CPCs were more tolerant to ischemia in the ischemic environment. This was further supported by differentiation analysis that about 16.93±1.5% of newly differentiated CMs were observed in the border area of the infarct hearts in CPC-treated mice 3M post-MI. It is also possible that sustained release of cytokines from the engrafted CPCs and differentiated cells might have facilitated the beneficial outcome. The engrafted CPCs not only survived but formed new CMs and blood vessels to replace scar tissue in ischemic myocardium as supported by the current data. Unfortunately we did not observe gap junctions between engrafted CPCs-CMs and host CMs.

In order to understand the possible underlying mechanism of cardiac differentiation induced by ISX-9, Applicant performed RNA-sequencing in hiPSC treated with ISX-9. Interestingly, with global transcriptome analysis, we discovered that ISX-9 upregulated genes related to multiple cardiac differentiation signaling pathways including WNT and cytoskeleton remodeling and TGF-β induced epithelial-mesenchymal transition (EMT) signaling, VEGF and activin A signaling, which strongly pointed out that ISX-9 targets multiple signaling pathways necessary for cardiac differentiation. Of note, TGF-β family members, BMP and activin, WNT and FGF family members are known to be crucial for cardiac development. For example, TGF-β is expressed early in the cardiac region of the mesoderm. (Puceat M et al. Cardiovascular research, 2007; Vol. 74; pgs. 256-261). Growth factors and small molecules have been used for induction of cardiac differentiation in stem cells via manipulation of these key developmental pathways. TGF-β has been reported to induce differentiation of bone marrow stem cells into immature CMs and induce cardiac differentiation in ESCs. (Li T S et al., Biochemical and biophysical research communications, 2008; Vol. 366; pgs. 1074-1080; Lim J Y et al., Molecules and cells, 2007; Vol. 107; pgs. 186-199). Inhibition of TGF-β activity inhibited induction of the cardiac transcription factor Nkx2.5. (Lim J Y et al., Molecules and cells, 2007; Vol. 107; pgs. 186-199). Applicant's result showed blockade of TGF-β signaling with LY2109761 decreased expression of cardiac transcription factors induced by ISX-9, which validated our RNA-sequencing data. WNT proteins have been shown to play multiple roles during cardiac development and differentiation. Wnt/β-catenin signaling enhanced early cardiac development but later it showed negative influences on cardiac specification. (Gessert S et al., Circulation research, 2010; Vol. 107; pgs. 186-199). It was demonstrated that temporal modulation of Wnt/β-catenin signaling using an inhibitor promoted robust cardiomyocyte differentiation. (Lian X et al., Proceedings of the National Academy of Sciences of the United States of America, 2012; Vol. 109; pgs. E1848-1857). In contrast, non-canonical Wnt signaling promoted cardiac specification. Wnt5a was upregulated by Mesp1 and may play a role in cardiogenesis. (Gessert S et al., Circulation research, 2010; Vol. 107; pgs. 186-199). Wn5a/Wnt11 could trigger a cardiogenic program in mesenchymal stem cells or bone marrow stem cells via activation of PKC. Gessert S et al., Circulation research, 2010; Vol. 107; pgs. 186-199). Of note, Wnt5a and Wnt11 mainly triggered non-canonical Wnt signaling, which have been shown to inhibit canonical Wnt signaling through multiple mechanisms. (Gessert S et al., Circulation research, 2010; Vol. 107; pgs. 186-199). Additionally, Wnt5a and Wnt11 were reported to inhibit canonical Wnt pathway and subsequently promoted cardiac progenitor generation via the caspase-dependent degradation of AKT. (Bisson J A et al., Developmental biology, 2015; Vol. 398; pgs. 80-96). Wnt5a and Wnt11 were essential for second heart field progenitor development. (Cohen E D et al., Development, 2012; Vol. 139; pgs. 1931-1940). Additionally, non-canonical Wnt pathways have been linked to cytoskeletal rearrangements. (Cohen E D et al., Development, 2012; Vol. 139; pgs. 1931-1940). The pathway enrichment analysis confirmed that genes related to WNT and cytoskeleton remodeling and Wnt5a were upregulated by ISX-9. Moreover, the real-time PCR results showed that ISX-9 induced Wnt3a upregulation in the initial stages of CPC differentiation while Wnt5a and Wnt11 were upregulated in the later stages. Inhibition of Wnt pathway at initial differentiation as well as non-canonical Wnt pathway at later stage resulted in decreased expression of cardiac transcription factors. Consistent with findings from a previous study. (Cohen E D et al., Development, 2012; Vol. 139; pgs. 1931-1940). Applicant's results showed that knock down of either Wnt5 or Wnt11 led to a dramatic decrease of cardiac transcription factor expression, suggesting their participation is required in cardiac progenitor generation. Taken together, these results further supported ISX-9 might promote cardiac differentiation and specification via temporal and sequential regulation of canonical and non-canonical Wnt signaling. Different cells might have different responses to ISX-9 treatment. It was demonstrated that in neural progenitor cells, ISX-9 treatment promoted them toward neurons. (Schneider J W et al., Nature chemical biology, 2008; Vol. 4; pgs. 408-410). Based on our results, ISX-9 might induce mesoderm and cardiac mesoderm differentiation in hiPSCs via activation of TGF-β induced EMT signaling and canonical Wnt signaling in the initial stage while continuous ISX-9 stimulation led to upregulation of Wnt5 and Wnt11 in the late stage of CPC formation. Inhibition of canonical Wnt signaling during the later phase of cardiac differentiation showed significant up-regulation of non-canonical Wnt expression. (Mehta A et al., Biochimica et biophysica acta, 2014; Vol. 1843; pgs. 2394-2402). Interestingly, loss of Wnt5a and Wnt11 led to a dramatic reduction of second heart field progenitors while an increase in canonical Wnt signaling in the developing heart was observed. Cohen E D et al., Development, 2012; Vol. 139; pgs. 1931-1940). These studies suggest a cross-talk between canonical and non-canonical signaling during cardiogenesis. More interestingly, ISX-9 induced time-dependent expression of cardiac transcription factors, and targeted multiple developmental signaling pathways which need further investigation. In addition, the role of ISX-9 in muscle gene development was further strengthened by upregulation of genes related to development of PIP3 signaling in cardiomyocytes, muscle contraction and NF-AT hypertrophy signaling pathways Thus, Applicant's results strongly suggest that ISX-9 is a strong promoter of cardiac differentiation in iPSCs.

ISX-9 in light of its role in cardiac differentiation through diverse signaling pathways also activated myogenesis miRNAs and cardiac hypertrophy related-miRNAs including miR-335, miR-21, miR-30c, and miR-214. These miRs are known to play a positive role in myogenic differentiation or muscle regeneration. (Guess M G et al., PloS one, 2015; Vol. 10; e0118229); (Wei Y et al., Gene, 2016; Vol. 592; pgs. 60-70); (Liu J et al., The Journal of biological chemistry, 2010; Vol. 285; pgs. 26599-26607); (Bai L et al., PloS one, 2015; Vol. 10; e0119396); (Meyer S U et al., PloS one, 2015; Vol. 10; e0135284). The most abundant miRNAs in cardiac muscle are miR-let-7, miR-30c (Yu S et al., Journal of cardiovascular translational research, 2010; Vol. 3; pgs. 241-245). miR-let-7 family played a key role in normal cardiac maintenance and it was required for maturation of stem cell-derived CMs (Kuppusamy K T et al., Proceedings of the National Academy of Sciences of the United States of America, 2015; Vol. 112; pgs. E2785-2794). Moreover, miR-21 was involved in cardiac hypertrophy and was highly expressed in the fetal heart (Romaine S P et al., Heart, 2015; Vol. 101; pgs. 921-928). miR-30c and miR-335 were shown to be upregulated in mouse stem cells and CMs, implying their potential roles during heart development (Thum T et al., Circulation, 2007; Vol. 116; pgs. 258-267). However, further studies are needed to better understand the underlying mechanism in regulation of miRNAs and signaling pathways important in cardiac differentiation of stem cells.

This study demonstrates a novel technique of producing pure CPCs from hiPSCs using a single cardiogenic small molecule, ISX-9 with robust muscle differentiation potential through diverse signaling pathways. These CPCs were multipotent and differentiated into three cardiac lineages to form new CMs and vessels in the infarcted heart, thus reducing fibrosis and improving functional indices.

Knockdown of Wnt5a and Wnt11 in Differentiated hiPSCs

Wnt5a and Wnt11 were knocked down in differentiated hiPSCs treated with ISX-9 as described previously. Briefly, at day3 of ISX-9 treatment, cells at 80% confluency were transfected with 10 nM silencer select siRNA Wnt5a (Sense: UAUCAAUUCCGACAUCGAAtt (SEQ ID NO. 1), Antisense: UUCGAUGUCGGGAAUUGAUAc (SEQ ID NO. 2)) and Wnt11 (Sense: ACUUCUGCAUGAAGAAUGAtt (SEQ ID NO. 3), Antisense: UCAUUCUUCAUGCAGAAGUca (SEQ ID NO. 4)) using Lipofectamine RNAiMAX (Thermo Fisher Scientific, Cat #13778075). Silencer™ Negative Control No. 1 siRNA was obtained from Thermo Fisher Scientific Silencer siRNA labeling kit (Thermo Fisher Scientific, Cat #AM1632) was used to determine transfection efficiency. After 48 h, verification of Wnt5a and Wnt11 knockdown in transfected cells was analyzed by real-time PCR. At day7, cells were harvested for analysis of cardiac transcription factors expression.

### Immunohistochemistry

For cell immunocytochemistry, the cells were fixed with 4% paraformaldehyde (PFA) for 10 minutes at room temperature. After washing with PBS, the cells were blocked for 1 h minutes at room temperature by a blocking buffer (PBS containing 10% fetal bovine serum and 0.1% triton). Cardiac progenitor cells (CPCs) were immunostained with respective specific primary antibodies Nkx2.5 (abcam, ab91196, 1:300), GATA4 (SC-1237, Santa Cruz, 1:100) and ISL-1 (ab86472, abcam, 1:300). hiPSC-CMs were characterized via the expression of α-sarcomeric actin, cTnT, cTnI, MLC2V and CX43; hiPSC-ECs were characterized via the expression of CD31 and VE-cadherin; hiPSC-SMCs were characterized via the expression of α-smooth muscle actin (SMA) and calponin. Briefly, hiPSC-CMs were immunostained with respective specific primary antibodies against α-sarcomeric actinin (A7811, Sigma, 1:200), cardiac troponin T (13-11, Thermo Fisher Scientific, 1:200), cardiac troponin I (701585, Thermo Fisher Scientific, 1:200), MLC2V (10906-1-AP, Protein Tech Group, 1:200), and CX43 (ab11370, abcam, 1:200). hiPSC-ECs were immunostained with antibodies against EC markers CD31 (ab24590, abcam, 1:200) and VE-cadherin (ab33168, abcam, 1:200). hiPSC-SMCs were immunostained with antibodies against SMC markers SMA (ab5694, abcam, 1:500) and calponin (C-2678, sigma, 1:600). The primary antibody-antigen reaction was detected with fluorescently conjugated specific secondary antibodies. Every time after incubation with antibody, the samples were washed three times with PBS. Nuclei were visualized after staining with 5 μg/ml by 4,6′-diamidino-2-phenylindole (DAPI; Life technologies). Fluorescence signals were observed and photographed using fluorescence microscope (Olympus, Tokyo, Japan).

Histological analysis was performed on randomly selected hearts from mice subjected to myocardial infarction (MI) with DPBS, hiPSCs or CPCs (n=6 per group). All hearts were fixed with 4% PFA for 1 hour at room temperature and replaced by 30% sucrose overnight at 4° C. These samples were embedded in an optical cutting temperature (OCT) compound (Tissue Tek) and sliced into 5-μm-thick frozen sections. α-sarcomeric actinin (A7811, Sigma, 1:200), human cardiac troponin T not cross reacting with mouse (ab45923, abcam, 1:200) staining, CD31 (MA5-13188, Thermo Fisher Scientific, 1:100), α-SMA (ab5694, abcam, 1:300), cardiac troponin I (701585, Thermo Fisher Scientific, 1:200), and human mitochondrial antigen (MAB1273, Millipore sigma, 1:200) were carried out. Signals were visualized with Dylight 405 (Thermo Fisher Scientific), Alexa Fluor 647 and Alexa Fluor 488 secondary antibodies (Life technologies). Image acquisition was performed on a confocal microscope (FV1000, Olympus, Japan). Nuclear was counterstained with DAPI or Qnuclear™ Deep Red Stain (Thermo Fisher Scientific). Masson trichrome staining was performed according to the manufacturer's protocol (HT-15, sigma).

### Infarct Size Measurement

Infarct size was determined as the average of 7 sections sampled at 4-mm intervals from the apex from the ratio of the Masson's trichrome-stained area to the total left ventricular area. Briefly, the total area of LV myocardium and infarct scar were traced by level set and thresholding methods. (Wang P et al, Neurochemical research, 2016; Vol. 41; pgs. 2627-2635); (Wang Y et al., IEEE International Conference on: IEEE, 2011; pgs. 1-4); (Chen Y et al., Medical image analysis, 2014; Vol. 18; pgs. 1-8), refined manually in the digital images, and then measured automatically. The level-set method with the ellipse is used in segmentation based on the observation that the epicardium is bounded by a circle-like contour. The level set formulation with shape priors has been demonstrated to be an efficient segmentation method and a number of techniques have been reported. (Wang P et al, Neurochemical research, 2016; Vol. 41; pgs. 2627-2635). In the present study, the general expression for the proposed ellipse refined level set segmentation energy formulation reads as follows,

E=E_{data}+αEₛₕₐₚₑ

where E_{data} represents the chosen data attachment term and Eₛₕₐₚₑ embeds the shape prior. The weight corresponds to a positive hyper-parameter that balances the influence between the two terms. As Wang et al. (Wang P et al, Neurochemical research, 2016; Vol. 41; pgs. 2627-2635) pointed out, E_{data} can be any of the data attachment terms and in the present study, the narrow band active contour is adopted as the data attachment term. After the segmentation of the epicardium, a color thresholding method was performed to measure the heart tissue and the infarct scar. The infarct size, expressed as a percentage, was calculated by dividing the sum of infarct areas from all sections by the sum of LV areas from all sections (including those without infarct scar) and multiplying by 100.

### Flow Cytometry

To detect purity of CPCs and hiPSC-CMs, hiPSC-ECs and hiPSC-SMCs, cells were dissociated with Collagen IV (stem cell technologies) for 2 hr following 10 min accutase digestion. Dissociated cells were fixed and permeabilized with a BD Cytofix/Cytoperm™ Fixation/Permeabilization Kit (BD Biosciences), and then incubated with antibody against Nkx2.5 (abcam, ab91196, 1:200), cTnT (13-11, Thermo Fisher Scientific; 1:200), CD31 and SMA (ab5694, abcam, 1:200) at 4° C. overnight. After three washes, cells were incubated with isotype-matched Alexa Fluorescence-conjugated secondary antibody (Life technologies) for lhr at room temperature and detected by an LSR II Flow Cytometer (BD Biosciences). Isotype-matched normal IgG was used as negative control.

### Functional Assays of Endothelial Cell In Vitro

To analyze tube formation on Matrigel in vitro, hiPSC-ECs were seeded on top of a thin layer of Matrigel at a density of 1.2×10⁵ cells/well of a 24-well plate. After 16 h, cells were labeled with Calcein AM (Corning) and visualized tube-like structures under the fluorescent microscope. Uptake of acetylated low-density lipoprotein was assessed by incubating cells with 5 μg/ml of ac-LDL conjugated with Alexa Fluor-594 (Invitrogen) for 4 h. After incubation, cells were counterstained with DAPI to visualize nuclei.

### Cytoprotection and TUNEL Staining

To analyze the cytoprotection of CPCs generated by ISX-9, hiPSCs treated with ISX-9 were replated in coverslips and subjected to simulated ischemia in vitro under hypoxic condition (5% CO2, 94% N2 and 1% 02) at 37° C. in hypoxic chamber (INVIVO₂500) for 12 h and 24 h. hiPSCs cultured in RPMI/B27 with or without 0.5% DMSO. To localize nuclear DNA fragmentation in cultured cells, in situ terminal deoxynucleotidyl transferase-mediated dUTP nick-end labeling (TUNEL) was performed using commercial apoptosis detection kit (Roche, USA). All procedures were performed according to the directions of the manufacturer. Cells were counterstained with DAPI to visualize nuclei. The number of TUNEL-positive cells was determined by randomly counting 10 fields (20×) from 3 coverslips in the same group and was expressed as a percentage of the cells with normal nuclei. To further determine the cytoprotection by CPCs in ischemic heart, CPCs were transplanted into the infarcted mouse heart 10 mins after coronary artery ligation. After 3 days, mice were sacrificed and heart tissue was processed for TUNEL staining. Cardiomyocytes were identified by α-sarcomeric actinin staining. Cells were counterstained with DAPI to visualize nuclei. The number of total TUNEL-positive cells and TUNEL-positive CMs was determined by counting 5 fields (20×) from border area in each heart (n=3). DPBS and hiPSCs treated hearts served as control groups.

### In Vitro Analysis of Paracrine Function

In order to determine whether the CPCs induced by ISX-9 expressed and secreted progangiogenic and prosurvival cytokines under ischemic conditions in vitro, we performed multiple angiogenesis factors and cytokines assays (Millipore) to identify paracrine factors in induced CPCs under normal and ischemic conditions. Briefly, induced CPCs were subjected to simulated ischemia in vitro under hypoxic condition (5% CO2, 94% N2 and 1% O2) at 37° C. or normal culture condition for 12 h and 24 h, then the supernatants (n=4/group) were collected and centrifuged at 10,000 g for 10 min at 4° C. and store at −80° C. Analysis of secreted factors was performed using a Luminex-based platform (Bio-Rad Bio Plex-100) as described. hiPSCs cultured in RPMI/B27 with or without 0.5% DMSO were served as control groups.

### RNA-Sequencing in CPCs Generated by ISX-9 Treatment

mRNA-Sequencing transcriptome analysis was performed to reveal differences in gene expression among undifferentiated hiPSCs, DMSO or ISX-9 treated hiPSCs (n=4). Global miRNA expression profiles in undifferentiated hiPSCs, DMSO or ISX-9 treated hiPSCs were also determined. mRNA-sequencing and miRNA-sequencing were performed by Core Genomics Facility and DNA services facility at University of Illinois at Chicago. Bioinformatic data analysis was conducted by Research Informatics Core at University of Illinois at Chicago. Briefly, total RNA containing miRNA was extracted from cells using Qiagen's RNasey Plus Mini kit according to manufacturer's instructions followed by DNAse treatment using RNase-free DNase kit (Qiagen). For mRNA-sequencing, Sequencing libraries were prepared using QuantSeq 3′mRNA-Seq Library Kit (Lexogen Inc, product #015.96). Input amount for the library preparation was 500 ng of total RNA per sample. Libraries were PCR amplified at 16 cycles and quantified using Qubit dsDNA HS assay. For miRNA-sequencing, Sequencing libraries were prepared using Illumina's TruSeq Small RNA Core solutions kit+Truseq Small RNA Indices A kit (Illumina product #s respectively: 15016911 and 15016912). Input amount for library preparation was 1 ug of total RNA per sample. Libraries were PCR amplified at 11 cycles and quantified using Qubit dsDNA HS assay. Sequencing libraries were pooled in equimolar concentrations and sequencing was performed on NextSeq 500 (Illumina), single read 75 nt, high output (about 400 million reads per lane).

In the analysis of 3′mRNA-seq data, the short reads of 12 samples were mapped to the UCSC human hg19 reference genome using BWA mem (Li H et al., Bioinformatics, 2009; Vol. 25; pgs. 1754-1760). The raw counts of each gene were quantified using featureCounts base on the mapping results, and transcriptome annotation of UCSC hg38. The genes with zero counts across all samples were filtered from further analysis. The raw counts were normalized to count per million (CPM) for each gene within each sample by using R Bioconductor Package (Robinson M D et al., Bioinformatics, 2010; Vol. 26; pgs. 139-140). The Differentially Expressed Genes (DEGs) were identified using edgeR. First, the General Linear Model (GLM) Likelihood Ratio Test (LRT) was performed for identifying DEGs where the mean expression value of a gene in any group is significantly different than in other sample groups. The raw p-values were adjusted by Benjamini-Hochberg correction (Benjamini Y et al., Journal of the Royal Statistical Society Series B-Methodological, 1995; Vol. 57; pgs. 289-300). Second, the pairwise comparisons were performed between DMSO vs. hiPSC, ISX-9 vs. hiPSC, and ISX-9 vs. DMSO. The exact test was performed on each gene between each pair of sample groups to identify to the significant pairwise DEGs (Robinson M D et al., Biostatistics, 2008; Vol. 9; pgs. 321-332). The p-values from the exact test were also corrected by Benjamini-Hochberg correction. Likewise, in the analysis of miRNA-seq data, the 3′ short reads of 12 samples were mapped to the NCBI human GRCh38 reference genome using BWA mem (Li H et al., Bioinformatics, 2009; Vol. 25; pgs. 1754-1760). The raw counts of transcripts of each miRNA were quantified using featureCounts base on the mapping results, and the miRNA annotation from miRBase (Kozomara A et al., Nucleic Acids Res, 2014; Vol. 42; pgs. D68-73). The normalization and differential analysis followed the same procedures as in mRNA-seq data analysis. Pathway enrichment analysis was processed using metacore online analysis.

### Accession Numbers

The accession numbers in GEO for the RNA-sequencing data reported in this experiment were GSE95389 and GSE 95390.

### Transmission Electron Microscopy

The differentiated cardiomyocytes (CMs) were fixed overnight at 4° C. in 2.5% (Vol/Vol) glutaraldehyde, 0.1M cacodylate buffer solution and then were postfixed with 1% osmium tetroxide in the same buffer, en-block stained with 2% aqueous uranyl acetate, dehydrated in acetone, infiltrated, and embedded in LX-112 resin (Ladd Research Industries, Burlington, Vt.). Ultrathin 60-nm sections were stained with uranyl acetate and lead citrate. Samples were examined on a JEOL JEM-1220 transmission electron microscope (TEM) (JEOL USA, Inc.)

### Echocardiography

The mice were anesthetized mildly with inhaled isoflurane and lightly secured in the supine position on a warm pad. After the hair was removed, Acoustic gel was applied and transthoracic echocardiography was performed using Philips iE33 ultrasound machine with L15-7io Transducer. The heart was imaged in the two-dimensional mode in the parasternal long-axis and/or parasternal short-axis views which were subsequently used to position the M-mode cursor perpendicular to the ventricular septum and left ventricle posterior wall, after which M-mode images were obtained. For each animal, measurements were obtained from 4-5 consecutive heart cycles. Measurements of left ventricular end diastolic diameter (LVEDD), and left ventricular end systolic diameter (LVESD) were made from two-dimensionally directed M-mode images of the left ventricle in both systole and diastole (Xuan W et al., Cardiovasc Res, 2011; Vol. 92; pgs. 385-393). The average value from all measurements in each animal were used to determine the indices of left ventricle contractile function, i.e. left ventricle fractional shortening (LVFS) using the following relations LVFS=(LVEDD−LVESD)/LVEDD×100, ejection fraction (EF) using the following relations EF=[(EDV−ESV)/EDV]×100. LVFS and EF were expressed as percentages.

qRT-PCR

Total RNA was isolated using RNeasy Mini Kit (Qiagen). Reverse transcription was performed using QuantiTect Reverse Transcription kit (Qiagen) or SuperScript™ IV VILO™ Master Mix (Thermo fisher Scientific Inc). qRT-PCR was performed on real-time system ViiA™ 7 (ABI) or Q3 real-time PCR machine (ABI) using Quantitate SYBR Green real-time PCR method or Taqman probe method as described elsewhere (Primer sequences are shown in Table 2). Probes for Wnt3a (Hs00263977_m1), Wnt5a (Hs00998537_m1); Wnt11 (Hs01045905_m1); ISL-1 (Hs00158126_m1); Nkx2.5 (Hs00231763_m1); Mef2c (Hs00231149_m1); GATA4 (Hs00171403_m1); TUBA1A (Hs03045184_g1) were purchased from Thermo Fisher Scientific GADPH and TUBA1A act as the loading control. The fold change of expression level for each gene was determined by the expression 2-ΔΔCT. The final values were averaged and results were represented as fold expression with the standard error of the mean (S.E.M.).

### Vessel Density Assessment

Vessel density was assessed in 9 animals (3 in each group) sacrificed at 3M after MI. The number of vessels was counted in blind on 27 sections (3 sections per heart) in the infarct and border areas of all mice after staining with an antibody, anti-CD31 or α-SMA using a fluorescence microscope at a 400× magnification. Vascular density was determined by counting CD31 positive vascular structures and arteriole density was determined by counting α-SMA positive vascular structures. Five high-power fields in each section were randomly selected, and the number of vessels in each field was averaged and expressed as the number of vessels per high-power field (0.2 mm²).

TABLE 2

| Gene | Forward 5′-3′ | Reverse 5′-3′ | Tm (° C.) |
|---|---|---|---|
| NKX2.5 | CATTTACCCGGGAGCCTACG | GCTTTCCGTCGCCGCCGTGCGCGTG | 60 |
| | (SEQ ID NO. 5) | (SEQ ID NO. 6) | |
| Mef2c | AGATACCCACAACACACCACGCGCC | ATCCTTCAGAGAGTCGCATGC | 60 |
| | (SEQ ID NO. 7) | (SEQ ID NO. 8) | |
| GATA4 | GGTTCCCAGGCCTCTTGCAATGCGG | AGTGGCATTGCTGGAGTTACCGCTG | 60 |
| | (SEQ ID NO. 9) | (SEQ ID NO. 10) | |
| TBX5 | TACCACCACACCCATCAAC | ACACCAAGACAGGGACAGAC | 60 |
| | (SEQ ID NO. 11) | (SEQ ID NO. 12) | |
| ISL1 | CACAAGCGTCTCGGGATT | AGTGGCAAGTCTTCCGACA | 60 |
| | (SEQ ID NO. 13) | (SEQ ID NO. 14) | |
| GADPH | CATCCATGACAACTTTGGTATC | CACCCTGTTGCTGTAGCCAA | 60 |
| | (SEQ ID NO. 15) | (SEQ ID NO. 16) | |

### Experiment No. 8

Comparative Effects of Exosomes or Microvesicles Derived from Different Cardiac progenitor Cells on Cardiac Injury and Function Following Coronary Artery Ligation.

### Methods

### Cell Culture

hiPSC maintenance: Human iPSC cells (ACS-1021, ATCC, USA) were maintained in mTeSR1 media (Stem Cell Technology) on vitronectin coated six-well plates with daily medium changes. Cells were passaged with ReLeSR™ reagent every 4-7 days according to the manufacturer's protocol (Stem Cell Technology).

CPC generation: Briefly, hiPSCs maintained on vitronectin coated six-well plate in mTeSR1 media (Stem Cell Technology) were dissociated into single cells using Accutase solution (Invitrogen) at 37° C. for 10 min and then were seeded on to a vitronectin-coated six-well plate at 1×106 cell/well in mTeSR1 supplemented with 5 μM ROCK inhibitor (Y-27632, Stem Cell Technology) for 24 h. The second day, cells were cultured in mTesR1 with daily change for 3 days. Afterwards the medium was switched to RPMI/B27 minus insulin supplemented with ISX-9 (20 uM, dissolved in DMSO, Stem Cell Technology) for 7 days.

### EB Generation

Applicant generated EBs using hanging drop method in RPMI/B27 minus insulin medium.

Generation and Isolation of Exosomes or Microvesicles from Cardiac Progenitor Cells, EB or hiPSCs

Exosomes or microvesicles were generated from Human iPSC cell line ACS-1021 (ATCC, USA), Embryoid bodies (EB) and CPCs induced by ISX-9. CPCs were generated as described in Experiment No. 10. Conditioned media was collected from hiPSC and CPCs. The conditioned media was centrifuged at 3000 rpm for 30 min to remove cells and debris, followed by filtration through a 0.22 μm filter to remove the remaining debris. Then the medium was further concentrated to 500 μl using Amicon Ultra-15 100 kDa centrifugal filter units (Millipore). Isolation of exosomes or microvesicles in the concentrated medium was used qEV size exclusion columns (Izon science). Exosome or microvesicle fractions were collected and concentrated by Amicon Ultra-4 10 KDa centrifugal filter units to a final volume of <100 μl. The purified exosomes or microvesicles were stored at −80° C. and subsequently characterized by nanotracking analysis, protein and ultrastructure analysis.

Particle Size and Concentration Distribution Measurement with Tunable Resistive Pulse Sensing

Particle size and concentration distribution of exosomes or microvesicles isolated analysis were performed using tunable resistive pulse sensing method with a qNano instrument (Izon Science). Briefly, the number of particle was counted at least 600 to 1000 events using 20 mbar pressure and NP200 nanopore membranes stretched between 46.5-47.5 mm. Calibration was performed using known concentration of beads CPC200 (diameter: 210 nm). Data were processed using Izon Control Suite software.

### Transmission Electron Microscopy

Exosomes or microvesicles pellet were fixed with 4% paraformaldehyde (PFA). Following a total of 8 washes using distilled water, grids were contrasted with a uranyl-oxalate solution for 5 minutes, and transferred to methyl-cellulose-uranyl acetate for 10 minutes on ice according to the description in previous study. Samples were examined on a JEOL JEM-1220 transmission electron microscope (TEM) (JEOL USA, Inc.)

### Experiment No. 9

Generation of Cardiac/Skeletal Myogenic Progenitors from Human Induced Pluripotent Stem Cells Using a Small Molecule from Unpublished Abstract: Generation of Cardiac/Skeletal Myogenic Progenitors from Human Induced Pluripotent Stem Cells Using a Small Molecule.

The aim of this experiment was to evaluate the conversion of human induced pluripotent stem cells (hiPSCs) into cardiac (CMPs) or skeletal myogenic progenitors (SMPs) with a single small molecule for cell therapy.

### Methods and Results

hiPSCs (purchased as cell line) were cultured as a monolayer and treated with a small molecule, Givinostat (GIV, a histone deacetylase inhibitor, 10-200 nM) for 7 days. Higher cell viability (CCK8 Assay) and lower cytotoxicity (LDH Assay) was observed with concentration of 150 nM GIV. GIV had dual effect by generating both CMPs and SMPs after 7 days. In addition to GIV, mTeSR™1 serum-free medium was used with Rho-associated kinase (ROCK) inhibitors (5 μM Thiazovivin or 1 μM Y27632) for SMPs specification; and mTeSR™1 serum-free medium was used with ALK4/5/7 (TGFβ type-I receptor) inhibitors (2 μM SB431542 or 1 μM A83-01) for CMPs specification; and was assessed after 4-8 days. GIV increased upregulation of skeletal myogenic genes (Real Time PCR-analysis) at first 4 days and then was further increased at a higher level after 8 days. Muscle genes included Meox1 (21.4-fold), Meox2 (3.9-fold), Tcf15 (3.5-fold), Pax3 (28-fold), Pax7 (3.2-fold), MyoD1 (1.7-fold), dystrophin (3.8-fold), myogenin (6-fold), Myh2 (5.6-fold), Myh6 (3.4-fold), Tbx1 (2.6-fold), Mesp1 (2.4-fold), desmin (1.9-fold) and β-catenin (1.6-fold) versus control. PCR results were confirmed with Western blots densitometry including Pax3 (7-fold), Pax7 (2.5-fold), Myf5 (8-fold), MyoD1 (2-fold), dystrophin (6-fold) and desmin (5-fold) versus control (P<0.05). Morphologically, the myotube or myocyte-like shape of differentiating cells were observed microscopically. GIV increased upregulation of cardiac myogenic genes after 4 days with a higher level after 8 days. Cardiac genes included Pitx2 (56-fold), ISl1 (6.9-fold), Nkx2.5 (48-fold), Hand1 (14.1-fold), GATA4 (5-fold), Tbx5 (3.8-fold), TnnT2 (5.7-fold), Myl7 (16.1-fold), MLC2v (8.9-fold), Myf2c (1.4-fold), Cdh4 (3.1-fold) and Lhx2 (2.7-fold) versus control.

Pretreatment of hiPSCs with GIV represents a viable strategy for producing both cardiac/skeletal myogenic progenitors in vitro for cell therapies against myocardial infarction and Duchenne muscular dystrophy.

### Experiment No. 10

This experiment discloses the comparative effects of exosomes derived from different cardiac progenitor cells on cardiac injury and function following coronary artery ligation. Briefly, human iPSC cells (ACS-1021, ATCC, USA) were maintained in mTeSR1 media (Stem Cell Technology) on vitronectin coated six-well plates with daily medium changes. Cells were passaged with ReLeSR™ reagent every 4-7 days according to the manufacturer's protocol (Stem Cell Technology). To generate CPCs, hiPSCs maintained on vitronectin coated six-well plate in mTeSR1 media (Stem Cell Technology) were dissociated into single cells using Accutase solution (Invitrogen) at 37° C. for 10 min and then were seeded on to a vitronectin-coated six-well plate at 1×106 cell/well in mTeSR1 supplemented with 5 μM ROCK inhibitor (Y-27632, Stem Cell Technology) for 24 h. The second day, cells were cultured in mTesR1 with daily change for 3 days. Afterwards the medium was switched to RPMI/B27 minus insulin supplemented with ISX-9 (20 uM, dissolved in DMSO, Stem Cell Technology) for 7 days. For EB generation, the hanging drop method in RPMI/B27 minus insulin medium was used. To generate and isolate exosomes from cardiac progenitor cells, EB or hiPSCs, exosomes were generated from Human iPSC cell line ACS-1021 (ATCC, USA), Embryoid bodies (EB) and CPCs induced by ISX-9. CPCs were generated as previously described. Conditioned media was collected from hiPSC and CPCs. The conditioned media was centrifuged at 3000 rpm for 30 min to remove cells and debris, followed by filtration through a 0.22 μm filter to remove the remaining debris. Then the medium was further concentrated to 500 μl using Amicon Ultra-15 100 kDa centrifugal filter units (Millipore). Isolation of exosomes in the concentrated medium was used qEV size exclusion columns (Izon science). Exosome fractions were collected and concentrated by Amicon Ultra-4 10 KDa centrifugal filter units to a final volume of <100 μl. The purified exosomes were stored at −80° C. and subsequently characterized by nanotracking analysis, protein and ultrastructure analysis. Particle size and concentration distribution of exosomes isolated analysis were performed using tunable resistive pulse sensing method with a qNano instrument (Izon Science). Briefly, the number of particle was counted at least 600 to 1000 events using 20 mbar pressure and NP200 nanopore membranes stretched between 46.5-47.5 mm. Calibration was performed using known concentration of beads CPC200 (diameter: 210 nm). Data were processed using Izon Control Suite software. For transmission electron microscopy, exosomes pellet were fixed with 4% paraformaldehyde (PFA). Following a total of 8 washes using distilled water, grids were contrasted with a uranyl-oxalate solution for 5 minutes, and transferred to methyl-cellulose-uranyl acetate for 10 minutes on ice according to the description in previous study. Samples were examined on a JEOL JEM-1220 transmission electron microscope (TEM) (JEOL USA, Inc.).

### EQUIVALENTS

Unless otherwise defined, all technical and scientific terms used herein have the same meaning as commonly understood by one of ordinary skill in the art to which this invention belongs. All nucleotide sequences provided herein are presented in the 5′ to 3′ direction.

The inventions illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

What is claimed is:

1. An isolated exosome or microvesicle isolated from a cell selected from an iPSC cell, an embryonic stem cell, or a stem cell that had been contacted with an effective amount of an isoxazole compound, a derivative or an equivalent thereof or Danazol, wherein the exosome or microvesicle overexpresses one or more of:
a. a microRNA (miRNA) selected from the group of mir-373, mir-210, mir-377, mir-367, mir-520, mir-548ah, mir-335, mir-21, mir-30c, mir-214 or mir-548q; and/or
b. one or more of a protein selected from: Tsg101, CD9, Hsp70, Flotillin-1 or GAPDH.

2. An isolated cell selected from the group of: a cardiac progenitor cell, a (CPC), a cardiomyocyte, a myocyte, an endothelial cell, a smooth muscle cell, or a skeletal muscle cell, each generated from a cell selected from the group of an iPSC, an embryonic stem cell, or a stem cell contacted with an effective amount of an isoxazole compound, a derivative or an equivalent thereof, wherein the isolated cell overexpresses:
a. a microRNA (miRNA) selected from the group of mir-373, mir-210, mir-377, mir-367, mir-520, mir-548ah, mir-335, mir-21, mir-30c, mir-214 or mir-548q; and/or
b. a muscle gene selected from the group of paZ3, pAX7, MYF5, MYOD, MYOG, or dystrophin.

3. A isolated cardiomyocyte of claim 1 or 2, expressing on or more of cTnT, cTnI, MLC2V and/or CX43.

4. An isolated endothelial cell of claim 1 or 2 expressing one or more of CD31 and/or VE-cadherin.

5. An isolated smooth muscle cell expressing α-smooth muscle actin (SMA) and calponin.

6. A population of isolated exosomes or microvesicles of claim 1, wherein the population of exosomes or microvesicles are substantially homogeneous.

7. The isolated exosome or microvesicle of claim 1 that is detectably labeled.

8. The composition of claim 4, and a carrier, wherein the carrier is optionally a non-naturally occurring carrier.

9. An isolated population of cells of claim 2, wherein the cell population is substantially homogeneous.

10. A composition comprising the isolated population of claim 9, and a carrier, wherein the carrier is optionally a non-naturally occurring carrier.

11. The composition of claim 7 or 9, further comprising a preservative or cryoprotectant.

12. The composition of claim 1, further comprising a protein that facilitates regeneration and/or improved function of a tissue or a nucleic acid that encodes the protein and/or an agent that inhibits the expression of an inflammatory protein, that is optionally a cytokine.

13. The composition of claim 12, wherein the protein is selected from the group of a transforming growth factor-β, a WNT protein, a cytokine or a histone deacetylase.

14. The isolated population of claim 11, wherein the composition is lyophilized.

15. The isolated exosome or microvesicle of claim 1, wherein the isoxazole compound is isoxazole-1 (isx-1) or isoxazole-9 (isx-9), or Danazol.

16. The isolated cell of claim 2, wherein the isoxazole compound is isoxazole-1 (isx-1) or isoxazole-9 (isx-9).

17. The isolated exosome or microvesicle of claim 1 or the isolated cell of claim 2, wherein the isoxazole derivative has the formula: 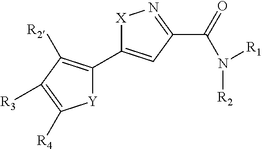
wherein R₁ and R₂ are both hydrogen or R₁ is hydrogen and R₂ is selected from the group consisting of substituted or unsubstituted C₁-C₆ alkyl, C₃-C₆ cycloalkyl, C₂-C₆ alkenyl, C₂-C₆ alkynyl, and benzyl, or where R₁ and R₂ may be joined together to form a ring selected from azetidinyl, pyrrolidinyl, piperidinyl, piperazinyl, or morpholinyl; R₂′, R₃ and R₄ are independently selected from the group consisting of hydrogen, halogen, C₁-C₆ alkyl, C₃-C₆ cycloalkyl, substituted or unsubstituted aromatic or heteroaromatic ring, cyano, nitro, and acyl; X is 0, NH or S; and Y is 0, NH or S.

18. The isolated exosome or microvesicle of claim 1 or the isolated cell of claim 2, wherein the isoxazole compound has the formula: 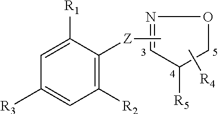
wherein R1 and R2 is each selected from C1-C4 alkyl, phenyl, benzyl, trifluoromethyl or halogen, R3 is selected from hydrogen, hydroxy, C1-C4 alkyl or alkoxy, R4, in position 3 or 5, is selected from hydrogen, trifluoromethyl, C1-C4 alkoxy, C1-C4 alkyl, or C1-C4 hydroxyalkyl, R5 is selected from hydrogen or C4-C4 alkyl or R4 and R5 together form a tetramethylene group, Z at position 3 or 5 on the heterocycle is selected from: —N(R6)-CO—, —CO—N(R6)-, —N(R6)-CO—N(R6)-, —CH(R6)-NH—CO—, or —NH—CO—CH(R6), in which R6 is selected from hydrogen or C1-C4 alkyl.

19. The isolated exosome or microvesicle of claim 1, or the isolated cell of claim 2, wherein the isoxazole compound or a derivative thereof is selected from the group: 5-(trifluoromethyl)-3-(4-methoxyphenyl)isoxazole-4-carboxylic acid, 5-(trifluoromethyl)-3-(4-fluorophenyl)isoxazole-4-carboxylic acid, 5-(thiophen-2-yl)isoxazole-3-carboxaldehyde, 5,6,7,8-tetrahydro-4h-cyclohepta[d]isoxazole-3-carboxylic acid, 4,5,6,7-tetrahydro-benzo[d]isoxazole-3-carboxylic acid, 3-amino-5-methylisoxazole, 4-amino-n-(5-methyl-3-isoxazolyl)benzenesulfonamide, 3-phenyl-isoxazole-5-boronic acid pinacol ester, 5-phenylisoxazole, 1-phenyl-1-cyclopentanecarboxylic acid, 3-phenyl-benzo[c]isoxazole-5-carboxylic acid, 5-methyl-3-phenylisoxazole-4-carboxylic acid, 3a,4,5,6,7,8,9,9a-octahydro-cycloocta[d]isoxazole-3-carboxylic acid, 5-(3-nitrophenyl)isoxazole, 3-(4-nitrophenyl)isoxazole, 3-hydroxy-5-aminomethyl-isoxazole, 5-(morpholinomethyl)isoxazole-3-carboxylic acid hydrochloride, 5-(morpholinomethyl)isoxazole-3-carbaldehyde, 3-methyl-5-(trifluoromethyl)isoxazole-4-carboxylic acid, methyl 5-(thiophen-2-yl)isoxazole-3-carboxylate, 3-(methylsulfonyl)-5-(2-thienyl)isoxazole-4-carbonitrile, 5-methyl-3-(2-pyrrolidinyl)isoxazole, 3-methyl-5-(2-pyrrolidinyl)isoxazole, 3-(1-methyl-1h-pyrazol-4-yl)-isoxazole-5-carboxylic acid, 3-(1-methyl-1h-pyrazol-4-yl)-4,5-dihydro-isoxazole-5-carboxylic acid, 5-(4-methylphenyl)isoxazole-3-carboxylic acid, 5-methyl-3-phenylisoxazole-4-carboxylic acid, 5-(4-methylphenyl)isoxazole-3-carboxaldehyde, 5-methyl-3-(4-phenoxyphenyl)isoxazole-4-carboxylic acid, 3-methyl-5-(4-methyl-1,2,3-thiadiazol-5-yl)isoxazole-4-carboxylic acid, 3-methyl-5-(5-methylisoxazol-3-yl)isoxazole-4-carboxylic acid, methyl 5-(4-methoxyphenyl)isoxazole-4-carboxylate, methyl 5-(4-methoxyphenyl)isoxazole-3-carboxylate, 5-methylisoxazole, methyl 5-(4-fluorophenyl)isoxazole-4-carboxylate, methyl 5-(4-fluorophenyl)isoxazole-3-carboxylate, methyl 5-(4-chlorophenyl)isoxazole-4-carboxylate, methyl 5-(4-bromophenyl)isoxazole-4-carboxylate, 5-(4-methoxyphenyl)isoxazole-3-carboxylic acid, 5-(3-methoxy-phenyl)-isoxazole-3-carboxylic acid, 3-(2-methoxyphenyl)isoxazole-5-carboxylic acid, 5-(4-methoxyphenyl)isoxazole-3-carboxaldehyde, 3-(4-methoxyphenyl)isoxazole-5-carbaldehyde, 3-(2-methoxyphenyl)isoxazole-5-carbaldehyde, 5-(4-methoxyphenyl)isoxazole, 3-(4-methoxyphenyl)isoxazole, 3-(2-methoxy-phenyl)-4,5-dihydro-isoxazole-5-carboxylic acid, 3-methoxy-isoxazole-5-carboxylic acid, isoxazole-5-carboxylic acid, isoxazole-4-carboxylic acid, isoxazole-5-carbothioamide, isoxazole-5-carbonyl chloride, isoxazole-3-carbonitrile, isoxazole-3-carbaldehyde, isoxazole-4-boronic acid, isoxazole, 5-cyclopropyl-4[2-(methylsulfonyl)-4-(trifluoromethyl)benzoyl]isoxazole, 6-(5-(thiophen-2-yl)isoxazole-3-carboxamido)hexyl 5-((3as,4s,6ar)-2-oxohexahydro-1h-thieno[3,4-d]imidazol-4-yl)pentanoate, isocarboxazid 5-methyl-3-isoxazole-carboxylic acid 2-benzylhydrazide, 5-isobutyl-isoxazole-3-carboxylic acid, 4-iodo-5-methyl-isoxazole, 3,3′-iminobis(n,n-dimethylpropylamine), 3-(3-hydroxy-phenyl)-isoxazole-5-carboxylic acid methyl ester, 5-(4-hydroxy-phenyl)-isoxazole-3-carboxylic acid, 5-(3-hydroxy-phenyl)-isoxazole-3-carboxylic acid, 5-(hydroxymethyl)-3-methylisoxazole, 3-hydroxy-5-methylisoxazole, 5-(1-hydroxyethyl)-3-(4-trifluoromethylphenyl)isoxazole, 3a,4,5,6,7,7a-hexahydro-benzo[d]isoxazole-3-carboxylic acid, 5-(2-furyl)isoxazole-3-carbaldehyde, 5-furan-2-yl-isoxazole-3-carboxylic acid, 6-fluoro-3-(4-piperidinyl)benzisoxazole, 5-(4-fluorophenyl)isoxazole-3-methanol, 3-(2-fluoro-phenyl)-isoxazole-5-carboxylic acid, 5-(4-fluorophenyl)isoxazole-3-carboxaldehyde, 3-(4-fluorophenyl)isoxazole-5-carbaldehyde, 3-(3-fluorophenyl)isoxazole-5-carbaldehyde, 3-(2-fluorophenyl)isoxazole-5-carbaldehyde, 5-(4-fluorophenyl)isoxazole, 3-(4-fluorophenyl)isoxazole, 5-(3-fluoro-4-methoxy-phenyl)-isoxazole-3-carboxylic acid, ethyl 5-(trifluoromethyl)-3-(4-methoxyphenyl)isoxazole-4-carboxylate, ethyl-5-(tributylstannyl)isoxazole-3-carboxylate, ethyl 5-(thiophen-2-yl)isoxazole-3-carboxylate, 5-ethyl-isoxazole-4-carboxylic acid, 5-ethyl-isoxazole-3-carboxylic acid, ethyl 5-(4-fluorophenyl)isoxazole-4-carboxylate, ethyl 5-(4-fluorophenyl)isoxazole-3-carboxylate, ethyl 5-(2,3-dihydrobenzo[b][1,4]dioxin-7-yl)isoxazole-3-carboxylate, ethyl 3-(4-chlorophenyl)-5-(trifluoromethyl)isoxazole-4-carboxylate, ethyl 5-(4-chlorophenyl)isoxazole-3-carboxylate, ethyl 3-(4-bromophenyl)-5-(trifluoromethyl)isoxazole-4-carboxylate, ethyl 5-(4-bromophenyl)isoxazole-3-carboxylate, ethyl 5-amino-4-(4-chlorophenyl)isoxazole-3-carboxylate, ethyl 5-amino-4-(4-bromophenyl)isoxazole-3-carboxylate, ethyl 6b-acetyl-2-(acetyloxy)-4a,6a-dimethyl-2,3,4,4a,4b,5,6,6a,6b,9a,10,10a,10b,11-tetradecahydro-1h-naphtho[2′,1′:4,5]indeno[2,1-d]isoxazole-9-carboxylate, 3,5-dimethyl-4-(tributylstannyl)isoxazole, 5-(1,5-dimethyl-1h-pyrazol-4-yl)-isoxazole-3-carboxylic acid, 5-(1,3-dimethyl-1h-pyrazol-4-yl)-isoxazole-3-carboxylic acid, 5-(1,5-dimethyl-1h-pyrazol-4-yl)-isoxazole, 3,5-dimethylisoxazole-4-boronic acid pinacol ester, 3,5-dimethylisoxazole, 3-(dimethylamino)-1-(2-pyridyl)-2-propen-1-one, 5-(3,5-difluorophenyl)isoxazole, [2,6-dichloro-4-(trifluoromethyl)phenyl]hydrazine, 5-(2,5-dichlorophenyl)isoxazole-3-carboxylic acid, danazol, 3-(4-chlorophenyl)-5-(trifluoromethyl)isoxazole-4-carboxylic acid, 5-(4-chlorophenyl)isoxazole-3-propionic acid, 5-(4-chlorophenyl)isoxazole-4-carboxylic acid, 5-(4-chlorophenyl)isoxazole-3-carboxylic acid, 3-(4-chlorophenyl)isoxazole-5-carboxylic acid, 3-(3-chlorophenyl)isoxazole-5-carboxylic acid, 5-(4-chlorophenyl)isoxazole-3-carboxaldehyde, 3-(4-chlorophenyl)isoxazole-5-carbaldehyde, 3-(3-chlorophenyl)isoxazole-5-carbaldehyde, 3-(2-chlorophenyl)isoxazole-5-carbaldehyde, 5-(4-chlorophenyl)isoxazole, 3-(4-chlorophenyl)isoxazole, 5-(chloromethyl)isoxazole-4-carboxylic acid, 3-(chloromethyl)-5-(2-furyl)isoxazole, 4-chloromethyl-3,5-dimethylisoxazole, 5-(chloromethyl)-3-(4-chlorophenyl)isoxazole, 5-(3-chloro-4-methoxy-phenyl)-isoxazole-3-carboxylic acid, 3-chloro-4-fluorobenzaldehyde, 3-(5-chloro-2,4-dimethoxy-phenyl)-4,5-dihydro-isoxazole-5-carboxylic acid, 5-tert-butyl-4,5,6,7-tetrahydro-benzo[d]isoxazole-3-carboxylic acid, 3-(4-bromophenyl)-5-(trifluoromethyl)isoxazole-4-carboxylic acid, 5-(4-bromophenyl)isoxazole-3-propionic acid, 5-(4-bromophenyl)isoxazole-3-carboxylic acid hydrazide, 5-(4-bromophenyl)isoxazole-4-carboxylic acid, 5-(4-bromophenyl)isoxazole-3-carboxylic acid, 3-(4-bromophenyl)isoxazole-5-carboxylic acid, 3-(4-bromophenyl)isoxazole-5-carboxaldehyde, 5-(4-bromophenyl)isoxazole, 5-(3-bromophenyl)isoxazole, 3-(4-bromophenyl)isoxazole, 5-(bromomethyl)-3-(4-methoxyphenyl)isoxazole, 4-(bromomethyl)isoxazole, 5-(bromomethyl)-3-(4-fluorophenyl)isoxazole, 5-(bromomethyl)-3-(4-chlorophenyl)isoxazole, 5-(bromomethyl)-3-(4-bromophenyl)isoxazole, 6-bromo-3-methylbenzo[d]isoxazole, 5-bromo-3-methylbenzo[d]isoxazole, 4-bromo-5-(4-methoxyphenyl)isoxazole, 3-bromo-isoxazole, 3-bromo-5-(2-hydroxyethyl)isoxazole, 4-bromo-5-(4-fluorophenyl)isoxazole, 3-bromo-5-(4-fluorophenyl)isoxazole, 4-bromo-5-(4-chlorophenyl)isoxazole, 4-bromo-5-(4-bromophenyl)isoxazole, 6-bromo-benzo[d]isoxazole-3-carboxylic acid, benzo[d]isoxazole-3-carboxylic acid, 3-amino-5-methylisoxazole, 5-amino-3-(4-methoxyphenyl)isoxazole, 3-aminoisoxazole, 3-amino-5-(4-fluorophenyl)isoxazole, 5-amino-3-(4-chlorophenyl)isoxazole, 5-amino-4-(4-bromophenyl)isoxazole, 3-amino-5-(4-bromophenyl)isoxazole, 5-acetyl-3-(4-fluorophenyl)isoxazole, 5-acetyl-3-(3-fluorophenyl)isoxazole, 3-methyl-5-[(2s)-1-methyl-2-pyrrolidinyl]isoxazole hydrochloride, 7-methoxy-5-methyl-4,5-dihydronaphtho[2,1-d]isoxazole, 5-methyl-3-phenyl-isoxazole-4-carboxylic acid methylamide, 5-methyl-3-phenyl-isoxazole-4-carbothioic acid methylamide, 5-methyl-3-phenyl-4-(1h-pyrazol-5-yl)isoxazole, 5-benzyl-3-furan-2-yl-2-phenyl-tetrahydro-pyrrolo(3,4-d)isoxazole-4,6-dione, 5-benzyl-3-[4-(dimethylamino)phenyl]-2-phenyldihydro-2h-pyrrolo[3,4-d]isoxazole-4,6(3h,5h)-dione, 5-benzyl-3-(5-br-2-ho-phenyl)-2-ph-tetrahydro-pyrrolo(3,4-d)isoxazole-4,6-dione, 5-benzyl-3-(4-nitro-ph)-2-ph-dihydro-2h-pyrrolo(3,4-d)isoxazole-4,6(3h,5h)-dione, 5-benzyl-3-(4-methoxy-phenyl)-2-ph-tetrahydro-pyrrolo(3,4-d)isoxazole-4,6-dione, 5-benzyl-3-(4-fluorophenyl)-2-(2-methylphenyl)dihydro-2h-pyrrolo[3,4-d]isoxazole-4,6(3h,5h)-dione, 5-benzyl-3-(3-nitro-ph)-2-phenyl-tetrahydro-pyrrolo(3,4-d)isoxazole-4,6-dione, 5-benzyl-2-ph-3-(2-pyridinyl)dihydro-2h-pyrrolo(3,4-d)isoxazole-4,6(3h,5h)-dione, 5-benzyl-2-ph-3-(2-ph-vinyl)dihydro-2h-pyrrolo(3,4-d)isoxazole-4,6(3h,5h)-dione, 5-benzyl-2-(4-chlorophenyl)-3-(2-thienyl)dihydro-2h-pyrrolo[3,4-d]isoxazole-4,6(3h,5h)-dione, 5-benzyl-2,3-diphenyldihydro-2h-pyrrolo(3,4-d)isoxazole-4,6(3h,5h)-dione, 5-benzyl-2(4-cl-ph)3-(2-furyl)dihydro-2h-pyrrolo(3,4-d)isoxazole-4,6(3h,5h)dione, 5-benzyl-2(4-cl-ph)-3-(4-f-ph)dihydro-2h-pyrrolo(3,4-d)isoxazole-4,6(3h,5h)dione, 5-(p-tolyl)isoxazole, 5-(4-methylphenyl)-3-(4-nitrophenyl)-2-phenyldihydro-2h-pyrrolo[3,4-d]isoxazole-4,6(3h,5h)-dione, 5-(4-methoxyphenyl)-2-phenyl-3-(4-pyridinyl)dihydro-2h-pyrrolo[3,4-d]isoxazole-4,6(3h,5h)-dione, 5-(4-methoxyphenyl)-2-phenyl-3-(3-pyridinyl)dihydro-2h-pyrrolo[3,4-d]isoxazole-4,6(3h,5h)-dione, 5-(4-methoxy-ph)-2,3-diphenyldihydro-2h-pyrrolo(3,4-d)isoxazole-4,6(3h,5h)-dione, 5-(4-fluorophenyl)-3-(4-methoxyphenyl)-2-phenyldihydro-2h-pyrrolo[3,4-d]isoxazole-4,6(3h,5h)-dione, 5-(4-fluorophenyl)-2-(2-methylphenyl)-3-(4-pyridinyl)dihydro-2h-pyrrolo[3,4-d]isoxazole-4,6(3h,5h)-dione, 5-(4-ethoxyphenyl)-3-(4-nitrophenyl)-2-phenyldihydro-2h-pyrrolo[3,4-d]isoxazole-4,6(3h,5h)-dione, 5-(4-ethoxyphenyl)-3-(4-fluorophenyl)-2-phenyldihydro-2h-pyrrolo[3,4-d]isoxazole-4,6(3h,5h)-dione, 5-(4-ethoxyphenyl)-2-methyl-3-(4-nitrophenyl)dihydro-2h-pyrrolo[3,4-d]isoxazole-4,6(3h,5h)-dione, 5-(4-ethoxy-ph)-2-ph-3-thiophen-2-yl-4h-pyrrolo(3,4-d)isoxazole-4,6-dione, 5-(4-cl-ph)-3-(3-nitro-ph)-2-phenyl-tetrahydro-pyrrolo(3,4-d)isoxazole-4,6-dione, 5-(4-bromophenyl)-3-(4-methoxyphenyl)-2-phenyldihydro-2h-pyrrolo[3,4-d]isoxazole-4,6(3h,5h)-dione, 5-(4-bromophenyl)-3-(2-furyl)-2-(2-methylphenyl)dihydro-2h-pyrrolo[3,4-d]isoxazole-4,6(3h,5h)-dione, 5-(4-bromophenyl)-2-phenyl-3-(2-pyridinyl)dihydro-2h-pyrrolo[3,4-d]isoxazole-4,6(3h,5h)-dione, 5-(4-br-ph)2-ph-3-(2-ph-vinyl)dihydro-2h-pyrrolo(3,4-d)isoxazole-4,6(3h,5h)dione, 5-(2-cl-ph)-3-(4-dimethylamino-ph)2-o-tolyl-4h-pyrrolo(3,4-d)isoxazole-4,6-dione, 5-(2-chlorophenyl)-3-[4-(dimethylamino)phenyl]-2-phenyldihydro-2h-pyrrolo[3,4-d]isoxazole-4,6(3h,5h)-dione, 5-(2-chlorophenyl)-3-(4-methoxyphenyl)-2-phenyldihydro-2h-pyrrolo[3,4-d]isoxazole-4,6(3h,5h)-dione, 4-((3-(2-cl-ph)-5-methyl-isoxazole-4-carbonyl)-amino)-benzoic acid ethyl ester, 4,5,6,6a-tetrahydro-3ah-cyclopenta[d]isoxazole-3-carboxylic acid, 3-phenyl-3a,6a-dihydrothieno[2,3-d]isoxazole 4,4-dioxide, 3-methyl-5-(3-phenylpropyl)isoxazole, 3-methyl-4-nitro-5-[(e)-2-phenylethenyl]isoxazole, 3-methyl-4,5,8,9-tetrahydrocycloocta(d)isoxazole, 3-methyl-4,5,5a,6a,7,8-hexahydrooxireno(2′,3′:5,6)cycloocta(1,2-d)isoxazole, 3-methyl-3a,4,5,8,9,9a-hexahydrocycloocta(d)isoxazole, 3-furan-2-yl-2-phenyl-5-p-tolyl-tetrahydro-pyrrolo(3,4-d)isoxazole-4,6-dione, 3-chloro-4,5-dihydro(1)-benzothiepino(5,4-c)isoxazole, 3-[4-(dimethylamino)phenyl]-5-(4-methoxyphenyl)-2-(2-methylphenyl)dihydro-2h-pyrrolo[3,4-d]isoxazole-4,6(3h,5h)-dione, 3-(5-br-2-ho-phenyl)-2,5-diphenyl-tetrahydro-pyrrolo(3,4-d)isoxazole-4,6-dione, 3-(5-br-2-ho-ph)-5-(2-cl-ph)-2-ph-tetrahydro-pyrrolo(3,4-d)isoxazole-4,6-dione, 3-(4-meo-phenyl)-5-phenyl-2-o-tolyl-tetrahydro-pyrrolo(3,4-d)isoxazole-4,6-dione, 3-(4-fluorophenyl)-5-(4-nitrophenyl)-2-phenyldihydro-2h-pyrrolo[3,4-d]isoxazole-4,6(3h,5h)-dione, 3-(4-fluorophenyl)-5-(4-methylphenyl)-2-phenyldihydro-2h-pyrrolo[3,4-d]isoxazole-4,6(3h,5h)-dione, 3-(4-dimethylamino-ph)-5-ph-2-o-tolyl-4h-pyrrolo(3,4-d)isoxazole-4,6-dione, 3-(4-fluorophenyl)-5-(4-methoxyphenyl)-2-phenyldihydro-2h-pyrrolo[3,4-d]isoxazole-4,6(3h,5h)-dione, 3-(4-br-ph)-2-ph-5-(2-trifluoromethyl-ph)-4h-pyrrolo(3,4-d)isoxazole-4,6-dione, 3-(3-nitro-phenyl)-2,5-diphenyl-tetrahydro-pyrrolo(3,4-d)isoxazole-4,6-dione, 3-(3-br-phenyl)-2,5-diphenyldihydro-2h-pyrrolo(3,4-d)isoxazole-4,6(3h,5h)-dione, 3-(3-br-ph)-5-(2-meo-ph)-2-o-tolyl-tetrahydro-pyrrolo(3,4-d)isoxazole-4,6-dione, 3-(2-furyl)-5-[4-(4-morpholinyl)phenyl]-2-phenyldihydro-2h-pyrrolo[3,4-d]isoxazole-4,6(3h,5h)-dione, 3-(2-furyl)-2-(2-me-ph)-5-ph-dihydro-2h-pyrrolo(3,4-d)isoxazole-4,6(3h,5h)-dione, 3-(2-furyl)-2,5-diphenyldihydro-2h-pyrrolo(3,4-d)isoxazole-4,6(3h,5h)-dione, 3-(2-cl-phenyl)-5-methyl-isoxazole-4-carboxylic acid (2,5-dichloro-phenyl)-amide, 3-(2-cl-ph)-5-me-isoxazole-4-carboxylic acid (4,5-dihydro-thiazol-2-yl)-amide, 3-(2-chloro-phenyl)-5-methyl-isoxazole-4-carboxylic acid cyanomethyl-amide, dichlorophenyl)-5-(4-methoxyphenyl)-2-phenyldihydro-2h-pyrrolo[3,4-d]isoxazole-4,6(3h,5h)-dione, 3-(2,4-di-cl-ph)-2,5-diphenyldihydro-2h-pyrrolo(3,4-d)isoxazole-4,6(3h,5h)-dione, 3-(2,2-dichloro-vinyl)-5-phenyl-isoxazole, 3,5-diphenyl-isoxazole, 3,5-dimethyl-4-(1-pyrrolidinylsulfonyl)isoxazole, 3(4-dimethylamino-ph)-5-(4-eto-ph)2-o-tolyl-4h-pyrrolo(3,4-d)isoxazole-4,6-dione, 2-(4-cl-ph)5-ph-3-(2-thienyl)dihydro-2h-pyrrolo(3,4-d)isoxazole-4,6(3h,5h)-dione, 2-(4-cl-ph)-5-(3-meo-ph)-3-(3-nitro-ph)-4h-pyrrolo(3,4-d)isoxazole-4,6-dione, 2-(4-cl-ph)-3-(4-meo-ph)-5-p-tolyl-tetrahydro-pyrrolo(3,4-d)isoxazole-4,6-dione, 2-(4-chlorophenyl)-5-(4-methylphenyl)-3-(2-thienyl)dihydro-2h-pyrrolo[3,4-d]isoxazole-4,6(3h,5h)-dione, 2-(4-chlorophenyl)-3-[4-(dimethylamino)phenyl]-5-phenyldihydro-2h-pyrrolo[3,4-d]isoxazole-4,6(3h,5h)-dione, 2-(4-chlorophenyl)-3-(4-fluorophenyl)-5-(4-nitrophenyl)dihydro-2h-pyrrolo[3,4-d]isoxazole-4,6(3h,5h)-dione, 2-(4-chlorophenyl)-3-(2-thienyl)-5-[3-(trifluoromethyl)phenyl]dihydro-2h-pyrrolo[3,4-d]isoxazole-4,6(3h,5h)-dione, 2-(4-chlorophenyl)-3-(2,4-dichlorophenyl)-5-phenyldihydro-2h-pyrrolo[3,4-d]isoxazole-4,6(3h,5h)-dione, 2,3-di-ph-5-(3-(tri-f-me)ph)dihydro-2h-pyrrolo(3,4-d)isoxazole-4,6(3h,5h)-dione, danazol, and n-cyclopropyl-5-(thiophen-2-yl)isoxazole-3-carboxamide.

20. The isolated cell of claim 2, further comprising culturing the cell in serum-free media.

21. The isolated exosome or microvesicle of claim 1, wherein the effective amount of the isoxazole, derivative or an equivalent there of comprises an amount that results in overexpression of the miRNA.

22. The isolated exosome or microvesicle of claim 21 wherein the effective amount is from about 5 μM to about 25 μM.

23. The isolated exosome or microvesicle of claim 1, wherein the iPSC is differentiated into a cell from the group of: a cardiac progenitor cell, a cardiomyocyte, an endothelial cell, a myocyte, a smooth muscle cell, or an iPSC-derived embryoid body.

24. A method for one or more of providing in a subject in need thereof:
a. regenerating damaged tissue;
b. improving the viability of damaged tissue;
c. facilitating the formation of new tissue, optionally wherein the new tissue is selected from the group of: cardiac tissue, muscle tissue, or skeletal muscle; blood vessel, capillary or a myocyte;
d. promoting cardiac regeneration;
e. promoting cardiac regeneration in a subject suffering from an acute cardiac event;
f. promoting cardiac regeneration in a subject suffering from a myocardial infarction;
g. promoting cardiac regeneration in subject suffering from duchenne muscular dystrophy or duchenne muscular dystrophy-associated cardiomyopathy;
h. promoting cardiac regeneration in a subject suffering from age-related diseases selected from the group of: COPD, arthritis, osteoporosis, osteoarthritis, diabetes, vascular dementia or macular degeneration, following age related diseases (such as Hoyeraal-Hreidarsson syndrome, dyskeratosis congenita, pulmonary fibrosis, aplastic anemia, liver fibrosis, dyskeratosis congenita, bone marrow failure, lung disease, endocrine diseases, polycystic ovary syndrome (PCOS), Cushing's syndrome, and acromegaly, Cerebrovascular Disease (Strokes), High Blood Pressure—Hypertension, Parkinson's Disease, Dementia, Alzheimer's Disease, Age-related hearing loss, Celiac disease (CD), COPD, bipolar disorder, hydroxyurea, sickle cell diseases, hypertension, atherosclerosis, arthritis, osteoporosis, osteoarthritis, vasculardementia or macular degeneration, cancer, type 2 diabetes, or diseases with telomerase dysfunction dealing with a shortened telomere length);
i. promoting tissue regeneration in tissue damaged from one or more of stroke, arthritis, Alzheimer's, memory loss disorders, cystoc fibrosis, inflammatory disorders or cancer;
j. decreasing cardiac wall thickness in a tissue damaged from a cardiac infarction;
k. altering gene expression of one or more of a protein kinase C, iL-6, mmp, or PDGF;
l. reducing or inhibiting the expression of an inflammatory protein, that is optionally a cytokine, a chemokine, or a macrophage;
m. directly or indirectly stimulating angiogenesis;
n. promoting cardiac regeneration in a subject suffering from a disease selected from the group of coronary artery disease, myocardial infarction, heart failure, hypoplasic left heart syndrome, peripheral artery disease (PAD), cardiac hypertrophy, valvular heart disease (aortic stenosis), myocardial hypertrophy mi hyperthrophy fibrosis, and/or;
o. directly or indirectly inhibiting cellular replication,
the method comprising administering an effective amount of the isolated exosome or microvesicle of claim 1 or the cell of claim 2 to the subject.

25. The method of claim 24, further comprising administering an effective amount of a non-embryonic stem cell or progenitor cell to the subject, that is optionally of the same type as the tissue in need of repair of an type different from the type of tissue of repair.

26. The method of claim 25, wherein the non-embryonic stem or progenitor cell is autologous or allogeneic to the subject.

27. The method of claim 25, wherein the exosomes or microvesicles are delivered locally or systemically to the tissue of the subject.

28. The method of claim 25, wherein the exosomes or microvesicles are delivered to the subject via an intramyocardial or a intracoronary route.

29. A method for one or more of:
a. Providing anti-oxidative therapy;
b. Promoting activation of local or resident cardiomyocytes;
c. Promoting the release of angiogenesis and/or paracrine factors;
d. Promoting activation of wnt, a BMP, and/or cytoskeleton remodeling;
e. Promoting TGF-β induced emt signaling and cardiac differentiation;
f. Increasing expression of wnt5 and wnt11 or a BMP family protein, optionally BMP4;
g. Increasing expression of a cardiac transcription factor selected from the group consisting of nkx2.5, mef2c, gata4 and isl-1;
h. Promoting expression of genes for development of pip3 signaling in cardiomyocytes, muscle contraction and nf-at hypertrophy signaling pathways;
i. Reducing fibrosis and apoptosis;
j. Promoting myoangeneis and muscle differentiation;
k. Promoting the release of a cytokine selected from the group consisting of angiopoietin-2, il-6nmp, pgfbb, timp 1;
l. Promoting upregulation of a gene selected from the group consisting of wnt3a, wnt5a, wnt11; and/or
m. Promoting cytoskeletal remodeling,
the method comprising administering an effective amount of the isolated exosome or microvesicle of claim 1 or the isolated cell of claim 2, to a subject in need thereof.

30. An isolated population of cells of any one of claims 3-5.

31. The cell if any of claims 3-5 or the population of claim 30, wherein the population is prepared by a method comprising culturing a population of iPSC in the presence of an effective amount of an isoxazole, a derivative or an equivalent thereof.

32. The population of claim 31, wherein the population is derived from a cell selected from the group of: a fibroblast, a skeletal myoblast, a smooth muscle cell, an endothelial cell, a blood vessel, a cardiomyocyte, a capillary or a myocyte.

33. The population of claim 31, wherein the isoxazole compound is isoxazole-1 (isx-1) or isoxazole-9 (isx-9).

34. The population of claim 31, wherein the isoxazole derivative is selected from the group of: 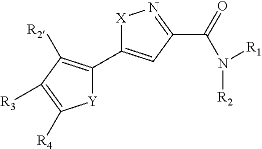
wherein R₁ and R₂ are both hydrogen or R₁ is hydrogen and R₂ is selected from the group consisting of substituted or unsubstituted C₁-C₆ alkyl, C₃-C₆ cycloalkyl, C₂-C₆ alkenyl, C₂-C₆ alkynyl, and benzyl, or where R₁ and R₂ may be joined together to form a ring selected from azetidinyl, pyrrolidinyl, piperidinyl, piperazinyl, or morpholinyl; R₂′, R₃ and R₄ are independently selected from the group consisting of hydrogen, halogen, C₁-C₆ alkyl, C₃-C₆ cycloalkyl, substituted or unsubstituted aromatic or heteroaromatic ring, cyano, nitro, and acyl; X is 0, NH or S; and Y is 0, NH or S.

35. The composition of claim 31, wherein the isoxazole compound has the formula: 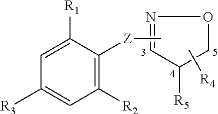
wherein R1 and R2 is each selected from C1-C4 alkyl, phenyl, benzyl, trifluoromethyl or halogen, R3 is selected from hydrogen, hydroxy, C1-C4 alkyl or alkoxy, R4, in position 3 or 5, is selected from hydrogen, trifluoromethyl, C1-C4 alkoxy, C1-C4 alkyl, or C1-C4 hydroxyalkyl, R5 is selected from hydrogen or C4-C4 alkyl or R4 and R5 together form a tetramethylene group, Z at position 3 or 5 on the heterocycle is selected from: —N(R6)-CO—, —CO—N(R6)-, —N(R6)-CO—N(R6)-, —CH(R6)-NH—CO—, or —NH—CO—CH(R6), in which R6 is selected from hydrogen or C1-C4 alkyl.

36. The composition of claim 33, wherein the isoxazole compound or a derivative thereof is selected from the group: 5-(trifluoromethyl)-3-(4-methoxyphenyl)isoxazole-4-carboxylic acid, 5-(trifluoromethyl)-3-(4-fluorophenyl)isoxazole-4-carboxylic acid, 5-(thiophen-2-yl)isoxazole-3-carboxaldehyde, 5,6,7,8-tetrahydro-4h-cyclohepta[d]isoxazole-3-carboxylic acid, 4,5,6,7-tetrahydro-benzo[d]isoxazole-3-carboxylic acid, 3-amino-5-methylisoxazole, 4-amino-n-(5-methyl-3-isoxazolyl)benzenesulfonamide, 3-phenyl-isoxazole-5-boronic acid pinacol ester, 5-phenylisoxazole, 1-phenyl-1-cyclopentanecarboxylic acid, 3-phenyl-benzo[c]isoxazole-5-carboxylic acid, 5-methyl-3-phenylisoxazole-4-carboxylic acid, 3a,4,5,6,7,8,9,9a-octahydro-cycloocta[d]isoxazole-3-carboxylic acid, 5-(3-nitrophenyl)isoxazole, 3-(4-nitrophenyl)isoxazole, 3-hydroxy-5-aminomethyl-isoxazole, 5-(morpholinomethyl)isoxazole-3-carboxylic acid hydrochloride, 5-(morpholinomethyl)isoxazole-3-carbaldehyde, 3-methyl-5-(trifluoromethyl)isoxazole-4-carboxylic acid, methyl 5-(thiophen-2-yl)isoxazole-3-carboxylate, 3-(methylsulfonyl)-5-(2-thienyl)isoxazole-4-carbonitrile, 5-methyl-3-(2-pyrrolidinyl)isoxazole, 3-methyl-5-(2-pyrrolidinyl)isoxazole, 3-(1-methyl-1h-pyrazol-4-yl)-isoxazole-5-carboxylic acid, 3-(1-methyl-1h-pyrazol-4-yl)-4,5-dihydro-isoxazole-5-carboxylic acid, 5-(4-methylphenyl)isoxazole-3-carboxylic acid, 5-methyl-3-phenylisoxazole-4-carboxylic acid, 5-(4-methylphenyl)isoxazole-3-carboxaldehyde, 5-methyl-3-(4-phenoxyphenyl)isoxazole-4-carboxylic acid, 3-methyl-5-(4-methyl-1,2,3-thiadiazol-5-yl)isoxazole-4-carboxylic acid, 3-methyl-5-(5-methylisoxazol-3-yl)isoxazole-4-carboxylic acid, methyl 5-(4-methoxyphenyl)isoxazole-4-carboxylate, methyl 5-(4-methoxyphenyl)isoxazole-3-carboxylate, 5-methylisoxazole, methyl 5-(4-fluorophenyl)isoxazole-4-carboxylate, methyl 5-(4-fluorophenyl)isoxazole-3-carboxylate, methyl 5-(4-chlorophenyl)isoxazole-4-carboxylate, methyl 5-(4-bromophenyl)isoxazole-4-carboxylate, 5-(4-methoxyphenyl)isoxazole-3-carboxylic acid, 5-(3-methoxy-phenyl)-isoxazole-3-carboxylic acid, 3-(2-methoxyphenyl)isoxazole-5-carboxylic acid, 5-(4-methoxyphenyl)isoxazole-3-carboxaldehyde, 3-(4-methoxyphenyl)isoxazole-5-carbaldehyde, 3-(2-methoxyphenyl)isoxazole-5-carbaldehyde, 5-(4-methoxyphenyl)isoxazole, 3-(4-methoxyphenyl)isoxazole, 3-(2-methoxy-phenyl)-4,5-dihydro-isoxazole-5-carboxylic acid, 3-methoxy-isoxazole-5-carboxylic acid, isoxazole-5-carboxylic acid, isoxazole-4-carboxylic acid, isoxazole-5-carbothioamide, isoxazole-5-carbonyl chloride, isoxazole-3-carbonitrile, isoxazole-3-carbaldehyde, isoxazole-4-boronic acid, isoxazole, 5-cyclopropyl-4[2-(methylsulfonyl)-4-(trifluoromethyl)benzoyl]isoxazole, 6-(5-(thiophen-2-yl)isoxazole-3-carboxamido)hexyl 5-((3as,4s,6ar)-2-oxohexahydro-1h-thieno[3,4-d]imidazol-4-yl)pentanoate, isocarboxazid 5-methyl-3-isoxazole-carboxylic acid 2-benzylhydrazide, 5-isobutyl-isoxazole-3-carboxylic acid, 4-iodo-5-methyl-isoxazole, 3,3′-iminobis(n,n-dimethylpropylamine), 3-(3-hydroxy-phenyl)-isoxazole-5-carboxylic acid methyl ester, 5-(4-hydroxy-phenyl)-isoxazole-3-carboxylic acid, 5-(3-hydroxy-phenyl)-isoxazole-3-carboxylic acid, 5-(hydroxymethyl)-3-methylisoxazole, 3-hydroxy-5-methylisoxazole, 5-(1-hydroxyethyl)-3-(4-trifluoromethylphenyl)isoxazole, 3a,4,5,6,7,7a-hexahydro-benzo[d]isoxazole-3-carboxylic acid, 5-(2-furyl)isoxazole-3-carbaldehyde, 5-furan-2-yl-isoxazole-3-carboxylic acid, 6-fluoro-3-(4-piperidinyl)benzisoxazole, 5-(4-fluorophenyl)isoxazole-3-methanol, 3-(2-fluoro-phenyl)-isoxazole-5-carboxylic acid, 5-(4-fluorophenyl)isoxazole-3-carboxaldehyde, 3-(4-fluorophenyl)isoxazole-5-carbaldehyde, 3-(3-fluorophenyl)isoxazole-5-carbaldehyde, 3-(2-fluorophenyl)isoxazole-5-carbaldehyde, 5-(4-fluorophenyl)isoxazole, 3-(4-fluorophenyl)isoxazole, 5-(3-fluoro-4-methoxy-phenyl)-isoxazole-3-carboxylic acid, ethyl 5-(trifluoromethyl)-3-(4-methoxyphenyl)isoxazole-4-carboxylate, ethyl-5-(tributylstannyl)isoxazole-3-carboxylate, ethyl 5-(thiophen-2-yl)isoxazole-3-carboxylate, 5-ethyl-isoxazole-4-carboxylic acid, 5-ethyl-isoxazole-3-carboxylic acid, ethyl 5-(4-fluorophenyl)isoxazole-4-carboxylate, ethyl 5-(4-fluorophenyl)isoxazole-3-carboxylate, ethyl 5-(2,3-dihydrobenzo[b][1,4]dioxin-7-yl)isoxazole-3-carboxylate, ethyl 3-(4-chlorophenyl)-5-(trifluoromethyl)isoxazole-4-carboxylate, ethyl 5-(4-chlorophenyl)isoxazole-3-carboxylate, ethyl 3-(4-bromophenyl)-5-(trifluoromethyl)isoxazole-4-carboxylate, ethyl 5-(4-bromophenyl)isoxazole-3-carboxylate, ethyl 5-amino-4-(4-chlorophenyl)isoxazole-3-carboxylate, ethyl 5-amino-4-(4-bromophenyl)isoxazole-3-carboxylate, ethyl 6b-acetyl-2-(acetyloxy)-4a,6a-dimethyl-2,3,4,4a,4b,5,6,6a,6b,9a,10,10a,10b,11-tetradecahydro-1h-naphtho[2′,1′:4,5]indeno[2,1-d]isoxazole-9-carboxylate, 3,5-dimethyl-4-(tributylstannyl)isoxazole, 5-(1,5-dimethyl-1h-pyrazol-4-yl)-isoxazole-3-carboxylic acid, 5-(1,3-dimethyl-1h-pyrazol-4-yl)-isoxazole-3-carboxylic acid, 5-(1,5-dimethyl-1h-pyrazol-4-yl)-isoxazole, 3,5-dimethylisoxazole-4-boronic acid pinacol ester, 3,5-dimethylisoxazole, 3-(dimethylamino)-1-(2-pyridyl)-2-propen-1-one, 5-(3,5-difluorophenyl)isoxazole, [2,6-dichloro-4-(trifluoromethyl)phenyl]hydrazine, 5-(2,5-dichlorophenyl)isoxazole-3-carboxylic acid, danazol, 3-(4-chlorophenyl)-5-(trifluoromethyl)isoxazole-4-carboxylic acid, 5-(4-chlorophenyl)isoxazole-3-propionic acid, 5-(4-chlorophenyl)isoxazole-4-carboxylic acid, 5-(4-chlorophenyl)isoxazole-3-carboxylic acid, 3-(4-chlorophenyl)isoxazole-5-carboxylic acid, 3-(3-chlorophenyl)isoxazole-5-carboxylic acid, 5-(4-chlorophenyl)isoxazole-3-carboxaldehyde, 3-(4-chlorophenyl)isoxazole-5-carbaldehyde, 3-(3-chlorophenyl)isoxazole-5-carbaldehyde, 3-(2-chlorophenyl)isoxazole-5-carbaldehyde, 5-(4-chlorophenyl)isoxazole, 3-(4-chlorophenyl)isoxazole, 5-(chloromethyl)isoxazole-4-carboxylic acid, 3-(chloromethyl)-5-(2-furyl)isoxazole, 4-chloromethyl-3,5-dimethylisoxazole, 5-(chloromethyl)-3-(4-chlorophenyl)isoxazole, 5-(3-chloro-4-methoxy-phenyl)-isoxazole-3-carboxylic acid, 3-chloro-4-fluorobenzaldehyde, 3-(5-chloro-2,4-dimethoxy-phenyl)-4,5-dihydro-isoxazole-5-carboxylic acid, 5-tert-butyl-4,5,6,7-tetrahydro-benzo[d]isoxazole-3-carboxylic acid, 3-(4-bromophenyl)-5-(trifluoromethyl)isoxazole-4-carboxylic acid, 5-(4-bromophenyl)isoxazole-3-propionic acid, 5-(4-bromophenyl)isoxazole-3-carboxylic acid hydrazide, 5-(4-bromophenyl)isoxazole-4-carboxylic acid, 5-(4-bromophenyl)isoxazole-3-carboxylic acid, 3-(4-bromophenyl)isoxazole-5-carboxylic acid, 3-(4-bromophenyl)isoxazole-5-carboxaldehyde, 5-(4-bromophenyl)isoxazole, 5-(3-bromophenyl)isoxazole, 3-(4-bromophenyl)isoxazole, 5-(bromomethyl)-3-(4-methoxyphenyl)isoxazole, 4-(bromomethyl)isoxazole, 5-(bromomethyl)-3-(4-fluorophenyl)isoxazole, 5-(bromomethyl)-3-(4-chlorophenyl)isoxazole, 5-(bromomethyl)-3-(4-bromophenyl)isoxazole, 6-bromo-3-methylbenzo[d]isoxazole, 5-bromo-3-methylbenzo[d]isoxazole, 4-bromo-5-(4-methoxyphenyl)isoxazole, 3-bromo-isoxazole, 3-bromo-5-(2-hydroxyethyl)isoxazole, 4-bromo-5-(4-fluorophenyl)isoxazole, 3-bromo-5-(4-fluorophenyl)isoxazole, 4-bromo-5-(4-chlorophenyl)isoxazole, 4-bromo-5-(4-bromophenyl)isoxazole, 6-bromo-benzo[d]isoxazole-3-carboxylic acid, benzo[d]isoxazole-3-carboxylic acid, 3-amino-5-methylisoxazole, 5-amino-3-(4-methoxyphenyl)isoxazole, 3-aminoisoxazole, 3-amino-5-(4-fluorophenyl)isoxazole, 5-amino-3-(4-chlorophenyl)isoxazole, 5-amino-4-(4-bromophenyl)isoxazole, 3-amino-5-(4-bromophenyl)isoxazole, 5-acetyl-3-(4-fluorophenyl)isoxazole, 5-acetyl-3-(3-fluorophenyl)isoxazole, 3-methyl-5-[(2s)-1-methyl-2-pyrrolidinyl]isoxazole hydrochloride, 7-methoxy-5-methyl-4,5-dihydronaphtho[2,1-d]isoxazole, 5-methyl-3-phenyl-isoxazole-4-carboxylic acid methylamide, 5-methyl-3-phenyl-isoxazole-4-carbothioic acid methylamide, 5-methyl-3-phenyl-4-(1h-pyrazol-5-yl)isoxazole, 5-benzyl-3-furan-2-yl-2-phenyl-tetrahydro-pyrrolo(3,4-d)isoxazole-4,6-dione, 5-benzyl-3-[4-(dimethylamino)phenyl]-2-phenyldihydro-2h-pyrrolo[3,4-d]isoxazole-4,6(3h,5h)-dione, 5-benzyl-3-(5-br-2-ho-phenyl)-2-ph-tetrahydro-pyrrolo(3,4-d)isoxazole-4,6-dione, 5-benzyl-3-(4-nitro-ph)-2-ph-dihydro-2h-pyrrolo(3,4-d)isoxazole-4,6(3h,5h)-dione, 5-benzyl-3-(4-methoxy-phenyl)-2-ph-tetrahydro-pyrrolo(3,4-d)isoxazole-4,6-dione, 5-benzyl-3-(4-fluorophenyl)-2-(2-methylphenyl)dihydro-2h-pyrrolo[3,4-d]isoxazole-4,6(3h,5h)-dione, 5-benzyl-3-(3-nitro-ph)-2-phenyl-tetrahydro-pyrrolo(3,4-d)isoxazole-4,6-dione, 5-benzyl-2-ph-3-(2-pyridinyl)dihydro-2h-pyrrolo(3,4-d)isoxazole-4,6(3h,5h)-dione, 5-benzyl-2-ph-3-(2-ph-vinyl)dihydro-2h-pyrrolo(3,4-d)isoxazole-4,6(3h,5h)-dione, 5-benzyl-2-(4-chlorophenyl)-3-(2-thienyl)dihydro-2h-pyrrolo[3,4-d]isoxazole-4,6(3h,5h)-dione, 5-benzyl-2,3-diphenyldihydro-2h-pyrrolo(3,4-d)isoxazole-4,6(3h,5h)-dione, 5-benzyl-2(4-cl-ph)3-(2-furyl)dihydro-2h-pyrrolo(3,4-d)isoxazole-4,6(3h,5h)dione, 5-benzyl-2(4-cl-ph)-3-(4-f-ph)dihydro-2h-pyrrolo(3,4-d)isoxazole-4,6(3h,5h)dione, 5-(p-tolyl)isoxazole, 5-(4-methylphenyl)-3-(4-nitrophenyl)-2-phenyldihydro-2h-pyrrolo[3,4-d]isoxazole-4,6(3h,5h)-dione, 5-(4-methoxyphenyl)-2-phenyl-3-(4-pyridinyl)dihydro-2h-pyrrolo[3,4-d]isoxazole-4,6(3h,5h)-dione, 5-(4-methoxyphenyl)-2-phenyl-3-(3-pyridinyl)dihydro-2h-pyrrolo[3,4-d]isoxazole-4,6(3h,5h)-dione, 5-(4-methoxy-ph)-2,3-diphenyldihydro-2h-pyrrolo(3,4-d)isoxazole-4,6(3h,5h)-dione, 5-(4-fluorophenyl)-3-(4-methoxyphenyl)-2-phenyldihydro-2h-pyrrolo[3,4-d]isoxazole-4,6(3h,5h)-dione, 5-(4-fluorophenyl)-2-(2-methylphenyl)-3-(4-pyridinyl)dihydro-2h-pyrrolo[3,4-d]isoxazole-4,6(3h,5h)-dione, 5-(4-ethoxyphenyl)-3-(4-nitrophenyl)-2-phenyldihydro-2h-pyrrolo[3,4-d]isoxazole-4,6(3h,5h)-dione, 5-(4-ethoxyphenyl)-3-(4-fluorophenyl)-2-phenyldihydro-2h-pyrrolo[3,4-d]isoxazole-4,6(3h,5h)-dione, 5-(4-ethoxyphenyl)-2-methyl-3-(4-nitrophenyl)dihydro-2h-pyrrolo[3,4-d]isoxazole-4,6(3h,5h)-dione, 5-(4-ethoxy-ph)-2-ph-3-thiophen-2-yl-4h-pyrrolo(3,4-d)isoxazole-4,6-dione, 5-(4-cl-ph)-3-(3-nitro-ph)-2-phenyl-tetrahydro-pyrrolo(3,4-d)isoxazole-4,6-dione, 5-(4-bromophenyl)-3-(4-methoxyphenyl)-2-phenyldihydro-2h-pyrrolo[3,4-d]isoxazole-4,6(3h,5h)-dione, 5-(4-bromophenyl)-3-(2-furyl)-2-(2-methylphenyl)dihydro-2h-pyrrolo[3,4-d]isoxazole-4,6(3h,5h)-dione, 5-(4-bromophenyl)-2-phenyl-3-(2-pyridinyl)dihydro-2h-pyrrolo[3,4-d]isoxazole-4,6(3h,5h)-dione, 5-(4-br-ph)2-ph-3-(2-ph-vinyl)dihydro-2h-pyrrolo(3,4-d)isoxazole-4,6(3h,5h)dione, 5-(2-cl-ph)-3-(4-dimethylamino-ph)2-o-tolyl-4h-pyrrolo(3,4-d)isoxazole-4,6-dione, 5-(2-chlorophenyl)-3-[4-(dimethylamino)phenyl]-2-phenyldihydro-2h-pyrrolo[3,4-d]isoxazole-4,6(3h,5h)-dione, 5-(2-chlorophenyl)-3-(4-methoxyphenyl)-2-phenyldihydro-2h-pyrrolo[3,4-d]isoxazole-4,6(3h,5h)-dione, 4-((3-(2-cl-ph)-5-methyl-isoxazole-4-carbonyl)-amino)-benzoic acid ethyl ester, 4,5,6,6a-tetrahydro-3ah-cyclopenta[d]isoxazole-3-carboxylic acid, 3-phenyl-3a,6a-dihydrothieno[2,3-d]isoxazole 4,4-dioxide, 3-methyl-5-(3-phenylpropyl)isoxazole, 3-methyl-4-nitro-5-[(e)-2-phenylethenyl]isoxazole, 3-methyl-4,5,8,9-tetrahydrocycloocta(d)isoxazole, 3-methyl-4,5,5a,6a,7,8-hexahydrooxireno(2′,3′:5,6)cycloocta(1,2-d)isoxazole, 3-methyl-3a,4,5,8,9,9a-hexahydrocycloocta(d)isoxazole, 3-furan-2-yl-2-phenyl-5-p-tolyl-tetrahydro-pyrrolo(3,4-d)isoxazole-4,6-dione, 3-chloro-4,5-dihydro(1)-benzothiepino(5,4-c)isoxazole, 3-[4-(dimethylamino)phenyl]-5-(4-methoxyphenyl)-2-(2-methylphenyl)dihydro-2h-pyrrolo[3,4-d]isoxazole-4,6(3h,5h)-dione, 3-(5-br-2-ho-phenyl)-2,5-diphenyl-tetrahydro-pyrrolo(3,4-d)isoxazole-4,6-dione, 3-(5-br-2-ho-ph)-5-(2-cl-ph)-2-ph-tetrahydro-pyrrolo(3,4-d)isoxazole-4,6-dione, 3-(4-meo-phenyl)-5-phenyl-2-o-tolyl-tetrahydro-pyrrolo(3,4-d)isoxazole-4,6-dione, 3-(4-fluorophenyl)-5-(4-nitrophenyl)-2-phenyldihydro-2h-pyrrolo[3,4-d]isoxazole-4,6(3h,5h)-dione, 3-(4-fluorophenyl)-5-(4-methylphenyl)-2-phenyldihydro-2h-pyrrolo[3,4-d]isoxazole-4,6(3h,5h)-dione, 3-(4-dimethylamino-ph)-5-ph-2-o-tolyl-4h-pyrrolo(3,4-d)isoxazole-4,6-dione, 3-(4-fluorophenyl)-5-(4-methoxyphenyl)-2-phenyldihydro-2h-pyrrolo[3,4-d]isoxazole-4,6(3h,5h)-dione, 3-(4-br-ph)-2-ph-5-(2-trifluoromethyl-ph)-4h-pyrrolo(3,4-d)isoxazole-4,6-dione, 3-(3-nitro-phenyl)-2,5-diphenyl-tetrahydro-pyrrolo(3,4-d)isoxazole-4,6-dione, 3-(3-br-phenyl)-2,5-diphenyldihydro-2h-pyrrolo(3,4-d)isoxazole-4,6(3h,5h)-dione, 3-(3-br-ph)-5-(2-meo-ph)-2-o-tolyl-tetrahydro-pyrrolo(3,4-d)isoxazole-4,6-dione, 3-(2-furyl)-5-[4-(4-morpholinyl)phenyl]-2-phenyldihydro-2h-pyrrolo[3,4-d]isoxazole-4,6(3h,5h)-dione, 3-(2-furyl)-2-(2-me-ph)-5-ph-dihydro-2h-pyrrolo(3,4-d)isoxazole-4,6(3h,5h)-dione, 3-(2-furyl)-2,5-diphenyldihydro-2h-pyrrolo(3,4-d)isoxazole-4,6(3h,5h)-dione, 3-(2-cl-phenyl)-5-methyl-isoxazole-4-carboxylic acid (2,5-dichloro-phenyl)-amide, 3-(2-cl-ph)-5-me-isoxazole-4-carboxylic acid (4,5-dihydro-thiazol-2-yl)-amide, 3-(2-chloro-phenyl)-5-methyl-isoxazole-4-carboxylic acid cyanomethyl-amide, dichlorophenyl)-5-(4-methoxyphenyl)-2-phenyldihydro-2h-pyrrolo[3,4-d]isoxazole-4,6(3h,5h)-dione, 3-(2,4-di-cl-ph)-2,5-diphenyldihydro-2h-pyrrolo(3,4-d)isoxazole-4,6(3h,5h)-dione, 3-(2,2-dichloro-vinyl)-5-phenyl-isoxazole, 3,5-diphenyl-isoxazole, 3,5-dimethyl-4-(1-pyrrolidinylsulfonyl)isoxazole, 3(4-dimethylamino-ph)-5-(4-eto-ph)2-o-tolyl-4h-pyrrolo(3,4-d)isoxazole-4,6-dione, 2-(4-cl-ph)5-ph-3-(2-thienyl)dihydro-2h-pyrrolo(3,4-d)isoxazole-4,6(3h,5h)-dione, 2-(4-cl-ph)-5-(3-meo-ph)-3-(3-nitro-ph)-4h-pyrrolo(3,4-d)isoxazole-4,6-dione, 2-(4-cl-ph)-3-(4-meo-ph)-5-p-tolyl-tetrahydro-pyrrolo(3,4-d)isoxazole-4,6-dione, 2-(4-chlorophenyl)-5-(4-methylphenyl)-3-(2-thienyl)dihydro-2h-pyrrolo[3,4-d]isoxazole-4,6(3h,5h)-dione, 2-(4-chlorophenyl)-3-[4-(dimethylamino)phenyl]-5-phenyldihydro-2h-pyrrolo[3,4-d]isoxazole-4,6(3h,5h)-dione, 2-(4-chlorophenyl)-3-(4-fluorophenyl)-5-(4-nitrophenyl)dihydro-2h-pyrrolo[3,4-d]isoxazole-4,6(3h,5h)-dione, 2-(4-chlorophenyl)-3-(2-thienyl)-5-[3-(trifluoromethyl)phenyl]dihydro-2h-pyrrolo[3,4-d]isoxazole-4,6(3h,5h)-dione, 2-(4-chlorophenyl)-3-(2,4-dichlorophenyl)-5-phenyldihydro-2h-pyrrolo[3,4-d]isoxazole-4,6(3h,5h)-dione, 2,3-di-ph-5-(3-(tri-f-me)ph)dihydro-2h-pyrrolo(3,4-d)isoxazole-4,6(3h,5h)-dione, danazol, and n-cyclopropyl-5-(thiophen-2-yl)isoxazole-3-carboxamide.

37. A method of preparing a population of cardiac or skeletal myogenic progenitors from a population of human induced pluripotent stem cells (hiPSCs), comprising contacting the hiPSCs with an effective amount of Givinostat (GIV), optionally cultured in serum free media.

38. The method of claim 37, wherein the effective amount of GIV comprises from about 100 nM to about 30 nM per 1×10⁶ cells.

39. The method of claim 37, further comprising culturing the cells in the presence of serum-free medium and Rho-associated kinase (ROCK) inhibitors.

40. The method of claim 39, wherein the Rho-associated kinase (ROCK) inhibitors are selected from Thiazovivin, Y27632, SR3677, or GSK429286.

41. The method of claim of claim 39, further comprising culturing the cells in the presence of serum-free medium and a TGF-beta type-I receptor inhibitor.

42. The method of claim 41, wherein the TGF-beta type-I receptor inhibitor is selected from the group consisting of SB431542, A8301 LY2157299, or LY2109761.

43. A population of cells prepared by the method of claim 37, wherein the cells overexpress skeletal myogenic genes selected from the group of: Meox1, Meox2, Tcf15, Pax3, Pax7, MyoD1, MYF5, dystrophin, or DESMIN.

44. A population of cells prepared by the method of claim 37, wherein the cells overexpress xESI myogenic genes selected from the group of: Pitx2, IS11, Nkx2.5, Hand1, GATA4, Tbx5, TnnT2, Myl7, MLC2v, Myf2c, Cdh4, or Lhx2.

45. A population of exosomes or microvesicles isolated from the population of cells of claim 43.

46. A population of exosomes or microvesicles isolated from the population of cells of claim 44.

47. The population of claim 45 or 46, wherein the population is substantially homogenous.

48. A method of regenerating skeletal muscle comprising administering to a subject in need thereof an effective amount of the population of cells of claim 43 and/or an effective amount of the population of exosomes or microvesicles of claim 46.

49. A method of regenerating cardiac muscle comprising administering to a subject in need thereof an effective amount of the population of cells of claim 44 and/or an effective amount of the population of exosome or microvesicle of claim 47.

50. A method for treating Duchenne Muscular Dystrophy (DMD), comprising administering to a subject in need thereof an effective amount of the population of cells of claim 43 and/or an effective amount of the population of exosome or microvesicle of claim 46.

51. A method for treating cardiac dysfunction associated with Duchenne Muscular Dystrophy (DMD), comprising administering to a subject in need thereof an effective amount of the population of cells of claim 44 and/or an effective amount of the population of exosome or microvesicle of claim 43.

52. The population of exosomes or microvesicles of any claim 45 or 46, wherein the exosomes or microvesicles have an average diameter of from about 20 nm to about 90 nm.

53. A composition for the repair or regeneration of damaged or diseased cardiac tissue or for the treatment of one or more of Hoyeraal-Hreidarsson syndrome, dyskeratosis congenita, pulmonary fibrosis, aplastic anemia, liver fibrosis, dyskeratosis congenita, bone marrow failure, lung disease, endocrine diseases, polycystic ovary syndrome (PCOS), Cushing's syndrome, and acromegaly, Cerebrovascular Disease (Strokes), High Blood Pressure—Hypertension, Parkinson's Disease, Dementia, Alzheimer's Disease, Age-related hearing loss, Celiac disease (CD), COPD, bipolar disorder, hydroxyurea, sickle cell diseases, hypertension, atherosclerosis, arthritis, osteoporosis, osteoarthritis, vasculardementia or macular degeneration, cancer, type 2 diabetes, or diseases with telomerase dysfunction dealing with a shortened telomere length, the composition comprising a synthetic microRNA-195 inhibitor.

54. A method of regenerating tissue or for the treatment of one or more of Hoyeraal-Hreidarsson syndrome, dyskeratosis congenita, pulmonary fibrosis, aplastic anemia, liver fibrosis, dyskeratosis congenita, bone marrow failure, lung disease, endocrine diseases, polycystic ovary syndrome (PCOS), Cushing's syndrome, and acromegaly, Cerebrovascular Disease (Strokes), High Blood Pressure—Hypertension, Parkinson's Disease, Dementia, Alzheimer's Disease, Age-related hearing loss, Celiac disease (CD), COPD, bipolar disorder, hydroxyurea, sickle cell diseases, hypertension, atherosclerosis, arthritis, osteoporosis, osteoarthritis, vasculardementia or macular degeneration, cancer, type 2 diabetes, or diseases with telomerase dysfunction dealing with a shortened telomere length in a subject in need thereof, comprising; administering an effective amount of one or more microRNA fragments, or derivatives thereof to the subject, wherein after administration of the one or more microRNA fragments, the one or more microRNA fragments alter gene expression in the damaged tissue, improve the viability of said damaged tissue, and facilitate the formation of new tissue in the subject, and wherein the administration optionally further comprises an effective amount of Danazol.

55. The method of claim 54, wherein the microRNA fragments, or derivatives thereof, are synthetically generated.

56. The method of claim 54, wherein the microRNA fragments, or derivatives thereof are synthesized with a sequence that mimics one or more endogenous microRNA molecules.

57. The method of claim 54, wherein the microRNA fragments, or derivatives thereof are modified to enhance their stability, optionally via oligonucleotides, and further optionally wherein the mRNA fragments are administered via liposome based nanoparticles without premature degradation, and further optionally wherein the miRNA are safely derived.

58. The method of claim 54, wherein the administration comprises administration of a plurality of synthetic liposomes that comprise said one or more microRNA fragments, or derivatives thereof.

59. A method of isolating a population of exosomes or microvesicles, comprising culturing a population of non-embryonic human regenerative cells in the presence of a hydrolase enzyme to induce the cells to secrete exosomes or microvesicles, thereby generating exosomes or microvesicles.

60. The method of claim 59, further comprising isolating the secreted exosomes or microvesicles from the culture media.

61. The method of claim 59, wherein said hydrolase comprises a member of the DNAse I superfamily of enzymes.

62. The method of claim 59, wherein said hydrolase comprises a sphingomyelinase.

63. The method of claim 62, wherein said sphingomyelinase is of a type selected from the group consisting of lysosomal acid sphingomyelinase, secreted zinc-dependent acid sphingomyelinase, neutral sphingomyelinase, and alkaline sphingomyelinase.

64. The method of claim 62, wherein said neutral sphingomyelinase comprises one or more of magnesium-dependent neutral sphingomyelinase and magnesium-independent neutral sphingomyelinase.

65. The method of claim 63, wherein said neutral sphingomyelinase comprises one or more of neutral sphingomyelinase type I, neutral sphingomyelinase type 2, and neutral sphingomyelinase type 3.

66. A composition comprising one or more of a synthetic microRNA-373, a micro-ma 373 mimic or micro-ma 373 exosome.

67. The composition of claim 66, further comprising a carrier.

68. A method to treat fibrotic diseases comprising administering an effective amount of a composition of claim 66 to a subject in need thereof.

69. The method of claim 68, wherein the fibrotic disease is myocardial fibrosis.


SEQUENCE LISTING (16 sequences)

SEQ ID NO: 1 - DNA, 21 bp, Artificial Sequence
  Synthetic siRNA
uaucaauuccgacaucgaatt

SEQ ID NO: 2 - RNA, 21 nt, Artificial Sequence
  Synthetic oligonucleotide
uucgaugucgggaauugauac

SEQ ID NO: 3 - DNA, 21 bp, Artificial Sequence
  Synthetic siRNA
acuucugcaugaagaaugatt

SEQ ID NO: 4 - RNA, 21 nt, Artificial Sequence
  Synthetic oligonucleotide
ucauucuucaugcagaaguca

SEQ ID NO: 5 - DNA, 20 bp, Artificial Sequence
  Synthetic oligonucleotide
catttacccgggagcctacg

SEQ ID NO: 6 - DNA, 25 bp, Artificial Sequence
  Synthetic oligonucleotide
gctttccgtcgccgccgtgcgcgtg

SEQ ID NO: 7 - DNA, 25 bp, Artificial Sequence
  Synthetic oligonucleotide
agatacccacaacacaccacgcgcc

SEQ ID NO: 8 - DNA, 21 bp, Artificial Sequence
  Synthetic oligonucleotide
atccttcagagagtcgcatgc

SEQ ID NO: 9 - DNA, 25 bp, Artificial Sequence
  Synthetic oligonucleotide
ggttcccaggcctcttgcaatgcgg

SEQ ID NO: 10 - DNA, 25 bp, Artificial Sequence
  Synthetic oligonucleotide
agtggcattgctggagttaccgctg

SEQ ID NO: 11 - DNA, 19 bp, Artificial Sequence
  Synthetic oligonucleotide
taccaccacacccatcaac

SEQ ID NO: 12 - DNA, 20 bp, Artificial Sequence
  Synthetic oligonucleotide
acaccaagacagggacagac

SEQ ID NO: 13 - DNA, 18 bp, Artificial Sequence
  Synthetic oligonucleotide
cacaagcgtctcgggatt

SEQ ID NO: 14 - DNA, 19 bp, Artificial Sequence
  Synthetic oligonucleotide
agtggcaagtcttccgaca

SEQ ID NO: 15 - DNA, 22 bp, Artificial Sequence
  Synthetic oligonucleotide
catccatgacaactttggtatc

SEQ ID NO: 16 - DNA, 20 bp, Artificial Sequence
  Synthetic oligonucleotide
caccctgttgctgtagccaa